(19) United States
(12) Patent Application Publication
(10) Pub. No.: US 2025/0066819 A1
(43) Pub. Date: Feb. 27, 2025

(54) CRISPR-ASSOCIATED TRANSPOSONS AND USES THEREOF

(71) Applicant: BOARD OF REGENTS, THE UNIVERSITY OF TEXAS SYSTEM, Austin, TX (US)

(72) Inventors: Ilya FINKELSTEIN, Austin, TX (US); Claus WILKE, Austin, TX (US); Kuang HU, Austin, TX (US); James RYBARSKI, Austin, TX (US); Alexis HILL, Austin, TX (US)

(21) Appl. No.: 18/683,988

(22) PCT Filed: Aug. 16, 2022
(86) PCT No.: PCT/US2022/075026

§ 371 Date: Feb. 15, 2024

Related U.S. Application Data
(60) Provisional application No. 63/233,460, filed on Aug. 16, 2021.

(51) Int. Cl. C12N 15/90; C12N 9/22; C12N 15/11

(52) U.S. Cl. CPC C12N 15/907; C12N 9/22; C12N 15/11; C12N 2310/20; C12N 2310/531; C12N 2800/90

(57) ABSTRACT

Disclosed herein are CRISPR-associated transposons (CASTs), which co-opt Cas genes for RNA-guided transposition. Disclosed herein are new families of CASTs, including a non-Tn7 CAST. These CASTs are useful in a variety of gene editing applications, so also disclosed herein are methods of using the CASTs.

### CROSS-REFERENCE TO RELATED APPLICATIONS

This application claims benefit of U.S. Provisional Application No. 63/233,460, filed Aug. 16, 2021, incorporated herein by reference in its entirety.

### GOVERNMENT SUPPORT CLAUSE

This invention was made with government support under Grant No. R01 GM124141 and Grant No. R01 GM088344, awarded by The National Institutes of Health. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Aug. 21, 2024, is entitled “10046-421US1_ST26.xml”, and is 634,360 bytes in size.

### REFERENCE TO AN ELECTRONIC SEQUENCE LISTING

The contents of the electronic sequence listing (10046-421WO1_ST26.xml; Size: 612 KB; and Date of Creation: Aug. 16, 2022) is herein incorporated by reference in its entirety.

### BACKGROUND

CRISPR-associated transposons (CASTs) are transposons that have delegated their insertion site selection to a nuclease-deficient CRISPR-Cas system. All currently-known CASTs derive from Tn7-like transposons and retain the core transposition genes TnsB and TnsC but dispense with TnsD and TnsE, which mediate target selection (Peters 2019; Peters 2017). Tn7 transposons site-specifically insert themselves at a single chromosomal locus (the attachment or att site) via the TnsD/TniQ family of DNA-binding proteins, while TnsE promotes horizontal gene transfer onto mobile genetic elements. In contrast, Class 1 CASTs replace TnsD and TnsE with a crRNA-guided TniQ-Cascade effector complex (Halpin-Healy 2020; Jia 2020). These CASTs can use the TniQ-Cascade complexes for both vertical and horizontal gene transfer (Klompe 2019). One notable exception is a family of Type I-B CASTs that retains TnsD for vertical transmission but co-opts TniQ-Cascade for horizontal transmission (Saito 2021). Similarly, Class 2 CASTs use the Cas12k effector to transpose to the attachment (att) sites or to mobile genetic elements (Hsieh 2021; Strecker 2019). CASTs also dispense with the spacer acquisition and DNA interference genes found in traditional CRISPR-Cas operons (Peters 2017). In short, these systems have merged the core transposition activities with crRNA-guided DNA targeting.

CASTs are exceedingly rare; only three sub-families of Tn7-associated CASTs have been reported bioinformatically and experimentally (Peters 2017; Klompe 2019; Saito 2021; Strecker 2019; Makarova 2020). These studies have identified that many, but not all, CASTs encode a self-targeting spacer flanked by atypical (privileged) direct repeats. However, the prevalence of such atypical repeats, the diversity of self-targeting strategies, and the molecular mechanisms of why CASTs have evolved these repeats has remained unresolved. Moreover, all CASTs that have been identified to-date have a minimal CRISPR array with as few as two spacers. These systems are also missing the Cas1-Cas2 adaptation machinery, raising the question of how CASTs target other mobile genetic elements for horizontal gene transfer. Another open question is whether non-Tn7 transposons have adapted CRISPR-Cas systems to mobilize their genetic information. What is needed in the art is an expanded catalog of CASTs to resolve these questions and for use in gene editing.

### SUMMARY

Disclosed herein is a system for RNA-guided DNA integration, the system comprising an isolated I-F CRISPR-Associated Transposon (CAST), wherein said CAST comprises TnsA-TnsB-TnsC; and TniQ-Cas8-Cas5-Cas7-Cas6; wherein TniQ-Cas8-Cas5 are fused.

Also disclosed herein is a system for RNA-guided DNA integration, the system comprising an isolated I-F CAST wherein said CAST comprises: TnsA-TnsB-TnsC; and TniQ-Cas8-Cas5-Cas7-Cas6.

Further disclosed is a system for RNA-guided DNA integration, the system comprising an isolated I-F CAST, wherein said CAST comprises: TnsA-TnsB-TnsC; TniQ; Cas8-Cas5 fusion; and Cas7-Cas7-Cas6.

Disclosed is a system for RNA-guided DNA integration, the system comprising an isolated I-F CAST, wherein said CAST comprises: TnsA-TnsB-TnsC; TniQ; Cas8-Cas5 fusion; and Cas7-Cas6.

Disclosed herein is a system for RNA-guided DNA integration, the system comprising an isolated I-B CAST, wherein said CAST comprises: TnsA-TnsB-TnsC; and TniQ-Cas6-Cas8-Cas7-Cas5; wherein the isolated CAST does not have a second TniQ sequence downstream.

Further disclosed herein is a system for RNA-guided DNA integration, the system comprising an isolated I-B CAST, wherein said CAST comprises: TnsA-TnsB-TnsC; and Cas6-Cas8-Cas7-Cas5-TniQ; wherein the isolated CAST does not have a second TniQ sequence upstream.

Disclosed herein is a system for RNA-guided DNA integration, the system comprising an isolated I-B CAST, wherein said CAST comprises: TniQ-Cas5-Cas7-Cas8-Cas6; and TnsB-TnsC-TniQ.

Also disclosed is a system for RNA-guided DNA integration, the system comprising an isolated IV CAST, wherein said CAST comprises: TnsA-TnsB-TnsC; and Csf2(Cas7)-Csf3(Cas5)-Cas8-Cas6.

Disclosed herein is a system for RNA-guided DNA integration, the system comprising an isolated Type I-C CAST, wherein said CAST comprises: TnsA-TnsB-TnsC; and TniQ-Cas7-Cas5-Cas8c.

Disclosed herein is a system for RNA-guided DNA integration, the system comprising an isolated Type V CAST, wherein said CAST comprises: TnsB-truncated TnsC-TniQ; wherein TnsC is truncated at the N-terminus; and Cas12k.

Further disclosed is a system for RNA-guided DNA integration, the system comprising an isolated Type V CAST, wherein said CAST comprises: TnsB-TnsB-TnsC-TniQ; and Cas12k.

Also disclosed is a system for RNA-guided DNA integration, the system comprising an isolated Type V CAST, wherein said CAST comprises: TnsB-TnsC-TniQ-TnsC-TniQ; and Cas12k.

Also disclosed is a system for RNA-guided DNA integration, the system comprising an isolated Type V CAST, wherein said CAST comprises: TnsB-TnsC-Cas12k-TnsB-TnsC-TniQ; and Cas12k.

Disclosed is a system for RNA-guided DNA integration, the system comprising an isolated Type V CAST, wherein said CAST comprises: TnsB-Cas12k-TnsB-TnsC-TniQ; and Cas12k.

Disclosed herein is a non-naturally occurring system for RNA-guided DNA integration, wherein the system is encoded by a nucleic acid, wherein the nucleic acid encodes a cas12a gene and a recombination-promoting nuclease A (RpnA) gene, wherein the nucleic acid encoding cas12a and rpnA are separated by about 1500-4500 nucleotides.

Also disclosed is a polypeptide comprising an isolated CAST, wherein the CAST is selected from any one of SEQ ID NOS: 1-458, or a combination of any of these sequences which results in a functional CAST. Also disclosed are nucleic acids which encode these polypeptides, and vectors which comprise the nucleic acid, as well as cells which comprise the vectors.

Disclosed herein is a method for sequence-specific modification of a target nucleic acid sequence in a prokaryotic cell, the method comprising providing to the cell a CAST, wherein the CAST comprises any of the CASTs disclosed herein, a crRNA, and a donor DNA comprising a nucleic acid cargo sequence under conditions for modification of the target nucleic acid, wherein the crRNA is specific for the target nucleic acid sequence, and further wherein the donor DNA comprises nucleic acid cargo sequence to be incorporated into the target nucleic acid sequence, thereby modifying the target nucleic acid in a sequence-specific manner.

### DESCRIPTION OF DRAWINGS

FIG. 1A-D shows a summary of Type I-F CASTs. (A) Gene architectures of Type I-F3a, I-F3b, and I-F3c systems. Unique gene architectures include TniQ-Cas8 fusions, split Cas8 and Cas5, and dual Cas7 systems. Purple: attachment site; blue: left (L) and right (R) transposon ends. Black diamonds: canonical direct repeats; gray diamonds: atypical direct repeats. Rectangles: protospacers; purple rectangle: self-targeting protospacer. Arrow indicates the target site. Slanted gapped lines indicate elided cargo regions. (B) The distribution of attachment site genes in the NCBI and the metagenomic databases. (C) (top) Sequence of a CRISPR array with a short, atypical spacer that may assemble a mini-Cascade. (bottom) Schematic of an atypical crRNA and its target DNA sequence. Sequences shown for repeat and spacer regions are SEQ ID NOS: 459-462, sequentially from top to bottom. SEQ ID NOS: 463-465 define the crRNA with the atypical repeat (463), where it hybridizes (464), and the target (465). (D) Weblogos of the PAM and right inverted repeat adjacent to each attachment site. The TnsB binding site and the self-targeting PAMs are conserved within sub-systems.

FIG. 2A-D shows analysis of Type I-B CASTs. (A) (left) Gene architectures of Type I-B systems. Systems can dispense with either the first or the second tniQ, suggesting alternative targeting lifestyles. Type I-B4 systems have a unique architecture that most resembles Type V CASTs. Colored rectangles correspond to phylogenetic groups in panel B. (right) The distribution of Type I-B sub-systems in the metagenomic database. (B) Phylogenetic tree with tniQ variants from Type I-B and I-F CASTs, as well as from the canonical Tn7 transposon. Type I-B tniQ1 is most similar to tniQ from Type I-F CASTs, whereas tniQ2 is closely related to canonical Tn7 tnsD. Values at branch points are bootstrap support percentages. (C) (top) Sequence of a Type I-B4 CRISPR array with a short, atypical spacer. (bottom) Schematic of an atypical crRNA basepaired with a target DNA sequence. Red bases are those that differ from the canonical repeat sequence. Sequences shown for repeat and spacer regions are SEQ ID NOS: 466-469, sequentially from top to bottom. SEQ ID NOS: 470-472 define the crRNA with the atypical repeat (470), where it hybridizes (471), and the target (472). (D) Domain maps of TniQ proteins. Regions homologous to the TniQ superfamily and the TnsD superfamily are indicated in pink and light green, respectively. The Type I-B4 system encodes the shortest TniQ variant.

FIG. 3A-C shows new Tn7 CASTs from metagenomic databases. (A) (top) Gene architecture of a Type IV CAST. This system lacks a CRISPR array but encodes a self-targeting spacer. Genes highlighted by colored rectangles correspond to genes in panel B (SEQ ID NOS: 473, 474, and 475, from top to bottom). (bottom) Schematic of a short, self-targeting spacer basepaired with its target DNA sequence. (B) Phylogenetic trees of cas6 and cas7 indicate that the Type IV CAST most closely resembles Type IV-A3 CRISPR-Cas systems. Values at branch points are bootstrap support percentages. (C) (top) Gene architecture of Type I-C systems. CRISPR arrays nor atypical self-targeting spacers were detected. (bottom) A phylogenetic tree of cas8 confirms that this system is closely related to Type I-C Cascades. Values at branch points are bootstrap support percentages.

FIG. 4A-E shows an analysis of Type V CASTs. (A) Gene architectures of Type V CASTs, including dual-insertion systems (bottom two rows). Colored rectangles around genes correspond to alignments in panels D and E. (B) Schematic of interactions between the target site DNA, a self-targeting crRNA, and a tracrRNA. Pictured are SEQ ID NOS: 476, 477, 478, and 479, from top to bottom. (C) Weblogo of PAM sequences found adjacent to spacer targets. (D) Aligned domain maps of truncated TnsC variants. Gray diagonal stripes indicate TnsD-interacting region. Truncated TnsCs lack the TnsA-and TnsB-interacting domains but generally retain the ATPase domain and most of the TnsD-interacting domain. The shortest TnsC has also lost its ATPase domain. (E) Aligned domain maps of truncated TnsB variants. Type V CAST TnsB is shorter than Tn7 TnsB but contains the functionally annotated domains. In some dual TnsB systems, the first TnsB encodes the N-terminal region and the second encodes the C-terminal portion.

FIG. 5A-D shows a family of putative non-Tn7 CASTs. (A) The defining features of this family of systems are an Rpn-family (PDDEXK domain-containing) nuclease/transposase near a nuclease-dead Cas12a or a Type I-E Cascade complex. The operon is enriched for nucleic-acid processing proteins. Self-targeting spacers were also observed (and short inverted repeats in some systems. (B) Multiple sequence alignment of Rpn proteins with the putative transposases from these systems. Residues critical for DNA cleavage in the PDDEXK domain are highlighted in red. The D165A mutant in RpnA more than doubles recombination in vivo; this aspartic acid is highlighted in red below the transposase_31 domain. The sequences are 480-488, from top to bottom. (C) Schematic of an atypical self-targeting spacer and its DNA target. The PAM is highlighted. SEQ ID NOS: 489-491, top to bottom. (D) Multiple sequence alignment of nuclease-active Cas12a and putative CAST Cas12as. Putative CAST Cas12as retain the conserved residues in the WED domain that are essential for crRNA processing, but lack an aspartic residue in the RuvC domain that is essential for DNA cleavage (SEQ ID NOS: 492-497, from top to bottom).

FIG. 6A-B shows a phylogenetic tree of (A) tnsB and (B) tnsC genes from each subtype of Tn7 CAST investigated, as well as from Tn7 and Tn5053. Values at branch points are bootstrap support percentages.

FIG. 7A-D shows cross-talking event detection and statistics. (A) A bioinformatic pipeline for the discovery of CRISPR-associated transposons (CASTs) and canonical Cas system in the same genome. (B) Distribution of repeat number of CAST I-F systems, CAST IB systems, CAST V systems and canonical Cas systems co-existing with them; (C) Percentage of each type CAST systems co-exist with canonical Cas systems; (D) Distribution of canonical Cas systems' subtype that co-existing with CAST systems in the NCBI microbial genome.

FIG. 8A-F shows an in vivo assays of cross-talking events. (A) Schematic of the conjugation-based transposition assay that can quantitively measuring the transposition efficiency. SEQ ID NOS: 498-501, from left to right. (B) Dilution assay for evaluating the transposition efficiency of CAST I-F system using repeat sequences from different canonical cas systems; (C) CAST I-F system can use CRISPR arrays from closely related canonical cas systems. Along the X axis we show in the integration efficiency. Along the Y axis we show the type of the CRISPR arrays that were assayed. (D) Box plots showing the distribution of insertion distances between the target site and the insertion site.

FIG. 9A-D shows a structure comparison between canonical and cross-talking type I-F CAST cascades. (A) Superimposition between type I-F CAST (PDB: 6PIG) and type I-F CAST cross-talking with canonical repeat. Typical crRNA structure is shown in blue; the cross-talking crRNA structure is shown in pink. (B) Structure of Cas6 with cross-talking crRNA, coloring with conservation score searched in the Consurf server. (C) The sequence logo representation of conservation in arginine-rich helix. (D) Integration efficiency of various Cas mutations.

FIG. 10A-B shows key repeat features that affect transposition efficiency. (A) Structures of the repeats from the CAST I-F system and the canonical Cas I-E system; (B) Four features were tested in the integration assay: changes in the nucleotide sequence, in the stem length, in the handle length, and in the loop length.

FIG. 11 shows a schematic of cross-talking.

FIG. 12 shows the CAST I-F systems using the repeat sequence from canonical cas III-B system and canonical I-F system that in the same organisms to do the transposition that target the LacZ gene in E. coli genome. Blue colony means off-target insertion, white colony means on-target inserton

FIG. 13 shows the cargo direction for each transposition using the repeat sequence from CAST I-F, canonical cas I-F, canonical cas III-B and canonical cas I-E system.

### DETAILED DESCRIPTION

General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single-or double-stranded, optionally comprising synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

“Open reading frame” is abbreviated ORF.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region.

“Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events; the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) o/Cell Biol 4:2253-8; Ayares et al., (1986) Proc. Natl. Acad. Sci. USA 83:5199-203; Liskay et al., (1987) Genetics 115:161-7.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign™ program of the LASERGENE bioinformatics computing suite (DNASTAR Inc., Madison, WI). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” Table in the same program. The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6.1 program of the LASERGENE bioinformatics computing suite (DNASTAR Inc., Madison, WI). Default parameters for multiple alignment (GAP PENALTY=10, GAP LENGTH PENALTY=0.2, Delay Divergen Seqs (%)=30, DNA Transition Weight=0.5, Protein Weight Matrix-Gonnet Series, DNA Weight Matrix=IUB). After alignment of the sequences using the Clustal W program, it is possible to obtain a “percent identity” by viewing the “sequence distances” Table in the same program. Unless otherwise stated, sequence identity/similarity values provided herein refer to the value obtained using GAP Version 10(GCG, Accelrys, San Diego, CA) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases.

“BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any percentage from 50% to 100%. Indeed, any amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms to highly similar fragments, such as genes that duplicate functional enzymes from closely related organisms. Post-hybridization washes determine stringency conditions.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. Isolated polynucleotides may be purified from a cell in which they naturally occur. Conventional nucleic acid purification methods known to skilled artisans may be used to obtain isolated polynucleotides. The term also embraces recombinant polynucleotides and chemically synthesized polynucleotides.

The term “fragment” refers to a contiguous set of nucleotides or amino acids. In one embodiment, a fragment is 2, 3, 4, 5, 6, 7 8, 9, 10, 11, 12, 13, 14, 15, 16, 17, 18, 19, 20, or greater than 20 contiguous nucleotides. In one embodiment, a fragment is 2, 3, 4, 5, 6, 7 8, 9, 10, 11, 12, 13, 14, 15, 16, 17, 18, 19, 20, or greater than 20 contiguous amino acids. A fragment may or may not exhibit the function of a sequence sharing some percent identity over the length of said fragment.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified plant. Genes can be designed for use in suppression by linking a nucleic acid fragment, whether or not it encodes an active enzyme, in the sense or antisense orientation relative to a plant promoter sequence.

“Gene” includes a nucleic acid fragment that expresses a functional molecule such as, but not limited to, a specific protein, including regulatory sequences preceding (5′ noncoding sequences) and following (3′ non-coding sequences) the coding sequence. “Native gene” refers to a gene as found in its natural endogenous location with its own regulatory sequences.

By the term “endogenous” it is meant a sequence or other molecule that naturally occurs in a cell or organism. In one aspect, an endogenous polynucleotide is normally found in the genome of a cell; that is, not heterologous.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

The terms “knock-out”, “gene knock-out” and “genetic knock-out” are used interchangeably herein. A knock-out represents a DNA sequence of a cell that has been rendered partially or completely inoperative by targeting with a Cas protein; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used) examples of knock-ins are a specific insertion of a heterologous amino acid coding sequence in a coding region of a gene, or a specific insertion of a transcriptional regulatory element in a genetic locus.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

A “codon-modified gene” or “codon-preferred gene” or “codon-optimized gene” is a gene having its frequency of codon usage designed to mimic the frequency of preferred codon usage of the host cell.

An “optimized” polynucleotide is a sequence that has been optimized for improved expression in a particular heterologous host cell.

A “plant-optimized nucleotide sequence” is a nucleotide sequence that has been optimized for expression in plants, particularly for increased expression in plants. A plant-optimized nucleotide sequence includes a codon-optimized gene. A plant-optimized nucleotide sequence can be synthesized by modifying a nucleotide sequence encoding a protein such as, for example, a Cas endonuclease as disclosed herein, using one or more plant-preferred codons for improved expression. See, for example, Campbell and Gowri (1990) Plant Physiol. 92:1-11 for a discussion of host-preferred codon usage.

A “promoter” is a region of DNA involved in recognition and binding of RNA polymerase and other proteins to initiate transcription. The promoter sequence consists of proximal and more distal upstream elements, the latter elements often referred to as enhancers.

An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al, (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. An RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “genome” refers to the entire complement of genetic material (genes and non-coding sequences) that is present in each cell of an organism, or virus or organelle; and/or a complete set of chromosomes inherited as a (haploid) unit from one parent.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

The terms “recombinant DNA molecule”, “recombinant DNA construct”, “expression construct”, “construct”, and “recombinant construct” are used interchangeably herein. A recombinant DNA construct comprises an artificial combination of nucleic acid sequences, e.g., regulatory and coding sequences that are not all found together in nature. For example, a recombinant DNA construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The term “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein may be entirely synthetic. In another example, a target polynucleotide for cleavage by a Cas endonuclease may be of a different organism than that of the Cas endonuclease. In another example, a Cas endonuclease and guide RNA may be introduced to a target polynucleotide with an additional polynucleotide that acts as a template or donor for insertion into the target polynucleotide, wherein the additional polynucleotide is heterologous to the target polynucleotide and/or the Cas endonuclease.

The term “expression”, as used herein, refers to the production of a functional end-product (e.g., an mRNA, guide RNA, or a protein) in either precursor or mature form.

A “mature” protein refers to a post-translationally processed polypeptide (i.e., one from which any pre-or propeptides present in the primary translation product have been removed). “Precursor” protein refers to the primary product of translation of mRNA (i.e., with pre-and propeptides still present). Pre-and propeptides may be but are not limited to intracellular localization signals.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus, and include adaptation molecules as well as interference molecules. An interference molecule of a bacterial adaptive immunity complex includes endonucleases. A Cas endonuclease described herein comprises one or more nuclease domains. A Cas endonuclease includes but is not limited to: the novel Cas-alpha protein disclosed herein, a Cas9 protein, a Cas12a (Cpfl) protein, a Cas12b (C2c1) protein, a Cas13a (C2c2) protein, a Cas12c (C2c3) protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. The Cas-alpha endonucleases of the disclosure include those having one or more RuvC nuclease domains. A Cas protein is further defined as a functional fragment or functional variant of a native Cas protein, or a protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity of the native sequence.

A “functional fragment”, “fragment that is functionally equivalent” and “functionally equivalent fragment” of a Cas endonuclease are used interchangeably herein, and refer to a portion or subsequence of the Cas endonuclease of the present disclosure in which the ability to recognize, bind to, and optionally unwind, nick or cleave (introduce a single or double strand break in) the target site is retained. The portion or subsequence of the Cas endonuclease can comprise a complete or partial (functional) peptide of any one of its domains.

The terms “functional variant”, “variant that is functionally equivalent” and “functionally equivalent variant” of a Cas endonuclease or Cas effector protein are used interchangeably herein, and refer to a variant of the Cas effector protein disclosed herein in which the ability to recognize, bind to, and optionally unwind, nick or cleave all or part of a target sequence is retained.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

The terms “Cascade” and “Cascade complex” are used interchangeably herein and include reference to a multi-subunit protein complex that can assemble with a polynucleotide forming a polynucleotide-protein complex (PNP). Cascade is a PNP that relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “cleavage-ready Cascade”, “crCascade”,” cleavage-ready Cascade complex”, “crCascade complex”, “cleavage-ready Cascade system”, “CRC” and “crCascade system”, are used interchangeably herein and include reference to a multi-subunit protein complex that can assemble with a polynucleotide forming a polynucleotide-protein complex (PNP), wherein one of the cascade proteins is a Cas endonuclease capable of recognizing, binding to, and optionally unwinding, nicking, or cleaving all or part of a target sequence.

The terms “5′-cap” and “7-methylguanylate (m7G) cap” are used interchangeably herein. A 7-methylguanylate residue is located on the 5′ terminus of messenger RNA (mRNA) in eukaryotes. RNA polymerase II (Pol II) transcribes mRNA in eukaryotes. Messenger RNA capping occurs generally as follows: the most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase.

The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export. One or more RNA components herein are uncapped.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “functional fragment”, “fragment that is functionally equivalent” and “functionally equivalent fragment” of a guide RNA, crRNA or tracrRNA are used interchangeably herein, and refer to a portion or subsequence of the guide RNA, crRNA or tracrRNA, respectively, of the present disclosure in which the ability to function as a guide RNA, crRNA or tracrRNA, respectively, is retained.

The terms “functional variant”, “variant that is functionally equivalent” and “functionally equivalent variant” of a guide RNA, crRNA or tracrRNA (respectively) are used interchangeably herein, and refer to a variant of the guide RNA, crRNA or tracrRNA, respectively, of the present disclosure in which the ability to function as a guide RNA, crRNA or tracrRNA, respectively, is retained.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example E1S20150059010A1, published 26 Feb. 2015), or any combination thereof.

As used herein, the terms “guide polynucleotide/Cas endonuclease complex”, “guide polynucleotide/Cas endonuclease system”,” guide polynucleotide/Cas complex”, “guide polynucleotide/Cas system” and “guided Cas system,” “Polynucleotide-guided endonuclease”, “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The term “transposon”, as used herein, refers to a polynucleotide (or nucleic acid segment), which may be recognized by a transposase or an integrase enzyme and which is a component of a functional nucleic acid-protein complex (e.g., a transpososome) capable of transposition. The term “transposase” as used herein refers to an enzyme, which is a component of a functional nucleic acid-protein complex capable of transposition and which mediates transposition. The transposase may comprise a single protein or comprise multiple protein sub-units. A transposase may be an enzyme capable of forming a functional complex with a transposon end or transposon end sequences. The term “transposase” may also refer in certain embodiments to integrases. The expression “transposition reaction” used herein refers to a reaction wherein a transposase inserts a donor polynucleotide sequence in or adjacent to an insertion site on a target polynucleotide. The insertion site may contain a sequence or secondary structure recognized by the transposase and/or an insertion motif sequence where the transposase cuts or creates staggered breaks in the target polynucleotide into which the donor polynucleotide sequence may be inserted. Exemplary components in a transposition reaction include a transposon, comprising the donor polynucleotide sequence to be inserted, and a transposase or an integrase enzyme. The term, “transposon end sequence” as used herein refers to the nucleotide sequences at the distal ends of a transposon. The transposon end sequences may be responsible for identifying the donor polynucleotide for transposition. The transposon end sequences may be the DNA sequences the transpose enzyme uses in order to form transpososome complex and to perform a transposition reaction.

The terms “target site”, “target sequence”, “target site sequence,” target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

Methods for “modifying a target site” and “altering a target site” are used interchangeably herein and refer to methods for producing an altered target site.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

A “polynucleotide of interest” includes any nucleotide sequence encoding a protein or polypeptide that improves desirability of crops, i.e. a trait of agronomic interest. Polynucleotides of interest include, but are not limited to: polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest may additionally be utilized in either the sense or anti-sense orientation. Further, more than one polynucleotide of interest may be utilized together, or “stacked”, to provide additional benefit.

A “complex trait locus” includes a genomic locus that has multiple transgenes genetically linked to each other.

The terms “decreased,” “fewer,” “slower” and “increased” “faster” “enhanced” “greater” as used herein refers to a decrease or increase in a characteristic of the modified plant element or resulting plant compared to an unmodified plant element or resulting plant. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the untreated control and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more higher than the untreated control.

As used herein, the term “before”, in reference to a sequence position, refers to an occurrence of one sequence upstream, or 5′, to another sequence.

### Systems, Compositions and Methods of Use

### General

The present disclosure provides for CAST systems, engineered nucleic acid targeting systems and methods for inserting a polynucleotide to a desired position in a target nucleic acid (e.g., the genome of a cell). In general, the systems comprise one or more transposases or functional fragments thereof, and one or more components of a sequence-specific nucleotide binding system, e.g., a Cas protein and a CRISPR-type guide molecule (referred to herein as a crRNA or gRNA). In some embodiments, the present disclosure provides an engineered nucleic acid targeting system, the system comprising: one or more CASTs and a guide molecule capable of complexing with the Cas protein and directing sequence specific binding of the guide-Cas protein complex to a target sequence of a target polynucleotide. The systems may further comprise one or more donor polynucleotides. The donor polynucleotide may be inserted by the system to a desired position in a target nucleic acid sequence. The present disclosure may further comprise polynucleotides encoding such nucleic acid targeting systems, vector (such as plasmid) systems comprising one or more vectors comprising said polynucleotides, and one or more cells transformed with said vector systems.

Specifically, recent surveys have reported Tn7-like transposons that co-opt Type I-F, I-B, and V-K CRISPR effectors. Disclosed herein are an expansion of known CAST systems, which have been obtained via bioinformatics. New architectures have been found for all known CASTs, including novel arrangements of the Cascade effectors, new self-targeting modalities, and minimal V-K systems. New families of CASTs are also described that have co-opted the Type I-C and Type IV CRISPR-Cas systems. A search for non-Tn7 CASTs identifies CASTs that co-opt Cas 12a for horizontal gene transfer. These new systems shed light on how CRISPR systems have co-evolved with transposases and expand the programmable gene editing toolkit.

In one aspect, the present disclosure includes systems comprise one or more transposases and one or more guide molecules. The guide molecules may be sequence-specific. The system may further comprise one or more additional transposases, transposon components, or functional fragments thereof. In some embodiments, the systems described herein may comprise one or more transposases or transposase sub-units that are associated with, linked to, bound to, or otherwise capable of forming a complex with a sequence-specific nucleotide-binding system. In certain example embodiments, the one or more transposases or transposase sub-units and the sequence-specific nucleotide-binding system are associated by co-regulation or expression. In other example embodiments, the one or more transposases and/or the transposase subunits and sequence-specific nucleotide binding system are associated by the ability of the sequence-specific nucleotide-binding domain to direct or recruit the one or more transposase or transposase subunits to an insertion site where one or more transposases or transposase subunits direct insertion of a donor polynucleotide into a target polynucleotide sequence. A sequence-specific nucleotide-binding system may be a sequence-specific DNA-binding protein, or functional fragment thereof, and/or sequence-specific RNA-binding protein or functional fragment thereof.

The nucleotide binding system may comprise a Cas protein, a fragment thereof, or a mutated form thereof. The Cas protein may have reduced or no nuclease activity. In some examples, the DNA binding domain comprises one or more Class 1 (e.g., Type I, Type III, Type VI) or Class 2 (e.g. Type II, Type V, or Type VI) CRISPR-Cas proteins. In certain embodiments, the sequence-specific guide molecule can direct a transposon to a target site comprising a target sequence and the transposase directs insertion of a donor polynucleotide sequence at the target site.

In certain embodiments, the system may comprise more than one Cas protein. In certain cases, one of the Cas proteins or a fragment thereof may serve as a transposase-interacting domain. For example, the system may comprise a Cas protein and a transposase-interacting domain of Casl2. Specific examples of these systems are given below.

The systems disclosed herein comprise “CRISPR-associated transposases” (also used interchangeably with Cas-associated transposases, CRISPR-associated transposase proteins, or CAST system herein) or functional fragments thereof. CRISPR-associated transposases may include any transposases or transposase subunits that can be directed to or recruited to a region of a target polynucleotide by sequence-specific binding of a CRISPR-Cas complex to the target polynucleotide. CRISPR-associated transposases may include any transposases that associate (e.g., form a complex) with one or more components in a CRISPR-Cas system, e.g., Cas protein, guide molecule etc.). In certain example embodiments, CRISPR-associated transposases may be fused or tethered (e.g. by a linker) to one or more components in a CRISPR-Cas system, e.g., Cas protein, guide molecule etc.).

A transposase subunit or transposase complex may interact with a Cas protein herein. In some examples, the transposase or transposase complex interacts with the N-terminus of the Cas protein. In certain examples, the transposase or transposase complex interacts with the C-terminus of the Cas protein. In certain examples, the transposase or transposase complex interacts with a fragment of the Cas protein between its N-terminus and C-terminus.

### Transposons and Transposases

The systems herein may comprise one or more components of a transposon and/or one or more transposases. Transposons employ a variety of regulatory mechanisms to maintain transposition at a low frequency and sometimes coordinate transposition with various cell processes. Some prokaryotic transposons can also mobilize functions that benefit the host or otherwise help maintain the element. Certain transposons have evolved mechanisms of tight control over target site selection, the most notable example being the Tn7 family (see Peters JE (2014) Tn7. Microbiol Spectr 2:1-20). Three transposon-encoded proteins form the core transposition machinery of Tn7: a heteromeric transposase (TnsA and TnsB) and a regulator protein (TnsC). In addition to the core TnsABC transposition proteins, Tn7 elements encode dedicated target site-selection proteins, TnsD and TnsE. In conjunction with TnsABC, the sequence-specific DNA-binding protein TnsD directs transposition into a conserved site referred to as the “Tn7 attachment site,” attTn7. TnsD is a member of a large family of proteins that also includes TniQ, a protein found in other types of bacterial transposons. TniQ has been shown to target transposition into resolution sites of plasmids. It is noted that any of the transposon elements TnsA, TnsB, or TnsC can be in any order in a nucleic acid encoding these proteins, so that they may be sequential, or any combination thereof which is not sequential, such as TnsC-TnsA-TnsB, or another other combination. In the case of fused proteins, these proteins may likewise appear in any order. Furthermore, as discussed herein, all three of TnsA, TnsB, and TnsC are not always needed with all systems.

In one example embodiment, the disclosure provides systems comprising a Tn7 transposon system or components thereof. The transposon system may provide functions including but not limited to target recognition, target cleavage, and polynucleotide insertion. In certain example embodiments, the transposon system does not provide target polynucleotide recognition but provides target polynucleotide cleavage and insertion of a donor polynucleotide into the target polynucleotide.

In certain example embodiments, CASTs disclosed herein comprise a multimeric protein complex. In certain example embodiments, the multimeric protein complex comprises TnsA, TnsB and TnsC. In other example embodiments, the transposase may comprise TnsB, TnsC, and TniQ. As used herein, the terms “TnsAB”, “TnsAC”, “TnsBC”, or “TnsABC” refer to a transposon complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. As discussed above, in relation to the order of the proteins in a fusion complex, they may appear in any order. Linkers, spacers, or other components may exist between the proteins, or they may immediately adjoin one another.

### Donor Polynucleotides

The system may further comprise one or more donor polynucleotides (e.g., for insertion into the target polynucleotide). A donor polynucleotide may be an equivalent of a transposable element that can be inserted or integrated to a target site. The donor polynucleotide may be or comprise one or more components of a transposon. A donor polynucleotide may be any type of polynucleotides, including, but not limited to, a gene, a gene fragment, a non-coding polynucleotide, a regulatory polynucleotide, a synthetic polynucleotide, etc. The donor polynucleotide may include a transposon left end (LE) and transposon right end (RE). The LE and RE sequences may be endogenous sequences for the CAST used or may be heterologous sequences recognizable by the CAST used, or the LE or RE may be synthetic sequences that comprise a sequence or structure feature recognized by the CAST and sufficient to allow insertion of the donor polynucleotide into the target polynucleotides. In certain example embodiments, the LE and RE sequences are truncated. In certain example embodiments may be between 100-200 bps, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length.

The donor polynucleotide may be inserted at a position upstream or downstream of a PAM on a target polynucleotide (PAMs are discussed in more detail below). In some embodiments, a donor polynucleotide comprises a PAM sequence. Examples of PAM sequences include TTTN, ATTN, NGTN, RGTR, VGTD, or VGTR.

The donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

In certain embodiments of the invention, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence. The donor polynucleotide to be inserted may have a size from 10 bases to 50 kb in length, e.g., from 50 to 40 kb, from 100 to 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, or from 2800 bases to 3000 bases in length.

The components in the systems herein may comprise one or more mutations that alter their (e.g., the transposase(s)) binding affinity to the donor polynucleotide. In some examples, the mutations increase the binding affinity between the transposase(s) and the donor polynucleotide. In certain examples, the mutations decrease the binding affinity between the transposase(s) and the donor polynucleotide. The mutations may alter the activity of the Cas and/or transposase(s).

In certain embodiments, the systems disclosed herein are capable of unidirectional insertion, that is the system inserts the donor polynucleotide in only one orientation.

### Cas Systems

The CAST systems herein may comprise one or more Cas components. The one or more components of the Cas portion of the CAST may serve as the nucleotide-binding component in the systems. In certain example embodiments, the transposon component includes, associates with, or forms a complex with a Cas complex. In one example embodiment, the Cas component directs the transposon component and/or transposase(s) to a target insertion site where the transposon component directs insertion of the donor polynucleotide into a target nucleic acid sequence.

The Cas systems herein may comprise a Cas protein to be used in a CAST system, and a guide molecule. Non-limiting examples of Cas proteins include Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csx1, Csx15, Csf1, Csf2, Csf1, Csf4, Cas9, Cas12 (e.g., Cas12a, Cas12b, Cas12c, Cas12d, Cas12k, etc.), Cas13 (e.g., Cas13a, Cas13b (such as Cas13b-t1, Cas13b-t2, Cas13b-t3), Cas13c, Cas13d, etc.), Cas14, CasX, CasY, or an engineered form of the Cas protein (e.g., an invective, dead form, a nickase form). In some examples, the CRISPR-Cas system is nuclease-deficient.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or” protospacer flanking sequence”.

In a preferred embodiment, a CAST protein may recognize a 3′ PAM. In certain embodiments, a CAST protein may recognize a 3′ PAM which is 5H, wherein His A, C or U.

In the context of formation of a CAST, “target sequence” refers to a sequence to which a guide sequence is designed to have complementarity, where hybridization between a target sequence and a guide sequence promotes the formation of a CAST. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the crRNA is designed to have complementarity and to which the effector function mediated by the CAST and a crRNA is to be directed. In some embodiments, a target sequence is located in the nucleus or cytoplasm of a cell.

In certain example embodiments, the CAST may be delivered using a nucleic acid molecule encoding the CAST protein. The nucleic acid molecule encoding a CAST protein, may advantageously be a codon optimized CAST protein. An example of a codon optimized sequence, is in this instance a sequence optimized for expression in eukaryote, e.g., humans (i.e. being optimized for expression in humans), or for another eukaryote, animal or mammal as herein discussed. Whilst this is preferred, it will be appreciated that other examples are possible and codon optimization for a host species other than human, or for codon optimization for specific organs is known. In some embodiments, an enzyme coding sequence encoding a CRISPR protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available. Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a transgenic cell in which one or more nucleic acids encoding one or more crRNAs are provided or introduced operably connected in the cell with a regulatory element comprising a promoter of one or more genes of interest. As used herein, the term “Cas transgenic cell” refers to a cell, such as a eukaryotic cell, in which a Cas gene has been genomically integrated. The nature, type, or origin of the cell are not particularly limited according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

The crRNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, HI, retroviral Rous sarcoma virus (RSV) LTR promoter, the cytomegalovirus (CMV) promoter, the SV40 promoter, the dihydrofolate reductase promoter, the b-actin promoter, the phosphoglycerol kinase (PGK) promoter, and the EFla promoter. An advantageous promoter is the promoter is U6.

### Guide Molecules (crRNA)

The system herein may comprise one or more guide molecules. As used herein, the term “guide molecule” in the context of a CAST system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences, also referred to herein as a crRNA, made using the methods disclosed herein, may be a full-length guide sequence or a truncated guide sequence. The guide sequence can comprise a full-length crRNA sequence, or a truncated crRNA sequence. The guide molecule, in addition to the guide sequence, can comprise a trans-activating CRISPR RNA (tracrRNA), which can be a full-length or truncated tracrRNA. In some embodiments, the degree of complementarity of the crRNA to a given target sequence, when optimally aligned using a suitable alignment algorithm, is about or more than about 50%, 60%, 75%, 80%, 85%, 90%, 95%, 97.5%, 99%, or more.

In certain example embodiments, the guide molecule comprises a crRNA that may be designed to have at least one mismatch with the target sequence, such that a RNA duplex is formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more.

In certain embodiments, the crRNA is from 10 to 50 nt. In certain embodiments, the spacer length of the crRNA is at least 10 nucleotides. In certain embodiments, the spacer length is from 12 to 14 nt, e.g., 12, 13, or 14 nt, 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer. In certain example embodiment, the guide sequence is 10, 11, 12, 13, 14, 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the crRNA has a canonical length (e.g., about 15-30 nt) and is used to hybridize with the target RNA or DNA. In some embodiments, a guide molecule is longer than the canonical length (e.g., >30 nt) and is used to hybridize with the target RNA or DNA, such that a region of the crRNA hybridizes with a region of the RNA or DNA strand outside of the Cas-guide target complex. This can be of interest where additional modifications, such as deamination of nucleotides is of interest. In alternative embodiments, it is of interest to maintain the limitation of the canonical guide sequence length.

The tracrRNA can include any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, 60, 70, 80, 90, 100, 110, 120, 130, 140, 150, 160, 170, 180, 190, 200, 210, 220, 230 or more nucleotides in length. In certain example embodiments the tracr is 210, 211, 212, 213, 214, 215, 216, 217, 218, 219, or 220 nucleotides in length. In some embodiments, the tracr sequence and crRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop corresponds to the tracr mate sequence, and the portion of the sequence 3′ of the loop corresponds to the tracr sequence. In certain example embodiments, guide molecule and tracr sequence are physically or chemically linked.

In some embodiments, the crRNA of the guide molecule (direct repeat and/or spacer) is selected to reduce the degree of secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm.

### Specific Systems

### I-F CASTS

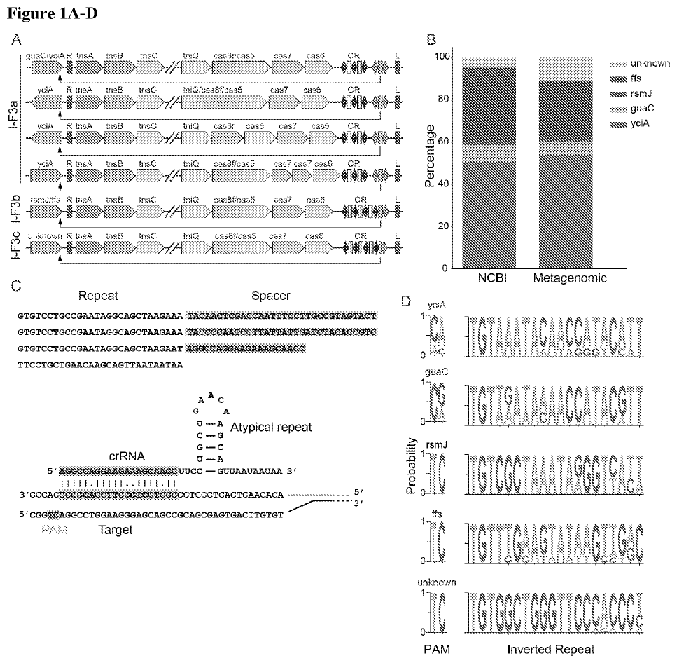

Disclosed herein are CASTS of the I-F type. These can be seen in FIG. 1, and specific examples are given in the Tables. Disclosed herein is a system for RNA-guided DNA integration, wherein the system comprises an isolated I-F CRISPR-Associated Transposon (CAST), wherein said CAST comprises: TnsA-TnsB-TnsC; and TniQ-Cas8-Cas5-Cas7-Cas6; wherein TniQ-Cas8-Cas5 are fused. The CAST components can be sequential or non-sequential. The system can further comprise a guide molecule comprising a crRNA. The system can also comprise donor DNA, such as nucleic acid cargo.

Also disclosed is a system for RNA-guided DNA integration, the system comprising an isolated I-F CAST wherein said CAST comprises: TnsA-TnsB-TnsC; and TniQ-Cas8-Cas5-Cas7-Cas6. The CAST components can be sequential or non-sequential. The system can further comprise a guide molecule comprising a crRNA. The system can also comprise donor DNA, such as nucleic acid cargo.

Further disclosed is a system for RNA-guided DNA integration, the system comprising an isolated I-F CAST, wherein said CAST comprises: TnsA-TnsB-TnsC; TniQ; Cas8-Cas5fusion; and Cas7-Cas7-Cas6. The CAST components can be sequential or non-sequential. The system can further comprise a guide molecule comprising a crRNA. The system can also comprise donor DNA, such as nucleic acid cargo.

Also disclosed is a system for RNA-guided DNA integration, the system comprising an isolated I-F CAST, wherein said CAST comprises: TnsA-TnsB-TnsC; TniQ; Cas8-Cas5 fusion; and Cas7-Cas6. The CAST components can be sequential or non-sequential. The system can further comprise a guide molecule comprising a crRNA. The system can also comprise donor DNA, such as nucleic acid cargo.

In the case if I-F CASTs, TnsA can be represented by a sequence with 90, 91, 92, 93, 94, 95, 96, 97, 98, 99, or 100% homology to any of SEQ ID NOS: 6, 10, 56, 66, 72, 84, 88, 95, 99, 109, 113, 120, 127, 139, 143, 150, 160, 163, 168, 178, 183, or 190. TnsB can be represented by a sequence with 90, 91, 92, 93, 94, 95, 96, 97, 98, 99, or 100% homology to any of SEQ ID NOS: 5, 11, 57, 65, 71, 83, 87, 94, 100, 108, 114, 121, 128, 138, 142, 149, 159, 163, 169, 177, 184, or 191. TnsC can be represented by a sequence with 90, 91, 92, 93, 94, 95, 96, 97, 98, 99, or 100% homology to any of SEQ ID NOS: 4, 12, 58, 64, 73, 82, 86, 93, 101, 107, 115, 122, 129, 137, 140, 148, 158, 162, 170, 176, 185, or 192. TniQ can be represented by a sequence with 90, 91, 92, 93, 94, 95, 96, 97, 98, 99, or 100% homology to any of SEQ ID NOS: 9, 13, or 59, 63, 70, 81, 85, 92, 102, 106, 116, 123, 130, 136, 146, 147, 157, 161, 171, 175, 186, or 193. TniQ-Cas8-Cas5 can be represented by a sequence with 90, 91, 92, 93, 94, 95, 96, 97, 98, 99, or 100% homology to any of SEQ ID NO: 1. Cas7 can be represented by a sequence with 90, 91, 92, 93, 94, 95, 96, 97, 98, 99, or 100% homology to any of SEQ ID NOS: 2, 15, 16, 61, 68, 75, 79, 90, 97, 104, 111, 118, 125, 132, 134, 144, 152, 155, 166, 173, 181, 188, or 195. Cas6 can be represented by a sequence with 90, 91, 92, 93, 94, 95, 96, 97, 98, 99, or 100% homology to any of SEQ ID NOS: 3, 17, 62, 67, 74, 78, 89, 96, 105, 110, 119, 126, 458, 133, 141, 151, 154, 165, 174, 180, 189, or 196. Cas5 can be represented by a sequence with 90, 91, 92, 93, 94, 95, 96, 97, 98, 99, or 100% homology to any of SEQ ID NOS: 7 or 14. Cas8 can be represented by a sequence with 90, 91, 92, 93, 94, 95, 96, 97, 98, 99, or 100% homology to any of SEQ ID NOS: 8, 60, 69, 76, 80, 91, 98, 103, 112, 117, 124, 131, 135, 145, 153, 156, 167, 172, 182, 187, or 194. It is noted that when multiple proteins of the same type (i.e., Cas7) are used in the same CAST, the proteins can be same or different. Again, using Cas7 as an example, if the CAST has two Cas7s, they can both be SEQ ID NO: 15, or one can be SEQ ID NO: 15 and one can be SEQ ID NO: 16. This example is illustrative, but this applies to all CASTs described herein.

### I-B CASTS

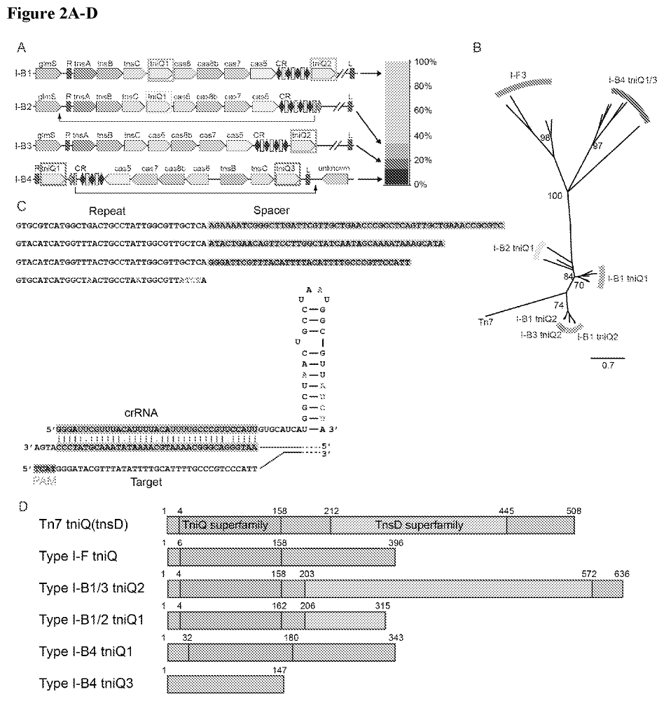

Also disclosed herein are I-B CASTS. These can be seen in FIG. 2, and specific examples are given in the Tables. Specifically, disclosed is a system for RNA-guided DNA integration, the system comprising an isolated I-B CAST, wherein said CAST comprises: TnsA-TnsB-TnsC; and TniQ-Cas6-Cas8-Cas7-Cas5; wherein the isolated CAST does not have a second TniQ sequence downstream. The CAST components can be sequential or non-sequential.

The system can further comprise a guide molecule comprising a crRNA. The system can also comprise donor DNA, such as nucleic acid cargo.

Also disclosed is a system for RNA-guided DNA integration, the system comprising an isolated I-B CAST, wherein said CAST comprises: TnsA-TnsB-TnsC; and Cas6-Cas8-Cas7-Cas5-TniQ; wherein the isolated CAST does not have a second TniQ sequence upstream. The

CAST components can be sequential or non-sequential. The system can further comprise a guide molecule comprising a crRNA. The system can also comprise donor DNA, such as nucleic acid cargo.

Further disclosed is a system for RNA-guided DNA integration, the system comprising an isolated I-B CAST, wherein said CAST comprises: TniQ-Cas5-Cas7-Cas8-Cas6; and TnsB-TnsC-TniQ. The CAST components can be sequential or non-sequential. The system can further comprise a guide molecule comprising a crRNA. The system can also comprise donor DNA, such as nucleic acid cargo.

In the case if I-B CASTs, TnsA can be represented by a sequence with 90, 91, 92, 93, 94, 95, 96, 97, 98, 99, or 100% homology to any of SEQ ID NOS: 198, 215, 218, 229, 239, 257, 267, 270, 280, 291, 307, 317, 327, 338, 347, 351, 367, 377, 387, 400, 410, 415, 422, or 438. TnsB can be represented by a sequence with 90, 91, 92, 93, 94, 95, 96, 97, 98, 99, or 100% homology to any of SEQ ID NOS: 20, 199, 214, 219, 220, 230, 240, 254, 256, 266, 271, 281, 290, 306, 316, 326, 337, 349, 352, 353, 368, 378, 388, 397, 399, 411, 423, 424, or 437. TnsC can be represented by a sequence with 90, 91, 92, 93, 94, 95, 96, 97, 98, 99, or 100% homology to any of SEQ ID NOS: 200, 213, 221, 231, 241, 255, 265, 272, 282, 289, 305, 315, 325, 336, 339, 354, 369, 379, 389, 398, 402, 425, 436, or 19. Cas6 can be represented by a sequence with 90, 91, 92, 93, 94, 95, 96, 97, 98, 99, or 100% homology to any of SEQ ID NOS: 21, 202, 211, 223, 233, 243, 252, 263, 274, 284, 297, 303, 313, 323, 334, 341, 356, 361, 371, 381, 395, 404, 420, 427, or 435. Cas8 can be represented by a sequence with 90, 91, 92, 93, 94, 95, 96, 97, 98, 99, or 100% homology to any of SEQ ID NOS: 22, 203, 210, 224, 234, 244, 251, 262, 285, 296, 302, 312, 322, 333, 342, 357, 362, 372, 382, 394, 405, 419, 428, or 434. Cas7 can be represented by a sequence with 90, 91, 92, 93, 94, 95, 96, 97, 98, 99, or 100% homology to any of SEQ ID NOS: 23, 204, 209, 225, 235, 245, 250, 261, 276, 286, 295, 301, 311, 321, 332, 342, 358, 363, 373, 383, 393, 406, 418, 429, or 433. Cas5 can be represented by a sequence with 90, 91, 92, 93, 94, 95, 96, 97, 98, 99, or 100% homology to any of SEQ ID NOS: 24, 205, 208, 226, 236, 246, 249, 260, 277, 287, 294, 300, 310, 320, 331, 344, 348, 359, 364, 374, 384, 392, 407, 417, 430, or 432.

### Type IV and I-C CASTS

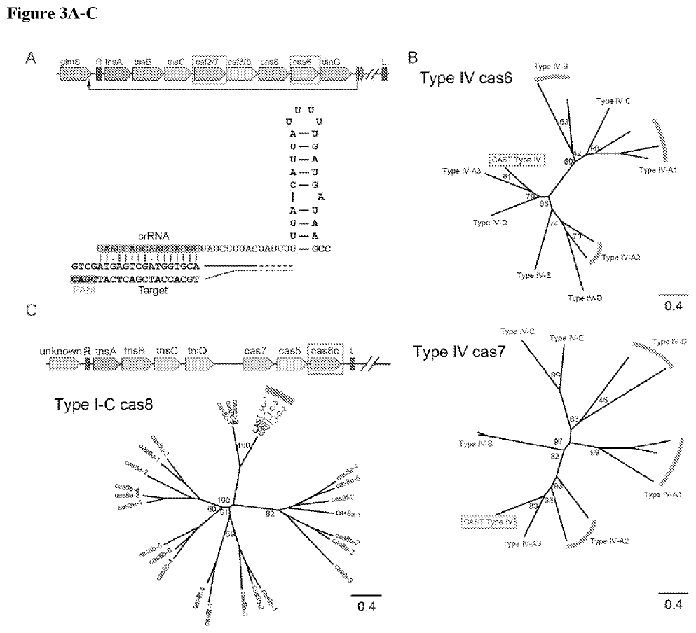

Also disclosed herein are Type IV CASTs. These can be seen in FIG. 3, and specific examples are given in the Tables. Specifically, disclosed is a system for RNA-guided DNA integration, the system comprising an isolated IV CAST, wherein said CAST comprises: TnsA-TnsB-TnsC; and Csf2 (Cas7)-Csf3 (Cas5)-Cas8-Cas6. It is noted that Csf2 and Cas7 are interchangeable, so it is contemplated herein that this CAST can comprise either Csf2 or Cas7. Similarly, Csf3 and Cas5 are interchangeable, so it is contemplated herein that this CAST can comprise either Csf3 or Cas5. The CAST components can be sequential or non-sequential. The system can further comprise a guide molecule comprising a crRNA. The system can also comprise donor DNA, such as nucleic acid cargo.

When referring to Type IV CASTs, TnsA can be represented by a sequence with 90, 91, 92, 93, 94, 95, 96, 97, 98, 99, or 100% homology to SEQ ID NO: 26. TnsB can be represented by a sequence with 90, 91, 92, 93, 94, 95, 96, 97, 98, 99, or 100% homology to SEQ ID NO: 27. TnsC can be represented by a sequence with 90, 91, 92, 93, 94, 95, 96, 97, 98, 99, or 100% homology to SEQ ID NO: 28. Csf2 (Cas7) can be represented by a sequence with 90, 91, 92, 93, 94, 95, 96, 97, 98, 99, or 100% homology to SEQ ID NO: 29. Csf3 (Cas4) can be represented by a sequence with 90, 91, 92, 93, 94, 95, 96, 97, 98, 99, or 100% homology to SEQ ID NO: 30. Cas8 can be represented by a sequence with 90, 91, 92, 93, 94, 95, 96, 97, 98, 99, or 100% homology to any of SEQ ID NO: 31. Caso can be represented by a sequence with 90, 91, 92, 93, 94, 95, 96, 97, 98, 99, or 100% homology to SEQ ID NO: 32.

Also disclosed herein are Type I-C CASTs. These can be seen in FIG. 3, and specific Examples are given in the Tables. Specifically, disclosed herein is a system for RNA-guided DNA integration, the system comprising an isolated Type I-C CAST, wherein said CAST comprises TnsA-TnsB-TnsC; and TniQ-Cas7-Cas5-Cas8c. The CAST components can be sequential or non-sequential. The system can further comprise a guide molecule comprising a crRNA. The system can also comprise donor DNA, such as nucleic acid cargo.

Also disclosed is a system for RNA-guided DNA integration, the system comprising an isolated Type I-C CAST, wherein said CAST comprises: TnsB-truncated TnsC-TniQ; wherein TnsC is truncated at the N-terminus; and Cas12k. By “truncated TnsC” is meant that that the TnsC polypeptide is 1, 2, 3, 4, 5, 6, 7, 8, 9, 10, 11, 12, 13, 14, 15, 16, 17, 18, 19, 20 or more amino acids shorter than the standard TnsC polypeptide length recognized by those of skill in the art. The CAST components can be sequential or non-sequential. The system can further comprise a guide molecule comprising a crRNA. The system can also comprise donor DNA, such as nucleic acid cargo.

When referring to Type I-C CASTs, TnsA can be represented by a sequence with 90, 91, 92, 93, 94, 95, 96, 97, 98, 99, or 100% homology to SEQ ID NO: 39. TnsB can be represented by a sequence with 90, 91, 92, 93, 94, 95, 96, 97, 98, 99, or 100% homology to SEQ ID NO: 38. TnsC can be represented by a sequence with 90, 91, 92, 93, 94, 95, 96, 97, 98, 99, or 100% homology to SEQ ID NO: 37. TniQ can be represented by a sequence with 90, 91, 92, 93, 94, 95, 96, 97, 98, 99, or 100% homology to SEQ ID NO: 36. Cas7 can be represented by a sequence with 90, 91, 92, 93, 94, 95, 96, 97, 98, 99, or 100% homology to SEQ ID NO: 35. Cas5 can be represented by a sequence with 90, 91, 92, 93, 94, 95, 96, 97, 98, 99, or 100% homology to SEQ ID NO: 34. Cas8 can be represented by a sequence with 90, 91, 92, 93, 94, 95, 96, 97, 98, 99, or 100% homology to any of SEQ ID NO: 33.

### Type V CASTS

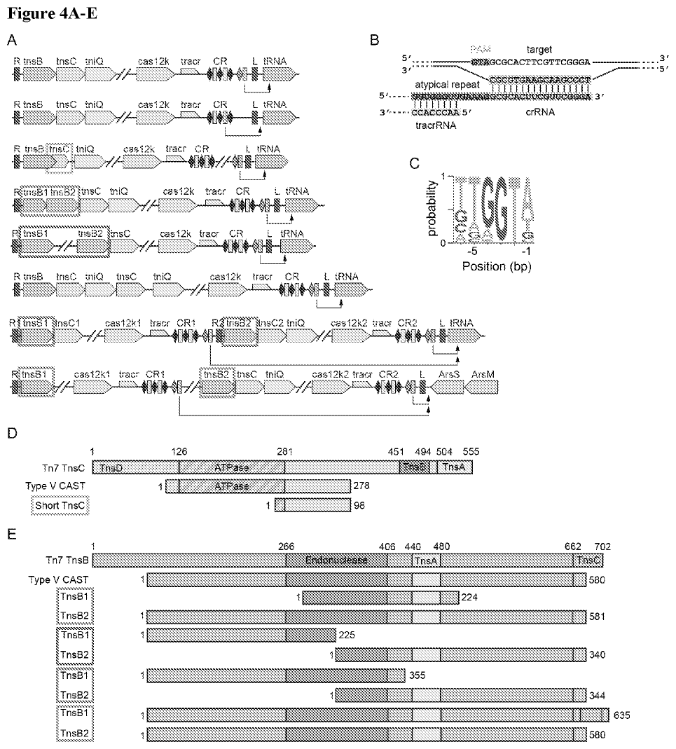

Also disclosed herein are Type V CASTs. These can be seen in FIG. 4, and specific examples are given in the Tables. Specifically, disclosed herein is a system for RNA-guided DNA integration, the system comprising an isolated Type V CAST, wherein said CAST comprises: TnsB-TnsB-TnsC-TniQ; and Cas12k. The CAST components can be sequential or non-sequential. The system can further comprise a guide molecule comprising a crRNA. The system can also comprise donor DNA, such as nucleic acid cargo.

Also disclosed is a system for RNA-guided DNA integration, the system comprising an Isolated Type V CAST, wherein said CAST comprises: TnsB-TnsC-TniQ-TnsC-TniQ; and Cas12k. The CAST components can be sequential or non-sequential. The system can further comprise a guide molecule comprising a crRNA. The system can also comprise donor DNA, such as nucleic acid cargo.

Further disclosed is a system for RNA-guided DNA integration, the system comprising an isolated Type V CAST, wherein said CAST comprises: TnsB-TnsC-Cas12k-TnsB-TnsC-TniQ; and Cas12k. One, or both, of the TnsB polypeptides of this CAST can be truncated as compared to a full length, or standard, TnsB polypeptides as recognized by one of skill in the art. For example, one or both of the TnsB polypeptides can be 1, 2, 3, 4, 5, 6, 7, 8, 9, 10, 11, 12, 13, 14, 15, 16, 17, 18, 19, 20 or more amino acids shorter than the standard TnsB. Furthermore, the TnsB proteins of this CAST can interact with each other. For example, they can form a dimer. The CAST components can be sequential or non-sequential. The system can further comprise a guide molecule comprising a crRNA. The system can also comprise donor DNA, such as nucleic acid cargo.

Also disclosed herein is a system for RNA-guided DNA integration, the system comprising an isolated Type V CAST, wherein said CAST comprises: TnsB-Cas12k-TnsB-TnsC-TniQ; and Cas12k. It is noted that the TnsB proteins of this CAST can interact with each other. For example, they can form a dimer. The CAST components can be sequential or non-sequential. The system can further comprise a guide molecule comprising a crRNA. The system can also comprise donor DNA, such as nucleic acid cargo.

When referring to Type V CASTs, TnsB can be represented by a sequence with 90, 91, 92, 93, 94, 95, 96, 97, 98, 99, or 100% homology to SEQ ID NOS: 40, 44, 48, 52, 440, 441, 445, 449, 453, or 454. TnsC can be represented by a sequence with 90, 91, 92, 93, 94, 95, 96, 97, 98, 99, or 100% homology to SEQ ID NOS: 41, 45, 49, 53, 442, 446, 450, or 455. TniQ can be represented by a sequence with 90, 91, 92, 93, 94, 95, 96, 97, 98, 99, or 100% homology to SEQ ID NOS: 42, 46, 50, 54, 443, 447, 451, or 456. Cas12k can be represented by a sequence with 90, 91, 92, 93, 94, 95, 96, 97, 98, 99, or 100% homology to any of SEQ ID NOS: 43, 47, 51, 55, 444, 448, 452, or 457.

### Non-Tn7 CASTs

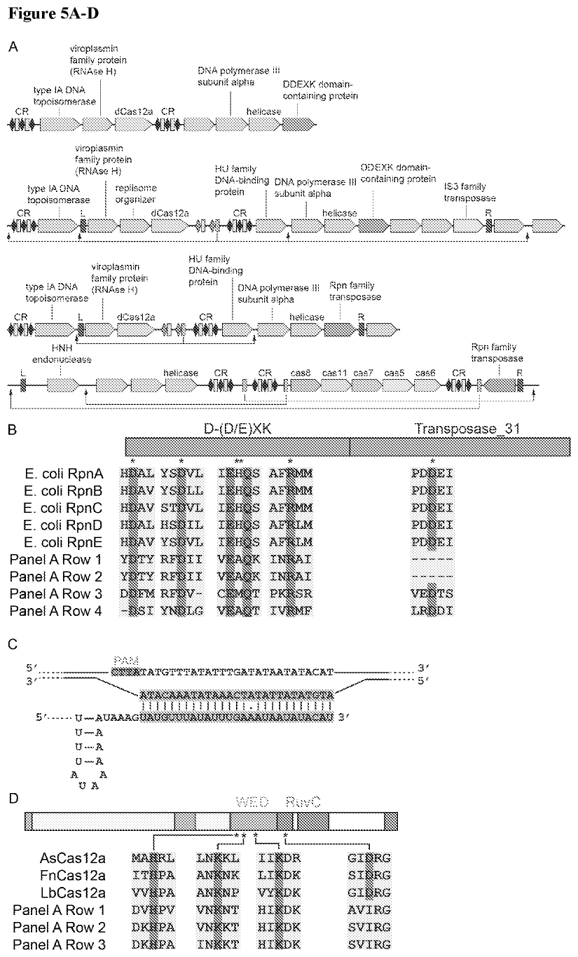

Also disclosed herein is a non-Tn7 CAST. An example can be seen in FIG. 5, and specific examples of such CASTs are given in the Tables. Specifically, disclosed herein is a non-naturally occurring system for RNA-guided DNA integration, wherein the system is encoded by a nucleic acid, wherein the nucleic acid encodes a cas 12a gene and a recombination-promoting nuclease A (RpnA) gene, wherein the nucleic acid encoding cas12a and rpnA are separated by about 3 genes, which is about 1500-4500 nucleotides. The cas12a gene is nucleolytically inactive but still binds its guide RNA. The cas12a gene, along with its specific guide RNA, functionally interacts with the RpnA-like gene to direct the insertion of a DNA into a genomic site that is complementary to the guide RNA. The RpnA-like gene assists the integration of a nucleic acid into the host cell. One of skill in the art will appreciate what “about 3 genes” means in terms of distance. The system can further comprise a guide molecule comprising a crRNA. The system can also comprise donor DNA, such as nucleic acid cargo. This CAST is further discussed in the Examples section.

### Specific Examples of CAST Sequences

Disclosed herein are specific CASTs and the nucleic acids which encode them. These CASTs are found in Appendix 1, and represent those CASTs whose components are detailed above. In other words, CASTS, and the genes that encode them, are specific examples of the general structure of CASTs described above. Proteins which encode the components of Tn7 CASTs are found in SEQ ID NOS: 458. This disclosure contemplates CASTs with a sequence that is 80, 85, 90, 91, 92, 93, 94, 95, 96, 97, 98, 99, or 100% identical to those sequences. The components that make up the protein units of CASTS can be combined in multiple ways to form a functional CAST. In other words, combinations of all of the CAST “building blocks” are found in SEQ ID NOS: 458. Specific orders, or combinations, of these sequences are given above. The proteins disclosed herein are interchangeable and capable of forming a CAST. In other words, these components can be rearranged, so that they do not necessarily occur sequentially, but the elements can result in a CAST that can function as a transposon.

This disclosure also contemplates nucleic acid sequences encoding CASTs disclosed herein with a sequence that is 80, 85, 90, 91, 92, 93, 94, 95, 96, 97, 98, 99, or 100% identical to those of SEQ ID NOS: 1-458. Also contemplated is that the individual genes encoding specific proteins of the CAST can be rearranged, so that they do not necessarily occur sequentially, but the genes themselves can be rearranged as long as the CAST can still function as a transposon.

### Vectors and Cells

Further disclosed herein are vectors comprising one or more CASTs disclosed herein. Also contemplated herein are cells comprising the vectors. In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids.

The present disclosure further provides methods of inserting a donor polynucleotide into a target site in a genome of a cell, which comprises introducing into the cell one or more CASTs or functional fragments thereof as disclosed herein, a guide molecule comprising crRNA specific for the target site, and the donor DNA (nucleic acid cargo).

The one or more components needed to insert a donor polynucleotide into the cell may be introduced into a cell by delivering a delivery polynucleotide comprising nucleic acid sequence encoding the one or more components. The nucleic acid sequence encoding the one or more components may be expressed from a nucleic acid operably linked to a regulatory sequence that is expressed in the cell. The one or more components may be encoded on the same delivery polynucleotide, on individual delivery polynucleotides, or some combination thereof. The delivery polynucleotide may be a vector.

Alternatively, the components may be delivered to a cell or population of cells as a pre-formed ribonucleoprotein (RNP) complex. In certain example embodiments, components CAST components are delivered as an RNP and the donor polynucleotide is delivered as a polynucleotide.

In certain example embodiments, the CAST system may be delivered to a cell or population of cells in vitro. In certain example embodiments, the CAST system may be delivered in vivo. A method for sequence-specific modification of a target nucleic acid sequence in a prokaryotic cell, the method comprising providing to the cell a CAST, wherein the CAST comprises any of the CASTs disclosed herein, a crRNA, and a donor DNA comprising a nucleic acid cargo sequence under conditions for modification of the target nucleic acid, wherein the crRNA is specific for the target nucleic acid sequence, and further wherein the donor DNA comprises nucleic acid cargo sequence to be incorporated into the target nucleic acid sequence, thereby modifying the target nucleic acid in a sequence-specific manner.

### Methods of Using CASTS

The nucleic acids-targeting systems, the vector systems, the vectors and the compositions described herein may be used in various nucleic acids-targeting applications, altering or modifying synthesis of a gene product, such as a protein, nucleic acids cleavage, nucleic acids editing, nucleic acids splicing; trafficking of target nucleic acids, tracing of target nucleic acids, isolation of target nucleic acids, visualization of target nucleic acids, etc. For example, the CASTs can be used for any purpose where it is beneficial to transfer genetic material to a cell.

The CAST systems disclosed herein are useful in a variety of applications known to those of skill in the art. For example, knock-in or knock-out gene editing. Also contemplated is chromosome engineering, which provides advantages in controlling the copy number and maintaining the stability of heterologous genes. Moreover, this system allows uninterrupted DNA integration while the cells grow and multiply, which is uniquely attractive for building multicopy libraries (Wang et al. Transposon-Associated CRISPR-Cas System: A Powerful DNA Insertion Tool, Trends in Microbiology, Volume 29, Issue 7, 2021, Pages 565-568,). Recently, Zhang et al. applied a CRISPR-associated transposase strategy to establish a library of E. coli strains carrying cargoes with successively increasing copy numbers (up to 10) within 5 days. Notably, this approach is independent of selective pressure (Zhang 2020).

Examples of methods of using CAST systems are known to those of skill in the art. For example, Piergentili et al. (Piergentili R, Del Rio A, Signore F, Umani Ronchi F, Marinelli E, Zaami S. CRISPR-Cas and Its Wide-Ranging Applications: From Human Genome Editing to Environmental Implications, Technical Limitations, Hazards and Bioethical Issues. Cells. 2021; 10 (5): 969. Published 2021 Apr. 21) disclosed multiple ways that CAST systems can be used to treat diseases and disorders, and for other purposes where gene transfer is useful, such as in environmental applications. The systems disclosed herein can be used in agriculture for crop upgrade and breeding including the creation of allergy-free foods, for eradicating pests, for the improvement of animal breeds, and to make bio-fuels. Applications in human health include the making of new medicines through the creation of genetically modified organisms, the treatment of viral infections, the control of pathogens, applications in clinical diagnostics and the cure of human genetic diseases, either caused by somatic (e.g., cancer) or inherited (mendelian disorders) mutations.

In one aspect, the invention provides a method of modifying a target polynucleotide in a cell. In some embodiments, the method comprises a CAST complex (including crRNA and a donor polynucleotide) capable of binding to the target to effect cleavage of said target and insertion of the donor polynucleotide.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In some cases, the donor polynucleotide includes multiple genes that embody the cell with additional functions, such as altered metabolic pathways, synthetic gene circuits, and the ability to create or modify organic biosynthetic compounds. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g. sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In other example embodiments, the systems disclosed herein may be used to augment healthy cells that enhance cell function and/or are therapeutically beneficial. For example, the systems disclosed herein may be used to introduce a chimeric antigen receptor (CAR) into a specific spot of a T cell genome—enabling the T cell to recognize and destroy cancer cells.

Additionally, the transposon-associated CRISPR-Cas system can potentially also be applied to non-isolated species, which can be particularly useful for genetic manipulations in mixed bacterial or eukaryotic cell communities. When the CAST was delivered from a donor E. coli strain into a bacterial community derived from the mouse intestinal tract by intergenic conjugation, target-and species-specific integration was successfully achieved (Vo et al. CRISPR RNA-guided integrases for high-efficiency, multiplexed bacterial genome engineering Nat. Biotechnol., 39, 2021, pp. 480-4).

### EXAMPLES

### Example 1: Metagenomic Discovery of CRISPR-Associated Transposons

CRISPR-Cas systems confer bacteria and archaea with adaptive immunity against mobile genetic elements. These systems also participate in other cellular processes. For example, CRISPR-associated Tn7 transposons (CASTs) have co-opted nuclease-inactive CRISPR effector proteins to guide their own transposition. Disclosed herein are novel CASTs, including systems with new architectures and ones that use distinct CRISPR subtypes. Also described herein is a non-Tn7 CAST that co-opts Cas12a. These findings disclose novel mechanisms for vertical and horizontal CAST targeting and shed light on how CASTs have co-evolved with CRISPR-Cas systems.

CASTs were systematically surveyed across metagenomic databases using a custom-built computational pipeline that identifies both Tn7-and non-Tn7 CASTs. Using this pipeline, unique architectures were discovered for Type I-B, I-F, and V CASTs. Type I-F CASTs show the greatest diversity in Cas genes, including TniQ-Cas8/5 fusions, split Cas7s, and even split Cas5 genes. Some I-F CASTs appear likely to assemble a Cascade around a short crRNA for self-targeting from a non-canonical spacer. Type I-B CASTs frequently encode two TniQ/TnsD homologs, one of which is used for self-targeting via a crRNA-independent mechanism. Remarkably, I-B systems were also found that encode two TniQ homologs and a self-targeting crRNA, suggesting additional unexplored targeting mechanisms. In addition, new Type I-C and Type IV-family Tn7-like CASTs were observed with unique gene architectures. Both of these sub-families lack canonical CRISPR arrays, suggesting that CASTs use distal CRISPR arrays, perhaps from active CRISPR-Cas systems, for horizontal gene transfer. Multiple self-insertions and gene loss in Type V systems were identified, indicating that target immunity—a mechanism that prevents transposons from multiple self-insertions at an attachment site—is frequently weakened. Finally, a set of Cas12a-associated Rpn-family transposases were found that can participate in crRNA-guided horizontal gene transfer. These findings shed additional light on how CASTs have co-opted CRISPR-Cas systems and further expand the precision gene editing toolbox.

### Diversity of Type I-F CASTs

1093 non-redundant I-F sub-systems were identified with the prototypical gene arrangement of TnsA-TnsB-TnsC separated from TniQ-Cas8/Cas5-Cas7-Cas6 by a large cargo region (FIG. 1A). Tn7 cargo genes are unrelated to the transposition mechanism, and often include antibiotic resistance genes (Parks 2009). Type I-F3a systems, defined as using the conserved genes guaC or yciA as their attachment site, comprise ˜61% of all I-F CASTs (FIG. 1B). I-F3b systems, which use the rsmJ or ffs attachment site, comprise ˜34% of I-F CASTs (Petassi 2020). The remaining 5% of I-F systems form a distinct group, termed I-F3c, with a unique attachment site and self-targeting mechanism.

The most common gene arrangement in the dataset for all three subtypes encodes the TnsA-C proteins in one operon, and the TniQ and Cas proteins in a second operon that is adjacent to the CRISPR repeats. A large cargo spanning ˜10-20 kbp either separates these operons or is present downstream of the cas genes. The stoichiometry of the Cascade effector has been previously reported to be (Cas6)₁: (Cas7)₆: (Cas8/Cas5)₁: crRNA₁: TniQ₂, based on cryo-electron microscopy of Type I-F CAST complexes (Halpin-Healy 2020; Jia 2020; Wang 2020; Zhang 2020). In these studies, TniQ interacts with Cas7 and is structurally distant from Cas8/Cas5. However, in two of the new I-F3a systems, TniQ is expressed as an N-terminal fusion with Cas8/Cas5. Four distinct I-F3a systems also have a split Cas7, and in one system, both the Cas5 and Cas7 proteins are split into two distinct polypeptides.

All I-F3 systems that were identified appear to use a crRNA-guided self-targeting mechanism that directs Cascade near the attachment site (Petassi 2020). The self-targeting crRNA is either in the leader-distal position of the CRISPR array or 80-85 nt away from the CRISPR array, as reported previously (Petassi 2020). These self-targeting crRNAs are flanked by an atypical direct repeat that has several substitutions relative to the direct repeats within the CRISPR array. I-F3c CASTs attach upstream of a protein of unknown function that encodes seven putative transmembrane regions (Methods). This attachment site has not been previously reported for any Tn7-family transposon. To determine how Type I-F3c systems use crRNA-guided transposition, the region around the CRISPR array was aligned with the sequence 500 bp upstream of tnsA. This identified a short 20 bp sequence immediately after the final canonical CRISPR repeat that matched the region approximately 64 bp upstream of the transposon end. This short, atypical spacer is followed by an atypical repeat (FIG. 1C), akin to the I-F3a and I-F3b systems.

Type I-F systems recognize a dinucleotide protospacer adjacent motif (PAM) (Rollins 2015). An analysis of the self-targeting PAMs highlighted that they vary with the attachment site and CAST sub-family (FIG. 1D). Next, the sequence composition of the inverted repeats that span Tn7 was analyzed. The right inverted repeat starts with a universally conserved 5′-TGT that is recognized by the essential TnsB recombinase (Choi 2013). The rest of this repeat varies but is most similar between CASTs that have the same attachment site (FIG. 1D). These results further confirm that I-F3c systems cluster into a distinct CAST sub-type.

### Type I-B CASTs Encode Multiple Integration Mechanisms

Four families of Type I-B CASTs were found that lack interference and adaptation genes. These systems either encode a single TniQ or dual TniQs of unequal length (FIG. 2A). Systems with dual TniQs comprise 79% of all identified systems. The most common Type I-B system (I-B1) encoded TniQ1 between TnsC and a Cas gene and TniQ2 is on the distal end of the CRISPR array (FIG. 2A, top and FIG. 2B). Systems with a single TniQ (I-B2 and I-B3) had two distinct gene architectures and self-targeting modalities. In I-B2 systems, where TniQ was sandwiched between TnsC and Cas6, a self-targeting spacer was identified that was complementary to a region downstream of glmS (FIG. 2A, middle). However, no self-targeting spacers in I-B3 systems were found, where TniQ was between the CRISPR array and cargo genes. In addition, this TniQ was nearly double the length of the shorter TniQs found between TnsC and Cas6 and bore a strong resemblance to the TnsD encoded in canonical Tn7 systems. Notably, single-TniQ systems that encode a TnsD-like TniQ but also lack self-targeting spacers were found.

An atypical Type I-B CAST (I-B4) was identified that had a unique gene architecture and self-targeting mechanism (bottom row, FIG. 2A). This system encodes TnsB and TnsC but lacks the TnsA gene, akin to Type V systems (Hsieh 2021; Strecker 2019). Two TniQ homologs of unequal length are immediately adjacent to the inverted repeats but distal from the Cas operon. TniQ₁ is sandwiched between the right transposon end and a short CRISPR array; TniQ₃ is only ˜450 bp long and is located between TnsC and the left transposon end. This short TniQ₃ can be aligned against the N-terminus of traditional CAST-associated TniQs (FIG. 2D). Notably, this is the only dual-TniQ CAST that encodes a self-targeting spacer with near-perfect complementarity to a region of DNA just outside the left transposon end. The attachment site is adjacent to a gene of unknown function near the left transposon end (FIG. 2A), akin to the attachment sites in Type V CASTs. The self-targeting spacer is flanked by an atypical direct repeat and is also 6 to 23 bp shorter than the other spacers in the CRISPR array (FIG. 2C). Based on these findings, and the observation of Type I-F CASTs with short self-targeting spacers, it appears that Type I-B systems can also assemble self-targeting mini-Cascades.

To better understand the roles of multiple TniQs in Type I-B CASTs, a phylogenetic tree of Type I-B, I-F3a, and I-F3b TniQs was constructed, along with TnsD from the canonical Tn7 transposon (FIG. 2B). Compared with the cluster of TniQ₂, the short TniQ₁ from Type I-B1 and B2 systems are closer to TniQ from other CASTs, while the Type I-B1 TniQ₂ clusters with canonical Tn7 TnsD. The close similarity between TniQ₂ and Tn7 tnsD, along with the apparent lack of self-targeting in most dual TniQ systems, suggests that TniQ₂ serves the same role as TnsD, namely that it is a sequence-specific DNA-binding protein that directs transposition downstream of glmS. TniQ₁, in turn, forms a complex with Cascade to guide TnsABC to a crRNA-directed target. An experimental study demonstrated that I-B CASTs with two tniQ genes use two separate pathways for target selection (Saito 2020).

### Novel Type I-C and IV CASTs from Metagenomic Sources

An analysis of the metagenomic contigs revealed a Type IV CAST (FIG. 3A). Type IV systems are primarily encoded by plasmid-like elements to mediate inter-plasmid conflicts Pinilla-Redondo 2020; Taylor 2021). Phylogenetic trees of Cas6 and Cas7 independently placed this CAST within the Type IV-A3 sub-family (FIG. 3B). These systems frequently shed their CRISPR repeats, instead of using distal CRISPR arrays (Pinilla-Redondo 2020). Although no CRISPR repeats were found in this system using CRASS or PILER-CR, a spacer-like DNA segment with strong complementarity to the C-terminus of glmS, the likely attachment site was found. This putative self-targeting spacer is adjacent to a hairpin that resembles the direct repeats in other Type IV CRISPR-Cas systems (FIG. 3A, bottom). It was concluded that this minimal spacer-repeat motif directs self-targeting by the Type IV system. Horizontal transfer may still occur via a distal CRISPR array, akin to the interference mechanism in other Type IV CRISPR-Cas systems (Pinilla-Redondo 2020).

Nine non-redundant Type I-C CASTs with Cas5, Cas7, and Cas8 downstream of TnsABC and TniQ (FIG. 3C) were found. TniQ is immediately adjacent to TnsABC rather than the Cas proteins. A phylogenetic analysis of Cas8 showed close similarity to Cas8c (FIG. 3C, right). A CRISPR array via CRASS or PILER-CR was not detected. No tRNA or common Tn7-associated attachment sites near either the left or right transposon ends were detected either, precluding a detailed analysis of the self-targeting mechanism.

### Architectural Diversity and Self-Targeting in Type V CASTs

Type V CASTs were likely formed when a Tn7-like transposon co-opted a Cas12 gene for RNA-guided DNA targeting (Makarova 2020; Faure 2019). Most Type V CASTs contain TnsB, TnsC, and TniQ at one end of the transposon with Cas12k, a small CRISPR array, and an atypical repeat-spacer on the other end. Cargo genes spanning 2 to 23 kb of additional DNA sequences are sandwiched between TniQ and Cas12k (FIG. 4A). In contrast to the Class 1 systems described above, all metagenomic Type V systems lacked TnsA, consistent with the proposal that Cas12k was captured by a Tn5053-family transposon, which contains TnsB, TnsC, and TniQ homologs but also lacks TnsA (Strecker 2020). The tracrRNA is upstream of the canonical CRISPR array with homology to the atypical repeat. The crRNA usually has good homology to the target DNA, with 98% of systems containing one or zero mismatches in the first ten basepairs (FIG. 4B). As previously observed (Hsieh 2021), atypical spacers generally targeted tRNA genes immediately adjacent to the transposon. One system was also found that attaches 104 bp downstream of ArsS, an arsenosugar biosynthesis radical SAM protein. Analysis of the DNA upstream of the self-targeting spacer revealed 5′-TGGTA as the most common PAM, with some variability in the −5, −4, and −1 positions. (FIG. 4C). Experimental evidence for two CASTs showed a preference for a smaller 5′-GTN PAM (Strecker 2019). Overall, this architecture and preference for tRNA attachment sites corroborate previous bioinformatic and experimental observations (Saito 2021; Faure 2019; Koonin 2020).

Type V CASTs with unusual TnsC and TnsB arrangements were also found. Notably, all Type V TnsC proteins lack the canonical TnsA-and TnsB-interacting domains and have partial truncations of the TniQ-interacting domain (Choi 2014; Park 2021; Peters 2015). The shortest CAST—only 6.6 kbp in total, including the cargo—encodes a 98 amino acid TnsC fragment whose sequence overlaps with TnsB by 115 bp (FIG. 4A, 4D). In addition to losing the TniQ, TnsB, and TnsA interacting domains, this TnsC has also lost its ATPase domain. It was hypothesized that the minimal TnsC encodes uncharacterized TnsB-and TniQ-interaction motifs. Because of its compact organization, this CAST is also a prime candidate for gene editing applications.

Multiple CASTs split TnsB into separate ORFs that encode just the N-or C-terminus. Alternatively, a full-length TnsB is encoded next to a TnsB fragment containing most of the catalytic domain (FIG. 4E). Strikingly, two unrelated systems encode the same N-terminal region of TnsB. It is possible that these split TnsBs form a heterodimeric TnsB₁:TnsB₂ transposition complex. These heterodimeric complexes retain the catalytic core while also maintaining the requisite TnsC interaction motifs via the longer TnsB subunit.

Multiple contigs were also observed with two CASTs inserted at the same attachment site (bottom rows, FIG. 4A). Tn7 transposons can prevent re-insertion at the attachment site by TnsB-mediated dissociation of TnsC from target DNA (Ae 2020; Skelding 2003). However, more distant Tn7-family transposases may still insert at a single attachment site, resulting in several transposons that are situated adjacent to each other (Peters 2015). Consistent with this idea, the dual-insertion Type V CASTs have distinct cargos, unique gene architectures, and divergent Cas12k sequences. In one scenario, the tRNA-distal CAST encoded an N-terminal TnsB truncation and lost TniQ (7th row, FIG. 4A). The tRNA-proximal CAST from the same organism encoded the C-terminal TnsB fragment and had a complete Type V-family TniQ. A second dual CAST system had lost both TnsC and TniQ from the tRNA-distal transposon (last row, FIG. 4A). Both systems encode a self-targeting spacer and a full Cas12k gene, suggesting that they are both still active.

### Phylogenetic Analysis of Tn7-CASTs

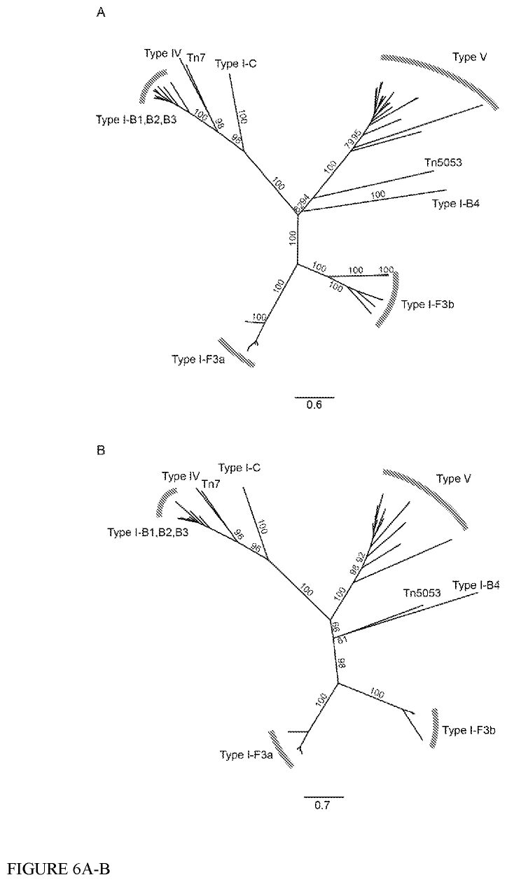

To clarify the evolutionary relationship between Tn7-CASTs, phylogenetic trees of the TnsB were built (FIG. 6A) and TnsC (FIG. 6B) proteins from all known CAST subtypes as well as Tn7-family transposons (Methods). The phylogenetic relationships between sub-systems were nearly identical for both TnsB and TnsC, suggesting that these proteins are co-evolving as a system. TnsA was omitted from this analysis because Type I-B4 and all Type V Tn7-CASTs lack this gene. It was confirmed that all metagenomic Type V-CASTs are phylogenetically closer to the Tn5053 transposon than Tn7. In contrast, Type I-B1-3, I-C, and IV CASTs are phylogenetically close to Tn7. Such limited evolutionary drift suggests a relatively recent co-opting of this CRISPR-Cas system. Type I-B4 CASTs are a notable exception because these systems also lack TnsA and cluster closer to Tn5053 than to the reference Tn7. Type I-F CASTs are highly divergent from both Tn7 and Tn5053, with a large phylogenetic separation between the I-F3a and I-F3b sub-types.

### Non-Tn7 CASTs that Co-Opt Cas12a and Type I-E Cascade

While transposons other than Tn7 may have co-opted CRISPR-Cas systems for attachment site recognition, this has never been reported. To explore this possibility, contigs that encode: (1) at least one non-Tn7 transposase gene (Methods), (2) a CRISPR array containing at least two spacers, and (3) Class 1 or Class 2 DNA-binding effectors (i.e., Cas9, Cas12, Cas13, or any three of Cas5/6/7/8) were identified. Excluded were contigs that encode interference (i.e., Cas3 or Cas10) or acquisition machinery (i.e., Cas1 or Cas2). Class 2 nucleases were additionally filtered by size to exclude truncated genes (Methods). Type II, V, and VI systems where the catalytic nuclease domain residues are mutated or deleted were prioritized, as these enzymes cannot participate in adaptive immunity. Nuclease-inactivating mutations or deletions were detected in 25% of Cas9 genes (in one or both nuclease domains), 8% of Cas12 genes, and none of the Cas13 genes.

40 non-redundant examples of a nuclease-inactive Cas12a or a Type I-E Cascade were found near a putative recombination-promoting nuclease/transposase (Rpn)-like protein (FIG. 5A). Rpn family proteins were originally investigated because of their close homology to the catalytic domain of transposase_31 (Kingson 2017). These proteins contain a PDDEXK nuclease domain, first discovered in restriction endonucleases, but also observed in T7 TnsA and other diverse DNA-processing enzymes (Aggarwal 1995; Hickman 2000; Steczkiewicz 2012) (FIG. 5B). E. coli RpnA promotes RecA-independent gene transfer in cells and is a Ca2-stimulated DNA nuclease in vitro (Kingston 2017).

The genetic context around Cas12a in these systems is highly enriched with nucleic acid-interacting proteins, including a topoisomerase, an RNAse, a DNA polymerase subunit, and one or two helicases. Likewise, the Cascade system encodes a helicase and an HNH endonuclease.

Three Cas12a systems encode an HU family DNA-binding protein, and one of those also contains a protein with homology to a phage replisome organizer (Missich 1997). Three systems with putative atypical self-targeting spacers adjacent to a canonical CRISPR array were detected (FIG. 5A). In the Cas12a systems, the self-targeting spacer is complementary to two or four nearby targets, all of which are positioned at intergenic sequences. The closest targets of these self-targeting sequences are adjacent to a 5′-CTTA PAM, which is recognized by conventional Cas12a nucleases (FIG. 5C) (Jacobsen 2020; Leenay 2016). There are 9 bp inverted repeats (with one mismatch) that flank Cas12a, the Rpn-family protein, and several other genes.

The Cas12a genes in these systems cover 90% of the well-characterized AsCas12a sequence (˜24% amino acid identity), including the critical crRNA-processing, DNA binding, and nuclease domains (Yamano 2016). Cas12a can process its own pre-crRNA via a dedicated RNAse domain (Fonfara 2016). Three residues in this domain have been identified as critical for pre-crRNA processing; all are conserved in Rpn-associated Cas12a variants (FIG. 5D) (Fonfara 2016). Cas12a degrades double-stranded DNA by first cleaving the non-target strand, followed by the target strand in its single RuvC nuclease active site (Jeon 2018; Singh 2018; Strohkendl 2018; Swarts 2018). Phosphate bond scission is catalyzed by two magnesium ions, one of which is coordinated by a critical aspartic residue (position 908 in Acidaminococcus (As) Cas12a). This residue is mutated to isoleucine in all Rpn-associated systems (FIG. 5D). Similarly, the Type I-E system encodes all the Cascade subunits but does not have Cas3. It was concluded that these systems bear striking resemblance to Tn7-associated CASTs and can potentially mobilize genomic information for crRNA-guided horizontal transfer.

### DISCUSSION

CASTs are rare in fully-sequenced prokaryotic genomes and are likely to be missed by traditional CRISPR detection pipelines due to their unusual operon structures and short CRISPR arrays. To address this gap, a set of Python libraries were developed that allow users to efficiently use BLAST to search for co-occurring genes and to perform subsequent searches for arbitrary gene architectures. Approximately 30 terabytes of metagenomic contigs were examined to identify ˜1476 high-confidence CASTs, including novel Type IV and Type I-C systems, as well as a Type I-B4 CRISPR system that co-evolved with a Tn5053-like element, a member of the Tn7 family of transposons that lacks TnsA. Systems were discovered that include a putative nuclease-inactive Cas 12a and a non-Tn7 transposase-like recombinase.

In the NCBI and metagenomic databases, the most abundant Tn7-associated CASTs are those that co-opt Class 1 CRISPR systems. Of these, Type I-F sub-systems are the most structurally diverse. Notably, some I-F CASTs were found that encode TniQ-Cas8/Cas5 fusions, duplicate Cas5s, and duplicate Cas7s. Gene duplication of the Cas5 and/or Cas7 could have allowed one of the paralogs to form a protein-protein interface with TniQ. The second paralog may have been subsequently lost. The remaining paralog resulted in the streamlined I-F CASTs that are most frequently found in bacterial genomes. The short atypical pre-crRNA in some I-F CASTs also suggests that these systems assemble a shortened Cascade for self-targeting. The size of Type I-E and I-F Cascades can be tuned by the length of the crRNA (Gleditzsch 2016; Songailiene 2019; Tuminauskaite 2020). Intriguingly, short I-F Cascades cannot recruit Cas3 but are still able to bind the target DNA, making them an ideal system for directed Tn7 transposition (Tuminauskaite 2020). Short Cascades, along with the atypical direct repeats, may differentiate self-targeting CASTs from those undergoing horizontal gene transfer in the I-F3c system.

Type I-B CASTs encode one or two TniQ/TnsD homologs. One report has uncovered that self-targeting in some systems proceeds via TnsD, whereas horizontal transfer is crRNA-guided (Saito 2021). Atypical systems were also identified that encode two short TniQ homologs along with a self-targeting spacer. Both homologs in the atypical dual-TniQ systems are related to the I-F CAST TniQ. Based on this observation, along with the crRNA-directed self-targeting, and the alignment of TniQ₃ to the N-terminus of TniQ₁, it appears that this CAST assembles a hetero-dimeric Cascade consisting of a single repeat of each subunit. Alternatively, this system may assemble TniQ₁- and TniQ₃-only Cascades for self-targeting and horizontal transfer.

How do CASTs target mobile genetic elements with minimal CRISPR arrays? No systems were found that retained the Cas1/Cas2 acquisition machinery, suggesting that strong evolutionary pressure is preventing the CAST-associated CRISPR arrays from expanding. CASTs encode CRISPR arrays that are significantly shorter than the corresponding canonical CRISPR-Cas systems and these arrays may also be transcriptionally silenced via xre elements that are frequently found adjacent to these arrays in CASTs (Petassi 2020). Moreover, no CRISPR arrays were found in Type IV and I-C CASTs. It appears that CASTs use CRISPR arrays that occur elsewhere in the genome—perhaps in functional CRISPR-Cas systems—for horizontal gene transfer. Evidence for such in trans CRISPR array usage has already been documented for a large set of canonical CRISPR-Cas systems (Hoikkala 2021; Vink 2021). CRISPR arrays that are associated with active interference machinery serve as an ever-updating record of the most likely mobile genetic elements that the CAST can use for horizontal gene transfer (Amitai 2016). A second possibility is that Cas1/2/4 from an active CRISPR-Cas system can act in trans to add spacers to the CAST CRISPR array. This may be an important secondary mechanism when horizontal transfer places the CAST into a host that lacks a compatible CRISPR array.

A search revealed Cas12a proteins associated with Rpn-family transposases, two of which appear to have atypical spacers that target two sites up- and downstream of Cas12a. This curious arrangement could be the result of a duplication of the target site that is originally present in only a single copy. It is noted that the HU family DNA-binding protein is only present in systems with putative self-targeting spacers and that a homolog of this protein is essential in bacteriophage Mu for transpososome assembly (Lavoie 1993). Two other proteins of viral origin—a replisome organizer and a ribonuclease-are also found near Cas12a in a self-targeting system, hinting at an intriguing evolutionary path for the creation of this putative CAST. In sum, the CAST identification pipeline and diversity of new systems described herein add to the understanding of CRISPR transposons and expand the gene-editing toolkit.

### Materials and Methods

### Database Acquisition and Contig Assembly

NCBI genomes were downloaded using NCBI Genome Downloading Scripts with the command:
ncbi-genome-download—formats fasta bacteria
ncbi-genome-download—formats fasta archaea

Raw FASTQ files were downloaded from the EMBL-EBI repository (Mitchell 2020) of metagenomic sequencing. For each sample, the quality of the raw data was assessed with FastQC (FastQC 2011) using the command:

fastqc tara_reads_*fastq.gz

Low quality reads were trimmed with Sickle (Joshi 2011) using the command:
sickle pe
f name_reads_R1.fastq.gz
r name_reads_R2.fastq.gz
t sanger
o name_trimmed_R1.fastq
p name_trimmed_R2.fastq
s/dev/null
   Megahit (Li 2015) was used to assemble the trimmed data with:
megahit
1 name_trimmed_R1.fastq
2 name_trimmed_R2.fastq
o name_assembly

### Analysis Pipeline

A Python library, Opfi (short for Operon Finder) was used to search genomic or metagenomic sequence data for putative CRISPR transposons. This library consists of two modules, Gene Finder and Operon Analyzer. The Gene Finder module enables the user to use BLAST to identify genomic neighborhoods that contain specific sets of genes, such as Cas9 or TnsA. It can also identify CRISPR repeats. The Operon Analyzer module further filters the output from Gene Finder by imposing additional user-defined constraints on the initial hits. For example, Operon Analyzer can be used to find all genomic regions that contain a transposase and at least two Cas genes but no Cas3.

Gene Finder was used to locate genomic regions of interest using the following logic. First, all regions containing at least one transposase gene were located. Within those regions, Cas genes were searched for, and those that were no more than 25 kilobase pairs away from a transposase were located. Transposase-containing regions without at least one nearby Cas gene were discarded from further analysis. Finally, the remaining regions were further annotated for Tn7 accessory genes (TnsC-TnsE and TniQ), and CRISPR arrays.

The Gene Finder hits were processed and categorized using Operon Analyzer. To identify Tn7-like CRISPR-transposons, each putative operon was required to contain TnsA, TnsB, TnsC, and at least one Cas gene from Cas5-13; the distance between TnsA, TnsB, and TnsC needed to be less than 500 bp; the Cas proteins need to be downstream of TnsA/B/C and the distance between any Cas protein and TnsB needed to be less than 15 kbp. This dataset was classified into putative Class 1 systems and Class 2 systems based on their Cas signature proteins. Class 1 systems were manually reviewed to confirm the loss of adaptation (Cas1, Cas2) and interference (Cas3) proteins.

To identify non-Tn7 CRISPR-transposons, each putative operon was required to contain a CRISPR array, a transposase, and at least one Cas gene from Cas5-13. Systems containing Tn7 proteins, Cas1, or Cas2 were excluded. This dataset was partitioned into putative Class 1 systems (defined as loci with any three of Cas5/6/7/8) or Class 2 systems (Cas9, Cas12, or Cas13). For Class 1 systems, those containing Cas3 or Cas10 were further excluded To eliminate systems with fragments of effector proteins or poor matches to unrelated proteins, it was required that Cas9 have a size of 2-6 kbp, Cas12 a size of 3-6 kbp, and Cas13 a size of 2.5-6 kbp. Class 2systems were eliminated that were nucleolytically active, and finally clustered all systems using mmseqs easy-cluster with a minimum sequence ID of 0.95 (Steinegger 2017) to simplify manual curation.

### BLAST Database Construction

To find as many systems as possible, separate databases were assembled for Cas proteins, Tn7-family proteins, and non-Tn7 transposases. Databases were also developed for common Tn7 attachment sites following a separate effort (Petassi 2020).

All available bacterial and archaeal transposase sequences were downloaded from UniRef50, excluding partial sequences and sequences annotated with the word “zinc” (which tended to be false positives) as well as Tn7-related proteins. All transposases associated with transposons listed in the Transposon Registry (Tansirichaiva 2018) were downloaded from NCBI. Finally, 100 transposases associated with each of the major families of insertion sequences were downloaded from NCBI, again excluding partial sequences, and using the ‘relevance’ sort parameter.

Amino acid sequences for Cas1-Cas13 and Tn7 family proteins (TnsA-TnsE, TniQ) were downloaded from UniRef50. Additional Cas12 and Cas13 sequences, representing recently identified variants (e.g. Cas12k), were downloaded from the NCBI protein database and from primary literature sources (Pausch 2020; Shmakov 2017; Yan 2019).

To eliminate redundant sequences, each database was clustered using CD-HIT (Lu 2012) with a 50% sequence identity threshold and 80% alignment overlap. The clustered datasets were converted to the BLAST database format using makeblastdb (version 2.6.0 of NCBI BLAST+) with the following arguments:
makeblastdb
in <sequence fasta file>
title <database name>
out <database name>
dbtype prot
hash_index

The full-length sequences of GuaC (PF00478), RsmJ (PF04378), YciA (PF03061) were downloaded. The attachment site SRP-RNA gene (ffs) (RF00169) was downloaded from RFAM. To assign putative Cas5-Cas8 proteins to specific Type I CRISPR-Cas subtypes, Cas proteins were manually collected and their assignments from reviews by Koonin and colleagues (Shmakov 2017; Burstein 2017; Makarova 2015. All Cas protein sequences were converted into BLAST databases using makeblastdb (version 2.6.0) with default parameters.

### De-Duplication of Putative Operons

Approximately 57% of the metagenomic systems that passed the initial filter were nearly identical at the nucleotide sequence level. However, exact nucleotide comparisons were too slow to de-duplicate this large dataset. Instead, two systems were considered to be identical if they met the following properties: (1) they had the same protein-coding genes and CRISPR arrays in the same order; (2) the genes had the same relative distances to each other; and (3) the translated sequences of all proteins were identical. This de-duplication was applied to all systems before the downstream analysis.

### Self-Targeting Spacer Identification

Spacer sequences that were identified with PILER-CR were pairwise aligned with the contig sequence that contained them, using the Smith-Waterman local alignment function from the parasail library (Daily 2016), with gap open and gap extension penalties of 8, and using the NUC44 substitution matrix. Spacers with at least 80% homology to a location in the contig were classified as self-targeting.

For Type V systems, the CRISPR array search was augmented with minCED 0.4.2(Skennerton 2019) after noticing transposons that were otherwise intact but seemingly lacked

CRISPR arrays. The region between Cas12k and 200 bp after the end of the nearest CRISPR array was used to search for spacers (both atypical and canonical). Targets were searched for in the 500 bp region immediately downstream of the spacer search region, using the method described above. For Type V systems with multiple Cas12k genes, each spacer region was aligned to each target region in order to discover systems where multiple transposons had inserted at the same attachment site.

### Phylogenetic Analysis

Alignments of protein sequences were constructed with MAFFT, version v7.310 (Katoh 2013). Phylogenetic analysis was performed on the aligned sequences using the IQ-TREE, version 1.6.12 (Nguyen 2015), with automatic model selection. Models used were as follows: FIG. 2B: JTT+F+R3, FIG. 3B cas6: PMB+G4, FIG. 3B cas7: PMB+G4, FIG. 3C: PMB+G4, FIG. 6 tnsB: LG+R5, FIG. 6 tnsC: LG+G4. Trees were visualized using the Figtree program version 1.4.4.

### Classification of Nuclease-Dead Systems

To identify catalytically inactive Class 2 nucleases, each nuclease was aligned to a reference protein with MAFFT (version v7.310, with the FFT-NS-2 strategy for Cas9 and Cas12, and FFT-NS-i for Cas13). Cas9 homologs were aligned to SpCas9 (UniProtKB Q99ZW2.1, residues D10 and H840), Cas12 homologs to AsCas12a (UniProtKB U2UMQ6, residues D908 and E993), and Cas13 homologs to LbuCas13a (UniProtKB C7NBY4.1, residues R472, H477,R1048, and H1053). Mutations of D/E to anything other than D/E, or H/R to anything other than H/R/K were considered nuclease dead. As a control, this technique was employed on all 279Cas 12k proteins from NCBI as well as LbCas12a and FnCas12a (two known nuclease-active Cas12a proteins) and found that they were all correctly categorized.

### Identification of Inverted Repeats and Target Site Duplications

To identify inverted repeats, Generic Repeat Finder (commit hash: 35b1c4d6b3f6182df02315b98851cd2a30bd6201) was used (Shi 2019) with default parameters except as follows:
c: 0
s: 15
min tr: 15
min_space <operon length>
max_space <buffered operon length>
where <operon length> is the length of the putative operon and <buffered operon length> is the length of the putative operon, plus up to 1000 bp to allow a 500 bp buffer on either side of the operon. This detected inverted repeats that were at least 15 bp long. In cases where one inverted repeat fell within the bounds of the putative operon, it was discarded.

### Example 2: CRISPR-Associated Transposons Co-opt Host Spacers for Horizontal Transfer

CRISPR-associated transposons (CASTs) have co-opted CRISPR-Cas proteins for RNA-guided vertical and horizontal transmission. CASTs encode a short CRISPR array but lack the spacer acquisition proteins Cas1 and Cas2. Disclosed herein is the mechanism by which CASTs target new invading mobile elements without updating their own CRISPR arrays. It is bioinformatically shown that all CAST sub-families co-exist with canonical CRISPR-Cas systems. Using a quantitative transposition assay, it was demonstrated that the prototypical type I-F CAST can use CRISPR RNAs (crRNAs) from canonical CRISPR-Cas systems for horizontal gene transfer. A high-resolution structure of the type I-F CAST-Cascade in complex with a type-IIIB crRNA reveals how Cascade tolerates diverse direct repeats. In vivo, type I-F CASTs only require a short crRNA hairpin for efficient transposition from heterologous CRISPR arrays. Type I-B systems co-opt canonical crRNAs via a similar mechanism, whereas type V-K systems can co-opt the entire canonical effector complex for transposition. These discoveries explain how CASTs horizontally transfer to new hosts without updating their own CRISPR arrays. More broadly, these discoveries inform further engineering principles for both type I-F and type V CASTs in biotechnological applications.

The typical Tn7 transposon mediates transposition via two separate paths: (1) vertical gene transfer, where tnsA, B, and C interact with tnsD, a site-specific DNA-binding protein, to achieve transposition into the house keeping gene glmS; and (2) horizontal gene transfer, where tnsA, B, and C interact with tnsE, a structure recognition protein that directs the transposon to mobile elements. The original study discovering CASTs proposed the following mechanism of action: Cas6, cas7, cas8, and the CRISPR array (cas12k and CRISPR array for CAST V-K) together form the Cascade that substitutes for both tnsD's and tnsE's functions to guide the new system. This was further supported by the discovery of self-targeting spacers. Indeed, almost all CAST systems maintain their homing spacer. The only exception is CAST I-B. CAST I-B instead retains tnsD performing its original homing function. In addition, it appears CRISPR arrays associated with CASTs tend to be short and often contain only between one and three repeats. This shows that there can be a path other than the CAST systems use the CRISPR array they bring with to target the mobile DNA to allow them do the cell-to-cell transfer.

Disclosed herein is how CASTs co-opt active CRISPR defense systems to mobilize themselves for horizontal dissemination. A bioinformatic analysis reveals that all known CAST families co-occur with active CRISPR-Cas defense systems. These systems are a ready source for an up-to-date history of prior mobile genetic element infection. Mate-out transposition assays demonstrate that prototypical type I-F and I-B CASTs can use crRNAs derived from CRISPR defense systems nearly as efficiently as their own spacers. A cryoelectron microscopy structure of a type I-F TniQ-Cascade complex in complex with a type III-B crRNA shows that Cas6 interacts with the direct repeat (DR) of the crRNA via sequence-independent, electrostatic and pi-pi stacking interactions. A pi-pi stacking interaction between an evolutionarily-conserved Cas6 residue and a nucleotide at the apex of the DR stem-loop is essential for transposition and acts as a molecular ruler for the length of the DR stem. In agreement with this structure, it is shown herein that the DR must include a five basepair stem and five nucleotide loop for efficient transposition. This study resolves the long-standing question of how CASTs can mobilize into novel MGEs without updating their own CRISPR array. More broadly, design principles for optimizing CAST crRNAs for precision gene insertion in diverse organisms is revealed.

### RESULTS

### CASTs Co-Exist with CRISPR-Cas Defense Systems in the Same Genome

The NCBI and EMBL genomic databases were surveyed to identify 737 type I-F, 40 type I-B, and 189 type-V CAST systems. These systems were all missing the cas1 and cas2 spacer acquisition genes. This observation indicates that CASTs do not update their own CRISPR loci. Consistent with this observation, CAST CRISPR loci are extremely short or undetectable even during manual curation. For example, the type I-F3c system only retains a single self-targeting (“homing”) spacer, raising the question of how it can also target invading mobile DNA. In addition, type I-C systems do not encode any recognizable CRISPR arrays. Based on these observations, it was hypothesized that CASTs must employ an additional non-autonomous mechanism for horizontal transmission.

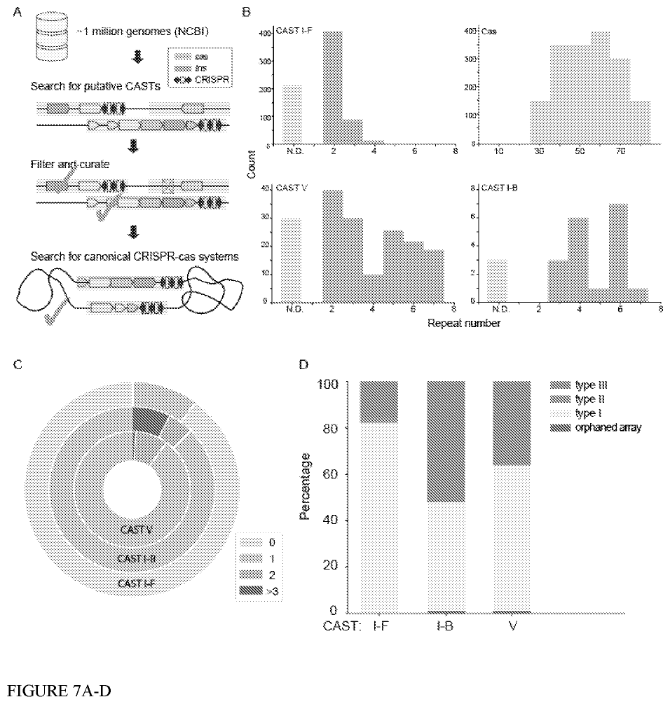

It was reasoned that CASTs may co-opt other CRISPR arrays that are scattered throughout the host genome for horizontal transmission. To test this hypothesis, additional CRISPR arrays were searched for in the genomes of all CAST-encoding organisms (FIG. 7). The 966 genomes that encoded a CAST were identified amongst the ˜1M high-quality assembled genomes in the NCBI reference sequences (RefSeq) database (FIG. 7A). Next, these CAST-encoding genomes were searched for co-occurring canonical CRISPR-Cas systems and orphaned CRISPR arrays. Ten percent of genomes that encode a type I-F CAST also encode additional CRISPR-Cas systems and 100% of organisms with a type I-B or type V CAST encode at least one additional CRISPR array (FIG. 7B). 12.5% of type I-B CASTs and 11% of type V CASTs also co-occurred with two or more additional CRISPR-cas systems (FIG. 7B). These CRISPR-Cas defense systems included an active nuclease (i.e., cas3), adaptation genes (i.e., cas1, cas2, cas4), and CRISPR arrays with 20-80 spacers, showing active spacer acquisition from mobile genetic elements (FIG. 7C). In contrast, all CASTs encoded very short or undetectable CRISPR arrays. Isolated examples of “orphaned” arrays were also observed that were not adjacent to a recognizable CRISPR-Cas defense system. Type I-F CASTs mainly co-occurred with type III-B, I-F, I-E CRISPR defense systems. In two genomes, the type I-F CAST co-existed with a type II-A defense system (FIG. 7D). In contrast, type I-B and V CASTs mainly co-occurred with type III-B and type I-D defense systems (FIG. 7D).

### Type I-F CASTs Catalyze Transposition from Heterologous CRISPR Arrays

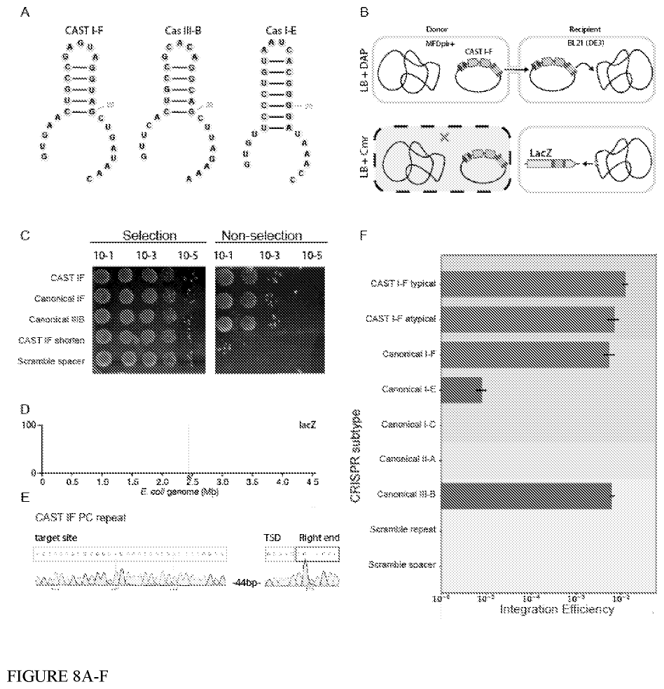

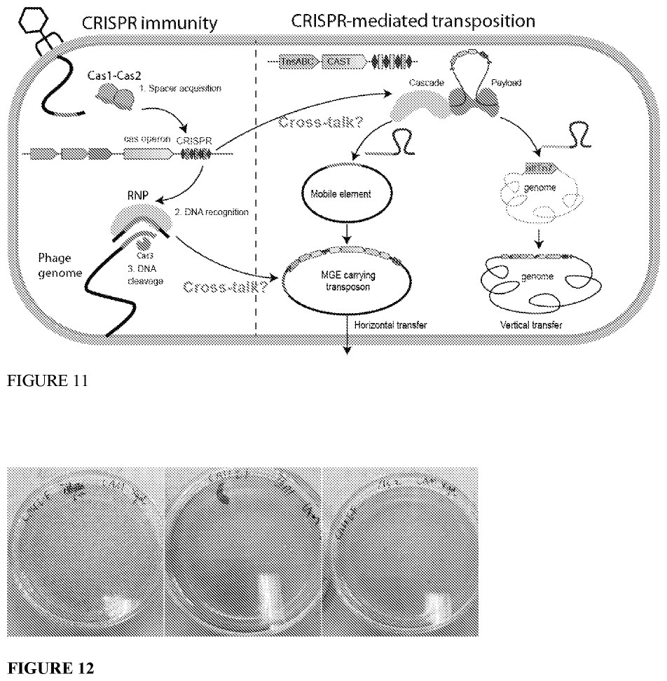

To determine whether CASTs can co-opt other CRISPR arrays, the sequences and secondary structures of their direct repeats (DRs) were compared. The CAST I-F, canonical I-F and III-B DRs have a broad RNA sequence diversity but are structurally identical with a five nucleotide (nt) loop, five basepair (bp) stem, and five nt 3′-handle (FIG. 8A). In contrast, the type I-E DR consists of a four nt loop, seven bp stem, and four nt 3′-handle. The type I-C and II-A DRs are even more divergent from the CAST I-F (FIG. 12).

To determine whether CASTs can exploit these heterologous CRISPR arrays, a conjugation-based chromosomal transposition assay was developed (FIG. 8B). In this assay, the CAST genes, a CRISPR array, and a chloramphenicol resistance marker surrounded by left and right inverted repeats are assembled in a conditionally replicative R6K plasmid that only grows on pir⁺ strains. In this case, the pir⁺ donor strain also includes a chromosomally integrated RP4 conjugation system. The donor cells are auxotrophic for diaminopimelic acid (DAP), allowing for counterselection on DAP-plates following conjugation with a recipient strain. The recipient cells are BL21 (DE3), a standard laboratory strain that supports CAST expression and transposition. Conjugative transfer of the R6K plasmid into the recipient cells and subsequent transposition of the CAST cargo into the host genome (targeting lacZ) results in chloramphenicol-resistant, ΔlacZ recipient BL21 (DE3) cells. Notably, the R6K plasmid is lost shortly after conjugation in the recipient cells (pir-) and the donor cells are also removed due the absence of DAP. Genomic transposition efficiency can be scored quantitatively via the ratio of recipient colonies on standard (DAP-) agar plates vs. plates that include chloramphenicol. The crRNA was designed to target the lacZ gene, resulting in white recipient colonies on Cm/X-gal plates; integration outside the lacZ gene produces blue colonies on the same plates. Finally, the insertion accuracy was scored via both Sanger- and whole-genome long-read sequencing of individual clones.

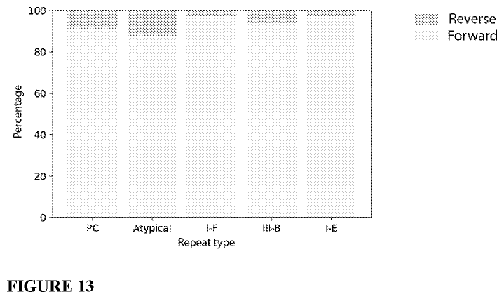

This assay was first tested with the native and atypical direct repeats from the well-characterized V. cholerae HE-45 Type I-F3a system (FIGS. 8C-D). This CAST encodes an atypical direct repeat and a homing spacer for site-specific integration into the host's genome. The homing spacer was removed to avoid spurious transposition events. The transposition efficiency was scored using a lacZ-targeting spacer, a scrambled spacer, or a scrambled direct repeat (the last two were included as negative controls; FIG. 8B). The native direct repeat transposition efficiency was 1.4±0.2% of all viable recipient cells (22800±12755 cfu, error denotes the mean of three biological replicates) This was suppressed below the limit of detection (<10⁻⁷ cfu) when either the spacer or the repeat was scrambled. All chloramphenicol-resistant colonies (n=953 across three biological replicates) appeared white on X-gal plates, showing transposition into the lacZ gene (FIG. 12). Sanger sequencing of the insertion junctions from 32 colonies showed that integration occurred in the forward direction in 91% of all cases and in the reverse direction in the remaining 9% (FIG. 13). All clones inserted 42-46 bp downstream of the end of target site. Whole-genome long-read sequencing of two clones indicated a single transposition event at the expected target size. HE-45 CAST's atypical direct repeat—typically adjacent to the homing spacer—supported a nearly identical transposition efficiency and insertion orientation. The atypical direct repeat differs from the typical repeat maintains the same overall stem-loop structure but has 12 mutated residues relative to the typical direct repeat. Because the typical and atypical direct repeats maintained a high transposition rate, it was concluded that the CAST effector complex can tolerated DRs with somewhat mutated RNA sequences.

Next, it was tested whether the V. cholerae HE-45 Type I-F3a CAST can use DRs from co-occurring CRISPR defense systems (FIGS. 8D,E). For this assay, the native CAST array targeted lacZ but was modified to encode the DR from CRISPR-Cas systems. All other protein and cargo components remained unchanged. Surprisingly, type I-F and III-B DRs supported transposition efficiencies that were comparable to those from the native CAST, despite having no RNA sequence similarity (FIG. 8E). CRISPR RNAs with type I-E DRs transposed ˜10⁻³-fold less efficiently than the native CAST crRNAs. In all cases, >99% of the resulting colonies were white on X-gal plates, indicating targeted transposition into the lacZ gene (N=543, 412, 93 for I-F, III-B, and I-E experiments, respectively). Sanger sequencing of the insertion junctions from 32 colonies for each of the heterologous CRISPR repeats showed that for all repeat types, integration occurred in the forward direction in 90% of all cases and in the reverse direction in the remaining 10% (FIG. 13). Integration events for all repeat types occurred primarily 43-45 bp from the 3′ end of the target to the integrated transposon (FIG. 8D). Long-read whole-genome sequencing of two independent clones confirmed that transposition was single copy and specific for the lacZ gene. In contrast, type I-C and II-A direct repeats did not show any transposition activity (<10⁷ cfus), indicating no crosstalk between type I-F CASTs and type I-C or II-A CRISPR arrays. The structures of these DRs differ substantially from the I-F DR, indicating that the DR stem loop structure is a major determinant of transposition activity.

### Cas6 Stabilizes Direct Repeats Via Sequence-Independent Electrostatic Interactions

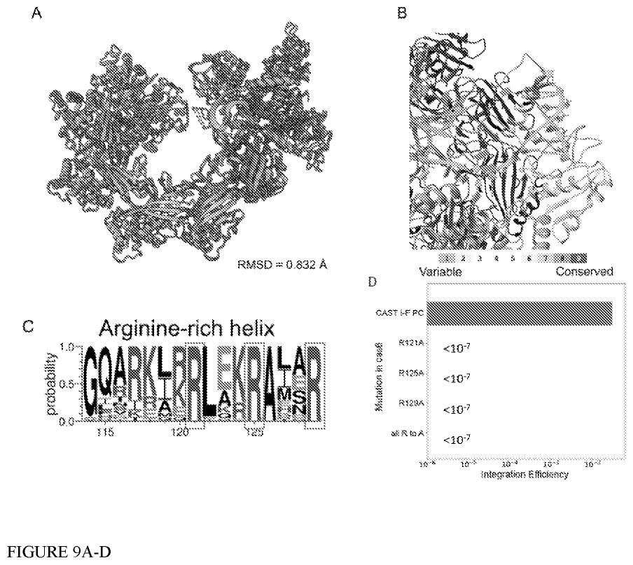

To investigate the molecular basis for how CASTs exploit heterologous CRISPR arrays, cryo-electron microscopy was used to solve the structure of the V. cholerae HE-45 Cascade co-purified with a type III-B crRNA. The crRNA contained a native direct repeat from the type III-B and a 32-bp spacer. The density for Cascade and the crRNA was refined with a prior model (PDB: 6PIG). (FIG. 9A).

The Cas6 subunit engages the crRNA direct repeat via sequence-independent interactions with the ribose phosphate backbone (FIGS. 9B-C). An arginine-rich helix with three highly conserved arginines (R117, R121, R125) forms a strong positive pocket to stabilize the crRNA handle. In addition, the guanidine (G54) at the apex of the stem-loop is flipped out of the plane and engages in a long-ranged pi-pi interaction with Cas6 (F138). These electrostatic interactions are crRNA-sequence independent, suggesting how Cascade engages diverse direct repeat sequences.

A multiple sequence analysis of I-F Cas6 proteins indicates that these electrostatic interactions are conserved across the entire CAST sub-family (FIG. 9D). The functional significance of these conserved residues was tested using the transposition assay described above (FIG. 9E). Mutating any of the arginines to an alanine suppressed transposition below the detection range (<10⁻⁷ cfus). Similarly, the Cas6 (F138A) mutant reduced transposition, indicating that the pi-pi interaction is also necessary for stably engaging the DR (FIG. 9D). It was concluded that Cas6 stabilizes diverse DRs via RNA sequence-independent electrostatic and pi-pi stacking interactions.

### Transposition Efficiency is Tuned by the Length of the DR Stem-Loop

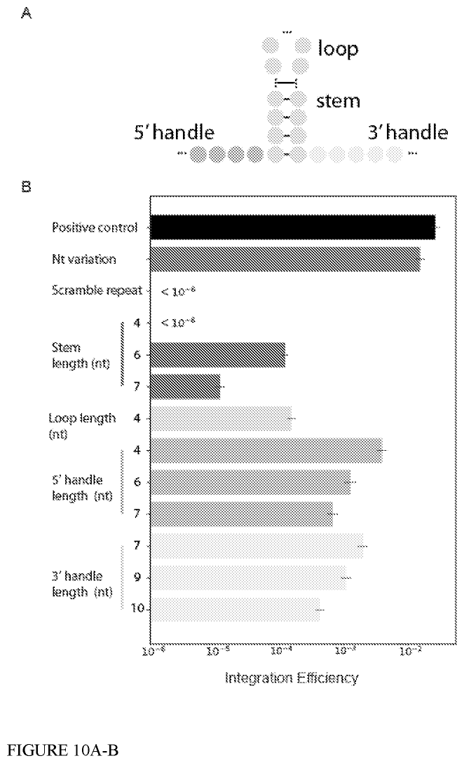

The reduced transposition efficiency with type I-E DRs indicates additional constraints on the CAST crRNA. To test these constraints, the DR sequence and/or structure was systematically varied and the resulting transposition efficiency was assayed (FIG. 8). The DR nucleotide sequence was scrambled at first, but retained the 5 bp stem, 5 nt loop, and 5 bp 5′ & 8 bp 3′ DR handles of the type I-F CAST. Surprisingly, this crRNA maintained wild type transposition efficiency (FIG. 10B). In contrast, scrambling the stem-loop sequence entirely abolished transposition. These results confirm that Cas6-DR contacts are sequence independent but require a structured DR to maintain activity.

Next, the length of the stem, loop, and the 5′ and 3′ handles were systematically varied to determine the key determinants of efficient transposition. Starting with the CAST I-F DR, changing the stem length by even a single basepair reduced transposition efficiency up to 5 fold (FIG. 8B). Increasing the length of the stem from five to seven basepairs (as in the type I-E DR, see FIG. 8B) decreased transposition efficiency 500-fold as compared with the type CAST I-F DR. Decrease the loop length by one nucleotide (as in the type I-E DR) also reduced transposition efficiency 100-fold (FIG. 10B). Changing the length of the 5′-and 3′-handles modestly reduced transposition efficiency.

It was also explored whether modifying the type I-E DR could improve transposition by the type I-F CAST. Consistent with prior findings, shortening the type I-E DR stem from seven to five basepairs significantly increased transposition activity. Adding one nucleotide from the loop to five nucleotides also improved transposition efficiency relative to the type I-E DR. These results further underscore that the DR structure is the key determinant for assembling a TniQ-Cascade effector complex on a heterologous crRNA. The stem must remain at five basepairs, whereas the loop can tolerate one nucleotide changes from the five-nucleotide native sequence. The structural basis for both effects likely arise from the base stacking interaction with Cas6 and will be further revisited in the Discussion.

### Type I-B CASTs Co-Opt Co-Occurring CRISPR Arrays for Horizontal Transfer

All type I-B CASTs co-occur with type I-D and III-B defense systems (FIG. 7C). To test whether the type I-B CAST can also use co-occurring CRISPR arrays, the mate-out transposition assay was adopted to the [Anabaena variabilis ATCC 29413] type I-B CAST. Transposition was first tested with the native CAST DR. Transposition efficiency with the native CRISPR sequence was ˜10\hat{ }3-fold lower than the type I-F CAST. 90% chloramphenicol-resistant colonies where white (n=152 colonies), indicating that most of the inserted cargo disrupted lacZ. Long-read and Sanger sequencing across the insertion junctions confirmed on-target integration 38-43 bp away from the target site. Scrambling the crRNA without preserving the DR structure ablated all transposition activity.

Type I-D and I-B DRs both have a 37 bp stem and 4 nt loop, whereas the type III-B DR has an extended 9 bp stem and a 4 nt loop. As expected, based on their structural similarities, the I-D DR supported transposition. Sanger sequencing of 9 clones and minion sequencing of 1 clone indicated that the cargo was inserted 37-44 bp away from the target site, as was observed with the native DRs. In contrast, the type III-B DR did not show any transposition within sensitivity. It was concluded that type I-B and I-F CASTs can both co-opt heterologous CRISPR arrays, so long as the crRNA DRs can be structurally accommodated within the Cascade effector complex

### Type V CASTs Transpose Via a CRISPR RNA-Independent Mechanism

Next, the transposition requirements of Type V CASTs was explored. Type V CASTs are a composite of the Tn5077-family transposons and a crRNA-guided Cas12k. Notably, these systems do not encode tnsA and insert their cargo via both cut-and-paste and replicative mechanisms.

The transposition assay described above was used for the Scytonema hofmannii Type V CAST, which is active in plasmid-based transposition assays. To maintain a single target site, the homing spacer that is naturally present in these systems was removed. The spacer targeted the lacZ gene, as described above. Surprisingly, chloramphenicol resistant colonies showed blue or light blue colonies, as would be expected from an incomplete disruption of lacZ. Whole-genome long-read sequencing of clones revealed a complex spectrum of insertion events. The extended integration range was confirmed via Sanger sequencing of the PCR amplicons that spanned the insertion junctions. Notably, blue and light-blue colonies displayed insertions that were downstream of a mutated LacZ, explaining the hypomorphic light blue colonies. Off-target integration was also observed in some of the clones without a discernable site preference. Taken together, these results highlight that type V systems use a distinct transposition mechanism that is not strictly defined by the distance from the target DNA sequence.

All type V CASTs co-exist with either canonical CRISPR-Cas system and majority subtype are I-D and III-B. Therefore, it was next investigated whether Type V CASTs systems can use CRISPR arrays from a canonical CRISPR-Cas system to carry out the transposition. The repeat sequence was chosen from canonical Cas I-D system, canonical Cas III-B system and CRISPR array far from any Cas proteins that co-existing with shCAST systems, re-program the spacer to target the lacZ gene in recipience cell's genome. Then the conjugation-based assay was performed to measure the transposition efficiency that CAST V systems using different repeat sequence. Though CAST V systems showed reasonable integration efficiency, we barely saw any difference between using different repeats. Even with a scrambled CRISPR array or without

CRISPR array, the CAST V systems can still perform the transposition. By plating the conjugation mixture on the Xgal plates, it was shown that only the 1% of chloramphenicol resistance colonies in the CAST V PC group are white colony and CAST V with others repeat don't have any white colony. The transposition efficiency drops below 10e-7 after the cas 12k gene was removed. These demonstrate that the transposition event is dependent on cas12k but not on a specific repeat sequence. The long-read (MinION) next-generation sequencing data showing both on and off target insertion contain the whole plasmid that follow the copy-and-paste. The genome also tends to have multiple insertion that happen at one site, which was observed in nature existing case. It is believed that the cross-talking at CRISPR array level is not necessary for CAST V-K systems because the random binding of cas12k is sufficient to allow CAST V-K systems to mobilize themselves.

It was intriguing to find the next degree of synteny between the defense Cas systems' cascade and the transposon element of CAST V system. The observation that the defense Cs systems' targeting modules can function with CAST V's transposition module implies unspecific communication between the cas12k and tnsB, tnsC, and tniQ in CAST V system. To test this assumption, the cas12k was substituted by dcas9 High fidelity I, dcas 12a and the cascade of CAST I-F systems, then assayed transposition activity. When the cas12k was substituted, Sanger sequencing result show that the transposon of the CAST V system can still perform the on-target transposition using either dcas9, dcas 12a, or the cascade of CAST I-F systems. In conclusion, these results demonstrate that the transposon component of the CAST V system does not specifically require the original cas 12k for transposition. Other proteins or protein complexes, such as for example dcas9, can form a stable R loop and effect the transposition.

### DISCUSSION

Disclosed herein is a comprehensive study about a possible path for CAST systems' horizontal gene transfer.

CAST systems need CRISPR array to mobilize themselves. Bioinformatic analysis of these systems indicate that the standard CRISPR array that CAST systems bind with are hardly able to facilitate their horizontal gene transfer because of their short length. CAST systems don't have the ability to obtain the novel spacer due to the lacking of cas1 and cas2. Unlike the most CAST systems maintain their homing spacer (One CAST I-B systems maintain tnsD for their homing), the loss of CRISPR array and spacer acquisition module suggest have other path for their mobilization. The CRISPR array canonical cas systems co-existing with CAST systems has been found to provide the resource for CAST systems to target novel mobile elements.

By using the conjugation-based transposition assay, it was shown that the type I-F CASTs can exploit heterologous type I-E, I-F, and III-B CRISPR arrays to direct their own transposition. With all three repeat CAST I-F system able to generate single on-target insertion products with a fairly high integration efficiency. It is likely that the CAST I-F systems not only have a flexible PAM requirement but also can tolerate some variation in repeat sequences. This offers the possibility that the CAST I-F systems using the canonical Cas systems' CRISPR array to facilitate their horizontal gene transfer. It was surprisingly observed that only 10% of CAST I-F systems co-existing with canonical Cas systems. There are several possibilities to explain this observation. First, the associate gene that CAST I-F systems bring with that could recognize the invading DNA such as the structure-specific DNA binding protein that senses features of replication associated primarily with conjugal plasmids as they enter a cell. Second, plasmids and phages may also encode their own CRISPR arrays or even full CRISPR systems, the CRISPR arrays that outside of bacteria genome could also be extra resource for CAST systems mobilization.

According to the cryo-EM structure, type I-F CAST Cascade with a type III-B crRNA complex is similar to type I-F CAST Cascade with its own crRNA and competent for initiating transposition with several nucleotide variation. It was shown that the crRNA direct repeat forms a pi-pi stacking interaction with the Cas6 subunit to stabilize the Cascade complex. It was further shown that CAST I-F systems can tolerate the variation in nucleotide change, handle's length, certain levels of loop length and 1 or 2 bp of extend in stem length, the order of importance to transposition efficiency: stem length, loop length, handle length and nucleotides sequences.

It was demonstrated that the CAST V-K system have a quite different transposition mechanism from the CAST I-F and I-B systems. The CAST V-K system can do the transposition even without a CRISPR array. The transposase in the CAST V-K system also shows no specific to the cas 12k in the system. The CAST V-k system substitute the cas 12k gene with dcas9, dcas 12a or even the cascade in CAST I-F systems could still be able to do on target insertion. Unlike CAST I-F or I-B systems, the distances between the target site and insertion site are also have large variation. It is therefore concluded that it is unnecessary for CAST V-K system to cross-talk with canonical cas systems at CRISPR array level to facilitate their horizontal gene transfer, either the random binding of the cas 12k or the stable R-loop forming by the canonical I-D or III-B systems' cascade could provide opportunities for CAST V-K system to move themselves.

### Materials and Methods

### Plasmid DNA Constructs

The DNA sequences of Scytonema hofmanni Cas 12k, TnsC, TniQ, and TnsB were obtained from pHelper_ShCAST (Addgene 127922); The DNA sequence of Anabaena variabilis ATCC 29413 cas5, cas6, cas7, cas8, tnsA, tnsB, tnsC, and tniQ were obtained from pHelper (Addgene 168137); The DNA sequences of Vibrio. cholerae HE-45 cas8/5, cas6, cas7, tnsA, tnsB, tnsC, and tniQ were obtained from pQCascade_crRNA-4 (Addgene 130637) and pTnsABC (Addgene130633). The DNA sequence of proteins of each system were PCR amplified and clone into the backbone of pTNS2 (Addgene 64968) by golden gate assembly. The repeat, spacer, chloramphenicol resistance cargo, left and right inverted repeat fragments were synthesized by IDT and also clone into the same plasmid by golden gate assembly.

### Cascade Expression and Purification

Plasmids carrying type I-F CASTs proteins and crRNA constructs were subcloned from conjugation assay using oligos. CAST type I-F cascade were co-expressed with TniQ and canonical type III-B crRNA in NiCo21 cells induced with 0.5 mM IPTG at O.D. of 0.6-0.8. Cells were cultured at 18 C for another 18-20 hours before harvesting. Cells were centrifuged and re-solubilized in lysis buffer containing 25 mM Tris pH 7.5, 200 mM NaCl, 5% glycerol, and 1 mM DTT. Protein was purified via its N-terminal maltose binding protein (MBP) tag using Amylose Beads and eluted by 10 mM maltose containing lysis buffer. The MBP tag with TEV cutting site on C-terminal was removed using TEV protease at 4° C. overnight. Sample was further diluted to 100 mM NaCl and put onto anion exchange column (5mL Q column HP) and eluted with 25column volume gradient of B buffer (A Buffer: 25 mM Tris pH 7.5, 100 mM NaCl, 5% glycerol, and 1 mM DTT. B Buffer: 25 mM Tris pH 7.5, 1000 mM NaCl, 5% glycerol, and 1 mM DTT.) TniQ-cascade was further purified by size-exclusion chromatography using a Superose 6 increase column (GE healthcare) in the SEC buffer composed of 25 mM Tris pH7.5, 200 mM NaCl, 5% glycerol, and 1 mM DTT.

### Scraped Colonies That Had Been Resuspended in LB Medium

Cells were pelleted by centrifugation at 16,000 g for 1 min and resuspended in 80 μl of H2O, before being lysed by incubating at 98° C. for 10 min in a thermal cycler. The cell debris was pelleted by centrifugation at 16,000 g for 1 min, and lysate supernatant was removed and serially diluted with 90 μl of H20 to generate lysate dilutions for PCR analysis. PCR products were generated with Q5 Hot Start High-Fidelity DNA Polymerase (NEB) using 1 ul diluted lysate per 10 μl reaction volume serving as template. Reactions contained 200 μM dNTPs and 0.5 μM primers and were generally subjected to 30 thermal cycles. PCR amplicons were resolved by 1% agarose gel electrophoresis and visualized by staining with Ethidium bromide (Thermo Scientific).

To map integration sites by Sanger sequencing, bands were excised after separation by gel electrophoresis, DNA was isolated by Gel Extraction Kit (Qiagen), and samples were submitted to and analysed by Eton.

### Conjugation-Based Transposition Assays

CRISPR array, Cas genes, transposon genes and Chloramphenicol resistance marker surrounding by left and right inverted repeat were cloned into conditionally replicative R6k plasmid (from Addgene: 111619). CAST I-F system's proteins and inverted repeat constructs were subcloned from #130637, #130634, #130633. CAST V system's proteins and inverted repeat constructs were subcloned from #127922, #127924. CAST I-B system's proteins and inverted repeat constructs were subcloned from #168137, #168146. R6k plasmid were transform into MFDpir which contain integrated rp4-based transfer machinery as donor strain. Donor strain grown in the presence of DAP (0.3 mM) and appropriate antibiotics at 37° C. overnight. Recipient strain grown in LB at 37° C. overnight. Gently spin down 1 mL culture (˜3000 rpm for 5 minutes) and wash donor and recipient cells in PBS. Measure optical density and combine 3:1 ratio of donor and recipient cells were plated on a non-selective plate which containing DAP (0.3 mM) for conjugation. The conjugation plate was incubated overnight at 37° C. Wash out each conjugation mixture into a micro centrifuge tube with 1 mL PBS, then vortex and gently spin down, repeat 4 times. Plate 100 uL of this mixture and 15 uL of a 10-fold dilution onto Chloramphenicol (12 ul/ml) plates and LB plate.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### REFERENCES

1. J. E. Peters, Targeted transposition with Tn7 elements: safe sites, mobile plasmids, CRISPR/Cas and beyond. Mol. Microbiol. 112, 1635-1644 (2019).
2. J. E. Peters, K. S. Makarova, S. Shmakov, E. V. Koonin, Recruitment of CRISPR-Cas systems by Tn7-like transposons. Proc. Natl. Acad. Sci. 114, E7358-E7366 (2017).
3. T. S. Halpin-Healy, S. E. Klompe, S. H. Sternberg, I. S. Fernández, Structural basis of DNA targeting by a transposon-encoded CRISPR-Cas system. Nature 577, 271-274 (2020).
4. N. Jia, W. Xie, M. J. de la Cruz, E. T. Eng, D. J. Patel, Structure-function insights into the initial step of DNA integration by a CRISPR-Cas-Transposon complex. Cell Res. 30, 182-184 (2020).
5. S. E. Klompe, P. L. H. Vo, T. S. Halpin-Healy, S. H. Sternberg, Transposon-encoded CRISPR-Cas systems direct RNA-guided DNA integration. Nature 571, 219-225 (2019).
6. B. Wang, W. Xu, H. Yang, Structural basis of a Tn7-like transposase recruitment and DNA loading to CRISPR-Cas surveillance complex. Cell Res. 30, 185-187 (2020).
7. M. Saito, et al., Dual modes of CRISPR-associated transposon homing. Cell (2021) https:/doi.org/10.1016/j.cell.2021.03.006 (Apr. 2, 2021).
8. S.-C. Hsieh, J. E. Peters, Tn7-CRISPR-Cas12K elements manage pathway choice using truncated repeat-spacer units to target tRNA attachment sites.bioRxiv, 2021.02.06.429022 (2021).
9. J. Strecker, et al., RNA-guided DNA insertion with CRISPR-associated transposases. Science 365, 48-53 (2019).
10. K. S. Makarova, et al., Evolutionary classification of CRISPR-Cas systems: a burst of class 2 and derived variants. Nat. Rev. Microbiol. 18, 67-83 (2020).
11. C. Camacho, et al., BLAST+: architecture and applications. BMC Bioinformatics 10, 421 (2009).
12. R. C. Edgar, PILER-CR: fast and accurate identification of CRISPR repeats. BMC Bioinformatics 8, 18 (2007).
13. V. Burrus, G. Pavlovic, B. Decaris, G. Guédon, Conjugative transposons: the tip of the iceberg. Mol. Microbiol. 46, 601-610 (2002).
14. A. Mark Osborn, D. Böltner, When phage, plasmids, and transposons collide: genomic islands, and conjugative-and mobilizable-transposons as a mosaic continuum. Plasmid 48, 202-212 (2002).
15. G. Faure, et al., CRISPR-Cas in mobile genetic elements: counter-defence and beyond. Nat. Rev. Microbiol. 17, 513-525 (2019).
16. NCBI Resource Coordinators, Database resources of the National Center for Biotechnology Information. Nucleic Acids Res. 46, D8-D13 (2018).
17. A. L. Mitchell, et al., MGnify: the microbiome analysis resource in 2020. Nucleic Acids Res. 48, D570-D578 (2020).
18. The UniProt Consortium, UniProt: a worldwide hub of protein knowledge. Nucleic Acids Res. 47, D506-D515 (2019).
19. E. V. Koonin, K. S. Makarova, Y. I. Wolf, M. Krupovic, Evolutionary entanglement of mobile genetic elements and host defence systems: guns for hire. Nat. Rev. Genet. 21, 119-131 (2020).
20. A. R. Parks, J. E. Peters, Tn7 elements: Engendering diversity from chromosomes to episomes. Plasmid 61, 1-14 (2009).
21. M. T. Petassi, S.-C. Hsieh, J. E. Peters, Guide RNA Categorization Enables Target Site Choice in Tn7-CRISPR-Cas Transposons. Cell 183, 1757-1771.e18 (2020).
22. Z. Li, H. Zhang, R. Xiao, L. Chang, Cryo-EM structure of a type I-F CRISPR RNA guided surveillance complex bound to transposition protein TniQ. Cell Res. 30, 179-181 (2020).
23. M. F. Rollins, J. T. Schuman, K. Paulus, H. S. T. Bukhari, B. Wiedenheft, Mechanism of foreign DNA recognition by a CRISPR RNA-guided surveillance complex from Pseudomonas aeruginosa. Nucleic Acids Res. 43, 2216-2222 (2015).
24. K. Y. Choi, Y. Li, R. Sarnovsky, N. L. Craig, Direct interaction between the TnsA and TnsB subunits controls the heteromeric Tn7 transposase. Proc. Natl. Acad. Sci. U. S. A. 110, E2038-E2045 (2013).
25. R. MH, et al., Type IV CRISPR-Cas systems are highly diverse and involved in competition between plasmids. Nucleic Acids Res. 48, 2000-2012 (2020).
26. H. N. Taylor, et al., Positioning Diverse Type IV Structures and Functions Within Class 1 CRISPR-Cas Systems. Front. Microbiol. 12 (2021).
27. J. Strecker, A. Ladha, K. S. Makarova, E. V. Koonin, F. Zhang, Response to Comment on “RNA-guided DNA insertion with CRISPR-associated transposases.” Science 368 (2020).
28. K. Y. Choi, J. M. Spencer, N. L. Craig, The Tn7 transposition regulator TnsC interacts with the transposase subunit TnsB and target selector TnsD. Proc. Natl. Acad. Sci. 111 ,E2858-E2865 (2014).
29. J.-U. Park, et al., Structural basis for target-site selection in RNA-guided DNA transposition systems. bioRxiv, 2021.05.25.445634 (2021).
30. J. E. Peters, “Tn7” in Mobile DNA III, (John Wiley & Sons, Ltd, 2015), pp. 647-667.
31. S. Ae, C. Nl, Avoiding Self: Two Tn7-encoded Proteins Mediate Target Immunity in Tn7 Transposition. EMBO J. 16 (1997) Available at: https://pubmed.ncbi.nlm.nih.gov/9362496/[Accessed May 19, 2020].
32. Z. Skelding, J. Queen-Baker, N. L. Craig, Alternative interactions between the Tn7 transposase and the Tn7 target DNA binding protein regulate target immunity and transposition. EMBO J. 22, 5904-5917 (2003).
33. A. W. Kingston, C. Ponkratz, E. A. Raleigh, Rpn (YhgA-Like) Proteins of Escherichia coli K-12 and Their Contribution to RecA-Independent Horizontal Transfer. J. Bacteriol. 199 (2017).
34. A. K. Aggarwal, Structure and function of restriction endonucleases. Curr. Opin. Struct. Biol. 5, 11-19 (1995).
35. A. B. Hickman, et al., Unexpected structural diversity in DNA recombination: the restriction endonuclease connection. Mol. Cell 5, 1025-1034 (2000).
36. K. Steczkiewicz, A. Muszewska, L. Knizewski, L. Rychlewski, K. Ginalski, Sequence, structure and functional diversity of PD-(D/E) XK phosphodiesterase superfamily. Nucleic Acids Res. 40, 7016-7045 (2012).
37. R. Missich, et al., The replisome organizer (G38P) of Bacillus subtilis bacteriophage SPP1 forms specialized nucleoprotein complexes with two discrete distant regions of the SPP1 genome. J. Mol. Biol. 270, 50-64 (1997).
38. T. Jacobsen, et al., Characterization of Cas12a nucleases reveals diverse PAM profiles between closely-related orthologs. Nucleic Acids Res. 48, 5624-5638 (2020).
39. R. T. Leenay, et al., Identifying and Visualizing Functional PAM Diversity across CRISPR-Cas Systems. Mol. Cell 62, 137-147 (2016).
40. T. Yamano, et al., Crystal Structure of Cpf1 in Complex with Guide RNA and Target DNA. Cell 165, 949-962 (2016).
41. I. Fonfara, H. Richter, M. Bratovič, A. Le Rhun, E. Charpentier, The CRISPR-associated DNA-cleaving enzyme Cpfl also processes precursor CRISPR RNA. Nature 532, 517-521 (2016).
42. Y. Jeon, et al., Direct observation of DNA target searching and cleavage by CRISPR-Cas12a. Nat. Commun. 9, 2777 (2018).
43. D. Singh, et al., Real-time observation of DNA target interrogation and product release by the RNA-guided endonuclease CRISPR Cpfl (Cas12a). Proc. Natl. Acad. Sci. 115, 5444-5449 (2018).
44. I. Strohkendl, F. A. Saifuddin, J. R. Rybarski, I. J. Finkelstein, R. Russell, Kinetic Basis for DNA Target Specificity of CRISPR-Cas12a. Mol. Cell 71, 816-824.e3 (2018).
45. D. C. Swarts, M. Jinek, Mechanistic Insights into the cis-and trans-Acting DNase Activities of Cas12a. Mol. Cell 73, 589-600.e4 (2018).
46. E. V. K. Sergey A. Shmakov, Systematic prediction of functionally linked genes in bacterial and archaeal genomes. Nat. Protoc. (2019).
47. S. A. Shmakov, K. S. Makarova, Y. I. Wolf, K. V. Severinov, E. V. Koonin, Systematic prediction of genes functionally linked to CRISPR-Cas systems by gene neighborhood analysis. Proc. Natl. Acad. Sci. 115, E5307-E5316 (2018).
48. D. Gleditzsch, et al., Modulating the Cascade architecture of a minimal Type I-F CRISPR-Cas system. Nucleic Acids Res. 44, 5872-5882 (2016).
49. K. Kuznedelov, et al., Altered stoichiometry Escherichia coli Cascade complexes with shortened CRISPR RNA spacers are capable of interference and primed adaptation.Nucleic Acids Res. 44, 10849-10861 (2016).
50. I. Songailiene, et al., Decision-Making in Cascade Complexes Harboring crRNAs of Altered Length. Cell Rep. 28, 3157-3166.e4 (2019).
51. D. Tuminauskaite, et al., DNA interference is controlled by R-loop length in a type I-F1 CRISPR-Cas system. BMC Biol. 18, 65 (2020).
52. V. Hoikkala, Cooperation between Different CRISPR-Cas Types Enables Adaptation in an RNA-Targeting System. mBio (2021) (Jun. 18, 2021).
53. J. N. Vink, J. H. Baijens, S. J. Brouns, Comprehensive PAM prediction for CRISPR-Cas systems reveals evidence for spacer sharing, preferred strand targeting and conserved links with CRISPR repeats. bioRxiv, 2021.05.04.442622 (2021).
54. G. Amitai, R. Sorek, CRISPR-Cas adaptation: insights into the mechanism of action. Nat. Rev. Microbiol. 14, 67-76 (2016).
55 B. D. Lavoie, G. Chaconas, Site-specific HU binding in the Mu transpososome: conversion of a sequence-independent DNA-binding protein into a chemical nuclease. Genes Dev. 7, 2510-2519 (1993).
56. FastQC, FastQC: A quality control tool for high throughput sequence data (2015).
57. N. A. Joshi, J. N. Fass, Sickle: A sliding-window, adaptive, quality-based trimming tool for FastQ files (Version 1.33) [Software] (2011).
58. D. Li, C.-M. Liu, R. Luo, K. Sadakane, T.-W. Lam, MEGAHIT: an ultra-fast single-node solution for large and complex metagenomics assembly via succinct de Bruijn graph. Bioinforma. Oxf. Engl. 31, 1674-1676 (2015).
59. M. Steinegger, J. Söding, MMseqs2 enables sensitive protein sequence searching for the analysis of massive data sets. Nat. Biotechnol. 35, 1026-1028 (2017).
60. S. Tansirichaiya, Md. A. Rahman, A. P. Roberts, The Transposon Registry. Mob. DNA 10, 40 (2019).
61. P. Pausch, et al., CRISPR-Cas@ from huge phages is a hypercompact genome editor. Science 369, 333-337 (2020).
62. S. Shmakov, et al., Diversity and evolution of class 2 CRISPR-Cas systems. Nat. Rev. Microbiol. 15, 169-182 (2017).
63. W. X. Yan, et al., Functionally diverse type V CRISPR-Cas systems. Science 363, 88-91 (2019).
64. L. Fu, B. Niu, Z. Zhu, S. Wu, W. Li, CD-HIT: accelerated for clustering the next-generation sequencing data. Bioinformatics 28, 3150-3152 (2012).
65. D. Burstein, et al., New CRISPR-Cas systems from uncultivated microbes. Nature 542, 237-241 (2017).
66. K. S. Makarova, et al., An updated evolutionary classification of CRISPR-Cas systems. Nat. Rev. Microbiol. 13, 722-736 (2015).
67. J. Daily, Parasail: SIMD C library for global, semi-global, and local pairwise sequence alignments. BMC Bioinformatics 17, 81 (2016).
68. C. Skennerton, MinCED (2019).
69. K. Katoh, D. M. Standley, MAFFT Multiple Sequence Alignment Software Version 7: Improvements in Performance and Usability.Mol. Biol. Evol. 30, 772-780 (2013).
70. L.-T. Nguyen, H. A. Schmidt, A. von Haeseler, B. Q. Minh, IQ-TREE: A Fast and Effective Stochastic Algorithm for Estimating Maximum-Likelihood Phylogenies. Mol. Biol. Evol. 32, 268-274 (2015).
71. J. Shi, C. Liang, Generic Repeat Finder: A High-Sensitivity Tool for Genome-Wide De Novo Repeat Detection1. Plant Physiol. 180, 1803-1815 (2019).

1. A non-naturally occurring system for RNA-guided DNA integration, the system comprising an isolated I-F CRISPR-Associated Transposon (CAST), wherein said CAST comprises:
a. TnsA-TnsB-TnsC; and
b. TniQ-Cas8-Cas5-Cas7-Cas6; wherein TniQ-Cas8-Cas5 are fused.

2. The system of claim 1, wherein the CAST components in a) are sequential or non-sequential.

3. The system of claim 1, wherein the CAST components in b) are sequential or non-sequential.

4. The system of claim 1, wherein the system further comprises a CRISPR RNA (crRNA), wherein the crRNA is specific for a target site.

5. The system of claim 1, wherein TnsA is represented by a sequence with 90% or more identity to SEQ ID NO: 6.

6. The system of claim 1, wherein TnsB is represented by a sequence with 90% or more identity to SEQ ID NO: 5.

7. The system of claim 1, wherein TnsC is represented by a sequence with 90% or more identity to SEQ ID NO: 4.

8. The system of claim 1, wherein TniQ-Cas8-Cas5 is represented by a sequence with 90% or more identity to SEQ ID NO: 1.

9. The system of claim 1, wherein Cas7 is represented by a sequence with 90% or more identity to SEQ ID NO: 2.

10. The system of claim 1, wherein Cas6 is represented by a sequence with 90% or more identity to SEQ ID NO: 3.

11. The system of claim 1, wherein the system further comprises a donor DNA to be integrated, wherein the donor DNA comprises a nucleic acid cargo sequence.

12. A non-naturally occurring system for RNA-guided DNA integration, the system comprising an isolated I-F CAST wherein said CAST comprises:
a. TnsA-TnsB-TnsC; and
b. TniQ-Cas8-Cas5-Cas7-Cas6.

13. The system of claim 12, wherein the CAST components in a) are sequential or non-sequential.

14. The system of claim 12, wherein the CAST components in b) are sequential or non-sequential.

15. The system of claim 12, wherein the system further comprises a crRNA, wherein the crRNA is specific for a target site.

16. The system of claim 12, wherein TnsA is represented by a sequence with 90% or more identity to SEQ ID NO: 6.

17. The system of claim 12, wherein TnsB is represented by a sequence with 90% or more identity to SEQ ID NO: 5.

18. The system of claim 12, wherein TnsC is represented by a sequence with 90% or more identity to SEQ ID NO: 4.

19. The system of claim 12, wherein Cas7 is represented by a sequence with 90% or more identity to SEQ ID NO: 2.

20. The system of claim 12, wherein Cas6 is represented by a sequence with 90% or more identity to SEQ ID NO: 3.

21. The system of claim 12, wherein Cas5 is represented by a sequence with 90% or more identity to SEQ ID NO: 7.

22. The system of claim 12, wherein Cas8 is represented by a sequence with 90% or more identity to SEQ ID NO: 8.

23. The system of claim 12, wherein TniQ is represented by a sequence with 90% or more identity to SEQ ID NO: 9.

24. The system of claim 12, wherein the system further comprises a donor DNA to be integrated, wherein the donor DNA comprises a nucleic acid cargo sequence.

25-182. (canceled)


SEQUENCE LISTING (458 sequences)

SEQ ID NO: 1 - protein, 1037 aa, synthetic construct
  REGION 1..1037: note = Description of sequence: tniQ-cas8-cas5 gene
MMLLQRPKSYPDESLESFFIRVANKNGYNDVHWFLVAVKRYLLDIDPRKFQTFPTDICCINPYSSKKHSISRTHALHHLSQLTFNEPVDLLGIALNRNQMQFSPSTTALIRGAEVIPRSLLRKGAIPCCPCCLGEHGYASYRWHFSGYEYCHEHDVKLIERCSCGAIYDYRYAGLSGVCTECGENISASQENHEPKATRIASWLAGDDVKPLPDVPLSYRWGFMHWWSQISSSCKTRNNGEFLAFWEHWPNSFHKLIGKEIDFNFEYCVLSKNDLRVKDILGKILFSSIQLPDRNFRSNIILKEMFQYIETHLWDDNGKLANLRMNMLEICVLLNCSREQVTSMIEQGLLPPNRQLGKREILIVTEYAFYLGDVYCLWLSEFQSDEFNRSFYLSRWRMATLAEILDNKTDDLNKDLRRAFRPLSAPVDISDTPIEALTILVNLTDRVIEQKNLLDRQKCKDKLRDEKWWANCFRTVKYRQSHNPKFPDIRANGVIRAAPVGHLPACMLSSSKLPQNSWAYANDSSQMNKSCFLTSEFIWNGDVHCLGQLLTELEHPLWNVLRKLGCYVKTAKYISKELALIPPLEINTSLVRNYLAQISLPNNEDSYISLSPVVSQSMQEDCYQVLSEHYRFSAITRFSRATNMGTLAMSCGGKFKMIRSLPPIEKYQHHHLDSVNWLTKRSVRAIRDYTESSVWVISPNKLALRKKSIIGDIKMMLSQWLRTTPTHEEKLDIRKLTERFNVDLAKTKFANRYAYDPLLTQLIYNCIGSIIHSPPQYAPKCEGNDDKYLLLPNLRISGASAMNTSVSIGIPSMMAFYGFVHAFQRNVQTANPNFKIESFAVCIHNIHVENRGLTREWVPNTKGQITAPATRDDWQCDVAVSLILRCSHYSQLIPRDFIRLLPGRIARGKVTVSISDIKHLGRCLSLADAIKAIPVETGRWLSLNNEVTLNSIQDVIDELKNNKLQTVNCIGYHRLETPCEKRGSLHGYKHAFVETILGIIKFLTISENTNPSQYFWQYHYSKQGPILLPRSVSDETS

SEQ ID NO: 2 - protein, 347 aa, synthetic construct
  REGION 1..347: note = Description of sequence: cas7 gene
MKLPNGLSYMKSIEASDVIFLVNWPDGRKTPLPYTSRVALGMKEGSKSAYKYDGQIDADVTAYSLAQGNPHEIDFCCVPYGAESIECEFSVSFASSLRKPFKCSDPEVKRTLVQLIKLYEEKVGWEELANRFLENICNGRWLWRNNECTYSTSIGIKPWPWEDEKAISPFHDIRKNYAGTNHFRDHKDWDNLIKLITDAFSQPNGLCIFEVSATFRLGTNAPIYPSQVFKDSVKGEKNRIYQSTDVDGESSPILGCYKTGAAIATIDDWYPDADKPIRISHYGAHREDVYCYRHPNTGKDLFTLLEKADQYLEQLQATDVLPDEMINDLHFIVANLIKGGLLQQKGT

SEQ ID NO: 3 - protein, 198 aa, synthetic construct
  REGION 1..198: note = Description of sequence: cas6 gene
MDWHYRTITFLPEYRNNEAIAAKCIKELHRFNYKYETRSIGVSFPLWGQETVGRKITFVSTNKMELDFLISRRYFVQMTKLGYFSISTTQTVPDDCSYVLFKRAHSIDKGTFAGRARELKRLERRALERGEIFDPIAYSKTTSHAFQSYHSLEEDSSSGNKFRLNIQMKERSGTVGTGKFSSYGLGNTDNSLQVVPLI

SEQ ID NO: 4 - protein, 249 aa, synthetic construct
  REGION 1..249: note = Description of sequence: tnsC gene
MEANDGSIIPILFTSLPANANPVTASERLLSSMGDPLAFSHGKDPAELMKIVNDLLRECRVELIIIDEFQHMIDRKSKDVLHSTADWLKMIIIDSKIPVVLFGMPYSTEILRVNNQLRGRFESQHHLKPFRVKDTSELIRYKTFMTMLDAALPFLEESGLASEDIMKRVYIFSKGNMRLIRRLINKAAKFALLENAPCISLKHFARAAPKVSRDACKSFNPFDTDTKKLKIIEPPEDVGWENYLAAKGD

SEQ ID NO: 5 - protein, 624 aa, synthetic construct
  REGION 1..624: note = Description of sequence: tnsB gene
MSDDSENLYAFGSFFPEKHSNTSVPKTSKGTRFGIELQESYQDLFSFDEKRRDEAIHRYNILDYLIELHGPSLTLKKISGSMKGLADKFHPNVPSAPSIYRYWTTFKKSGFVLSSLIPGVTRGNTKQRKTLELEEYIERAIKSYFSAESPTIQQAFTLLETEIDRHNECNDTQLSFEYESFRKRIVKKTDYERLLIKKGKKAADTYYKKVGQRPETTRVLQRVEADHTRLDLFVIDDARKLPLGRPWLTLLFDTHTKSVVGFYLGFEPPGYLSVSLALENAILPKYYVKELYPEVKGEWPCYGLPEHLIVDNGAEFNSKDFVTACKNLRIKVKKNPVKKPWLKGSVERYFRTINNKLLSGIPGKSFSNIFARGDYNPQKNAIITRSDLMKVIHVWLIDIYQSSPNGLENNIPNLSWADAMRSAFPPRSFNGSIDELRFNLGKHVEISLDRNGIRLKKTLRYTSSYLAQYFGKHTYDGKSIKVKIKYNPICMGSIYVLDEDKHEFFAVESVDPDYAYSVSEWLHKVCCDYARNHIRNNYRHNDVIKAWRVIYDIIDEALHLSGNGKQANVGIRQASKLERVREHAERTKSHQKPELHMSSNDDIDWDVEVNTDGWKIDSVRGTNK

SEQ ID NO: 6 - protein, 207 aa, synthetic construct
  REGION 1..207: note = Description of sequence: tnsA gene
MKKRIIKNSKVKNISRFVSLKTDSVQTTESDLEFDACFHFEFASHVKSFETQPLGFEYRLNGRLRRYTPDMLCYFNDGYATYYEVKPKWVTERDEFKKKFDAQKQQAIANGYDLLVLTEDDIQTYPLLDNLKIIHRYACSDSLDDVQVRILKLFQNYGEMRISQVINASQGQSASILPALYDLIAKKILEFDWHCPISHDSLVWRVS

SEQ ID NO: 7 - protein, 420 aa, synthetic construct
  REGION 1..420: note = Description of sequence: cas5 gene
MQEDCYQVLSEHYRFSAITRFSRATNMGTLAMSCGGKFKMIRSLPPIEKYQHHHLDSVNWLTKRSVRAIRDYTESSVWVISPNKLALRKKSIIGDIKMMLSQWLRTTPTHEEKLDIRKLTERFNVDLAKTKFANRYAYDPLLTQLIYNCIGSIIHSPPQYAPKCEGNDDKYLLLPNLRISGASAMNTSVSIGIPSMMAFYGFVHAFQRNVQTANPNFKIESFAVCIHNIHVENRGLTREWVPNTKGQITAPATRDDWQCDVAVSLILRCSHYSQLIPRDFIRLLPGRIARGKVTVSISDIKHLGRCLSLADAIKAIPVETGRWLSLNNEVTLNSIQDVIDELKNNKLQTVNCIGYHRLETPCEKRGSLHGYKHAFVETILGIIKFLTISENTNPSQYFWQYHYSKQGPILLPRSVSDETS

SEQ ID NO: 8 - protein, 180 aa, synthetic construct
  REGION 1..180: note = Description of sequence: cas8 gene
MATLAEILDNKTDDLNKDLRRAFRPLSAPVDISDTPIEALTILVNLTDRVIEQKNLLDRQKCKDKLRDEKWWANCFRTVKYRQSHNPKFPDIRANGVIRAAPVGHLPACMLSSSKLPQNSWAYANDSSQMNKSCFLTSEFIWNGDVHCLGQLLTELEHPLWNVLRNSDVTLKQPNTYQRS

SEQ ID NO: 9 - protein, 396 aa, synthetic construct
  REGION 1..396: note = Description of sequence: tniQ gene
MMLLQRPKSYPDESLESFFIRVANKNGYNDVHWFLVAVKRYLLDIDPRKFQTFPTDICCINPYSSKKHSISRTHALHHLSQLTFNEPVDLLGIALNRNQMQFSPSTTALIRGAEVIPRSLLRKGAIPCCPCCLGEHGYASYRWHFSGYEYCHEHDVKLIERCSCGAIYDYRYAGLSGVCTECGENISASQENHEPKATRIASWLAGDDVKPLPDVPLSYRWGFMHWWSQISSSCKTRNNGEFLAFWEHWPNSFHKLIGKEIDFNFEYCVLSKNDLRVKDILGKILFSSIQLPDRNFRSNIILKEMFQYIETHLWDDNGKLANLRMNMLEICVLLNCSREQVTSMIEQGLLPPNRQLGKREILIVTEYAFYLGDVYCLWLSEFQSDEFNRSFYLSRW

SEQ ID NO: 10 - protein, 207 aa, synthetic construct
  REGION 1..207: note = Description of sequence: tnsA gene
MFDQTKKSSHVHNICKFMSLKNDAVVRTLSILEFDFCFHLEYNADIKSFTSQPFGFHYQFNNRKCRYTPDFLATDHDDHSTFFEVKHSSQILKPDFRDRFKEKQRVALNDFNRRLVLVTEKQIRVGPTLDNFKLLHRYSGLRTVTEFQKLVLAFIQCKQMVKLQEVSLYFGLSEQDTLISTLPWISSGQVKTDLNGINFGLETYVWC

SEQ ID NO: 11 - protein, 621 aa, synthetic construct
  REGION 1..621: note = Description of sequence: tnsB gene
MSSDSNNIFGFFDEFEVSEGESQSLPIELFQEPVEILSTLDSQPERIQQEVIRRIKIIAFVEKRLKGGWIEKNLNPILSLVESELQLTPPSWRTLATWKKSYFEAGKDPCALIPKHAFKGNRQKEMDSQSLIDEAIQNVYLTRERLSVADAYRYYKSRVIQINRGIVEGKIKPISERAFYNRIHDLPPYEVAIARFGKRYADREFRSVGQQVAATKPMEYVEIDHTPVPVILIDDELDIPLGRPYLTMLYDRFSKCIVGCSINFREPSFDSVRKALLNSLLDKSWVKQRYPCIDNEWPCHGKIDCLVVDNGAEFWSQSLEDSLRPLVSDIQYSQAAKPWRKSGIEKLFDQMNKGLVNALPGKTFTNPTQLQDYNPKKDAVVRVSVFLELLHKWIVDYYHMAPDSRERDIPYHKWNQSEWTPSYYSGAEKEQLRVELGLLRHRTIGVAGIRLHNLRYQSSELIEYRKYWASNNGKKLFVKTKTDPSDISYIHVYLESEKRYIKVPAADNSGYTKGLSLFEHERIQKVRRLNTKQLADDEGLADTFLYLKKRIQEETDRLHSAKSNKSSLPKTGNTSKLAKFNDVGSEGPSSINTTPVKQETTVVFDVSERLSDDDFEDIEGY

SEQ ID NO: 12 - protein, 371 aa, synthetic construct
  REGION 1..371: note = Description of sequence: tnsC gene
MSQSGYLMMILKILRGINLNLTSRQFEQLRSFETCFIEYPAIAEIYSIFDQLRFNHSLGGEPESFLLTGETGSGKTALINNYLSRFPASSTWSKQPVLSTRVPSRINEQSTLTQFLVDLNGKSGGRGTRRLNEIALGEAVVTQLKRKSVELIIVNEIQELVEFSTAEERQAIANTFKYISEEAKVSFVLVGMPYADVIATEPQWNSRLSWRRKIHYFKLLKANSHLSGSGPYGFDLEQKKHFAKFVAGLSARMGFDEPPLLTKNEVLFPLFVMCRGECRALKHFLKDALLMSFNENVDTIDKALLSRTFSFKFPYLDNPFNCPVEDLCFHQIDSGSSYNVNAIALEDKILAPRFTDAIPLSMLLTKNGLKA

SEQ ID NO: 13 - protein, 335 aa, synthetic construct
  REGION 1..335: note = Description of sequence: tniQ gene
MRVRVLIHLENQLKLINFGVLRLALSHSKAQFSPQYKAVHRFGVDYPYAFLRKRFTPICPLCIDETPYIRQQWQFISHQACEHHGCKLVHHCPECKSRLEYQSTDSINQCECGFELRNSPVEDAPEAEILVARWLSGNDSKPLGLLTGEMTLSERYGFLLWYVNRYGDIDDLSFESFVEYCSAWPTALWQDLDTLKEKAELVRVKDWKKVFFNEAFEALLKDCRQLPNRQLSHNTVLTLVLAYFTQLMATVPSSAKGNIGDVLLSPIEASTLLSCTTDEVYRLYEFGEIKAAIRPRMHTKIASHESAFTLRSVIETKLTRMSSESDGLSLYLPEW

SEQ ID NO: 14 - protein, 695 aa, synthetic construct
  REGION 1..695: note = Description of sequence: cas5 gene
MTKLSGLLAIEDEAVKQVTLKKMFMPYTEDVCVEGFEKEALTILLNLSSSHQADRCSDWLDLARAKRHLKAAENLEASLDEIKWFHTHNLKFPDCRVKDQRIVAQPLTTTEVFISSGVLEQRLGWAHNSAVYRHTLWLLNPFNWQSQPVCILSLIQQESPVWVELLKEFGLGAKSLARLKHTIEEQLLDNHFPDSVSPYSKQLRFPWGDGYISVTPVVSHAIQREFEVRSRSRESKLSFVSSSLPNSASIGNLCGSLGGHMKVLNHPLDVKPAQGGTLTESRKKSGRYFDDYQVTNAKICKVLNHLIGSEPSKIQKQRESARKVRSKILRKQIALWMLPLIELRDIVDADPNQQQLEHDDTLAQAFLALPESDLGSLASEFNRRLHLVFQNNKYTAKFTYHPKLMQLVKAQIVWILERLSKPSGNEDKVTGEQYIYLSSMKVQDAVAMSSPYLCGAPSLTAIWGFMHRYQREFNKLVNSNSLFEFSSFSFYVRSENIQSTAKLTEPNSVAKARTVSNAKRPTIRSERLADLEIDLVIRVHSESRISDFKAALKTALPVAFAGGALYQPQLSTQIEWLRTFTSRSELFHVIKGLPAYGRWLYPSDNQPNDFDDLEHLITKDADNLSVSIGYHLLERPTKRCNSITDCHAYAENAIGLAKKVNPIEVRFSGRDHFFNHAFWSIECSSETILIKNYRD

SEQ ID NO: 15 - protein, 217 aa, synthetic construct
  REGION 1..217: note = Description of sequence: cas7-1 gene
MELCTQLNYVRSLSAGKAYFYYLSKSGEMCPLEIDRTRLRAPKGGYAEAYKGGKFVEKNVAPQDLAYSNPQFIEECYVKPGVDDIYCAFPLRIRANSLTPDTCSDYEVRSKLSLLAKTYEELNGYQELALRYAKNILLGSWLWRNRECRTLSIEVTTSDSQTLIVENAARLSWYGHWDEASAECLEKLTAYLTRALSDPTEYFYMDVKAKIGVGWGG

SEQ ID NO: 16 - protein, 138 aa, synthetic construct
  REGION 1..138: note = Description of sequence: cas7-2 gene
MSKLKLESVGGDEVYPSQEFLDSREDDVPTKQLATVELLNGKETVAFHGQKIGAALQSIDDWWHEDADKPLRVNEYGADREYVIARRHVAHGNDFYQLLRNTEKWIETMIASQTIPNDVHFIMSVLIKGGLFNCSKAK

SEQ ID NO: 17 - protein, 203 aa, synthetic construct
  REGION 1..203: note = Description of sequence: cas6 gene
MIMTKRYYFCIRYTPVQADYELLAGRCISQMHLFMVNNRQTINKIGVSFPDWSDVTVGQTIAFVAEDKEMMIGLSFQPYFSLMVNEGLFEISSVFEVPDNAIEVRFTRNQTIGKSFLGSKKRRVKRSMVRAELLDAGTSLPVTNEERIVDSFHRIPISSGSSGENYMLFVQKELVSERGAANFNSYGLATNQERKGTVPELRV

SEQ ID NO: 18 - protein, 147 aa, synthetic construct
  REGION 1..147: note = Description of sequence: tniQ gene
MKPDSYEPLEWYDLKLLILPKFLYLIFLTINFSSSVFASPENLAKAQPHQQKRISPRSCDLNQIENFLNQHLEQSPDLISPLTIIAKNLKVDKKFISRYFRDLCQKIVAKRLSYKKDCYLDSLKQCCNEVEQVTKMLFKQGEYPTED

SEQ ID NO: 19 - protein, 401 aa, synthetic construct
  REGION 1..401: note = Description of sequence: tnsC gene
MVMALQGFPPELLNQSPQKRLDFFKSPDITFMHKILHKTYNQLYRKVCKPAGASTILLIGPSGVGKSTLMDLLKKRILEESLPQMELEPAWIPIICVEADAPGKGTFRWKKFYQQVLLEVDEPGIKYKIDYNKINYEEEGIRRGNNGRLIIGARATEDSLKISMKQALKYRHPYTLAIDEFQHIGIRANEDLLKAHMDCLKSVVNGTKIPLTGFGTYELLEFLELSPQLSRRTLKIHFHRYRWENDDECKEFKRVLYNLIHRMPFPNQPTITKQIWEFCFERSIGNIGTLIDWLTDAYDLALANDAPSLSFDYLKETAKSRLDCEQMIEEAIEGEARLEETEDSTNRLKVLLGMKSASKPSKSNANSTDPNSTAQKTKKDVKNKPFKRKPVRDQVGGQDET

SEQ ID NO: 20 - protein, 735 aa, synthetic construct
  REGION 1..735: note = Description of sequence: tnsB gene
MVKANPGIYLSELQQKIEIASIDDFHVLILTDKIYVDLFAIPLSCPEQVQVFLSATEAFTHSQLTTIVPSNWETFNSPLNVAIGTQISWDGQPWTIVNLGNNYVGLQGENNTFQHLPNSVFSTLVHQEKIVGLSSPNFSSLNPKIEEIFSYTSQADLQEAHRRYQQIEAFLKGTASTPTNRTQRRWLASYRKAEQLYGRGFIGLIPQHRHKGNSLSKMDKDVHQLMEKHIQEEYETHKQPSIRHAFQAFKELCAHTGLRPPSLETYRRSIRNRPVVEQVKKRQGSRAAYQEETFYEFLDRNTPIHGERPFEIAHIDHTLADVQLLSLIMLNLGMEMNEGEHQANLGKAWVTLLIDAYSRRILAAYMTFDSPSYRSNMMVLRICVQRFGRLPQILVVDGGRDFQSGNFEMLLAYSHVTKKERANTRDGSVIESFFGVADREFWHNLKGNTQIMKKVRQVTKSVNPKYHAVWTLDKLYRYFCEYCYEIYDTCPHPTLRMTPREAFEIGLARGGLRGHLLIPYEEFKLLSLPAPTDNDGMRRITPKGIKINYLYYGHPSFERVRNTRVAVKYEPFDVTIAYAYIKGEWVQCRSGYIRELQGRSVKELITASDELKKLNQLQNQQFSDITGKNLAQFFTRIEREEAILTPSWREAKKAVQAQHLRDEELKEVLNEIEGKPSTKETSNDSSEIDLFTFENREYIALSSSISPPSPIQQTQVDEDDDNDEFEELFEPLEEW

SEQ ID NO: 21 - protein, 194 aa, synthetic construct
  REGION 1..194: note = Description of sequence: cas6 gene
MEAISIHPIAGIPQPLNQLRLTNRSRLRIRLPIDQIPLVYPLAGQTITIDWGIERVSHQAKFQLGIPYPTALQPASTLYSRLVLIHRAETPEAVLEVAQRQLNRKGIKGTIELPTRANGEVQCRLLTIKKKHQLFRLRGFGMKVTKLSEKDSIKLQQSGIGGKHKMMCGVFVPTWRETTEKDENSNVLVIYSGR

SEQ ID NO: 22 - protein, 324 aa, synthetic construct
  REGION 1..324: note = Description of sequence: cas8 gene
MLDKYNKIQEKPEFALVLLFVPVVCQYFIFHPYYEKTDNNNPEKYLIVIPEILDLEEAAYQRWQSGNLAYKNYHVSSLGEAALITYSWHEVETQRSCERTCQVLLYEKFNKDSHQRSLIETRDITINLNILNTYFLIKQFFQENQFDSYGKNRWIKVNFIRGIIADNLVKGVPWWSDFWKILNQEDIDGELAQQLIDNKEGLIAMIEQDKEMEDYKLFVQAFHEALRRNYAKIQKRTQEGERARFDREYERIRGELNRCYDEHSFSDFLADFLARAGFLSTLYDEWDSVLPFITESVPWQKARNIALFALATYKPVSKNQLEGE

SEQ ID NO: 23 - protein, 301 aa, synthetic construct
  REGION 1..301: note = Description of sequence: cas7 gene
MFYLFGTVITHYGPASLNHEKARGNVSPLQKTYWDGHIHSLVSGDAIRWGLRYTLQKEGYPINRRWNEDEFTNDLVNENFDPVEFVDDDLFGYMYAEAAKEDIQEEEPSSTGKERRNQTKGSIVQRLGALGVNRGVSLIPYTGALTFNAKSGKIKDRTALHFLEYHNTRYQYTFALDPNHLKVKERILAFIDALMNIRKVGGNNNVFCYKFTPESFVLCWTQRETPGIFYCFEEGDLIPKMKSEVLEDIEVGDIKASELWIGGRVAKGLKIKNAHIFRGKEQAIEDLKKVIVKDLELSYPY

SEQ ID NO: 24 - protein, 211 aa, synthetic construct
  REGION 1..211: note = Description of sequence: cas5 gene
MSSIAVYIDVPFASFRQSHSREYGKTYPVPPPATVYGMLLSLVGEWDVYLHCGVQLAMAMISQPQISKVLRRIRRFKVKNFSDDRNTNPEYQEVLSNIKFIVWVASKNEQAQPTLRERIEQALIYPESVKRVGCLYLGESNDLVNKIKLISNDYCFEERWWLMQDEDGLIILPYWVDHVGSKNTRWLRYGLKKISVEFPPESCWTTIQAIR

SEQ ID NO: 25 - protein, 343 aa, synthetic construct
  REGION 1..343: note = Description of sequence: tniQ gene
MSNYNELDDGLEIVSLKSPETPKRSRLFSLEPVGIGTSQTESLSSYVNRLAEAHCVKSQKLIMLEIAPQILGKDYQSPLHSKDVSSLFGNTDAKPALNGMRDMKLLLVQALEKLTQRQDLKYLTCLSWKEVINKRGLFRQYRAWCPQCYEQCKQDKVSLYDPLLWSFKDVKFCLHHQCQLVDECPKCGSHLPVIANFLPLGHCFYCGEWLGDNKNKNPKCNFKNFPESLLIIKNIGDLIAVTPRLGSQPTLDEFIQKLQLILFCFEKVISQDLKHIKVLGKIIEHLKLNLSQHGDKPLNLIKIIIPVCSKAHISVSELFLEDLTGLSKILFDNFQINYQLPIL

SEQ ID NO: 26 - protein, 60 aa, synthetic construct
  REGION 1..60: note = Description of sequence: tnsA gene
MDTSKSVEPKQKKKLKTRGRGSGKEYEPFIKIHEISSSGESVRIRSATVGRTHHLLSGIE

SEQ ID NO: 27 - protein, 60 aa, synthetic construct
  REGION 1..60: note = Description of sequence: tnsB gene
MRCSMHLNLVYTDPSTKCRFRVVREQISELMLIDIDDANAWPFPIEEEQLKALEYERIDD

SEQ ID NO: 28 - protein, 60 aa, synthetic construct
  REGION 1..60: note = Description of sequence: tnsC gene
MMMTRLVNAQYREAEILDYEGHPLITALPPINSPQATAEMLSLTPNIPEAEKTLPPHIRR

SEQ ID NO: 29 - protein, 60 aa, synthetic construct
  REGION 1..60: note = Description of sequence: cas7/csf2 gene
MNTTPLAYQLSGHITAEAPLNVSYFGMDERLPRTPHGQVFLNGGTLRGPLRKAGLNMLIR

SEQ ID NO: 30 - protein, 60 aa, synthetic construct
  REGION 1..60: note = Description of sequence: cas5/csf3 gene
MAVPSQGGDPALMQPFKLILQLHTPLILPRVVPRLDTLLHEAACRRALDWEHPPSLPLTY

SEQ ID NO: 31 - protein, 60 aa, synthetic construct
  REGION 1..60: note = Description of sequence: cas8 gene
MLVGSGGIITPEAELYLTLPPKLFEKYRDGEWGQQGRLSIEKPFERLISLLIQGVLDPIE

SEQ ID NO: 32 - protein, 60 aa, synthetic construct
  REGION 1..60: note = Description of sequence: cas6 gene
MQVHYYEASALCHPGNTSERPVTLNDYHRTAWHLFSGDTHYPQRERPFIFRAQWLTQEQH

SEQ ID NO: 33 - protein, 684 aa, synthetic construct
  REGION 1..684: note = Description of sequence: cas8 gene
MNWIKNLAELYDMMDRKEDRDPRLLPLYHSAVNADITVTIDSQGNFLRAEKLDKVEFGEKDFRMTRVPGTPDSLSRSNQIAPRAMMDRLMYVAGDFDDLLPAAYEGAYRKHHNSYMKQMQDWISSPFSCEAPETVCRYLEKNTFLHDIAGPILLPMLSKATDKEQAEQAVKEDRVFEFVKELYAKGCEQKGTVAKKKTGMALDKLNVRWCIAADEKDEHPAETWRLDNVANCWGAYMHDVMERIGTRGFDHISCKDNVVLVKNWFPWVMAPTCNLSVISGNRQDCQYRFGPYSNSKQMYGVGLETADKAMRMLRYLIEVQGKRYYDRNGITGAIAIWANGSSGCEMPDLDTIFEVEKPDEETVWGRLYHSRMSRTLSGIRDITQAEGAKTVGLMEFRKTSPGRVAIVQCVEISVDELCSNLQQWYENAEWLQTMYQQKKWFQAMGVPTALTIARYGTLARGTCMVGKKNDNTNITYNWYSQLVKCIFFHEQVPMYMVDNLVSLATAIPNSSEKKVGQVYFERKIGIACSMAAAYYNYKEKVYEPMLNREIMDRNYLFGRAWACVEFICSEHRYYQGRKGKHQVNMTFAEKNISGFSNQPCDVLGQALKMVKGVYVPQYPFLEWAANELEEIISCLDAESEYTNSPLRANWILGYSSEKMDLKARKKAIVEAKTQKEKMPEAIAN

SEQ ID NO: 34 - protein, 244 aa, synthetic construct
  REGION 1..244: note = Description of sequence: cas5 gene
MENDKDYTKFCIRATGVTALFTSPETKLPGERRSSWYPYYEQLKNMIQAIFWKPEICIVVDRCRIMNKARFQTGGVLETDFGGAGHLDGHQPVAQTMLCNVEYIIEFHIVENMSDYAQKRIAERVKNGGEPWSRKKYEDIMERAITKGTHRIPYFGTSDCPAVIRPCDDFFAGEGYYDHTSCDIGLVYHGMNYPEQAGDGIKAAADNTVERRFTHLKMENGIIEFPTPEKCEFVQVIDLGKKRS

SEQ ID NO: 35 - protein, 292 aa, synthetic construct
  REGION 1..292: note = Description of sequence: cas7 gene
MKDYSFISVIECRNCNPNGDPLNDNIPRNDGRGHAEMSPVCIGHKLRVEAEFAKEKIFLTGSGADGHNCLNDRTQAAYPDFNKEKKQSLVAAQKKQAEALNKAFWDCRMFGFLVPSSNNGEKKALSIQMAQTVDPVDFRDIQITKCVGLEAPKAVEKKNANGDGEGEYNLTRSSDTMGMLHIIDFGLMVIRGRICGDFAEEHGVTEKDIELLKDCLVNMFEHDGCLARPAGSMMVRNVFWFEPENKRLMESKVYDAFHAEHKEGVDKPQRYQDYNIWVDDIPGVRCTDLTEG

SEQ ID NO: 36 - protein, 415 aa, synthetic construct
  REGION 1..415: note = Description of sequence: tniQ gene
MKRVPMLPTPYPDELWYSVVCRYHRRAGNIRLWTTFEELFGNLSKKTVSIAGLDQTIDSYIKVRHLPDDARTSYISENTLVPFLLRYYTPESRREVTQTAWTGKTKLNVSFIRPERYKQALVLRYCPECCAEDEDTYGEMYWHRSHQIPMMSTCPKHYCLLCDSKVSWRQASMKLFCADEMVCPKIDAQFPPKNSIENKLDKYLLVCLQAPYSENDITPTSEISNTLLMNGYAVWNQKGYRINTTAVYQIVSQQFDLRGEKGGYGKLLTHILNEERNSKAEYVAMLAAAINVPVSSFMVKGENNNHKTVEARVLELSKRIGDRQASKIRIEQKLGISTEQLNAAVKRLGIKPFWKRHALDLDGEKRTHKVLIHITQAERAEMEKRAEDLCMGTVAEYVRSCIMEDLKNAAGTAGR

SEQ ID NO: 37 - protein, 444 aa, synthetic construct
  REGION 1..444: note = Description of sequence: tnsC gene
MEYTNDVNAVYTNALTGNPLLDALPGRLTLSELYEKVSYIPTLPDNYKTMPPNDRYLLAEKLTMMYMPLDYTAIIYNLIYSGIRSAYQGKTPKGIIKRTNECGQCIFYKTPNRLSDSITQAECFSVLGESGMGKTQTITRILKLFPQVIHHTEYDGQPFEHDQIVYIKIESPANNSPRGACLQILAAVDELLGTDLCLEERKKSSNVDMLITRIAQTCIRYSIGCIVIDEIQNILSTRSNYIQTTGGRMVNFLVELANKTGVCLAFIGTPIVSKVFDSEPHLLRRTRGPRIPALEKSTAFEKLLEIMWNNIPVLNPVPLDAGTIDLVYKITGGVIAKMQKEILLSTQNAIYFEKETVTRELLKKIAKQYNIIPGKSTLETAAPEVLTVKATKEKNETSLDSKGKGRPKLSWDDDDILQIFAECQEKGWSVAKALIQHGLAERGD

SEQ ID NO: 38 - protein, 601 aa, synthetic construct
  REGION 1..601: note = Description of sequence: tnsB gene
MVKFYGDENATYRLLAKENGIWLINVSGKTAPVCISKEVFAQMHEVPADEVIPPHTPPQWVREEANKRVEIIRPLTESEQCIVDPVKRANISNQIAKEQEISSRTVYRYYYSYLAKGIYGLFPTQRKRKTNKKTPDQTNIQKALNQFFYSPYRMSLRMAYEMMLLNYYKDESGILGKEYPTYNQFRYYFRKHRDSKKESILREGIGEHQKNRRPLMGAGNSGVDYIGLYEMDATLADVYLVSHYDRQLIGRPYIYMAIDVASRLIAGIYVGLEGNSNSVMACLANAAMDKVQFCQKYGIDIEESQWPSQGMPGCICTDRGNEFTGEVTKSLCEMYRVEIIGLPPYRPDLKGYVEKAFDCLQNRYKPLLHGMGVIEDPSTANGAPDYRKQACLDLHEFTKILIQCVLYYNSSNVLNDFVRTPEMKQDNVQPFACNVWNWYKKAGATRLVNLDPEGLRLMLLPRGVAQITRYGLKFNSLFYTNEKYEAMFIKGASKGNVTIAYDESDTGTIYIIENGEYIPFSLTMASRKYDGLTMMEMKALLDGEKEVIRGARNRQIEGSLSCNEHILQITKSARQRKQEESSGKKDTSTQTMKLKRKKEKV

SEQ ID NO: 39 - protein, 137 aa, synthetic construct
  REGION 1..137: note = Description of sequence: tnsA gene
MEGKIMRKKDDKTRTISFYSQKNHQVIVVANAMAKKYALAMEKDDSVIKYEVNIPLTDFAILVDGKGLRRKLLETEWVSDFMVVRQSGTKIVEIVDEEKLENKRSCSERLELSRRYWKAAGAVDWQAVIIQKGETAW

SEQ ID NO: 40 - protein, 301 aa, synthetic construct
  REGION 1..301: note = Description of sequence: tnsB gene
MYMQEAPSSKTPANSNSTLTEANVIVSELSDEAKLKMEVIQSLLSAGDRTTYAERLKEAAEKLGKSVRTVRRLIDKWEQEGLVGLVQGQRADKGKHRVDENWQEFILKTYKEGNKGSKRMTRQQVAVRVKARADELGVKPPSHMTVYRVLKPLIDKEEKAKSIRSPGWRGSRLSVKTRDGKDLQVEHSNQVWQCDHTLVDVLLVDSSGKILSRPWLTTVIDSYSRCIMGINLGYDAPSSTVVALALRHAILPKQYSSEYALNEEWGTSGLPQHFYTDGGTVLGKCRGKILSGRWIFGRSKF

SEQ ID NO: 41 - protein, 98 aa, synthetic construct
  REGION 1..98: note = Description of sequence: tnsC gene
MGYFWFAATFLYGWGYRFGKMSGEDFKRTVDIWEKQILKLPVASNLSSKTMLKTLGEATGGYIGLMDMILRESAIRALKNGLQKIDLETLKEVATEYK

SEQ ID NO: 42 - protein, 167 aa, synthetic construct
  REGION 1..167: note = Description of sequence: tniQ gene
MEAEDIKPWLFQVEPLEGESLSHFLGRFRRANELTPTGLGKAAGLGGAIARWEKFRFNPPPSRQQLEALATVVGVDADRLEQMLPPAGVGMKIEPIRLCAACYAQSPCHKIKWQFKVTQGCASHNLSLLSECPNCGARFKVPALWMDGWCHRCFLNFVEMVGYQKLV

SEQ ID NO: 43 - protein, 656 aa, synthetic construct
  REGION 1..656: note = Description of sequence: cas12k gene
MLLVYIYVSKRMSQITIQCRLVASESTRQQLWKLMAEKNTPLINELLLNVPQHQDFETWRQKGKHPTGIVKELCQPLKTDPRFISQPARFYASAIATVNYIYFSWLALMKRLQYKLEGKTRWLEMLRSDEQLVEASGVTLDSLRIKANEILAQLSPQSALVEAKEKKGNTVKKTRKSQNSDNNRSLSATLFQAYRDTEDILTRCAISYLLKNGCKVSDKEEDPEKFAQRHRKIEIQIERLREQLEARIPKGRDLTDTKWLDTLFVATHQVPNNQAQFISWQDSLLRQSSSVPFPIAYETNEDMTWFKNHQGRICVKFNGLSEHTFEVYCDQRYLHWFQRFLEDQQIKHESKNQHSSSLFTLRSGRITWFQEEGKRVRGSAAAAVPWNIHRLSLYCCVDTRLWTDEGTGLVRQEKAEEIAKTITKTKEKGDLNEKQLAHIKRKNSTLARISNPFPRPSKPLHKGQSHVLVGVSLGLEKPATVAVVDATTGKVLIYRSIKQLLGNNYKLLNRQQKQKHSLSHKRQIAQTLAAPNQFGESELGQYVDRLLASEIVAIAQTYSAGSIVLPKLSDMREQVQSEIQAKAEQKSDLIEVQQKYAKEYRVSVHQWSYGRLISSVQSQAAKAGIVIESAIQPIRGSPHQKAKELVIAAYHSRQKT

SEQ ID NO: 44 - protein, 560 aa, synthetic construct
  REGION 1..560: note = Description of sequence: tnsB gene
MLDDHLNGEQEPEINEIVTELSADDRHLLDLIQKLLEPCDRITYGERQREVAAKLGKSVRTVRRLVKKWEEEGLAALQTTTRVDKGKHRIDSDWQQFIIKTYKEGNKGSKRITPQQVAIRVQARAAELGQQKYPSYRTVYRVLQPIIEEQEQKAGVRSRGWHGSRLSVKTRDGKDLSVEYSNHVWQCDHTRVDLLLVDQHGELLGRPWLTTVVDTYSRCIIGINLGFDAPSSQIVALALRHAILPKHYGSEYGLHEEWGTYGKPEHFYTDGGKDFRSNHLQQIGVELGFVCHLRDRPSEGGIVERPFGTFNTDLFSTLPGYTGSNVQERPEQAEKEACLTLRELERLLVRYIVDKYNQRPDARLGDQTRYQRWEAGLIASPNLISEEELRICLMKQTRRSIYRGGYLQFENLTYRGENLAGYSGETVVLRYDPRDITTVLVYRQKGNKEEFLARAFAQDLETEQLSLDEAKASSRKIRQAGKMISNHAMLAEVRDRETFITRKKTKKERQKAEQAVVEKAKKPVPVQPEEEIEVASVDGETEYQMPEVFDYEQMREDYGW

SEQ ID NO: 45 - protein, 279 aa, synthetic construct
  REGION 1..279: note = Description of sequence: tnsC gene
MTLKQAQTVAQQLGDIPVNGEKLQVEIDRLNRKTFILLEQVKILNDWLEGKRQARQSGRIVGESRTGKTMGCDAYRLRHKPKQEVGKPPLVPIAFLEDIPSECSAKDLFNEILKHLKYQMTKGTVAEIRERTFRVLKGCGVEMLIIDEADRLKPKTFAEVRDIFDKLEIAVILVGTDRLDAVIKRDEQVYNRFRACHRFGKFSGEDFKRTVEIWEKQVLKLPVASNLSNKTMLKTLGEATGGYIGLLDMILRESAIRALKKGLPKIDLETLKEVAAEYK

SEQ ID NO: 46 - protein, 171 aa, synthetic construct
  REGION 1..171: note = Description of sequence: tniQ gene
MEVPEIQPWLFQIEPLEGESLSHFLGRFRRANDLTPTGLGKAAGLGGAIARWEKFRFNPPPTPQQLAALAVVVAVDVDRLKQMLPPLGVGMKMEPIRLCAACYVESPCHKIDWQLKVTQRCASHNLTLLSECPNCGARFKHPALWVYPWCHRCFFRFTDMVKYQKFVYSSL

SEQ ID NO: 47 - protein, 719 aa, synthetic construct
  REGION 1..719: note = Description of sequence: cas12k gene
MCFIDERMSQITIQCRLVASESTRQQLWKLMAEKNTSLINELLVQVAQNPEFETWRQKGKHPSSIVKELCQPLKTDPRFIGQPARFYTSAIATVNYIYKSWFALIKRSHFQLEGKIRWWEMLKSDTELVELSGMTIESLRSKAAEILAQFTLKSDTVEAKPATENKRKKTKKSKASDSDRSMSKTLFEAYHDTKDILTSCAISYLLKNGCKINDKEEDTQKFAKRRRKLEIQIERLREQLEARIPKGRDLTDAKWLETLLLATRNVPKSELEAKSWQDDLLKKSSKLPFPVGYESNEDMTWFWQFSLYNIAINLRFFWLWIYIDYLIAIVFLRDVLQIEKQWLHNLEINNTSLLIKLWNLALELNCLASILFLHESLHKYKRRLCVQFNGLSKHTFKVYCDFRDLHWFQRFFEDQRIKKNSKNQHSSSLFTLRSGCICWQEHEGKGKEWIVNRLILYCCVDTRLWTAEGTNQVREEKAEEIAKAITRTIARGELNEKQLAFIRRKNSTLARINNPFPRPSQLLYKGQSHILVGVSLGLEKLATVAVVDGSTGKVLTYRSIKQLLGDNYKLLNRQRQQKHTLSHQRQIAQILAAPNQLGESELGQYVDRLLAKEIVAIAQTYKAGSIVLPKLGDMREQVQSEIQAKAEQKSDLIEVQQKYAKEYRVSVHQWSYGRLIANIQTQAAKVGIVIEEAKQPVRVSPQEKAKELAIAAYNYRQKT

SEQ ID NO: 48 - protein, 580 aa, synthetic construct
  REGION 1..580: note = Description of sequence: tnsB gene
MDEMPIFNQNDESLLFENNADIDEIQDDESEEANLIFTELSAEAKIKMEVIQGLFEPCDRKTYGQKLRTAAEKLGKTVRTVQRLVKKYQQDGLSAIVDTQRNDKGSYRIDPEWQKFIITTFKEGNKGSKKMTPAQVAMRVQVRAEQLGLKKYPSHMTVYRVLNPIIERQEQKQKQRNIGWRGSRVSHKTRDGQTLDVRYSNHVWQCDHTKLDVMLVDQYGEPLARPWLTKITDSYSRCIMGVHVGFDAPSSQVVALALRYAILPKQYSAEYKLLSEWRTSGIPENLFTDGGRDFRSEHLKQIGFQLGFECHLRDRPSEGGIEERSFGTINTEFLSGFYGYLGSNIQERSKTAEEEACLTLRELHLLLVRYIVDNYNQRLDARTKDQTRFQRWEAGLPALPKMVRERELDICLMKKTRRSIYKGGYLSFENIMYRGDYLAAYAGENIVLRYDPRDITTVWVYRIEKGKEVFLSAAHALDWETEQLSLEEAKAASRKVRSVGKTLTNKSILAEIHDRDTFIKQKKKSQKERKKEEQAQVHSVYEPINLSKTEPLENLQETPKPETRKPRVFNYEQLRQDYDE

SEQ ID NO: 49 - protein, 278 aa, synthetic construct
  REGION 1..278: note = Description of sequence: tnsC gene
MKDDYWQKWIQNLWGDEPIPEELQLEIERLLTPSVVELEHIQKIHDWLDGLRLSKQCGRIVAPPRAGKSVTCDVYRLLNKPQKRGGKRDIVPVLYMQVPGDCSSGELLVLILESLKYDATSGKLTDLRRRVQRLLKESKVEMLIIDEANFLKLNTFSEIARIYDLLRISIVLVGTDGLDNLIKKEPYIHDRFIECYRLPLVSEKKFPELVKIWEEEVLCLPLPSNLIRNETLLPLYQKTGGKIGLVDRVLRRASILALRKGLKNIDKDTLAEVLDWFE

SEQ ID NO: 50 - protein, 167 aa, synthetic construct
  REGION 1..167: note = Description of sequence: tniQ gene
MEIGAEEPRFFEVEPLNGESLSHFLGRFRRENYLTSSQLGKLTGLGAVISRWEKLYFNPFPTRQELEALATVVRVNADRLTEMLPLKGVTMKPRPIRLCAACYAEYPCHRIEWQFKDKMKCDRHNLRLLTKCINCETPFPIPADWVEGECSHCFLPFATMAKRQKSR

SEQ ID NO: 51 - protein, 635 aa, synthetic construct
  REGION 1..635: note = Description of sequence: cas12k gene
MSVITIQCRLVAEEETLSQLWELMADKNTPLINELLAQVGKHPDFETWLEQGKIPTELLKTLVNSLKTQERFAGQPGRFYTSAIAIVDYVYKSWFALQKRRKHQIEGKERWLTILKSDQQLEQESQCSLNVIRTKAIEILSQFTPQSDQNKNQRKSKKTKKSAKLHKSSLFQILLNTYEQTQDPLTRCAVAYLLKNNCQISELHEDPEKFTRNRRKKEIEIERLKDQLQGRLPKGRDLTGEEWLETLEIATDNVPQNENEAKAWQAALLRKSAEVPFPVAYESNEDMTWLKNDKGRLFVRFNGLGKLTFEIYCDKRHLHYFQRFLEDQEIKRNSKNQYSSSLFTLRSGRLAWLPGEEKGEPWKVNQLHLYCALDTRMWTTEGTQKVINEKSIKITETLTKAKQKEDLNDKQQAFITRQQSTLDRIHNPFPRPSKPNYQGQPSILVGVSFGLEKPVTVAVVDVVKNEVLAYRSVKQLLGKNYNLLNRQRQQQQRLSHERHKAQKQNAPNSFGESELGQYVDRLLADAIVAIAKSYQAGGIVIPKLHDMREQISSEIQSRAENKCPGYKEAQQKYAKEYRMSVHRWSYGRLIDSIKSQAAKVGISTEIGTQPIRGSPQEKARDLAVFTYQERQAALI

SEQ ID NO: 52 - protein, 344 aa, synthetic construct
  REGION 1..344: note = Description of sequence: tnsB gene
MGFYLGFDNPSSQIDALALRHAILPKSYDSEYQLHDEWPAYGKPNYFYTDGGKDFTSIHTTEQVAVEIGFSCALRRRPSDGGIVERFFKTLNDQLISTLPGYTGSNVQERPDDVDKYACLTLKNLKICLVRYIVAEYNRHADSRMKNQSRIGRWEAGLMTEPYLYDELDLAICLMKQERRKVQKYGCVQFENLTYRADHMKGRDGETVVLRYDPADVTTLLVYKINTDGTEEFLDYAHAQGLETEHLSLRELKAINKQLKEAREEINNGSILEAMLDRQAFVEQTVKQNRKQRRQAANEQVNPVESVAKKFAIPEPKEVETENELNTELPKYQVRYMDEFFEED

SEQ ID NO: 53 - protein, 279 aa, synthetic construct
  REGION 1..279: note = Description of sequence: tnsC gene
MTNERALAKQFGVLEEPTPEIQAEIERLSREPYLELDQVKRCHAWMYELVISKMTGLLVGESRCGKTVTCKTFALSYNKLRQAKGQRMKPVVYIQAPKNCGSRDLFTKILKALNKPSNGTISDLRERTLDSLAIHQVEMLIIDEANHLKLETFSDVRHIYDDDDLRIAVLLVGTTSRLLAVVKRDEQVVNRFLEQFELDRLEDTQFKQMIQIWERDVLRLPEESKLASGDNLKLLRQATKKLIGRLDMILRKAAIRSLLRGQKKVDKDVLKEVIAASKL

SEQ ID NO: 54 - protein, 173 aa, synthetic construct
  REGION 1..173: note = Description of sequence: tniQ gene
MEKNLDNKRQFWLTRVEPFDGESISHFLGRFRRAKGNKFSAPSGLGHVVGLGAVLVRWEKFYFHPFPTQQELEALAAVVELDSDRLRQMLPPVGVGMKHSPIRLCGACYAESPCHKIEWQFKKTGGCDRHQLRLLSKCPVCEKPFVIPALWVEGQCSRCFTSFAEMAQYQKPY

SEQ ID NO: 55 - protein, 646 aa, synthetic construct
  REGION 1..646: note = Description of sequence: cas12k gene
MSQITIQCRLVASESTRQQLWKLMADLNTPLINELLEQLAKHPDFEKWRQKGKLPASVVMKLCQPLKNDPRFAGQPSRFYISAIHIVDYIYKSWLAIQKRLQQQLDGKTRWLEMLNSDPELVEASASSLEVIRTKAAEILAIAMTASDSDNSSPNGKKSKKAKKASSNPKRKLFNTLFDAYQETEDVLSRSAISYLLKNGCKLSDKDEDPEKFAQRRRQLEIQIQRLTEKLTSRIPKGRDLTHAKWLETLFTATTTVPKDNAEAKHWQDILLTRSSSVPFPVVFETNEDLVWSTNEKSRLCVHFNGLSDLTFEVYCDSRQLHWFKRFLEDQQTKRKSKNQHSSGLFTLRNGRLAWQQGEGKGEPWQLHRLTLYCCVDNRLWTAEGTEQVRQEKAEEITKFITKMNEKSDLSDTQFAFIKRKESTLTRINNSFDRPSKPLYQGQSHILVGVSLGLEKPATVAVVDAMPDGKADANAGKVLTYRSIRQLLGDNYKLLNRYRREQRSLSHERHKAQKSFSPNQFGASELGQYIDRLLAKEIIAIAQTYKVGSIVLPKLGDMREIVQSEIQAIAEAKCPGSLEIQQKYAKQYRVNVHSWSYGRLIQSIQSKAAQAGIMIEAGKQPIRGSPQDKAKELALSAYHDRLARRS

SEQ ID NO: 56 - protein, 208 aa, unidentified
  source 1..208: note = Vibrionaceae family
MYVRNLRKPSATKNVYKFASSKNRSVILCESSLERDCCYHLEYSKDVFSFQSQPEGFYYSSSGNKRCPYTPDFLVRNQDGSEYYLEVKPLAKTFSEDFKRSFALKRIAAQHQGKPLVLVTDKQIRNGVYLENLNLIHRYSGLVDFSLSSTKIVEELSAAGRMCIRSLADNLKLSIGEVIAVVFRLIGLGRVNVPLDSAINEMSVISVN

SEQ ID NO: 57 - protein, 360 aa, Pseudoalteromonas sp.
MVMPFDDEFESINDDTQAEYDSTSEAKLVRKQYLPLDSVTIHERDLSSFSEEQKNKALERYKLISAVAKEISGGWTPKNINPLIDKYGLNLSIKRPSYKSVIRWYKSFCGSDGNIVCLVDHNHSKGNRTKRIIDDESFFVEATERFLDAKRPNYSQAYQFYCDRIEIENSNIISGQISKVSYQAFKERLKKLPPYEVALKRFGQNYANKLFNYYQSSVPTTRILERVELDHTPLDLILLDDDLLIPLGRAYLTLLVDVFSGCIVGFHLGFNPPSYVSVAKAIIHSVKSKDYVHDLNIELTNDWLCHGKMETLVVDNGAEFWSKSLDQACMEAGIHYEYCKVGQPWEKPRVERKFLEIIQG

SEQ ID NO: 58 - protein, 328 aa, Rhodococcus sp.
MNLSAKQEIAVDELLTQYHNSFVIYPDVQQIFDGLDWIVRRSQFGNFTPSMLITGGTGAGKTSLINHYAKYHFNDNEVLITRVRPSFIETLIWAIDKLGIPYNTRSKRSEIGLQDYFINSVKKSNLKLLVIEEAQELFECASPKERQKIRDRLKMISDECRLPIVFIGIPTAKLILEDSQWDRRIMVKRELPYIRITSESSLDVYIDLLEELEKQLPISVQPELSEMDIAMRLLSATKGMLGAIKELVGYALELALLSGKSAITNDEFALGFERINGPDVTNPFTTELEKLLVPQVIEYEGFIIDPENGEIKFTKQIFKDIPLAALLG

SEQ ID NO: 59 - protein, 335 aa, Vibrio navarrensis
MRVRVLIHLENQLKLNNFGVLRLALSHSKAQFSPEYKAVHRLGSDYPVVFLGKRFTPICPLCISEAPYIRQQWQFLSQQACERHGCKLVHHCPECQSRLEYQTTESISQCECGFELRNSPIEDAPVAALLVARWLSGNDSKPLGLLKAEMTLSERYGFLLWYVNRYGDIENISFESFVEYCSCWPRALEEELDELVNKADLIRIKDWKKTFFNEVFGTLLKDCRQLPSRQLERNSVLTQVLAYFTKLMATVPLSSKGNLGDVLLSPLEASTLLSCTTDEVYRLYEFGEIKAAIRPRMHTKIASHESAFTLRSVIETKLTRMCSENDGLSVYLPEW

SEQ ID NO: 60 - protein, 540 aa, synthetic construct
  REGION 1..540: note = Description of sequence: Cas protein
MTKLSDLLAIEDEAIKQTALKKMFMLYTEDVCVDGYEQETLTILLNLSSSHQADRCSDWLDVARAQRYLKDRENLDASLAEIQWFHTHNLKFPDCRVKDQRIIARPLSTAEEFISSAVLDQRLGWAHNSAVYRHTLWLLNPFKWQSQPVCILSLIQQKNPVWLDLLKEFGLDVKSLAHLQSAIEEQLPENSFPDSVSTYSKQLRFPWGDDYVSITPVVSHAIQCELEIRARSPENKLSFVSSSLPNSASIGNLCGSLGGYMRVLNYPLGVKQAKGGTLTASRQKSGHYFDDYQVTNAKICQVLNRLIGSEPSKTQRQRERARQVRSKILRKQIALWMLPLIELRDIAESEPNQQQLEHDDTLAQAFLSLPELELGSLASEFNRRLHLTFQNNMYSAKFAYHPKLMQVAKAQVTWVLEQLSKPINNQDTVTGEQYIYLSSMRVQDAAAMSNPCLCGVPSLTAIWGFMHDYQRKFNQLVNNDSPVEFSSFAFYVRNENIQSTAKLTEPNSVAKARTVSNAKRPTIRSERLVDLEIDLVIRVH

SEQ ID NO: 61 - protein, 343 aa, synthetic construct
  REGION 1..343: note = Description of sequence: Cas protein
MELCTQLNYVRSLSAGKAYFYYLSESGEMCPLNVDRTRLRAPKGSYSEAYKGNKFVDKNVAPQDLAYSNPQFIEECYVKPGVDEIYCAFSLRIRANSLTPDICSDDEVRSKLSMFAKIYKELNGYKELANRYAKNILLGTWLWRNRECRNITIEVTTSELDTFVVEHAQKLSWYGHWDGDSTECLERLTAYLERALSDPTEYFYMDVKAKMRVGWGDEVYPSQEFLDSREDGIPTKQLATVELLSGKETVAFHGQKVGAALQSIDDWWHEEADKPLRVNEYGADREYVIARRHVAYGNDFYQLLRNTENWIESMTASESIPNDVHFIMSVLIKGGLFNCAKAN

SEQ ID NO: 62 - protein, 212 aa, synthetic construct
  REGION 1..212: note = Description of sequence: Cas protein
MSQRYYFLIRYTNANADYGLLAGRCISQMHLFMVNHHQAMNRVGVSFPDWNESSVGQTIAFVSEDKEMMIGLSFQPYFSLMVNEGLFEISSVYEVPDTSEEVRFVRNQTIGKNFLGSKKRRIKRSMARAELFGIQQSLPVTNEDRVIDSFHQIPISSGSSRQDYILFIQKELADERAESGFNSYGFATNQEKRATVPDLRFNPFDEDSFLVQ

SEQ ID NO: 63 - protein, 363 aa, Vibrio tapetis
MDQHEAMAGAFPLELNRVNIYHAQTTSQMRVRVLIHLENQLKLNNFGVLRLALSHSKAQLSPQYKAVHRFGVDYPYAFLRKRFTPICPLCINEAPYIHQQWQFISHQACEHHGCKLTHHCPECSSRLEYQSTESIGQCECGYELRNSPVDDAPEAETLVARWLSGNDSKPLGLLKAEMTIPERYGFLLWYVNRYGDIDDLSFESLIEYCGSWPTALWQDLDALKEKAELVRVKDWKKMFFNEAFGALLKDCRQLPSRQLSHNIVLAQVLAYFTKLMATVPSSAKGNIGDVLLSPLEASTLLSCTTDEVYRLYEFGEIKAAIRPRMHTKIASHESAFTLRSVIETKLTRMSSESDGLSVYLPEW

SEQ ID NO: 64 - protein, 338 aa, Rhodococcus sp.
MESTQKREEQLVSQPMSNSHIDLVLSGYHDSFTIYPEIKKVFSGLDWLIKRRKFGAFVPSMLLTGGTGAGKSALIKYYVANNLSDKEVLITRVRPTLHETLLWIAKELSVYKNSKAKASDIGLETHVSTCIKRTDLKLLVIEECQELFECTTHNARQNIRDRLKMISDDCRLPIVFVGLPSAKLILEDSQWKRRIMVKRELPYIKITDEPSMDRYMDLLEAMEKSVPMPFEIDLTDVDLAMRLLAASHGMVGMLKELIASALESALLAKQSVIRVDDFAIGHEAIFGIDVSNPFTVEIDHLAIKQIESYEEYVPDAETGELKFVGQIFNNLSMKQLLG

SEQ ID NO: 65 - protein, 360 aa, Pseudoalteromonas sp.
MSRHFKDEFDPIHSEDAEQEFLSLPETKRTQLQYDSLDNTKTYERDLASFPPEQKEKALQRYKLLCLVHKDLKGGWTPKNLNPLIDQHIDKTWLPKPSYKTLIRWHQAFSGSHGSFTSLVDKNYLKGNREPRVTGDEVYYERALERFLDAVRPSINAAYQVYCDEIIIANETIISGKIPTVSYQSFKNRIKKEQPYSVALARHGKYYADKLYHYYQSVQMPTRILERVEIDHTPLDLILLHDDLLIPLGRAYLTLLVDVFSGCIIGFHLGFKAPSYVSASKAILHSIKKKDYISSLPIEFQNEWPCSGKIENLVVDNGAEFWSKSLEDACTESGINIVFNKVRKPWLKPFVERKFGEIIQ

SEQ ID NO: 66 - protein, 260 aa, unidentified
  source 1..260: note = Vibrionaceae family
MTCVPGCGWVSTQPSVAEIISCPKKTITCPFKSHKVSYALFGCFFTTKIGTRMYIRNLRKPSPNKNIFKFASAKNKDTIMCEGSLEKDCCYHFEYDADVVSFESQPKGFLYDFDGKQLPYTPDFFVIYTDGCYSFVETKPYSKTLSKEFKLKFRARKQAAKKLGFDLILVTDKQIRTGYFLKNSEMVHRYSGCIEGDSLTDAVLEELLISKAIKIYELAQRISSSIGEVFASVLRLISVGKARTDFDLEMLNENSIISVN

SEQ ID NO: 67 - protein, 201 aa, synthetic construct
  REGION 1..201: note = Description of sequence: Cas protein
MTKRYYFYIRYIPAHADFELLAGRCIHQMHMFMVNNPQVMNRIGVSFPSWGESSVGQTIAFVAEDKDMMIGLSFQPYFSLMTKEGIFEIFNVCEVPENLGEVRFIRNQTIEKNFLGSKKRRIKRSIARAELSGGEQSLPVAVEERVVDHFHRVPISSGSSGQDYILFTQKEFAEERTARSFNSYGLATNEERRGTVPDLRF

SEQ ID NO: 68 - protein, 348 aa, synthetic construct
  REGION 1..348: note = Description of sequence: Cas protein
MELCTQLNYVRSLSAGKAYFYHLSKSGEMCPLEIDRTWLRAPKGGYAEAYKEGNKFVAKNVAPQDLAYANPQFIEECYVKPGVDDIYCAFSLRIRANSLNPDVCSDDEVRYKLSSLAKTYKELNGYSELAYRYAKNILLGTWVWRNRECRKLTIEVKTSDSETVVIENATRLSWYGHWDEASTECLEKLTAYLKQALSDPSEYFYMDVKAKIGVGWGDEVYPSQEYLDSREDGVPTKQLATIELTSGKETVAFHGQKVGAALQSIDDWWHEEADKPLRVNEYGADREYVIARRHVTLKNDFYQLLRNTENWIESMAASQTIPNDVHFIMSVLIKGGLFNRSKSKPKAK

SEQ ID NO: 69 - protein, 540 aa, synthetic construct
  REGION 1..540: note = Description of sequence: Cas protein
MTKLSDLLAIEDEVVKQASLKKMFMPYTEDVCVEGFEKEALTILLNLSSSHQSDRCSDWLDVARTKRHLKAAENLEASLDEIKWFHTHNLKFPDCRVKDQRIVAQPLATAEVFISSAVLDQRLGWAHNSAVYRHTLWLLNPFSWQSQPVCILSLIQQESPVWIELLKEFGLGAKSLARLKHTIEEQLPDNHFPDSVSTYSKQLRFPWGDDYVSVTPVVSHAIQSELEVRSRSQESQLSFVSLSLPNSASIGNLCGSLGGYMKVLNYPLNVKPAQGGTLPESRKKSGHYFDDYQVTNAKICQVLNHLIGSEPSKTQKQRESARKVRSKILRKQIALWMLPLIELRDIVDADPNQQQLEHDDTLAQAFLTLPESDLGSLASEFNRHLHLTFQNNKYAAKFAYHPKLMQVVKAQIVWVLEQLSKPNGNEDKVTGEQYIYLSSMRVQDAVAMSNSYLCGAPSLTAIWGFMHHYQREFNKLVNCDSPFEFSSFSFYVRSENIQPTAKLTEPSSVAKARTVSNAKRPTIRSERLADLEIDLVIRVH

SEQ ID NO: 70 - protein, 333 aa, Rhizobium mesoamericanum
MLTDKQKEKLNEFRDVFIEYPIISTVFNDFDRLRLGKGLAGEKPCMLLNGDTGTGKTAIIKQYKERHLPQFINGVMNHPVLVSRIPSNPTLESTLAELLKDLGQVGSTERKLRINGTRLTTSLIKCLKTCGTELIIIDEFQELIEHNQGKKRREIANRLKYINDEAGVSIVLVGMPWAEKIADEPQWSSRLLVRRQLPYFKLSENPKHFVQLIIGLANRMPFIEKPNLSEQAIVFALFSLSKGCFRTLKYFLDDAVLYALMDNAKTLTTKHLVKAFDVLFPDVPNLFTLPVSEITATEVERYSLYKPESALDEDPFIATKFTDRMPISQLLRK

SEQ ID NO: 71 - protein, 360 aa, Pseudoalteromonas sp.
MFNDGLFDDEFNQPLPKVETKLPQNYAKDLQALPEKIKNTTFAKLKYIQWLEANIQGGWTQKNLEPLLKVMPEVEGEKKPSWRTAARWYSAYTNADKNIMALIPSHQKKGNRERDTATDKFFEKALERYLVKEKPSVASAYKYYADLVIIENDSVVGSVLKPLTYKAFKNRIDNLPQYEVMIARYGKRLADIAFNKVEGHTRPTRVLEKVEIDHTPLDLILLDDELHIPLGRPTLTMLVDVYSHCIVGYYFSFSEPSYDAVRRAMLNAMKPKSEVVKLYPDTINEWKCAGKIETLVVDNGAEFWSNSLELACEEIGINTQYNPVAKPWLKPFVERMFGTINTELLDPIPGKTFSNILQKH

SEQ ID NO: 72 - protein, 216 aa, synthetic construct
  REGION 1..216: note = Description of sequence: Unknown origin
MVRWLTHMYRRKLKHSRVKNLHKFASQKNKSTCLVESSLEFDACFHFEFSPLIAAFEAQPLGYEYEFDNRTCRYTPDFLLTHTDGTQKFIEVKPQSKIADEDFRARFIEKQAIAKQDGRDLILVTDKQIRVYPTLNNLKLLHRYSGFQSLTELQASVLALVKQCGSIKVGQLVSFLKVTAGELLATVLRLLSLGQLFADLTTNEISIETAIWSNNV

SEQ ID NO: 73 - protein, 360 aa, Clostridium homopropionicum
MTTNPRPNRAQQQSLRAFLKQQSKPAAMWLKLSIALGTVNAILMIAGAYLLAQTIHDVMFEGRNLAQVTHFLWPLAGIILLRAIFLALSERLSAFAALKIKSAIRQTLLDKLTLLGPSYIEQHGQGATLNTLHNGVEALHDYYAKYLPGVAYSALIPLAILVVIFPTDYKAGLIFLLTAPLIPFFMILVGHKAEALNQKRWQQLAVLGNYFFDRIQGLTQLKLFNATRKELKQIAQISDDFRHATLSVLKIAFLSSFALEFLATISVALVAVIIGFRLFFGTLDFATGFVVLLLAPEFYLPLRQLGSHYHARLQGISAAADMLIILNAPLPSKNNNHTAHINMEGNSTISVHDLNFSYPN

SEQ ID NO: 74 - protein, 169 aa, synthetic construct
  REGION 1..169: note = Description of sequence: Cas protein
MSSREISNIGVCFPKWNEQTIGNQIAFVSTDKKQLTNLSQQSYFEMMAHDKLFDLSKILEVPTNQSDVMFVRNQSVAKAFVGEKQRRLKRAKKRAEARGEVYNPKYKFEAKDIGHFHSIPVSSKGNGQSYILHIQKIEKTEPINNQFNNYGFATNQTFQGTVPSLNTLF

SEQ ID NO: 75 - protein, 342 aa, synthetic construct
  REGION 1..342: note = Description of sequence: Cas protein
MQLPRHLSYSRSLSPSKAVFFYKTPESDFEPLQIEQNKLVGQKSGFGDAYQKQGEAKNLAPQDLAFGNPQTIDVCYVPPTVNELFCRFSLRVEANSNEPHVCDDPKVIYWLKRFFETYKKHNGFNDVATRYAKNILMGNWLWRNRQSPNVDIEILTEHAAPIIVEGVQKLKWQGNWQNNQTALLTLSEAIQEGLSNPQNYCYLDITAKIKNAFSQEVHPSQKFVDNVEQGMSSKQLAYTQVGDKKAASLNSQKVGAAIQTIDDWYEGGYKPLRTHEYGADKQILVAHRTPKSHSDFYSLLPRIAMHIKHMEKHGLGQSEQSDAVHFIAAVLIKGGLFQRSKG

SEQ ID NO: 76 - protein, 540 aa, synthetic construct
  REGION 1..540: note = Description of sequence: Cas protein
MNLQDAFAIELLKEKTTAIRKLFAPYMPHVAVDGFEEQALTVLINLVYKRSEIDDLTSTRTAQSVLRDEVLLSKCINEVKWFHTHNLKYPDIRVSHQRLISDVISEDIAGICSRSLPLSFGWSHNSAEINHAKLFLTSFSWQGEVTCLAKLLITEEPVWINLIRAYGFTKKTVLDIAGKIKQQLPVAELPLEVSSFSPQLQMPFQQSYLAVTPVVSHAMLAKIQQLRTDRKLNFGFVEHSRPANVGDLASSVGGNIRVLRYFPKTYSKAINRSKVANNDIEKAFKIRALLSSQFQQALLVLVGIKQFNTLRQKRLARVAAIRQVRVSLQLWLDNILEAKNNAHEQAYPEWAKHYLDQSITNCISQFSNVLNESLGNLSKLKRFAYHPNLMGLFKSQLNYVFTHCATEKETLNDEQIVYVHCQDMRVFDAEAMANPYIQGMPSLTALNGLAHNFERKLKNFIDPSIKCIGSAIYIENYQLHKGKPLPEPSKLKQVAGRSHVIRSGIIDKPKCDITLDLVFRFFVPNEEILNKLNSQLIKPA

SEQ ID NO: 77 - protein, 95 aa, Comamonas sp.
MPAKKAPNAIDKPKEYVSNPANKDTNNVVNTNSSSLRASATSLNTLGKTHLPIAHSNTHTNAPLVSANSNAQPILLGSLLANKGKIMSNGTTAMS

SEQ ID NO: 78 - protein, 212 aa, synthetic construct
  REGION 1..212: note = Description of sequence: Cas protein
MSQRYYFLIRYTNANADYGLLAGRCISQMHLFMVNHHQAMNRVGVSFPDWNESSVGQTIAFVSEDKEMMIGLSFQPYFSLMVNEGLFEISSVYEVPDTSEEVRFVRNQTIGKNFLGSKKRRIKRSMARAELFGIQQSLPVTNEDRAIDSFHQIPISSCSSRQDYILFIQKELADERAESGFNSYGFATNQEKRATVPDLRFNPFDEDSFLVK

SEQ ID NO: 79 - protein, 343 aa, synthetic construct
  REGION 1..343: note = Description of sequence: Cas protein
MELCTQLNYVRSLSAGKAYFYYLSESGEMCPLNVDRTRLRAPKGSYSEAYKGNKFVEKNIAPQDLAYSNPQFIEECYVKPGVDEIYCAFSLRIRANSLTPDICSDDEVRSKLSMFAKIYKELNGYKELANRYAKNILLGTWLWRNRECRNITIEVTTSELDTFVVEHAQKLSWYGHWDGDSTECLERLTAYLERALSDPTEYFYMDVKAKLRVGWGDEVYPSQEFLDSREDGIPTKQLATVELLRGKETVAFHGQKVGAALQSIDDWWHEEADKPLRVNEYGADREYVIARRHVTYGNDFYQLLRNTENWIESMTASESIPNDVHFIMSVLIKGGLFNCAKAN

SEQ ID NO: 80 - protein, 540 aa, synthetic construct
  REGION 1..540: note = Description of sequence: Cas protein
MTKLSDLLAIEDEAIKQTALKKMFMPYTEDVCVDGYEQETLTILLNLSSSHQADRCSDWLDVARAQRYLKDRENLDASLAEIQWFHTHNLKFPDCRVKDQRIIARPLSTAEEFISSAVLDQRLGWAHNSAVYRHTLWLLNPFKWQSQPVCILSLIQQKNPVWLDLLKEFGLDVKSLAHLQSAIEEQLPENSFPDSVSTYSKQLRFPWGDDYVSIPPVVSHALQCELEIRARSPENKLSFVSSSLPNSASIGNLCGSLGGYMRVLNYPLGVKQAKGGTLTENRQKSGHYFDDYQVTNAKICQVLNRLIGSEPSKTQRQRERARKVRSKILRKQIALWMLPLIELRDIAESEPNQQQLEHDDTLAQAFLSLPEWELGSLASEFNRRLHLTFQNNMYSAKFAYHPKLMQVAKAQVTWVLEQLSKPINNQDTVTGEQYIYLSSMRVQDAVAMSNPCLCGVPSLTAIWGFMHDYQRQFNQLVNNDSPVEFSSFAFYVRNENIQSTAKLTEPNSVAKARTVSNAKRPTIRSERLSDLEIDLVIRVH

SEQ ID NO: 81 - protein, 401 aa, Vibrio tapetis
MDIQLYPDESLESFLLRLSQEQGYERFSHFAEDIWFDTMEQHEAIAGAFPLELNRINIYHAQTTSQMRVRVLIHLENQLKLNNFGTLRLALSHSKAQFSPEYKAVHRFGSDYPFVFLGQRFTPICPLCISEAPYIRQQWQFLSQQACERHGCKLLHHCPECQSRLEYQTTESISQCECGFELRNSPIEDAPVAALLVARWLSGNDSKPLGLLKAEMTLSERYGFLLWYVNRYGDIENISFESFVEYCSCWPRVLKEELDELVNKADLIRIKDWKKTFFNEVFGALLKDCRQLPSRQLERNSVLTQVLAYFTKLMAAIPSSSKGNVGDVLLSPLEASTLLSCTTDEVYRLYEFGEIKAAIRPRMHTKIASHESAFTLRSVIETKLTRMCSENDGLSVYLPEW

SEQ ID NO: 82 - protein, 328 aa, Rhodococcus sp.
MNLSAKQEIAVDELLTQYHNSFVIYPDVQQIFDGLDWIVRRSQFGNFTPSMLITGGTGAGKTSLINHYAKYHFNDNEVLITRVRPSFIETLIWAIDKLGIPYNTRSKRSEIGLQDYFINSVKKSNLKLLVIEEAQELFECASPKERQKIRDRLKMISDECRLPIVFIGIPTAKLILEDSQWDRRIMVKRELPYIRITSESSLDVYIDLLEELEKQLPISVQPELSEMDIAMRLLSATKGMLGAIKELVGYALELALLSGKSAITNDEFTLGFERINGPDVTNPFTTELEKLLVPQVIEYEGFIIDPENGEIKFTKQIFKDIPLAALLG

SEQ ID NO: 83 - protein, 360 aa, Pseudoalteromonas sp.
MVMPFDDEFESITDNTQAEYDSTSEAKLTRKQYLPLDSVTIHERDLSSFSEEQKNKALERYKLISSVAKEISGGWTPKNINPLIDKYSPNLSIKRPSYKSVIRWYKSFCENDGNIVCLVDHNHSKGNRTKRIIDDEAFFVEATERFLDAKRPNYSQAYQFYCDRIEIENSNIISGQISKVSYQAFKERLKKLPPYEVALKRFGPNYANKLFNYYQSSVRTSRILERVELDHTPLDLILLDDDLLIPLGRAYLTLLVDVFSGCIIGFHLGFNPPSYVSVAKAIIHSVKSKDYVHDLNIELTNDWLCHGKMETLVVDNGAEFWSKSLEQACMEAGIHVEYCKVGQPWEKPRVERKFLEIIQG

SEQ ID NO: 84 - protein, 207 aa, unidentified
  source 1..207: note = Vibrionaceae family
MYVRNLRKPSATKNVYKFASSKNRSVILCESSLERDCCYHLEYSKDVFSFQSQPEGFYYSSGYKRCPYTPDFLVRNQDGSEYYLEVKPLAKTFSEDFKRSFALKRIAAQHQGKSLVLVTDKQIRNGVYLENLNLIHRYSGLVDFSLPSTKIVEELSAAGRMCIRSLADNLKLSIGEVIAAVFCLIGLGRVNVPLDSAINEMSVISVN

SEQ ID NO: 85 - protein, 335 aa, Vibrio navarrensis
MRVRVLIHLENQLKLNNFGALRLALSHSKAQFSPEYKAVHRFEADYPFVFLAKRFTPICPLCISEAPYIRQQWQFLSQQACERHGCKLVHHCPECQSRLEYQTTESISQCECGFELRNSPVEDAPVAALLVARWLSGNDSKPLGLLKAEMTLSERYGFLLWYVNRYGDIENISFESFVEYCSCWPRVLQEELDELVNKADLIRVKDWKKTFFNEVFGALLKDCRQLPSRQLNRNSVLTQVLAYFTKLMATLPSSSKGNVGDVLLSPLEVSTLLSCTTDEVYRLYEFGEIKAAIRPRMHTKIASHESAFTLRSVIETKLTRMCSENDGLSVYLPEW

SEQ ID NO: 86 - protein, 328 aa, Rhodococcus sp.; identical to SEQ ID NO: 58

SEQ ID NO: 87 - protein, 360 aa, Pseudoalteromonas sp.
MVMPFDDEFESINDDTQAEYDSTSEAKLVRKQYLPLDSVTIHERDLSSFSEEQKNKALERYKLISAVAKEISGGWTPKNINPLIDKYGLNLSIKRPSYKSVIRWYKSFCGSDGNIVCLVDHNHSKGNRTKRIIDDESFFVEATERFLDAKRPNYSQAYQFYCDRIEIENSNIISGQISKVSYQAFKERLKKLPPYEVALKRFGPNYANKLFNYYQSSVPTTRILERVELDHTPLDLILLDDDLLIPLGRAYLTLLVDVFSGCIVGFHLGFNPPSYVSVAKAIIHSVKSKDYVHDLNIELTNDWLCHGKMETLVVDNGAEFWSKSLDQACMEAGIHYEYCKVGQPWEKPRVERKFLEIIQG

SEQ ID NO: 88 - protein, 207 aa, unidentified
  source 1..207: note = Vibrionaceae family
MYVRNLRKPSATKNVYKFASSKNRSVILCESSLERDCCYHLEYSKDVFSFQSQPEGFYYSSGNKRCPYTPDFLVRNQDGSEYYLEVKPLAKTFSEDFKRSFALKRIAAQHQGKLLILVTDKQIRNGVYLENLNLIHRYSGLVDFSLSSTKIVEELSAAGRMCIRSLADNLKLSIGEVIAVVFRLIGLGRVNVPLDSAINEMSVISVN

SEQ ID NO: 89 - protein, 201 aa, synthetic construct
  REGION 1..201: note = Description of sequence: Cas protein
MSQRYYFLIRYTNANADYGLLAGRCISQTHLFMVNNHQAMNRVGVSFPDWNESSVGQTIAFVSEDKEMMIGLSFQPYFSLMVKEGLFELSSICEVPDNLGEVRFVRNQTINKSFLGSKKRRIKRSMVRAELSGAEQRLPVTNEDRVIDSFHRIPISSGSSRQDFILFIQKELADERAESGFNSYALATNQERRGTVPDLRF

SEQ ID NO: 90 - protein, 343 aa, synthetic construct
  REGION 1..343: note = Description of sequence: Cas protein
MELCTQLNYVRSLSAGKAYFYYLSESGEMCPLNVDKTRLRAPKGSYSEAYKGNKFVDKNVAPQDLAYSNPQFIEECYVKPGVDEIYCAFSLRIRANSLTPDICSDDEVRSKLSMFSKIYKELNGYKELANRYAKNILLGTWLWRNRECRNITIEVTTSELDTFVVEHAQKLSWYGHWDGDSTECLERLTAYLERALSDPTEYFYMDVKAKMRVGWGDEVYPSQEFLDSREDGIPTKQLATVELLRGKETVAFHGQKVGAALQSIDDWWHEEADKPLRVNEYGADREYVIARRHVTHGNDFYQLVRNTENWIEAMTASQTIPNDVHFIMSVLIKGGLFNCAKAN

SEQ ID NO: 91 - protein, 540 aa, synthetic construct
  REGION 1..540: note = Description of sequence: Cas protein
MTKLSDLLVIEDEAIKQTALKKMFMPYTEDVCVDGYEQETLTILLNLSSSHQADRCSDWLDVARAQRYLKDRENLDASLAEIQWFHTHNLKFPDCRVKDQRIIARPLSTAEEFISSAVLDQRLGWAHNSAVYRHTLWLLNPFKWQSQPVCILSLIQQKNPVWLDLLTEFGLDVKSLARLQRAIEEQLPENSFPNSVSAYSKQLRFPWGDDYVSITPVVSHALQCELEIRARSPENKFSFVSSSLPNSASIGNLCGSLGGYMRVLNYPLGVKQAKGGTLTGNRQKSGHYFDDYQVTNAKICQVLNRLIGSEPSKTQRQRERARQVRGKILRKQIALWMLPLIELRDIAESEPNQQQLEHDDTLAQAFLSLPELELGSLAGEFNRRLHLTFQNNIYSAKFAYHPKLMQVAKAQVTWVLEQLSKPINNQDKVTGEQYIYLSSMRVQDAVAMSNPCLCGVPSLTAIWGVMHDYQRKFNQLVNNGSPVEFSSFAFYVRNENIQSTAKLTEPNSVAKARTVSNAKRPTIRSERLSDLEIDLVIRVH

SEQ ID NO: 92 - protein, 335 aa, Vibrio navarrensis; identical to SEQ ID NO: 85

SEQ ID NO: 93 - protein, 328 aa, Rhodococcus sp.; identical to SEQ ID NO: 58

SEQ ID NO: 94 - protein, 360 aa, Pseudoalteromonas sp.; identical to SEQ ID NO: 87

SEQ ID NO: 95 - protein, 207 aa, unidentified; identical to SEQ ID NO: 88
  source 1..207: note = Vibrionaceae family

SEQ ID NO: 96 - protein, 201 aa, synthetic construct; identical to SEQ ID NO: 89
  REGION 1..201: note = Description of sequence: Cas protein

SEQ ID NO: 97 - protein, 343 aa, synthetic construct; identical to SEQ ID NO: 90
  REGION 1..343: note = Description of sequence: Cas protein

SEQ ID NO: 98 - protein, 540 aa, synthetic construct; identical to SEQ ID NO: 91
  REGION 1..540: note = Description of sequence: Cas protein

SEQ ID NO: 99 - protein, 207 aa, unidentified; identical to SEQ ID NO: 88
  source 1..207: note = Vibrionaceae family

SEQ ID NO: 100 - protein, 360 aa, Pseudoalteromonas sp.; identical to SEQ ID NO: 87

SEQ ID NO: 101 - protein, 328 aa, Rhodococcus sp.; identical to SEQ ID NO: 58

SEQ ID NO: 102 - protein, 335 aa, Vibrio navarrensis; identical to SEQ ID NO: 85

SEQ ID NO: 103 - protein, 540 aa, synthetic construct; identical to SEQ ID NO: 91
  REGION 1..540: note = Description of sequence: Cas protein

SEQ ID NO: 104 - protein, 343 aa, synthetic construct; identical to SEQ ID NO: 90
  REGION 1..343: note = Description of sequence: Cas protein

SEQ ID NO: 105 - protein, 201 aa, synthetic construct; identical to SEQ ID NO: 89
  REGION 1..201: note = Description of sequence: Cas protein

SEQ ID NO: 106 - protein, 335 aa, Vibrio navarrensis; identical to SEQ ID NO: 85

SEQ ID NO: 107 - protein, 328 aa, Rhodococcus sp.; identical to SEQ ID NO: 58

SEQ ID NO: 108 - protein, 360 aa, Pseudoalteromonas sp.; identical to SEQ ID NO: 87

SEQ ID NO: 109 - protein, 207 aa, unidentified; identical to SEQ ID NO: 88
  source 1..207: note = Vibrionaceae family

SEQ ID NO: 110 - protein, 201 aa, synthetic construct; identical to SEQ ID NO: 89
  REGION 1..201: note = Description of sequence: Cas protein

SEQ ID NO: 111 - protein, 343 aa, synthetic construct; identical to SEQ ID NO: 90
  REGION 1..343: note = Description of sequence: Cas protein

SEQ ID NO: 112 - protein, 540 aa, synthetic construct; identical to SEQ ID NO: 91
  REGION 1..540: note = Description of sequence: Cas protein

SEQ ID NO: 113 - protein, 260 aa, unidentified
  source 1..260: note = Vibrionaceae family
MTCVPGCGWVSTQPSVAEIISCPKKTITCPFKSHKVSYALFGCFFTTKIGTRMYIRNLRKPSPNKNIFKFASAKNKDTIMCEGSLEKDCCYHFEYDADVVSFESQPKGFLYDFDGKQLPYTPDFFVIYTDGCYSFVETKPYSKTLSKEFKLKFRARKQAAKKLGFDLILVTDKQIRTGYFLKNSEMVHRYSGCIEGDSLTDAVLEELLISKAIKIYELAQRISSSIREVFASVLRLISVGKARTDFDLEMLNENSIISVN

SEQ ID NO: 114 - protein, 360 aa, Pseudoalteromonas sp.
MSRHFKDEFDPIHSEDAEQEFLSLPETKRTQLQYDSLDNTKTYERDLASFPPEQKEKALQRYKLLCLVHKDLKGGWTPKNLNPLIDQHIDKTWLPKPSYKTLIRWHQAFSGSHGSFTSLVDKNYLKGNREPRVTGDEVYYERALERFLDAVRPSINAAYQVYCDEIIIANETIISGKIPTVSYQSFKNRIKKEQPYSVALARHGKYYADKLYHYYQSVQMPTRILERVEIDHTPLDLILLHDDLLIPLGRAYLTLLVDVFSGCIIGFHLGFKAPSYVSASKAILHSIKKKDYISSLPIEFQNEWPCSGKIENLVVDNGAEFWSKSLEDACTESGINIVFNRVRKPWLKPFVERKFGEIIQ

SEQ ID NO: 115 - protein, 338 aa, Rhodococcus sp.
MESTQKREEQLVSQPMSNSHIDLVLSGYHDSFTIYPEIKKVFSGLDWLIKRRKFGAFVPSMLLTGGTGAGKSALIKYYVANNLSDKEVLITRVRPTLHETLLWIAKELSVYKNRKAKASDIGLETHVSTCIKRTDLKLLVIEECQELFECTTHNARQNIRDRLKMISDDCRLPIVFVGLPSAKLILEDSQWKRRIMVKRELPYIKITDEPSMDRYMDLLEAMEKSVPMPFEIDLTDVDLAMRLLAASHGMVGMLKELIASALESALLAKQSVIRVDDFAIGHEAIFGIDVSNPFTVEIDHLAIKQIESYEEYVPDAETGELKFVGQIFNNLSMTQLLG

SEQ ID NO: 116 - protein, 363 aa, Vibrio tapetis
MDQHEAMAGAFPLELNRVNIYHAQTTSQMRVRVLIHLENQLKLNNFGVLRLALSHSKAQLSPQYKAVHRFGVDYPYAFLRKRFTPICPLCINEAPYIHQQWQFISHQACEHHGCKLTHHCPECNSRLEYQSTESIGQCECGYELRNSPVDDAPEAETLVARWLSGNDSKPLGLLKAEMTIPERYGFLLWYVNRYGDIDDLSFESLIEYCGSWPTALWQDLDALKEKAELVRVKDWKKMFFNEAFGALLKDCRQLPSRQLSHNIVLAQVLAYFTKLMATVPSSAKGNIGDVLLSPLEASTLLSCTTDEVYRLYEFGEIKAAIRPRMHTKIASHESAFTLRSVIETKLTRMSSESDGLSVYLPEW

SEQ ID NO: 117 - protein, 540 aa, synthetic construct
  REGION 1..540: note = Description of sequence: Cas protein
MTKLSDLLTIEDEAVKQSALKKIFMPYTEDVCVEGFEKEALTILLNLSSSHQSDRCSDWLDVARAKRHLKAAENLEASLDEIKWFHTHNLKFPDCRVKDQRIVAQPLTTTEVFISSAVLEQRFGWAHNSAVYRHTLWLLNPFRWQSQSVSLLSLIQQETPVWLDLLKEFGLGVKSLDRLQNTLAEELPENSFPDSVSTYSKQLRFPWGDDYISVTPVVSHAIQSELEVRSRSQESQLSFVSLSLPNSASIGNLCGSLGGYMKVLNYPLNVKPAQGGTLPESRKKSGHYFDDYQVTNAKICQVLNHLIGSEPSKTQKQRESARKVRSKILRKQIALWMLPLIELRDIVDADTNQQQLEHDDTLAQAFLVLPESGLGSLASEFNRRLHLMFQNNKYAAKFAYHPKLMQVVKAQIVWVLEQLSKPNGNEDKVTGEQYIYLSSMRVQDAVAMSSSYLCGAPSLTAIWGFMHHYQREFNKLVNCDSPFEFSSFSFYVRSENIQPTAKLTEPSSVAKARTVSNAKRPTIRSERLADLEIDLVIRVH

SEQ ID NO: 118 - protein, 347 aa, synthetic construct
  REGION 1..347: note = Description of sequence: Cas protein
MELCTQLNYVRSLSAGKAYFYHLSKSGEMCPLEIDRTRLRAPKGGYAEAYKGGKFVAKNVAPQDLAYANPQFIEECYVKPGVDDIYCAFSLRIRANSLNPDVCSDDEVRYKLSSLAKTYKELNGYSELAYRYAKNILLGTWVWRNRECRKLTIEVKTSDSETVVIENATRLSWYGHWDEASTECLEKLTAYLKQALSDPSEYFYMDVKAKIGVGWGDEVYPSQEYLDSREDGVPTKQLATIELTSGKETVAFHGQKVGAALQSIDDWWHEEADKPLRVNEYGADREYVIARRHVTLKNDFYQLLRNTENWIESMAASQTIPNDVHFIMSVLIKGGLFNRSKSKPKAK

SEQ ID NO: 119 - protein, 201 aa, synthetic construct
  REGION 1..201: note = Description of sequence: Cas protein
MTKRYYFYIRYIPAHADFELLAGRCIHQMHMFMVNNPQVMNRIGVSFPSWGESSVGQTIAFVAEDKDMMIGLSFQPYFSLMTKEGIFEIFNVCEVPENLGEVRFIRNQTIEKNFLGSKKRRIKRSIARAELSGGEQPLPVAVEERVVDHFHRVPVSSGSSGQDYILFTQKEFAEERTARSFNSYGLATNEERRGTVPDLRF

SEQ ID NO: 120 - protein, 260 aa, unidentified; identical to SEQ ID NO: 113
  source 1..260: note = Vibrionaceae family

SEQ ID NO: 121 - protein, 360 aa, Pseudoalteromonas sp.; identical to SEQ ID NO: 114

SEQ ID NO: 122 - protein, 338 aa, Rhodococcus sp.; identical to SEQ ID NO: 115

SEQ ID NO: 123 - protein, 363 aa, Vibrio tapetis; identical to SEQ ID NO: 116

SEQ ID NO: 124 - protein, 540 aa, synthetic construct; identical to SEQ ID NO: 117
  REGION 1..540: note = Description of sequence: Cas protein

SEQ ID NO: 125 - protein, 347 aa, synthetic construct; identical to SEQ ID NO: 118
  REGION 1..347: note = Description of sequence: Cas protein

SEQ ID NO: 126 - protein, 201 aa, synthetic construct; identical to SEQ ID NO: 119
  REGION 1..201: note = Description of sequence: Cas protein

SEQ ID NO: 127 - protein, 207 aa, unidentified; identical to SEQ ID NO: 88
  source 1..207: note = Vibrionaceae family

SEQ ID NO: 128 - protein, 360 aa, Pseudoalteromonas sp.; identical to SEQ ID NO: 87

SEQ ID NO: 129 - protein, 328 aa, Rhodococcus sp.; identical to SEQ ID NO: 58

SEQ ID NO: 130 - protein, 335 aa, Vibrio navarrensis; identical to SEQ ID NO: 85

SEQ ID NO: 131 - protein, 540 aa, synthetic construct; identical to SEQ ID NO: 91
  REGION 1..540: note = Description of sequence: Cas protein

SEQ ID NO: 132 - protein, 343 aa, synthetic construct; identical to SEQ ID NO: 90
  REGION 1..343: note = Description of sequence: Cas protein

SEQ ID NO: 133 - protein, 203 aa, synthetic construct
  REGION 1..203: note = Description of sequence: Cas protein
MTMTKRYYFLIRYKPVQVDNELLAGRCISQMHLFMVNNRPAMNRIGVSFPDWNESTVGQTIAFVAEDKDMMIGLSFQPYFSLMVNEGLFEISSVCEVPDIASEVRFVRNQTIGKSFLGSKKRRIKRSMARAELRGVEPSLPVTNEGRIIDNFHRIPISSGSSGEDYILFVQKELADGGTATSFNSYGLATNQERRGTVPDLGF

SEQ ID NO: 134 - protein, 343 aa, synthetic construct
  REGION 1..343: note = Description of sequence: Cas protein
MELCTQLNYVRSLSAGKAYFYHLSKSGEMCPLEIDRTRLRAPKGGYAEAYKGGKFVEKNVAPQDLAYSNPQFIEECYVKPGVDDIYCAFPLRIRANSLTPDTCSDDEVRSKLSLLAKTYEELNGYQELAFRYAKNILLGTWLWRNRECRKLSIEVTTSDSQTLIVENATRLSWYGHWDEASAECLEKLTAYLTRALSDPTEYFYMDVKAKIGVGWGEEVYPSQEFLDDREDGVPTKQLATVELLTGEETVAFHGQKIGAALQSIDDWWHEEADKPLRVNEYGADREYVIARRHVSHGNDFYQLVRNTESWIETMKASQTIPNDVHFIMSVLIKGGLFNCSKAK

SEQ ID NO: 135 - protein, 540 aa, synthetic construct
  REGION 1..540: note = Description of sequence: Cas protein
MTKLSDLLAIEDEAVRQVTLKKMFMPYTEDVYVEGYEKEALTILLNLSSSHQSDRCSDWLDIARAKRHLKAAENLEASFDEIKWFHTHNLKFPDCRVKDQRIVAQPLTTTEVFISSGVLEQRLGWAHNSAVYRHTLWLLNPFSWQSQSVCIFSLIQQESHVWIELLKEFGLGAKSLARLKHTIEEQLPENHFPDSVSTYSKQLRFPWGDDYVSVTPVVSHAIQSELEVRSRSRESKLRFVSSSLPNSASIGNLCGCLGGNMKVLNYPLDVKPSANKTLSASRKISGDYLDGYQITNAKICQVLNHLIGSEPSKTQKQRESARKVRSKILRKQIALWMLPLIELRDIVDADSNQQQLEHYDTLVHAFLALPESDLGSLASEFNRRLHLTFQNNKYTAKFAYHPKLMQVVKAQIVWVLEQLSKPIGNEDKVTGEQYIYLSSMRVQDAVAMSSPYLCGAPSLTAIWGFMHRYQREFNKLANSDSPFEFSSFSFYVRSEKIQSTAKLTEPNSVAKARTVSNAKRPTIRSERLADLEIDLVIRVH

SEQ ID NO: 136 - protein, 363 aa, Vibrio tapetis
MDQHEAIAGAFPLELNRVNIYHAQTTSQMRVRVLIHLENQLKLNNFGVLRLALSHSKAQFSPQYKAVHRFGADYPYAFVRKRFIPICPLCIDEAPYIRQQWQFISHQACEYHGCKLVHYCPECKSRLEYQSTESISQCECGYELRNSPVEDALEAEVLVAQWLSGKDSKHLGSLNGEMTISERHGFLLWYVNRYGDIDALSFESFIEYCSAWPMALWQDLDALKEKAELVRVKDWKKVFFNEAFGVLLKDCRQLPSRQLSRNIVLTQVLAYFTQLMATVPSSAKGNIGDVLLSPLEASTLLSCTTDEVYRLYEFGEIKASIRPRMHTKIASHESAFTLRSVIETKLTRMSSESDGLNVYLPEW

SEQ ID NO: 137 - protein, 360 aa, Duganella sp.
MMWALMDQVQLMRLRLKINLRLSLKPQNNQLMMILKILRGINLNLTSRQFEQLQSFETCFIEYPAITEIYSIFDQLRLNHSLGGEPESFLLTGEAGSGKTALIKNYLSRFPSGSTWSKQPVLSTRVPSRINEQNTLTQFLVDLDGKSGGRGTRRRNEIALGEAVVKQLERKSVELIIVNEIQELVEFSTAEERQAIANTFKYISEEAKVSFVLVGMPYADVIATEPQWNSRLSWRRKIPYFKLLKANSHSSETASYAFDLEHKKHFAKFVAGLSARMGFAEPPVLTKNEVLYPLFVMCRGECRALKHFLKDALLMSFNENVDTIDKAILSRTFSFKFPYLDNPFDCPIEELRLHQIDSGS

SEQ ID NO: 138 - protein, 360 aa, Vibrio anguillarum
MPSDSNGIFGFFDEFEASEGESEQSSLELYQEPVEIVPTLDSQPKKVQQEVLRRIKIIAFVDKRLKGGWTEKNLTPILSLVESELQLTPPSWRTLATWKKSYFESGKDPCALIPKHAFKGNRQKEMDSQSLIDEAIKNIYLTRERLSVAEAYRYYKSRVIQINREIVEGKIKPIAERSFYNRIHDLPPYEVAIARFGKRYADREFRAVGQQVAAAKPMEYVEIDHTPVPAILIDDELGIPLGRPYLTMLYDRFSKCIVGCSINFREPSFDSVRKALLNSLLDKSWIKQRYPSIENEWPCHGKIDCLVVDNGAEFWSQSLEDSLRPLVSDIQYSQAAKPWRKSGVEKLFDQMNKGLVNALP

SEQ ID NO: 139 - protein, 207 aa, unidentified
  source 1..207: note = Gammaproteobacteria class
MFDQTKKSSHVHNICKFMSLKNDAVVRTLSILEFDFCFHLEYNADIKSFTSQPFGFQYKFNNRKCRYTPDFLATDHDDQSTFFEVKHSSQILKPDFRDRFKEKQRVALNEFNRRLVLVTEKQIRVGPTLNNFKLLHRYSGLRTVTEFQKLVLTFIQHKQMVKLEEVSLYFGLSEQDTLISTLPWISSGQVKTDPNSIGFGLETYVWC

SEQ ID NO: 140 - protein, 360 aa, Duganella sp.
MMILKILRGINLNLTSRQFEQIQSFETCFIEYPAITEIYSIFDQLRFNHSLGGEPESFLLTGEAGSGKTALIKNYLSRFPSGSAWSKQPVLSTRVPSRINEQNTLTQFLVDLDGKSGGRGTRRRNEIALGEAVVKQLERKSVELVIVNEIQELVEFSTAEERQAIANTFKYISEEAKVSFVLVGMPYADVIATEPQWNSRLSWRRKIPYFKLLKANSHSSETASYAFDLEHKKHFAKFVAGLSARMGFAEPPVLTKNEVLYPLFVMCRGECRALKHFLKDALLMSFNENVDTIDKAILSRTFSFKFPYLDNPFDCPIEELRLHQIDSGSAYNLNAIAAEDKILAPRFTDAIPLSMLLSKN

SEQ ID NO: 141 - protein, 201 aa, synthetic construct
  REGION 1..201: note = Description of sequence: Cas protein
MTKRYYFLIRYKPVQADNELLAGRCISQMHLFMVNNRQAMNRIGVSFPDWNESTVGQTIAFVAEDKDMMIGLSFQPYFSLMVNEGLFDIFRVCEVPDTVGEVRFVRNQTIGKSFLGSKKRRIKRSMARAELRGAETLLPIANEERVINSFHRIPISSGSSAQDYVLFIQKEFVDERVAANFNSYGLATNQERKGTVPELRF

SEQ ID NO: 142 - protein, 360 aa, Vibrio anguillarum
MPSDSNGIFGFFDEFEASEGESEQSSLELYQEPVEIVPTLDSQPKKVQQEVLRRIKIIAFVDKRLKGGWTEKNLTPILSLVESELQLTPPSWRTLATWKKSYFESGKAPCALIPKHAFKGNRQKEMDSQSLIDEAIKNIYLTRERLSVAEAYRYYKSRVIQINREIVEGKIKPIAERSFYNRIHDLPPYEVAIARFGKRYADREFRAVGQQIAATKPMEYVEIDHTPVPAILIDDELDIPLGRPYLTMLYDRFSKCIVGCSINFREPSFDSVRKALLNSLLDKSWIKQRYPSIENEWPCHGKIDCLVVDNGAEFWSQSLEDSLRPLVSDIQYSQAAKPWRKSGIEKLFDQMNKGLVNALP

SEQ ID NO: 143 - protein, 207 aa, unidentified
  source 1..207: note = Gammaproteobacteria class
MFDQTKKSSHVHNICKFMSLKNDAVVRTLSILEFDFCLHLEYNADIKSFTSQPFGFQYKFNNRKCRYTPDFLATDHDDQSTFFEVKHSSQILKPDFRDRFREKQRVALNEFNRRLVLVTEKQIRMGPTLDNFKLLHRYSGLRTVTEFQKLVLMFIQHKQMVKLEEVSLYFGLSEQDTLISTLPWISSGQVKTDPNSISFGLETYVWC

SEQ ID NO: 144 - protein, 343 aa, synthetic construct
  REGION 1..343: note = Description of sequence: Cas protein
MELCTQLNYVRSLSAGKAYFYHLSKSGEMCPLEIDRTRLRAPKGGYAEAYKGGKFVEKNVAPQDLAYSNPQFIEECYVKPGVDDIYCAFPLRIRANSLTPDTCSDDEVRSKLSLLSKTYEELNGYQELALRYAKNILLGTWLWRNRECRTLSIEVTTSDSQTLIVENATRLSWYGHWSEASAECLEKLTAYLTRALSDPTEYFYMDVKAKIGVGWGDEVYPSQKFLDSREDGVPTKQLATVELLNGKETVAFHGQKIGAALQSIDDWWHENADKPLRVNEYGADREYVIARRHVSHGNDFYQLLRNTENWIEKMTASKTIPNDVHFIMSILIKGGLFNCSKAK

SEQ ID NO: 145 - protein, 540 aa, synthetic construct
  REGION 1..540: note = Description of sequence: Cas protein
MTKLSDLLAIEDEAVKQVTLKKMFMPYTEDVCVEGCEKEALTILLNLSSSHQSDRCSDWLDVARAKRHLKAAENLEASLDEIKWFHTHNLKFPDCRVKEQRIVAQPLTTTEVFISSGVLEQRLGWAHNSAVYRHTLWLLNPFSWQSQSVCILSLIQQESLVWIELLKEFGLGDKSLARLKHTIEEQLPDNYFPDSVCTYSKQLRFPWGDDYVSVTPVVSHAIQSELEVRSRSRESKLRFVSSSLPNSASIGNLCGSLGGHMKTLNYPLDIKPAKGGTLPESREKSGHYFDDYQVTNAKVCQVLNHLVGSEPSKTQKQRESARKVRSKILRKQIALWMLPLIELRDIVDGDPNQQQLEHDDILAQAFLALPESDLGSLASEFNRRLHLTFQNNKYAAKFAYHSKLMQVVKAQIVWILEQISKPNGNEDKVTGEQYIYLSSMRVQDAVAMSSPYLCGAPSLTAIWGFMHHYQREFNKLVNSDSPFEFSSFSFYVRSEKIQSTAKLTEPNSVAKARTVSNAKRPTIRSERLADLEIDLVIRVH

SEQ ID NO: 146 - protein, 335 aa, Vibrio navarrensis
MRVRVLIHLENQLKRNNYGLLGLALSHSKSQFSPDFKAVHRFGVDYPYGFIRKRYTPICPQCLEDAPYIRQPWQFITHQACERHGCKLIHHCPGCHSRLEYRNTESIYQCECGFDLRNSPVEAAPEAELQLARWLSGSDSKPLGQLKTEMTPSERYGFLLWYVHRYGDVENISFESFVEYCSCWPKVLWQELDDLVKRADLVRVKDWKKTFFNEAFGALLKECRQLPSRQLSRNSVLTQLLAYFTKLMATVPSSVKGNIGDVLLSPLEASTLLSCTTDEVYRLYEFGEIKASIRPRMHTKIASHESAFTLRSVIETKLTRMSSESDGLSVYLPEW

SEQ ID NO: 147 - protein, 392 aa, Pseudoalteromonas sp.
MHFLVQTKPFRDETLESYLLRLTRDNAYTDYHELADIIWQRLVECDHELEGAFPLELKTANLYHASQSSCFRVRAFKLVAQWAALEPLELIRLSWLRSNTQFGHLSALIRDQLLIPRTLLRESHIPVCLSCLKEEGYIPYYWHLKPYKACHKHKMQLQSHCAECGELIDYRASERFSQCNCGAKLKTTAPASEADIAVSEGLSGADAHDLVGKLTWFAYQYGQDQEHASFNEAFIAYFKYWPDNFFAELDEKASRAREKQLRPFNHTKFDSVFGDVIKVSKVAGPNVLRTNIIVDSLLEFFSDLVEKNPKQKHPNIADLLVNSSDAATLIGTTLEQVFRLYQEGQLTCCERLNKNERLQPDRCVFYLRQVVELAQSQGRYVGDFKNQLITPW

SEQ ID NO: 148 - protein, 333 aa, Rhodococcus sp.
MLSEIQTQRLNEFIDVFIEYPLMTTILNDFERLRTGQKLAGEKQCMLLTGDTGCGKSSVINHYKNKHPACHIDGVYHHPVLVSRIPSRPTLESTIAQLLSDLGQVGAAKRKLKRNDANLTDSLITNLKSCGTELIIINEFQELVESNQGKKRNEIANRLKYLNEEAGIPIVLVGMPWAEQIAEEPQWSSRLMVRRFIPYFKLSKDLTLFVRVLMGLATRMPFRDKPRLQEQDIVYALFAVSKGCFRTLKYFLNEAVKQALMYGSETLTKQHLSAAFEVFYPGIEDPFKLSVDEITACEVESYSVYHTDSSSDNEAHNRTRFTEKMPINQLLKK

SEQ ID NO: 149 - protein, 360 aa, Pseudoalteromonas sp.
MNSKSLGLFDDEFQHPIPQVEAPKSTKAEKDLSAYPQEIQQTTYARLKYIRWLKERIVGGWTEKNLAPLINEMPKVEGADKPNWRTAARWFKSYSESDESIFALIPKHQKKGKRTQRTDTDKFFEKALERFLVKERPSVSSAYQYYKDLVVIENEKVIGEDLRPLTYKAFKNRIDKLPHYEVLRARYGKRIADLAYNKVESHKRPTRVLERVEVDHTPLDLILLDDELQIPLGRPTLTILIDVYSHCIVGFYLGFNDPSYDAIRRAMLNAMKPKNWIKEQYPDITHDWHCCGKIETLVVDNGVEFWSNSFESACAQIGISLQYNPVGKPWLKPFVERIFGVINTQLLDPIPGKTFSNMMK

SEQ ID NO: 150 - protein, 210 aa, synthetic construct
  REGION 1..210: note = Description of sequence: Unknown origin
MYRRNPKSSRVKHQFKFASQKSKNTCFAESALEFDACFHFEYSSAIVAFETQPLGFKYDFEGRTCPYTPDFLLTHSDGTQKYIEIKPVKELAKDEFRQRFEQKQLASKQLGIELILVTDKQIRVFPVLDNLKLLHRYSGFQVLTELHTVVVGLVKSTGLVKVAQLVNYLKVSAGEVLSIVARLLCIGQLATDLTTDALSLDSVIWASDEQ

SEQ ID NO: 151 - protein, 201 aa, synthetic construct
  REGION 1..201: note = Description of sequence: Cas protein
MKRSYFTVTYLPINCDYSLLAGRCIGILHGYLSRFELDGIGVSFPKWNEETVGNQIAFVSESESHLQTLRQQNYFQMMSHDGLFKLSVTRTVPEQVDEIQFVRNQSISKAFVGERRRRMVRAKLRAEARGEEYNPIYEFEPTEIEHFHSIPITSKSNNNEFVLHIQRRECSGTAGGFNKYGFATNEHHLGTVPSLSGLLIS

SEQ ID NO: 152 - protein, 342 aa, synthetic construct
  REGION 1..342: note = Description of sequence: Cas protein
MRLPRHLSYTRSLSPGKAVFFYQTPESDFEPLQIEQNKLIGQKSGFTDAYQKLGEPKSLAPQDLAFGNPQTIDVCYVPPTVNEIFCRFSLRVEANSVEPYVCDDRKVSYWLKRFVETYKEHGGFKELASRYAKNILMGNWLWRNKQSPGFDVEVLTEHSEPIVIEGAQRLKWNGVWGNDEAALATLTEAIENGLANPQAYCYLDITSRIKTAFCQEIHPSQKFVDNVEQGMSSKQLAYTQVDGKQVASLNAQKVGAAIQTIDDWYTEDLKRLRIHEYGADKQVLVAHRTPKNGLDFYSLLPKVAVYIRHMERDGLEDDEESKHIHYLASILLKGGLFQRSKG

SEQ ID NO: 153 - protein, 540 aa, synthetic construct
  REGION 1..540: note = Description of sequence: Cas protein
MDLAEALAIKSVREKSDALRKLFAPYASSVAVDGYEQQVATVLINLVYKRNEQIDLASIKAAKDVLKDTELMAKCISEVKWFHTHNLKYPDIRVSHQRLIAQVIDKDIPAITSASLPKLLGWSHNSAEINHAKLFLTSFKWQGEVNNLAGVLVDEHPAWIEAMRNLGLTKKSVMALATHIRELLPATDLPGEVSEFTPQLMMPYGKDYLSVSPVLSHAMLVRVQQLARDKVVNWGVVEHTRPANVGDLPSSLGGNVRVLRYFPATSLKGSLGEYIENDPNERLFKHKVLTSKSFKTSLIILTGKSVYNTLRQKRLARIAAIRQLRSTLMHWLDKIIEFQNNGNHNLVPEPMKSFIENLKPNLNGVVCELSGLLNEQLSSHHSLKKFAYHPDLMSVLKRQLQYVLTHGETVDETLVDSQILYVHCKGLRIFDAEGMPNPYLQGIPSLTALAGVAHKFQRKLQSIVDPSIECIGSAIFVHSYQLHTGKPLPEQNQLKTNQGITSAKRPGIVDKPKCDMTIDLVFRLFVPHESTRTQLTDNMF

SEQ ID NO: 154 - protein, 201 aa, synthetic construct; identical to SEQ ID NO: 151
  REGION 1..201: note = Description of sequence: Cas protein

SEQ ID NO: 155 - protein, 342 aa, synthetic construct; identical to SEQ ID NO: 152
  REGION 1..342: note = Description of sequence: Cas protein

SEQ ID NO: 156 - protein, 540 aa, synthetic construct; identical to SEQ ID NO: 153
  REGION 1..540: note = Description of sequence: Cas protein

SEQ ID NO: 157 - protein, 392 aa, Pseudoalteromonas sp.; identical to SEQ ID NO: 147

SEQ ID NO: 158 - protein, 333 aa, Rhodococcus sp.; identical to SEQ ID NO: 148

SEQ ID NO: 159 - protein, 360 aa, Pseudoalteromonas sp.; identical to SEQ ID NO: 149

SEQ ID NO: 160 - protein, 210 aa, synthetic construct; identical to SEQ ID NO: 150
  REGION 1..210: note = Description of sequence: Unknown origin

SEQ ID NO: 161 - protein, 335 aa, Vibrio navarrensis
MRVRVLIDLENQLRRNNFGVLRLALSHSKAQFSPQYKAVHRFGSDYPYAFLRKRFTPICPLCIDKDPYIRQQWQFISHQACEHHACKLVHHCPECKSRLEYQSTESISQCECGYKLRNSPVEDAPEAEVLVARWLSGNASTPLGLLKAEMTLSERYGFLLWYVNRYGDIDDLSFESFIEYCRAWPTTLWQDLDALKVKAELIRVKDWKKVFFSEAFGALLKDCRQLPSRQLIHNTVLAQVLGYFTQLMATEPSSAKGNIGDVLLSPLEASTLLSCTTDEVYRLYEFGEIKATIRPRMHTKIASHESAFTLRSVIETKLTRMSSESDGLNVYLPEW

SEQ ID NO: 162 - protein, 322 aa, Rhodococcus sp.
MNLDRDEVLANYHDSFSIYPEVEKVFSGLDWIMRRRKFGSFAPSMLLTAGTGAGKSATINYYIENNLSRNEVLITRVRPTLLETLLWMAKELGAYRNSRAKPSDIGLTDRVIETSKRVGLKLLVIEECQELFERTSHNQRQDIRDRLKMISDECRLPIVFVGLHSAELILEDSQWNRRIMVRRTLPYIKITDESAIDNYLDVLEALEKTVPLPFKVPLTDVDFAMRLLSASKGILGEIKELIAAALEVALENNKRCISEDDFAIVYQKINGFIDTNPFTVEIDELTIEQIASYENYVTDADTGELRFVKQAFSKLSIQQLVG

SEQ ID NO: 163 - protein, 360 aa, Pseudoalteromonas sp.
MVGRFHDESEYNEDSELKHEFLPEIQAERLKYSRLQSTQIIERDLSSYPEEQKNKALERYKLLCLVANELSGGWTSQNLTPLIEKHFEKIHLTQKPSYKSLQRWHNLFSDSGGSFTSLVDKNHRKGNRKARVVGDEKYYDEALRMFLDARRQSIRAAYTFYCDRITIANETIVTGRIPKVSYQSFKNRIRKEEPYSVALARHGKYYADKLFNYYQAVEMPTRILERVEMDHTPLDLILLHDELMVPLGRAHLTLLVDVFSGCIIGFHLGFKAPSYVSASRAVIHAIKSKTYISEMSIEFNNEWLCDGKIENLVIDNGAEFWSQSWEDACREVGINVAFNKVRKPWLKPFVERKFGEIIQG

SEQ ID NO: 164 - protein, 208 aa, unidentified
  source 1..208: note = Vibrionaceae family
MYIRNLRKPSPNKNIYKFSSLKNRDAVMCESRLEKDCCYHFEYDPDVVQYESQPEGFYYDFNGKERPYTPDFLVTYHDGTFEYVEVKPYSKTLSKTFKQEFSARKEAATQRGFRLVLVTDKQIRYGYFLKNVELVHRYSGCIVGDELTTRVYSYLIIKNTIKISDLADLTGESVGRVFASVLRLIAIGKVGVDLDIAQLSESTTVSVR

SEQ ID NO: 165 - protein, 201 aa, synthetic construct
  REGION 1..201: note = Description of sequence: Cas protein
MTKRYYFLIRFIPAQADYELLAGRCISQMHLFMVNNRQAMNKIGVSFPDWSDATVGQTIAFVAEDKEMMIGLSFQPYFSLMVNEGLFEISSICEVSVNALEVRFVRNQTIGKSFLGSKKRRIKRSMARSELFGAGTSLPVTNEERVIDSFHRIPVSSGSSAQDYILFVQKELVGEHVTANFNSYGLATNQERRGTVPELRF

SEQ ID NO: 166 - protein, 343 aa, synthetic construct
  REGION 1..343: note = Description of sequence: Cas protein
MELCTQLNYVRSLSAGKAYFYYLSKSGEMCPLEIDRTRLRAPKGGYAEAYKGGKFVEKNVALQDLAYSNPQFIEECYVKPGVDDIYCAFSLRIRANSLNPDSCNDDEVRSKLSLLAKTYNELNGYQELALRYAKNILLGTWLWRNRACRKLSIEVVTSDSQTLIVENVARLSWYGHWDEGSAECLEKLTAYLMRALSDPTEYFYMDVKAKIGVGWGDEVYPSQEFLDEREGGVPSKQLATVELLTGDETVAFHGQKIGAAIQTIDDWWHEDADKPLRVNEYGADREYVIARRHVAYGNDFYQLVRNAENWIETMAASQTIPNDVHFIMSVLIKGGLFNCSKAK

SEQ ID NO: 167 - protein, 540 aa, synthetic construct
  REGION 1..540: note = Description of sequence: Cas protein
MTKLSDLLEIEDEAVKQVTLKKMFMPYTENVCVEGFEKEALTILLNLSSSHQSDRCSDWLDVARAKRHLKATENLEASLDEIKWIHTHNLKFPDCRVKDQRIVAQPLTTTEVFISSAVLEQRLGWAHNSAVYRHTLWLLNPFCWQSQPVCILSLLQQENPIWLELLKEFGLGAKSLTRLKHTIGKHLPDNHFPDSVSIYSKQLRFPWGDDYVSVTPVVSHAIQSELEVRSRSRESKLRFVSSSLPNSASIGNLCGSLGGHMKVLNYPLDVKLAQGKTLAESRKKSGHYFDDYQVTSAKICQVLNHLIGSEPLKTQKQREGARKVRSKILRKQIALWMLPLIELRDIVDADPNQQQLEHNDTLAQAFLVLPESDLASLANEFNRRLHITFQNNKYAAKFAYHPKLMQVVKAQIVWILEQLSKPIGNEDKVTGEQYIYLSSMRVQDAVAMSCPYLCGVPSLTAIWGFMHRYQREFNNLANCDSLFEFSSFSFYIRSERIQLTAKLTEPNSVAKARTVSNAKRPTIRSELLADLEIDLVIRVH

SEQ ID NO: 168 - protein, 210 aa, unidentified
  source 1..210: note = Gammaproteobacteria class
MRTMFDQTKKSSHVHNICKFMSLKNDAVVRTLSVLEFDFCFHLEYNPDVKRYISQPHGFYYYFNGRKCRYTPDFLADDHKDQSTLIEIKHSSQILKPDFRQRFAEKQRVALEEQGKRLVLVTEKQIRIDPIFSNLKLLHRYSGLHTVTKVQKSVLGFIQRKQKVQLREVSEYLGLSEHETLISTLCWLSSGRVRTDIRSSDFGLNSYVWC

SEQ ID NO: 169 - protein, 360 aa, Vibrio anguillarum
MASDFDDVFGFIDEMEASAQEAELATKLPRDLFEEDTSYLPTIDTFPEKTQKEVFRRLKVIQFTEKRLKGGWTEKNLVPILNQVEQELALSPPSWRVLAEWKKVYFESGRSIHSLVPAHAQKGNRNQHTDSQLLIDEAINKKYLTKERLSVAEAYRYYKSRVIKTNQEIVEGKIKLIKERSFYNRVKGLPPYDVAVARYGKRYADREFRPVGQQIEATKPMEYVEIDHTPVPVILIDDELDIPLGRPYLTMLYDRFSKCIVGFSINFREPSFDSVRKALLSTLLEKNWIKDKFPSITNDWPCHGKIDYLVVDNGAEFWTPSLEDSLRPLVSDIHYSQAAKPWRKTGIEKLFDQLNKGLVN

SEQ ID NO: 170 - protein, 357 aa, Duganella sp.
MVNLTSMQHEQLKAYDNCFIEYPEITEIYSIFDQLRFNQSLGGEPESFLLTGETGSGKTALINNYLKRFEASSRSSWSTQTVLSTRIPSRVNEQSTLNQFLVDLNSKSGGRSTRRRNEIALGESVVRHLKRKSVELIIVNEIQELVEFSNADERQTIANTFKYISEESGVSFVLVGMPYADVIANESQWNSRLSWRREINYFTLFKDDRGPKDTGPKYRIDAVQKKHFAMFVAGLASRMGYENKPLLTNNEILYPLFSMCRGECRRLKHFLKDAMLMSFKEDKNTMDKEVLSSTFALKFPSLDNPFECSLDKLELDQIDIGSAYNTRAITAEDKILAPRFTDAIPLSILLSKSGLKV

SEQ ID NO: 171 - protein, 363 aa, Vibrio tapetis
MDQHEAIAGAFPLELNRVNIYHAQTTSQMRVRVLIHLENQLKLNNFGVLRLALSHSKAQLSPQYKAVHRFGVDYPYAFLRKRFTPICPLCINEAPYIHQQWQFISHQACEHHGCKLTHHCPECSSRLEYQSTESIGQCECGYELRNSPVDDAPEAETLVARWLSGNDSKPLGLLKAEMTIPERYGFLLWYVNRYGDIDDLSFESFIEYCCAWPTALWQDLDALKEKAELVRIKDWKKVFFNEAFGALLKDCRQLPSRQLSHNIVLAQVLAYFTKLMATVPSSAKGNIGDVLLSPLEASTLLSCTTDEVYRLYEFGEIKAAIRPRMHTKIASHESAFTLRSVIETKLTRMSSESDGLSVYLPEW

SEQ ID NO: 172 - protein, 540 aa, synthetic construct
  REGION 1..540: note = Description of sequence: Cas protein
MTKLSDLLAIEDEVVKQASLKKMFMPYTEDVCVEGFEKEALTILLNLSSSHQSDRCSDWLDVARAKRHLKAAENLEASLDEIKWFHTHNLKFPDCRVKDQRIVAQPLATAEVFISSAVLDQRLGWAHNSAVYRHTLWLLNPFRWQSQSVSLLSLVQQETPVWVELLKEFGLGAKLLARLQNTMAEQLPENSFPVSVNTYSKQLRFPSGDDYVSVTPVVSHAIQRELEVRSRSRESKLSFVSSSLPNSASIGNLCGSLGGHMKVLNYPLDVKPAQGGTLTESRKKSGRYFDDYQVTNAKICQVLNHLIGSEPSKTQKQRESARKVRSKILRKQIALWMLPLIELRDIVDADPNQQQLEHDDTLAQAFLTLPESDLGSLASEFNRHLHLTFQNNKYAAKFAYHPKLMQVVKAQIVWVLEQLSKPNGNEDKVTGEQYIYLSSMRVQDAVAMSSSYLCGAPSLTAIWGFMHHYQREFNKLVNCDSPFEFSSFSFYVRSENIQPTAKLTEPSSVAKARTVSNAKRPTIRSERLADLEIDLVIRVH

SEQ ID NO: 173 - protein, 347 aa, synthetic construct
  REGION 1..347: note = Description of sequence: Cas protein
MELCTQLNYVRSLSAGKAYFYHLSKSGEMCPLEIDRTRLRAPKGGYAEAYKGGKFVAKNVAPQDLAYANPQFIEECYVKPGVDDIYCAFSLRIRANSLNPDVCSDDEVRYKLSSLAKTYKELNGYSELAYRYAKNILLGTWVWRNRECRKLTIEVKTSDSETVVIENATRLSWYGYWDEASTECLEKLTAYLKQALSDPSEYFYMDVKAKIGVGWGDEVYPSQEYLDSREDGVPTKQLATIELTSGKETVAFHGQKVGAALQSIDDWWHEEADKPLRVNEYGADREYVIARRHVTLKNDFYQLLRNTENWIESMAASQTIPNDVHFIMSVLIKGGLFNRSKSKPKAK

SEQ ID NO: 174 - protein, 201 aa, synthetic construct
  REGION 1..201: note = Description of sequence: Cas protein
MTKRYYFYIRYIPAHADFELLAGRCIHQMHMFMVNNPQVMNRIGVSFPTWGESSVGQTIAFVAEDKDMMIGLSFQPYFSLMTKEGIFEIFNVCEVPENLGEVRFIRNQTIEKNFLGSKKRRIKRSIARAELSGGEQPLPVAVEERVVDHFHRVPISSGSSGQDYILFTQKEFAEERTARSFNSYGLATNEERRGTVPDLRF

SEQ ID NO: 175 - protein, 335 aa, Vibrio navarrensis
MRVRVLIHLENQLKLNNFGALRLALSHSKAQFSPEYKAVHRLGADYPFVFLAKRFTPICPLCISEAPYIRQQWQFLSQQACERHGCKLLHHCPECQSRLEYQTTESISQCECGFELRNSPVEDAPVAALLVARWLSGNDSKPLGLLKAEMTLSERYGFLLWYVNRYGDIENISFESFVEYCSCWPRVMQEELDELVNKAYLIRIKDWKKTFFNEVFGALLKDCRQLPSRQLERNSVLTQVLAYFTKLMATLPSSSKGNVGDVLLSPLEVSTLLSCTTDEVYRLYEFGEMKAAIRPRMHTKIASHESAFTLRSVIETKLTRMCSENDGLSVYLPEW

SEQ ID NO: 176 - protein, 328 aa, Rhodococcus sp.
MNLSAKQEIAVDELLTQYHNSFVIYPDVQQIFDGLDWIVRRSQFGNFTPSMLITGGTGAGKTSLINHYVKYHFNDNEVLITRVRPSFIETLIWAIDKLGIPYNTRSKRSEIGLQDYFINSVKKSNLKLLVIEEAQELFECASPKERQKIRDRLKMISDECRLPIVFIGIPTAKLILEDSQWDRRIMVKRELPYIRITSESSLDVYIDLLEELEKQLPISVQPELSEMDIAMRLLSATKGMLGAIKELVGYALELALLSGKSAITNDEFALGFERINGPDVTNPFTTELEKLLVPQVIEYEGFIIDPENGEIKFTKQIFKDIPLAALLG

SEQ ID NO: 177 - protein, 360 aa, Pseudoalteromonas sp.
MVMSFDDEFESITDNTQAEYDSTSEAKLTRKQYLPLDSVTIHERDLSSFSEEQKNKALERYKLISSVAKEISGGWTPKNINPLIDKYSPNLSIKRPSYKSVIRWYKSFCENDGNIVCLVDHNHSKGNRTKRIIDDEAFFVEATERFLDAKRPNYSQAYQFYCDRIEIENSNIISGQISKVSYQAFKERLKKLPPYEVALKRFGPNYANKLFNYYQSSVRTSRILERIEIDHTPLDLILLDDDLLIPLGRAYLTLLVDVFSGCIIGFHLGFNPPSYVSVAKAIIHSVKSKDYVHDLNIELTNDWLCHGKMETLVVDNGAEFWSKSLEQACMEAGIHVEYCKVGQPWEKPRVERKFLEIIQG

SEQ ID NO: 178 - protein, 207 aa, unidentified
  source 1..207: note = Vibrionaceae family
MYVRNLRKPSATKNVYKFASSKNRSVILCESSLERDCCYHLEYSKDVFSFQSQPEGFYYSSGNKHCPYTPDFLVRNQDGSEYYLEVKPLAKTFSEDFKRSFALKRIAAQHQGKSLVLVTDKQIRNGVYLENLNLIHRYSGLVDFSLPSTKIVEELSVAGRMCIGALADNLKLSIGEVIAAVFCLIGLGRVNVSLDSAINEMSVISVN

SEQ ID NO: 179 - protein, 365 aa, Dickeya sp.
MFMYKSHIPEDCEEEVQITAAKLILLVDDDPVFRRMTKGYLQKYGYQVIEAENGLDGLRKLRDHEPDLILCDLSMPVLNGIEFVEEVSSSYPSLPLVVVSATDEMADVAKALRFGIKDFLPKPITNHSHLASAIEKVLANDDHQRDFASCWYGADRQGSIPEEQELHWHLQYLKDNPNAAKELLEALLPEKDSQLGSWKCSYRLLQSAELMPLVFDYAWLMNGQFVFYMVDAASHRECGAASTLLVRALFHDYLRNLRNFSVELKDLAEIIERGIHCSECIGKVSALFGVADLADGTLSLLPAGLDCQWSNGYVHNRITSGVQLGDNSLRNFITKDLPIRAACQISLSKPSAYSFTLDLYQGVSG

SEQ ID NO: 180 - protein, 212 aa, synthetic construct
  REGION 1..212: note = Description of sequence: Cas protein
MSQRYYFLIRYTNANADYGLLAGRCISQMHLFMVNHHQAMNRVGVSFPDWNESSVGQTIAFVSEDKEMMIGLSFQPYFSLMVNEGLFEISSVYEVPDTSEEVRFVRNQTIGKNFLGSKKRRIKRSMARAELFGIQQSLPVTNEDRAIDSFHQIPISSGSSRQDYILFIQKELADERAESGFNSYGFATNQEKRATVPDLRFNPFDEDSFLVQ

SEQ ID NO: 181 - protein, 343 aa, synthetic construct
  REGION 1..343: note = Description of sequence: Cas protein
MELCTQLNYVRSLSAGKAYFYYLSESGEMCPFNVDRTRLRAPKGSYSEAYKGNKFVDKNVAPQDLAYSNPQFIEECYVKPGVDEIYCAFSLRIRANSLTPDICSDDEVRSKLSMFAKIYKELNGYKELANRYAKNILLGTWLWRNRECRNITIEVTTSELDTFVVEHAQKLSWYGHWDGDSTECLERLTAYLERALSDPTEYFYMDVKAKMRVGWGDEVYPSQEFLDSREDGIPTKQLATVELLSGKETVAFHGQKVGAALQSIDDWWHEEADKPLRVNEYGADREYVIARRHVAYGNDFYQLLRNTENWIESMTASESIPNDVHFIMSVLIKGGLFNCAKAN

SEQ ID NO: 182 - protein, 540 aa, synthetic construct
  REGION 1..540: note = Description of sequence: Cas protein
MTKLSDLLAIEDEAIKQTALKKMFMPYTEDVCVDGYEQETLTILLNLSSSHQADRCSDWLDVARAQRYLKDRENLDASLAEIQWFHTHNLKFPDCRVKDQRIIARPLSTAEEFISSAGLDQRLGWAHNSAVYRHTLWLLNPFKWQSQPVCILSLIQQKNPVWLDLLTEFGLDVKSLARLQRAIEEQLPENSFPNSVSAYSKQLRFPWGDDYVSITPVVSHALQCELEIRARSPENKFSFVSSSLPNSASIGNLCGSLGGYMRVLNYPLGVKLAKGGTLTENRQKSGHYFDDYQVTNAKICQVLNRLIGSEPSKTQRQRERARQVRGKILRKQIALWMLPLIELRDIAESEPNQQQLEHDDTLAQAFLSLPEWELGSLASEFNRRLHLTFQNNMYSAKFAYHPKLMQVAKAQVTWVLEQLSKPINNQDIVTGEQYIYLSSMRVQDSVAMSNPCLCGVPSLTAIWGFMHDYQRQFNQLVNNDSPVEFSSFAFYVRNENIQSTAKLTEPNSVAKARTVSNAKRPTIRSERLADLEIDLVIRVH

SEQ ID NO: 183 - protein, 207 aa, unidentified
  source 1..207: note = Vibrionaceae family
MYVRNLRKPSATKNVYKFASSKNRSVILCESSLERDCCYHLEYSKDVFSFQSQPEGFYYSSGNKRCPYTPDFLVRNQDGSEYYLEVKPLAKTFSEDFKHSFALKRIAAQHQGKPLVLVTDKQIRNGVYLENLNLIHRYSGLVDFSLSSTRIVEELSAAGRMCIRSLADNLKLSIGEVIAVVFRLVGLGRVNVPLDSAINEMSVISVN

SEQ ID NO: 184 - protein, 360 aa, Pseudoalteromonas sp.
MVIPFDDEFEFITDDTQAEYDSTSEAKLARKQYLPLDSVTIHERDLSSFSEEQKNKALERYKLISAVAKEISGGWTPKNINPLIDKYSPNLSIKRPSYKSVIRWYKSFCENDGNIVCLVDHNHSKGNRTKRIIDDEAFFVEATERFLDAKRPNYSQAYQFYCDRIEIENSNIISGQISKVSYQAFKERLKKLPPYEVALKRFGPNYANKLFNYYQSSVRTSRILERVEIDHTPLDLILLDDDLLIPLGRAYLTLLVDVFSGCIVGFHLGFNPPSYVSVAKAIIHSVKSKDYVHDLNIELTNDWLCHGKMETLVVDNGAEFWSKSLDQACMEAGIHYEYCKVGQPWEKPRVERKFLEIIQG

SEQ ID NO: 185 - protein, 328 aa, Rhodococcus sp.
MNLSAKQEIAVDELLTQYHNSFVIYPDVQQIFDGLDWIVRRSQFGNFTPSMLITGGTGAGKTSLINHYVKYHFNDNEVLITRVRPSFIETLIWAIDKLGIPYNTRSKRSEIGLQDYFINSVKKSNLKLLVIEEAQELFECASPKERQKIRDRLKMISDECRLPIVFIGIPTAKLILEDSQWDRRIMVKRELPYIRITSESSLDVYIDLLEELEKQLPISVQPELSEMDIAMRLLSATKGMLGAIKELVGYALELALLSGKSAITNDEFALGFERINGPGVTNPFTTELEKLLVPQVIEYEGFIIDPENGEIKFTKQIFKDIPLAALLG

SEQ ID NO: 186 - protein, 335 aa, Vibrio navarrensis
MRVRVLIHLENQLKLNNFGALRLALSHSKAQFSPEYKAVHRLGSDYPFVFLAKRFTPICPLCISEAPYIRQQWQFLSQQACERHGCKLVHHCPECQSRLEYQTTESISQCECGFELRNSPVEDAPVAALLVARWLSGNDSKPLGLLKAEMTLSERYGFLLWYVNRYGDIENISFESLVEYCSCWPRALEEELDELVNKADLIRIKDWKKTLFNEVFGTLLKDCRQLPSRQLERNSVLTQVLAYFTKLMATLPSSSKGNVGDVLLSPLEVSTLLSCTTDEVYRLYEFGEMKAAIRPRMHTKIASHESAFTLRSVIETKLTRMCSENDGLSVYLPEW

SEQ ID NO: 187 - protein, 540 aa, synthetic construct
  REGION 1..540: note = Description of sequence: Cas protein
MTKLSDLLAIEDEAIKQTALKKMFMPYTEDVCVDGYEQETLTILLNLSSSHQADRCSDWLDVARAQRYLKDRENLDASLAEIQWFHTHNLKFPDCRVKDQRIIARPLSTAEEFISSAVLDQRLGWAHNSAVYRHTLWLLNPFKWQSQPVCILSLIQQKNPVWLDLLTEFGLDVKSLARLQRAIEEQLPENSFPDSVSTYSKQLRFPLGDDYVSITPVVSHALQCELEIRARSPENKLSFVSSSLPNSASIGNLCGSLGGYMRVLNYPLGVKQAKGGTLTENRQKSGHYFDDYQVTNAKICQVLNRLIGSEPSKTQRQRERARQVRGKILRKQIALWMLPLIELRDIAESEPNQQQLEHDDTLAQAFLSLPELELGSLAGEFNRRLHLTFQNNMYSAKFAYHPKLMQVAKAQVTWVLEQLSKPINNQDTVTGEQYIYLSSMRVQDAVAMSNPCLCGVPSLTAIWGFMHDYQRQFNQLVNNDSPVEFSSFAFYVRNENIQSTAKLTEPNSVAKARTVSNAKRPTIRSERLSDLEIDLVIRVH

SEQ ID NO: 188 - protein, 343 aa, synthetic construct
  REGION 1..343: note = Description of sequence: Cas protein
MELCTQLNYVRSLSAGKAYFYYLSESGDMCPLNVDRTRLRAPKGSYSEAYKGNKFVDKNVAPQDLAYSNPQFIEECYVKPGVDEIYCAFSLRIRANSLTPDICSDDEVRSKLSMFAKIYKELNGYKELANRYAKNILLGTWLWRNRECRNITIEVTTSELDTFVVEHAQKLSWYGHWDGDSTECLERLTAYLERALSDPTEYFYMDVKAKMRVGWGDEVYPSQEFLDSREDGIPTKQLATVELLSGKETVAFHGQKVGAALQSIDDWWHEEADKPLRVNEYGADREYVIARRHVAYGNDFYQLLRNTENWIESMAASESIPNDVHFIMSVLIKGGLFNCAKAN

SEQ ID NO: 189 - protein, 212 aa, synthetic construct
  REGION 1..212: note = Description of sequence: Cas protein
MSQRYYFLIRYTNANADYGLLAGRCISQMHLFMVNHHQAMNRVGVSFPDWNESSVGQTIAFVSEDKEMMIGLSFQPYFSLMVNEGLFEISSVYEVPDTSVEVRFVRNQTIGKNFLGSKKRRIKRSMARAELFGIQQSLPVTNEDRVIDSFHQIPISSGSSRQDYILFIQKELADERAESGFNSYGFATNQEKRATVPDLRFNPFDEDSFLVR

SEQ ID NO: 190 - protein, 207 aa, unidentified
  source 1..207: note = Vibrionaceae family
MYVRNLRKPSATKNVYKFASSKNRSVILCESSLERDCCYHLEYSKDVFSFQSQPEGFYYSSGNKRCPYTPDFLVRNQDGSEYYLEVKPLAKTFSEDFKRSFALKRIAAQHQGKPLVLVTDKQIRNGVYLENLNLIHRYSGLVDFSLSSTKIVEELSAAGRMCIRSLADNLKLSIGEVIAVVFRLIGLGRVNVPLDSAINEMSVISVN

SEQ ID NO: 191 - protein, 360 aa, Pseudoalteromonas sp.; identical to SEQ ID NO: 57

SEQ ID NO: 192 - protein, 328 aa, Rhodococcus sp.
MNLSAKQEIAVDELLTQYHNSFVIYPDVQQIFDGLDWIVRRSQFGNFTPSMLITGGTGAGKTSLINHYVKYHFNDNEVLITRVRPSLIETLIWAIDKLGIPYNTRSKRSEIGLQDYFINSVKKSNLKLLVIEEAQELFECASPKERQKIRDRLKMISDECRLPIVFIGIPTAKLILEDSQWDRRIMVKRELPYIRITSESSLDVYIDLLEELEKQLPISVQPELSEMDIAMRLLSATKGMLGAIKELVGYALELALLSGKSAITNDEFALGFERINGPDVTNPFTTELEKLLVPQVIEYEGFIIDPENGEIKFTKQIFKDIPLAALLG

SEQ ID NO: 193 - protein, 363 aa, Vibrio navarrensis
MDQHEAIPGAFPLELNRINIYHAQTTSQMRVRVLIHLENQLKLNNFGALRLALSHSKAQFSPEYKAVHRLGSDYPFVFLGKRFTPICPLCISEAPYIRQQWQFISHQACEHHGCKLVHHCPECQSRLEYQTTESISQCECGFELRNSPVADAPLAALLVARWLSGNDSKPLGLLKAEMTLSERYGFLLWYVNRYGDIENISFESFVEYCSCWPRVLKEELDELVNKADLIRIKDWEKTFFNEVFGALLKDCRQLPSRQLERNSVLTQVLAYFTKLMATLPSSSKGNVGDVLLSPLEVSTLLSCTTDEVYRLYEFGEIKAAIRPRMHTKIASHESAFTLRSVIETKLTRMCSENDGLSVYLPEW

SEQ ID NO: 194 - protein, 540 aa, synthetic construct
  REGION 1..540: note = Description of sequence: Cas protein
MTKLSDLLAIEDEAIKQTALKKMFMPYTEDVCVDGYEQETLTILLNLSSSHQADRCSDWLDVARAQRYLKDRENLDTSLAEIQWFHTHNLKFPDCRVKDQRIIARPLSTAEEFISSAVLDQRLGWAHNSAVYRHTLWLLNPFKWQSQPVCILSLIQQKNPVWLDLLTEFGLDVKSLARLQRAIEEQLPENSFPNSVSAYSKQLRFPWGDDYVSITPVVSHALQCELEIRARSPENKFSFVSSSLPNSASIGNLCGSLGGYMRVLNYPLGVKQAKGGTLTENRQKSGHYFDDYQVTNAKICQVLNRLIGSEPSKTQQQRERARQVRGKILRKQIALWMLPLIELRDIAESEPNQQQLEHDDTLAQAFLSLPELELGSLAGEFNRRLHLTFQNNMYSAKFAYHPKLMQVAKAQVTWVLEQLSKPINNQDTVTGEQYIYLSSMRVQDSVAMSNPCLCGVPSLTAIWGFMHDYQRKFNQLVNNGSPVEFSSFAFYVRNENIQSTAKLTEPNSVAKARTVSNAKRPTILSERLADLEIDLVIRVH

SEQ ID NO: 195 - protein, 343 aa, synthetic construct
  REGION 1..343: note = Description of sequence: Cas protein
MDLCTQLNYVRSLSAGKAYFYYLSESGEMCPLNVDRTRLRAPKGSYSEAYKGNKFVDKNVAPQDLAYSNPQFIEECYVKPGVDEIYCAFSLRIRANSLTPDICSDDEVRSKLSMFAKIYKELNGYKELANRYAKNILLGTWLWRNRECRNITIEVTTSELDTFVVEHAQKLSWYGHWDGDSTECLERLTAYLERALSDPTEYFYMDIKAKLRVGWGDEVYPSQEFLDSREDGIPTKQLATVELLSGKETVAFHGQKVGAALQSIDDWWHEEADKPLRVNEYGADREYVIARRHVTHGNDFYQLVRNTESWIETMTASQTIPNDVHFIMSVLIKGGLFNCAKAN

SEQ ID NO: 196 - protein, 212 aa, synthetic construct
  REGION 1..212: note = Description of sequence: Cas protein
MSQRYYFLIRYTNANADYGLLAGRCISQMHLFMVNHHQAMNRVGVSFPDWNESSVGQTIAFVSEDKEMMIGLSFQPYFSLMVNEGLFEISSVYEVPDTSAEVRFVRNQTIGKNFLGSKKRRIKRSMARAELFGVEQSLPVTNEDRVIDSFHRIPISSGSSRQDFILFIQKELADERAKSGFNSYGFATNQEKRATVPDLRFNPFDEDSFLVR

SEQ ID NO: 197 - protein, 360 aa, synthetic construct
  REGION 1..360: note = Description of sequence: Unknown origin
MCGIVGYIGTQAATEILLAGLEKLEYRGYDSAGIATIWEGDVQCVRAKGKLHNLRSKLEQLETPAQIGIGHTRWATHGKPEEYNAHPHLDTAMRVAVVQNGIIENYRELREELKSKGHQFRSETDTEVIPHLIAEIIKKSPASLLEAVRQAVSHLEGAFALAVISADYPDELIVVRQQAPLVIGFGQGEFFCASDTPAIVAYTRAVLPLDNGEIARLTPLGVEVYNFAGDRLKKQPRLLNLNPTMVEKQGFKHFMLKEIYEQPGVVRASLEAYFNNDANAAGASQSPINLGLSESFYADIEQIQIVACGTSWHAALVGKYLLEQLAGISTQVHYASEYRYAPSPLMANTLIIGVTQSGET

SEQ ID NO: 198 - protein, 272 aa, Desulfovibrio zosterae
MVRGRREWTQAKYERYIKEGRGRGSGKQYKPWLTIQDVPSKGRSSRTPGWKTNRVHHFFSDHEKRLFYLFEWSDIVTDIREQFPLDDLDLAMSIATDMDIKYPVDTKSGTPYVLTSDFMLTVNRDGKQMQIARTVKPSTELEKKRVNEKLEIERQYYLAKGIEWGIITEKEIPRLLVENVEWVHSAYRLEANPEMNTAQLRELSTILKYKLQEEDTAINTITSALDKEMNISYGTSLYVFKHLIAIKEVIVDLVKIKLSACPSTKVFEKVIF

SEQ ID NO: 199 - protein, 144 aa, Denitrovibrio sp.
MVKDLFVNDLIEWIVYDNAHVVERIIWIDEGYIIAFVIDINAKKGLPEPKKISDILEAISEGVAFKQQQDPWARIIRDENLTDREKEYRDKAWEIISSLVIEEPSIYYRNFRGLLVKKIVENYNEERDKNKLVEKTVYGYLRRF

SEQ ID NO: 200 - protein, 360 aa, unidentified
  source 1..360: note = Cyanobacteria phylum
MTGYLARNILKPEYASRSRQIYNAIKHGGGKNLENYVNLPTSASGLTLIGPSGMGKSTNFQNILNLYPQVILHPDYSVYQIVWLKVDCPHAGSLKGLCTDIFLAVDRLLGTNYFKKFGSSRNSEDYMLAQVAQIAHTHHLGVLVIDEMQNLVTARRSSSDMLNFLVKMDNTVGIPVIRVGTNEAFPILQGNFRNARRGTGEGSVIWDRMVNDDEWYFFMEGMWEYQWTKTFVPFSDEINEVFYDMCQGIIDIAVKLYKMVQWRAISIGDKEIITVDLIRQAAKDGLYLVKPMLDAIRSGDKEWMIKYKDIAPLDTTEYQNKCLSELDYKDLKEIRRLARKQQNQQQVSPKLNYVILELLN

SEQ ID NO: 201 - protein, 324 aa, Nostoc sp.
MPPYLFSFSMLSFFPTPYPDELLYSALARYHIRSGNKTFKQTDLELFGYSSQQLCKVTLTNNLNYLVKNLSLFSQLTVSDLLQNYTLYPFYSTFLIPQQAWQLQDSMRNKLSGSILEVAKVANHPTENSTKFLKFCPVCLKKDTQEYGEPYWHRIHQIPGILVCPTHRIILHDSSVAVDSKGIHYHAASLENCLCNQNQVKYTDSTVEKLLILAQDIWDTSNINISFKGLAWLRNQYQSYLINQKFMNILPGGKFRFNQKSFSNSIRDCYGQEVLEIINPNLIKNPDKYFSSYLLGCDLNPVIDRITHILIIKFLAKSVKTFFT

SEQ ID NO: 202 - protein, 222 aa, unidentified
  source 1..222: note = Cyanobacteria phylum
MGDHFHLSTQNHVKDVLNPQLIYVDLSFRVIGQTLPIDHGYGLYAALTHLQDKLHSLDDLSIQTIPGIPDGKDSLNLNNHSRLRIRLPVEKIPLVYPFAGKSLTIGRHKIRLEIPQMYLLQAVEKLRSRIVVIKGYEEPDAFLAAAQRQLERLGIQGIASIPTKADGKPKRRSIKIKKFTVVGFGLEVTNLSNEDSITLQKYGVGGKQKMGCGVFVPVNEKR

SEQ ID NO: 203 - protein, 360 aa, Geitlerinema sp.
MINTTANSKIQLNLGESSTTLLHRAGIAGFWMTLKQLEKIYPNPDQRTGNLTWVLTPYSISLNWEGQDFAALDWLLKQSFQISDEGLISLIGSNLQAMDIERQIIIHQGITGTFLQHNQFFKSSGKKLKSIIVDSKKFVTEYKSAISYAHQHFAIKLCDESGQFLQEPIGIVGWLYPGAVVRHYAFKEQTQFEEKPEYALALLFAPVACHYFILRSHTKPQSTNYILLVPEVNNLELYAQYCWNLSNLSYRDFNVSSLREAGLKFLTTQTAVQVNQNCVKRCQVFSFGTLIWSEQQKVRTEIATLKLTEKVSFYYKLSRSCFPNYRIVYYGNQHFILTSLVQGIIANNLGLGLPWWNDFG

SEQ ID NO: 204 - protein, 331 aa, synthetic construct
  REGION 1..331: note = Description of sequence: Unknown Bacteria sp.
MSHLFGNILTIYGTAANNRGENEGNITTLQKINWKGEVHTTVSAEAIRWALRYYWQNNGYEVNRRWDENAQPLADHILQDLNFNNVRFIDDDVLGFMQTEAARVEAGDQSGGEVQEASAGEIQNVSQDEEVTSSKGKRKKTSQRKGRERPQGRVAAKRGVLEVTRAVSLIPYEGNITFNAVSGKKGRTSLYGTEVHATRYQYGFALTPNSLQDKSRSNAVLNGLISIGEVGGNHARFLYDFSPDSIILRWTHDFSPRFLYCFEQDEWGNISVPDLVRRVEAEDIDPKELWIGGAIARDLGNLGANVFPGVKKVVEELLKVITRDLQLLPRS

SEQ ID NO: 205 - protein, 173 aa, unidentified
  source 1..173: note = Cyanobacteria phylum
MLLSLVGEVDRYKHCGVKLAIALISEPAKSTVIRTFHRLKTKNIHDPKNNKPDYQEVLTNIEFVVWVDAGVDKAKPNLVQRLEEAFTNPGSINRFGGLCLGESRDLVNDITLLPKNYYAESMRWLIRDEYGLLTLPYWVNHVGSRGTRWLSYEIQECQVLQPPELSWTSVQSD

SEQ ID NO: 206 - protein, 360 aa, Microcoleus sp.
MLGFFPSPYPDEILYSILARYHIRSGNTSPKITLRELFNSENTIATVDLPSNLNSLMQNIKLVSNYQVEDFIYKHTLFPFYNVFLPPKRASQVIDSMKAEFGGDIHTRAGIMASSIKMLRSLRFCPQCLQEDLQKYGEAYWHRIHQIPGVLVCPFHCITVQDSIISTQGLNRHEYYAASSENCPISSIETYSEETLNKLLLLAQDSWYLINSKFVSRELEWFQKKYIALLISKGFSTASGRVNQKKLLDNFLFFYGHEILTAVNSMVSYEKSQNWLFSIFRKHRKSFHPIRHLLTIRFLSNSLDEFFSNHYEYKPFGDAPWLCLNAATEHYLQAVVNHLVISHCLDNKKPLGTFSCSCGM

SEQ ID NO: 207 - protein, 360 aa, Microcoleus sp.
MVSFFPKPYPEEILYSVIARYHIRSGNTSPKITLQELFNSQTTVATVDLPSNLNNLIQNLQFISNYQVEDLIYKHTLYPLYSPFLSANRSSQVLESMKGDYGGDIHTRAGIMASSITMPKYFRFCPTCLEEDLKTYGEPYWHRIHQIPGVLVCPFHAEVLQNSLVPLHGFNKHEYHAASTDNCSITKESTFFSTDILKKLLLLAKDFWWLLNSEIPCQEPQWFRKQYTNLMIEQRLATATGRIYQKRLLDNFLFFYGREMLSAVDSMVNHNDSNNWVTSIVRKHRKSFHPLRHLLIISFLKTSVEEFFKQDTEYKPFGESPWLCLNAAAEHYLQPVITSLDITLCCDTKKPVGTFSCSCG

SEQ ID NO: 208 - protein, 173 aa, unidentified
  source 1..173: note = Cyanobacteria phylum
MLLSLVGEENRYTHCGVKLAITLVSEPAKSTVIRTFHRLKTKNIHDPKNNKPDYQELLTNIEFVVWVDPGVDKARPNLVQRLEEAFTNPASINRFGGLCLGESRDLVNDITLLPKNYYAESMRWLIKDEHGLLTLPYWVDHVGSQGTRWLRYELQECQVLQPPELSWTSVQSD

SEQ ID NO: 209 - protein, 332 aa, synthetic construct
  REGION 1..332: note = Description of sequence: Unknown Bacteria sp.
MSHLFGNILTIYGTAANNRGENEGNITTLQKINWKGEVHTSVSAEAIRWAVRYYWQNNGYEVNRRWDENAQPLADHIWQDTNFNSVRFIDDDVLGFMQAEAARVEAGEQSGGEVQEASAGEIQNVSQDEETASPKGKGKKTSQRKGRERSQGRVTAKRGVLEVTRAVSLIPYGGNITFNAVSGTKGRTSLYGTEVHATRYQYGFAMTPNSLQDKSRINAVLDGLISIGEVGGNHARFLYDFSPDSIILRWTHDFSPRFLYCFEQDEWGTISVPDLVRRVEAEDIDPKELWIGGAIARDLGNLGANVFPGVKKVVEELLKVITRDLQLPPRSS

SEQ ID NO: 210 - protein, 360 aa, Geitlerinema sp.
MINTTANSKIQLNLSDSSTTLLHRAGIAGFWMTLKQLEEIYPNPAQRTGNLTWVLTPHSISLNWEGQDFAALDWLLKQSFQISDEGLIFLTGLNPKAIHIERQIIIHQGITGTFLQHNKFFKFSDKKLKSIIVDGRQFVTEYKSAMSYAHQHFAKKLCDESGQLLQEPIGIVGWLYPGAVVRHSAFPKQTKFEEKPEYALALLFVPVACHYFILRSHTKPQSTNYILVVPEVNNLEFYAQHCWNLSNLSYRDFHVSSLRDAGLKFLTNEAAIQIKQNCVKRCQVFSFGTLIWSEQQKVRTETATLELTEKVSFYYKFSRSCFSNYRIVDYENQHFILTSLVQGIIANNLGLGLPWWNDFG

SEQ ID NO: 211 - protein, 222 aa, unidentified
  source 1..222: note = Cyanobacteria phylum
MGDNFHVSTQSHATEVLNPQLIYVDLSFRVIGQTLPIDHGYALYAALTHLQDKLHSLDDLSIQTIPGIPDGNDSLHLNNHSRLRIRLPVDKIPLVYPFAGKSLTIGRHKIHLEIPQMYLLQAVEKLRSRIVVIKGYEEPKGFLAAAQRQLEKLGVQGIASIPTKADGKPERRSIKIKKFTVVGFGLEVTNLNDEDSITLQKYGVGGKQKMGCGVFVPVNEKR

SEQ ID NO: 212 - protein, 315 aa, Nostoc sp.
MLSFFPTLYPDELLYSALARYHIRSGNKTFKQTDLELFGYSSQQLCKVTLTNNLNYLVKNLSLFSQLTVSDLLQNHTLYPFYASFLIPQQAWQLQDSMRKKLGGSILEVAKVANHSADNSTKFLKFCPICLEQDTQEYGEPYWHRIHQIPGILVCATHRVVLHDSSVAVDSKGIHYYAASPENCLTNQNQVKCADNTVEKLLILAHDIWNISTINIPFKGLTWLRNQYQSYLINQKFMTLLPGEKFRFNEKSFSNSICDFYGQEFLEIINPNLIRNPDKYFSGYLLACDLNPVIDRITHILIIKFLANSVEIFFT

SEQ ID NO: 213 - protein, 360 aa, unidentified
  source 1..360: note = Cyanobacteria phylum
MDAHRRFHCVERLSRYFQPLNKTVNLQQVMCALLMQGYIARNISKPEYARRSRQIYEAIKAGDGRNLDVYVNVPTSTSASGLTLIGPSGMGKSTNFINILNLYPQVILHPEYSTYQIVWLKVDCPHAGSLKGLCTDIFLGIDRLLGTNYFKKFGASRNSEDYMLAQVAQMAHTHHLGVLVIDEMQNLVTARRSCSDMLNFLVKMDNIIGVPVIRIGTNEAFPILQGNFRNARRGTGEGSVIWDRMRNDDEWYFFMETMWEYQWTKISTDFSDEFNDVFYDESQGIIDIAIKLYKMVQWRAISLGGKEAINANLIKQSVKDGLFLVKPMLNAIRSGDKEWMIKYKDIAPIDTTEYYNNCLS

SEQ ID NO: 214 - protein, 480 aa, Bacillus akibai
MNNNLFVNDLIEWIDESGNNFVERIIWIDEAYIIAFTIDINIKTGFPVSKRVSDIQEAIYEGRALKLRTDPWARIVRDEDLSAKEKEIRDKYWDIISSIVIQEPSIYYRDKRGSLIKQVIEKYNNNKSEGKLIERSVYQIIRRYWQRGKDKNSLLPDYANSGGKGKVRASREKKRGRPRKYAHEPDIGVGINITDEDKKIFRLAVTKFYHNQKENFLTTAYDLMIKNYYSEETIYDENGVKKHILIPPDKRPTLTQFKYWYDVEQGDIRKKIISRRGAKKYALEHRAITGNSTMETIGPGSRYQIDATIADVYLVSAYNRNWIVGRPVIYVVIDVFSRMITGVYVGFEGPSWIGAMMALANAASDKVAFCKEYGIEITEDEWACHHIPDAILGDRGELAGMAVETLIPNLGIRIENAAPYRADWKGLVERHFGIIHGHIKPFVPGYIDKDFRERGARDYRLDSKLDIEQFTEIIIKIILY

SEQ ID NO: 215 - protein, 272 aa, Desulfovibrio zosterae
MARTKQEWTQAKFERYMKEGRGQGSGKNYQPWIKIQDFPSKGRVSRPPGWKTNREHHLFSDHEKRLFYLFEWSDSIIDIREQFPLIDLDLAMNIATEMAIEYPKDVNNNTPYVLTTDFMLSVKQGKQIVEKARTFKLVKDLGIKSVAEKFELEKRYYTAKGIDWGIITEKEIPTLLVKNVEWIHSSYKLEAAAEINVDELNNIANILKSKLQESDSTINKITNCLDKEMNLEIGTSLYIFKHLLANKKIIMDMQTNAISSSISAREIQKIIF

SEQ ID NO: 216 - protein, 360 aa, synthetic construct
  REGION 1..360: note = Description of sequence: Unknown origin
MCGIVGYIGTQAATEILLSGLEKLEYRGYDSAGVATLWEGEVHCVRAKGKLHNLRSKLEQLVTSSQIGIGHTRWATHGKPEEHNAHPHLDTARRIAVVQNGIIENYRELREELKQKGYEFRSETDTEVIPHLIAELSKNASSFLEAVRQAVSHLHGAFAIAVISADYPDELIVVRQQAPLVIGFGQGEFFCASDTPAIVSHTRAVLPLENGEIARLTPLGVEIYNFAGDRLKKQPRMLNLSPTMVEKQGFKHFMLKEIYEQPGVVRASLDAYFNTEVNSGKSFQSPVKLGLPEQFYADLEQIHIVACGTSWHAALVGKYLLEQLAGISTQVHYASEYRYAPSPLTANTLIIGVTQSGETA

SEQ ID NO: 217 - protein, 360 aa, synthetic construct
  REGION 1..360: note = Description of sequence: Unknown origin
MCGIVGYIGTQAATEILLAGLEKLEYRGYDSAGIATVWEGDVNCVRAKGKLHNLRSKLEQLATPSQIGIGHTRWATHGKPEEYNAHPHLDTAMRIAVVQNGIIENYRELRDELKQKGHEFRSETDTEVIPHLIAEFLKNLPSPALPTSSSSLLEAIRQAVNHLQGAFAIAVISADYPDELIVVRQQAPLVIGFGQGEFFCASDTPAIVSHTRAVLPLENGEIARLTPLGVEIYNFAGDRLKKQPRLLNLNPTMVEKQGFKHFMLKEIYEQPGVVRASLEAYFNPDTNTDESFTSPVNLGLSEEIYADLEQIHIVACGTSWHAALVGKYLLEQLAGISTQVHYASEYRYAPSPLTANTLII

SEQ ID NO: 218 - protein, 272 aa, Desulfovibrio zosterae
MVRGRREWTQAKYERYIKEGRGRGSGKHYKPWLTIQDVPSKGRSSRTPGWKTNRVHHFFSDHEKRLFYLFEWSDIVTDIREQFPLDNLDLAMSIATDIGIKYPVDTQSGTPHVLTSDFMLTVNRDGKQMQIARTVKPSTELEKKQVNEKFEIERQYYLAKGIDWGIITEKEIPRLLAENVEWVHSAYRLEANSEMDTAQLSELSTILKSKLQEEDTTINRVTTALDKEMNINSGTSLYVFKHLIARKEILVDLVAIKLSACPSTKVIEKIIF

SEQ ID NO: 219 - protein, 480 aa, Bacillus akibai
MVKDLFVNDLIEWIVYQNTHIVERIIWIDEGYIIAFVIDINAKKGLPEPRKISDILEAITEGIAFKQQQDPWARIIRDENLTDKEKEYRDKAWEIISSLIIEEPSIYYRNFRGSMVKEAVEKYNEERDKDKLVEKTVYGYLRRFWQRGKTINALIPDFINSSGKGKIRGQSGKKRGRPRKYAHDPDIGEGVNVTEEDRKIFRVSISKFYNNPKENYLTTAYELMVKEYYKEGIRYDENGVQKSILTPTDKIPTITQFKYWYQLEQDDVTKTLTSRKGAKRVALENRAILGNSKMETIGPGSRYQIDATVADVYLVSKYNRDWIIGRPVIYVIIDVFSRMITGVYVGLEGPSWIGAMMALANVATDKTLFCQEYNINITENEWPCNQMPDAILGDRGELAGMTVETLIPNLYVRIENAASYRADWKGLVERHFRIIHEYVKPFLPGYVDTDFRQRGARDYRLDGKLDIDQFTEIIIRLIVH

SEQ ID NO: 220 - protein, 458 aa, Bacillus akibai
MTGYLSRNLLKPEYANRSRQIYNAIGNGGGKNLENYVNLPTSASGLTLIGPSGIGKTTNFKNILELYPQIIFHPDYSVYQIVWLRVDCPKTLKGLCIDTFAGIDKLLGTNLLKKFNPARTSEDYLLAQLAQTAHTHHLGVLVIDEIQNLATAKRNRDEILNFLVKLDNTIGIPVIRVGTNEAISFLQGNLRNARRNTGEGSRMWDRMVNNDEWCYFLEGIWEYQWTKTPVHFSDEIKEVFYDESQGIIDIAIKLYKMVQWRAISIGDQEIITVDLIRQVAKEGLYLVKPMLDAIRLGDREWMIKYKDIAPLDITEYHNRCVSEIDAKDLTEIKRLAKKQQNQQKVSPKLNYVISELLKLEVESGKAKECAEQIVYSHEDPDIPIPSLVNKAYALALQGCQVNESLAKTSTRKAKLKTKYQENDIRRIVENAKKDKKSAYEDLKLAGIIKNPVEDFINI

SEQ ID NO: 221 - protein, 360 aa, unidentified
  source 1..360: note = Cyanobacteria phylum
MTGYLSRNLLKPEYANRSRQIYNAIGNGGGKNLENYVNLPTSASGLTLIGPSGIGKTTNFKNILELYPQIIFHPDYSVYQIVWLRVDCPKTLKGLCIDTFAGIDKLLGTNLLKKFNPARTSEDYLLAQLAQTAHTHHLGVLVIDEIQNLATAKRNRDEILNFLVKLDNTIGIPVIRVGTNEAISFLQGNLRNARRNTGEGSRMWDRMVNNDEWCYFLEGIWEYQWTKTPVHFSDEIKEVFYDESQGIIDIAIKLYKMVQWRAISIGDQEIITVDLIRQVAKEGLYLVKPMLDAIRLGDREWMIKYKDIAPLDITEYHNRCVSEIDAKDLTEIKRLAKKQQNQQKVSPKLNYVISELLKLE

SEQ ID NO: 222 - protein, 316 aa, Nostoc sp.
MLSFFPTLYPDELLYSTLARYHIRSGNKSFKQTDLELFGYSSQQICKITLTNNLNYLVKNLPLRSQQKVENLLQNHTLYSFYATLLMPQEAWLLKNSMSNKFSGSILDFAKVANNSSDDSKKFLKFCPECLEKDTQKYGEPYWHRIHQVPGILVCPIHRVVLHNSCIEVESKGIHYHAASIDNCLLANNVKKLTNKTVETLSILAHDINWLIHSSFAFQGLAWLRAQYQRYLLDKGLLKTFAGGKFKFNDCGFAQLIFEFYGQESLEALNPKLTQNQGKYFSHCLFGCDLNPAIDRITHILMIKFLANSIEEFFQS

SEQ ID NO: 223 - protein, 227 aa, unidentified
  source 1..227: note = Cyanobacteria phylum
MIIDSNLVTQLPSESSSKPRELLMSQFPYMDLTFSLTGETLPFDHGYELFSAIAHFEPKLHTLDTLGIQTIAGIPKDGVINLTQNSRLRIRIPVNQVRLVYPLAGKSLRIGKHTIRLGIPDICLLQPAEKLRSRIVVIRGYEEPESFLVVAQRQLEQLGIQAIASIPTKANGKPIRRTIKIKRFTVVGFGLEVINLSDEDSLRLQIHGVGGKQKMGCGLFMPIKERQ

SEQ ID NO: 224 - protein, 360 aa, Geitlerinema sp.
MMKRADNPKIQLNLGDPSMTLLHRAGVAGLWMTLKKLEKIYPTPAERIGNLTWLLTPHYINLDWEGQDFTVLDWLLKQSFQISDEGLISLTGLDSQNMDIQNQIIIHQGITATFTQHNKFFKSAGQQSKQLIIDGIQVRVDYKKAASYAHQHFAKHLCDQYGQLLKEPIGIRGWLYPGAVVRHYAFKEQTAFEEKAEYALALLFAPMACQYFVLRSHTEPSHTNYVLVIPEVIDLELYAHFLWCLGNLDYKHFHVSSLGDAGLKFLNYKTTQMPTSNFLKRCQIIFFSTKAWSEQQKTRAEIAIIELNSSVNYLYKLSCICFPECQIVLYKNQHYLLPSLIKGIIANNLAMGRSWWANWI

SEQ ID NO: 225 - protein, 324 aa, synthetic construct
  REGION 1..324: note = Description of sequence: Unknown Bacteria sp.
MFHLFGNILTSYGTAANNRGENEGNTTTLQKLIWKGEVHSTVSSEAIRWALRYYWQNAGYPVNRRWDENAQPVPDHILQDPNFDDIRFIDDDVLGFMQAEAAKVEAAVESEVEIQNSTQDEDKKLSQGKEKETAKRKRKEKPKGKITTRRGVLEVTRAVSTIPYAGDLTFNAMSGKKGRTSLYSTEVHATRYQYGFALTPNSLKDKSRINAVLDSLISIGEVAGNHARFLYDFSPDSIILRWTHDFSPRLLYCFEEDELRNISTPDLVRRVESGDIDPKELWIGGAISSTLEDLGANVFPGVKTTVEALKQVIIEDLQLSLRSS

SEQ ID NO: 226 - protein, 210 aa, unidentified
  source 1..210: note = Cyanobacteria phylum
MTTIALKVEVPIACFRQSRAREYGETYPVPPPSTVYGMLLSLVGEVDRYKHCGVKLAIALLSEPEKSTVIRTFHRFKTKNIHDSKNNKPDYQELLTNIEFIVWVDAGVDKAKPNLVERLAEALTNPASINRFGGLCLGESRDLVNDVTLLPKNYYAESMRWLIRDEYGLLTLPYWVDHVGSRGTRWLRYEIQKCPVFQPPELSWTSVQSN

SEQ ID NO: 227 - protein, 360 aa, Microcoleus sp.
MVSFFPQPYPDEILYSVIARYHIRSGNTSPKITLQDLFKSQTTVATVDLPSNLSNLIKNLQFISNYQVEELIYKHTLYPLYSPFLPANRSSQVLESMKGDYGGDIHTRAGIMASSITMPKYLRFCPTCWEEDLKNYGEPYWHRIHQIPGILVCPFHGEVLQDSLAPLHGFNKHEYYAASTDNCSITKEPSILSPDTLKKLLILAKDFWWLLNSEIPCQEPQWFRKQYTNLMIEQGFATPTGRIYQNKLLDNFLFFYGQEMLSAVDSMVNHNDSNNWVTSIVRKYRKSFHPLRHLLMMRFLKTSVEEFFQKDTEYKPFGEPPWLCLNAAAEHYLQPVITNLVITLCCDTKKPVGTFSCACG

SEQ ID NO: 228 - protein, 360 aa, synthetic construct
  REGION 1..360: note = Description of sequence: Unknown origin
MCGIVGYIGTQAATDILLAGLEKLEYRGYDSAGIATVWEGEVNCVRAKGKLHNLRSKLEQIETPAQIGIGHTRWATHGKPEEYNAHPHLDTAKRVAVVQNGIIENYRDLREELKGKGHQFVSETDTEVIPHLIAEFLKHIPPSSTSSPFLEAIRQAVNHLHGAFAIAVISADYPDELIVVRQQAPLVIGFGQGEFFCASDTPAIVAYTRAVLPLENGEIARLTPLGVEIYNFAGDRLKKQPRLLNFNPAMVEKQGFKHFMLKEIHEQPGVVRASLDAYFNPVESTESPINLGLPADFYTDLEQIQIVACGTSWHAALIGKYLIEQLAEISTQVHYASEYRYAPSPITANTLIIGVTQSGE

SEQ ID NO: 229 - protein, 281 aa, Desulfovibrio zosterae
MRSLSGVIFMVRGRGEWTQAKFERYRKEGRGRGSGKDYKPWITIQDFPSKGRSSRTPGWKSNRVHHFLSDQEKRLFYLFEWSDVVTDTREQFPLDDLDLAMSIATEMGIKYPVDLQSGTPYVLTTDFMLTVNQNGKQVQIARTVKQSTELEKKRVIEKLELERRYYLAKSIDWGIVTENEIPRLLAENVEWIHSAYNLEANSEMDIPQLHELGTILKSKLQEHDTAINRVTTALDKEMNIKSGTSLYMFKHLIAAKKIIINMLETKISACPSSKVIEKIIF

SEQ ID NO: 230 - protein, 456 aa, Bacillus akibai
MQGYIARNILKPEYTRRSRQIYDAIKDGGGKTLENYVYVPTSASGLTLIGPSGMGKSTNLLNILSLYPQVILHPEYTTFQIVWLKVDCPRSLKGLCTDIFLAVDRLLGTSYFKKFGSRGNSEDYMLAQVAQIAHTHHLGVLVIDEMQNLVNARRNRDDLLNFLVKMDNTIGVPVIRVGTNEAFPILQGNFRNARRGTGQGSVIWERMLQDDDWDFFLTGIWEYQWTKTPVDFSDEINEAFYYECQGIIDIAIKLYKMIQWRAISLGGDEKITVDLIHQSAKDGLYLVKPMLDAIRSGDQDWMIKYKDIAPINAIEYKNKCLSELESRDLEEVRRLARKQQNRGNQSVKLRHIIVELVKLDVEPSVAKECAEKVLEINEDNADIPSLLKEAYIYALQGEPVKKGTAKTNIRKTKPKYQSNDIRQIVENAKNKKNPAYQDLKAADIIKNPLQDFINIE

SEQ ID NO: 231 - protein, 360 aa, unidentified
  source 1..360: note = Cyanobacteria phylum
MQGYIARNILKPEYTRRSRQIYDAIKDGGGKTLENYVYVPTSASGLTLIGPSGMGKSTNLLNILSLYPQVILHPEYTTFQIVWLKVDCPRSLKGLCTDIFLAVDRLLGTSYFKKFGSRGNSEDYMLAQVAQIAHTHHLGVLVIDEMQNLVNARRNRDDLLNFLVKMDNTIGVPVIRVGTNEAFPILQGNFRNARRGTGQGSVIWERMLQDDDWDFFLTGIWEYQWTKTPVDFSDEINEAFYYECQGIIDIAIKLYKMIQWRAISLGGDEKITVDLIHQSAKDGLYLVKPMLDAIRSGDQDWMIKYKDIAPINAIEYKNKCLSELESRDLEEVRRLARKQQNRGNQSVKLRHIIVELVKLD

SEQ ID NO: 232 - protein, 315 aa, Nostoc sp.
MLSFFPQLYPDELLYSGLARYHIRSGNTSFRQTEIELFGYYSQQICRVILPSNLNYLVNNLPLRSKQTVENIIQNHSLYPFYATFLIPQEAWVVRESMKKKLNGSILEMAKIAFNSTDRAKKFLKFCPICMEEDTQKFGEGYWHRFHQIPGVMVCSVHNSALYDSSVIVETRNTHYHAASPDNCILNTNTRNYTEGTLRNLIVLAREVEWLISNNMDFKGLAWLRSQYKHYLVNKKLITLFPGRKFIFKEKDFSKSIFNFYGQNFFETINQNCIKNPDKYFSNCLLACDINPVIDRIIHILIINFLANSLENFFR

SEQ ID NO: 233 - protein, 238 aa, unidentified
  source 1..238: note = Cyanobacteria phylum
MVKEMSSYFSLPTQTPITETLTQTYPKETLTPKLPWVELRFSIIGQTIPVDHGYGLYSAIAHFQSQVHDIDTLGIHTIAGIPSEDGQIHLTDNSRLHIRLPVDKISLIYPLAGKSLKIGKHTIRLGIPEIYLLEPAERLRSRIVIIQGYQKPESFLAAAQRQLEQLGIQGKAFITKNTDGSPKRRSIKIKRFTVVGFGLEIINLSNEDSLTLLRYGIGGKRKMGCGVFVPVKIKGNAS

SEQ ID NO: 234 - protein, 360 aa, Geitlerinema sp.
MSVVASVVNPKIQLNLGDPSITWIHRVGIAGLWMTLKQLEKLYPIPAERPGNLTWLLTPRSISLEWQGQDFVVLDWLLKQSFQINEEGLISLTGLNTHSINIETQINIHLGITGTFLQHNQVFKFDGGKSWSLIVDGIEIDVEYKKAVSYAHQHFAKQLCDKQGQLLGQSIGVAGWLYPGSVVRHVAFTKETRFEEKPQLAFALLYALVACHYFVLRSHTKLEQTQYALVIPEVVDLEIYAQEYWNLGNLDYKDFHVSSLGDAALKFLTYETIVELAIPDQEKRCQVMLFGTVIWSKQQKTRIEIAVVEATEIMDFIYKLSRISFPEYQIIEYKNKNFIISNMFRGTIADNLVKGFPWWA

SEQ ID NO: 235 - protein, 324 aa, synthetic construct
  REGION 1..324: note = Description of sequence: Unknown Bacteria sp.
MFHLFGNILTSYGTAANNRGENEGNITPLQKLNWKGEVHTIVSSEAIRWALRYYWQNVGYPVNRRWDENAQPVADHLWQDLNFDDIRFIDDDVLGFMQAEAAKVEASDESGGDAEEASQDENKKSSKGKDKQTSPRKGKQKPKGKITARRGVLEVTRAVSMIPFMGDITFNAVSGVKRRNSLYATEIHATRYQYGFALTPDSLKDKSRIHAVLDGLISIGEVAGNHARFLYDFSPESIVLRWTHDFSPRFLYCFEQDELGNMSISNLVRQVEVGDIDPKELWIGGLISHGLEDLGANVFPGVKSAVKALKQVIAEDLQLSSGSD

SEQ ID NO: 236 - protein, 208 aa, unidentified
  source 1..208: note = Cyanobacteria phylum
MTTVALKVEVPIACFRQSRAREYAETYPVPPPSTVYGMLLSLIGETDRYKHCGTQLAIALLSKPEKSIVIRTFRRFKTKDIHHPKNAKPDYQELLTNVEFVVWVKTDKAKPTLAELLQQAFANPASINRFGGLCLGESRDLVNTVTLLPEKYHSESLWWLVTDEDGSLTLPYWVDHVGSKRTRWQCYELQESQVWQPPDLSWTTIQTS

SEQ ID NO: 237 - protein, 360 aa, Microcoleus sp.
MLGFFPAPYPDEILYSILARYHIRSGNTSPKITLRELFNSQDVIATVDLPSNLNSLIENLQFISNYQVEDLIYKYTLYPFYSVFLPPKRASQVMESMKADFGGDIHTRTGIMASSLKMQRYLRFCPKCLEEDLQNYGESYWHRLHQTPGVLVCPVHGVTLQDSTIPIQGFNRHEYHAASQENCPVVPIQQAYSQETLKKLRLLAQDISWLITSNLFSRELEWFQRKYIALLTEKDFATATGRVNQKRLLDNFLFFYGAEVLEAVNSMVNYENEQNWLFSIVRKHRKSFHPIRHLLIIRFLTNSIEEFFNTDYQYKPFGEGPWLCLNAAVDHYLKPVITNLVVTHCLDNKKPLGTFSCSCG

SEQ ID NO: 238 - protein, 360 aa, synthetic construct; identical to SEQ ID NO: 217
  REGION 1..360: note = Description of sequence: Unknown origin

SEQ ID NO: 239 - protein, 272 aa, Desulfovibrio zosterae; identical to SEQ ID NO: 218

SEQ ID NO: 240 - protein, 458 aa, Bacillus akibai; identical to SEQ ID NO: 220

SEQ ID NO: 241 - protein, 360 aa, unidentified; identical to SEQ ID NO: 221
  source 1..360: note = Cyanobacteria phylum

SEQ ID NO: 242 - protein, 316 aa, Nostoc sp.; identical to SEQ ID NO: 222

SEQ ID NO: 243 - protein, 227 aa, unidentified; identical to SEQ ID NO: 223
  source 1..227: note = Cyanobacteria phylum

SEQ ID NO: 244 - protein, 360 aa, Geitlerinema sp.; identical to SEQ ID NO: 224

SEQ ID NO: 245 - protein, 324 aa, synthetic construct; identical to SEQ ID NO: 225
  REGION 1..324: note = Description of sequence: Unknown Bacteria sp.

SEQ ID NO: 246 - protein, 210 aa, unidentified; identical to SEQ ID NO: 226
  source 1..210: note = Cyanobacteria phylum

SEQ ID NO: 247 - protein, 360 aa, Microcoleus sp.; identical to SEQ ID NO: 227

SEQ ID NO: 248 - protein, 360 aa, Microcoleus sp.
MISFLPKPYPDEILYSILARYHIRSGNTSPKTTLKELFNSQTTIATIDLPSNLDYLIKNLQFVSNYQIEDLIYKHTLYPLYSPFLPTERASQVLESMKGDDGGDIHTRAGIMASSITMPKYFRFCPTCLEEDLHNYGESYWHRIHQTPGVLVCPLHRQVLQNSIVSLQGFNRHEYYASSTRNCHINKQDNIYSRETLEKLLVLAKDIFWLINSNLTSQAINWFQKRYVNLMIEHGLATATGRIYQKKLLDSFLLFYGREVLNAVNSIVSFTNKNNWVSSIVRKHRKSFHPIRHLLMMRFLQTSPEEFFQHNYEYKPFGNPPWLCLNAAADHYFQPVIKNLTITLCCDAKKPVGTFSCACG

SEQ ID NO: 249 - protein, 173 aa, unidentified
  source 1..173: note = Cyanobacteria phylum
MLLSLVGEENRYKHCGVKLAIALLSEPEKSVVIRTSRRFKKKNIHDLSNSRPDYQELLTNIELVVWLKTGEDQATPTLLERLQQAFNNPASVNRFGGLCLGESRDLVNAVSFVPENYQGESLLWLIQDETGFLTLPYWVDHVGSKGTRWQRYTLQESQIWQPPDLSWTVIQSV

SEQ ID NO: 250 - protein, 311 aa, synthetic construct
  REGION 1..311: note = Description of sequence: Unknown Bacteria sp.
MLHLFGNILTSYGTAANNRGENEGNITSLQKIIWKGEVHTTVSAEAIRWALRYYWQNSGYPVNRIWNENAQPESEHVWQDINFDNVRFIDDDVLGFMRAEAAKNEISEESEDELQEETLDQNKKTRKAKSKQKPKGKKTARRGALEVTRAVSMIPFAGDITFNAVSGKKERTSLYGTEVHATRYQYGFALIPQSLTDQVRINAILDGLISIGEVAGNHARFLYDFSPESIILRWTHDFSPRILYCFETDELGNISVPDLVRRIEAGDIEPNELWIGGAISRDLEDLGTHVFPGVKAAVAALKQVINQDLHL

SEQ ID NO: 251 - protein, 360 aa, Geitlerinema sp.
MPIVESLVKPKIQLNLSDSNMTWLHGAGMAGLWMTLKQLEKIYPICTQRVGNLTWSLTPRSISLFWEGQDFIVLDWLLKQSFQISDNGLIFLMGLNNPLDLQTQIVIHQGITATFLQHNKFFKSAGTASSLITIDKIKFLVKYKKAAYYANQHFAKHLCDNHGQFLKNPIGVKGWLYPGCVVRHYAFEKETTFKEKPEYALILLFAPMACQYFALLSPISPDETQYALIIPKVLDLELYAQYYWQLENLSYKYFHASSIGDAGLKFLTYANAGEMNQLNPVKQCQVILFGTADWSERQKTRIEIAIVELNEKIDYYYQISRRYFADTGFFQYKNQNCLYISFVRGMIADNLAKGFPWWSN

SEQ ID NO: 252 - protein, 232 aa, unidentified
  source 1..232: note = Cyanobacteria phylum
MKNMSSNFSLQTCKSSVQMYPNEALNPQLTYVDLSFSIIGQTLPIDHGYGLYAALSHIKSQIHALDNLSIQTIPGIPDKQGLLQLQLNDHSRLRMRLQAEKIPLVYSFAGKSLTIGKHSIRLGIPQIYLLQPVENLRSRIVVIKGYEQPETFLAAAQRQLEKLGIQGTVRIPTNTDGQPERRTIKIKKFTVVGFRLEVKNLSDEDSLMLQVYGIGGKHKMGCGVFVPIKNSR

SEQ ID NO: 253 - protein, 313 aa, Nostoc sp.
MLSFFPKLYPDELLYSGLARYHIRSGNKSFRQSHLELFQSSLQKGRLILPNNLNKLIKILPFGTSITIENIIQGHTLYPFYTTFLTQNEAWLLKDVMKKQVNQSIFQVAKVALNSTDNLKFLRFCPLCLEEDMKNYGEAYWHRTHQFPSVMVCAIHNVLLYDSLVSLKSAGINYYAAHLGNCLINQQNNDNTEQFLQQLLMIARTTCELINLNFSFKGLAWLRSQYKSYLVKQKFMSLLPGGKFIFQDQYFSDCMVDFYGYNCLKAINPNLLKKPDTFFINCLLSCDMNQVIDRTTHILIIKFLATSIEDFFK

SEQ ID NO: 254 - protein, 459 aa, Bacillus akibai
MQGYLARNILKPDYARRSRQIYDAIKDGGGKTLDNYVDVPTSASGLTLIGPSGMGKSTNLLNILALYPQVILHPEQSTFQIVWLKVDCPHAGSLKGLCVDIFLAIDRLLGTNYFKKFGSRGNSEDYMLAQVAQISHTHNLGVLVIDEMQNLVNARRNKDDLLNFLVKMDNTIGVPVIRVGTNEAFPILQGNFRNARRGTGEGSVIWDRMLQDDEWDFFMEGIWEYQWTKTSVSFSNEINKYFYSESQGIIDIAIKLYKMVQWRAISLGGDEKITVDLIRQAAKDGLYLVKPMLDAIRSGDQEWMLKYKDIAPLDTTEYYKQCLSNLEGRDLKEIRRLARKKQGNTVSPKLNYVIIELLNLDVEASTAKECAEIVVASNEDDEDITSLVKKAYTLALSGGQSQVSPTKTTTSKTKVKPKYQTDDMRQIVEKAKKDQTSAYEDLKSLGIIKDPVQDFIKTY

SEQ ID NO: 255 - protein, 360 aa, unidentified
  source 1..360: note = Cyanobacteria phylum
MQGYLARNILKPDYARRSRQIYDAIKDGGGKTLDNYVDVPTSASGLTLIGPSGMGKSTNLLNILALYPQVILHPEQSTFQIVWLKVDCPHAGSLKGLCVDIFLAIDRLLGTNYFKKFGSRGNSEDYMLAQVAQISHTHNLGVLVIDEMQNLVNARRNKDDLLNFLVKMDNTIGVPVIRVGTNEAFPILQGNFRNARRGTGEGSVIWDRMLQDDEWDFFMEGIWEYQWTKTSVSFSNEINKYFYSESQGIIDIAIKLYKMVQWRAISLGGDEKITVDLIRQAAKDGLYLVKPMLDAIRSGDQEWMLKYKDIAPLDTTEYYKQCLSNLEGRDLKEIRRLARKKQGNTVSPKLNYVIIELLNL

SEQ ID NO: 256 - protein, 480 aa, Bacillus akibai
MDSNLFVNDLIEWTNESSENLIERIIWIDEGYIIAFVLDINADKGFPEPKNITATIEAIHDGRALKLKSDPWARIVRDEDLSDKEKEIRDKAWNIISTLVIQEPEIYYRDKRGSLIKNIINQYTKQKQEERLTEKTVYKYLRKFWQRGKNKNALLPDYVNSGGKGKLKASGDKKRGRPRKYAHISEIGSGINITEDDKKVFRFAISKFYNNPKQNFLTTAYNLMIKEYYAEEIIFDENGVRRSILISPEQRPTFTQFKYWYEVEQTDIRKTIISRKGAKKYDLEHRAILGTSQMETIGPGSRYQIDATIADVYLVSQYNRNWIIGRPIIYVIIDVFSRMITGIYVGLEGPSWTGAMMALANAATNKVKFCQEYGIEITEEEWLCQHIPDAILGDRGELAGMKVETLIPNLNVRIENAAPYRADLKGLVERHFRIIHGYVKPFVPGYIDTDFRQRGGHDYRLDSRLDINQFTEIVINLILY

SEQ ID NO: 257 - protein, 207 aa, Desulfovibrio zosterae
MFYVFEWSDVVIDTREQFPLLDLDLIMSIAEDMGFKYPKDPNSDTPYILTTDFMLSVKENGKNVQKARTVKQVKDISSTSVANKFELERRYYAAKDIDWGIITEKEIPRLLAENVEWVHSAYKLEATNEINIEELRDIAQILKSRLQESDTTINRVTTSLDKEMNLESGTSLYIFKHLIARKEILMDMLGTKISSCPSTTVIQKIVF

SEQ ID NO: 258 - protein, 360 aa, synthetic construct
  REGION 1..360: note = Description of sequence: Unknown origin
MCGIVGYIGTQAATDILLAGLEKLEYRGYDSAGIATILEGDVHCVRAKGKLLNLRSKLAQIENPAQIGIGHTRWATHGKPEEYNAHPHLDTALRIAVVQNGIIENYRELREHLKALGHEFRSETDTEVIPHLIAECLKHTSENLASPSMFLDAVQDAASKLQGAYAIAAISADFPDELIVVRQQAPLVIGFGQGEFFCASDTPAIAPYTRAVLSLENGEIARLTPLGVEVYNFAGHRLKKHPRTLNWNPIMVEKQGFKHFMLKEIYEQPGVVRDCLEAYFPTGENAQITPESPVKLDLPAEFYADLEQVQIVACGTSWHAALVGKYLLEQLAGIPTQVQYASEFRYAPSPLTANTLTIGV

SEQ ID NO: 259 - protein, 360 aa, Microcoleus sp.
MLGFFPAPYPDEILYSILARYHIRSGNTSPKITLRELFNSQDVIATVDLPSNLNSLIENLQFISNYQIEDLIYKYTLYPFYSVFLPSKRASQVMESMKADFGGDIHTRTGIMASSLKMQRYLRFCPKCLEEDLQNYGESYWHKLHQTPGVLVCLVHGVTLQDSTIPMQGFNRHEYYAASLENCPVIPIQETYSQETYSQETLKKLRLLAQDISWLINSNLFSRELEWFQRKYIALLIEKNFATATGRVNQKRLLDNFLFFYGAEVLEAVNSMVNYENEQNWLFSIVRKHRKSFHPIRHLLVIRFLTNSIEEFFNTDYQYKPFGEGPWLCLNAAVNHYLKPVITNLVVTHCLDNKKPLGTF

SEQ ID NO: 260 - protein, 208 aa, unidentified
  source 1..208: note = Cyanobacteria phylum
MTTIALKVEVPIACFRQSRAREYAETYPVPPPSTVYGMLLSLIGETDRYKHCGTQLAIALLSKPEKSIVIRTFRRFKTKDIHDPKNTKPDYQELLTNVEFVVWVKTDKAKPTLPELLQQAFANPASINRFGGLCLGESRDLVNTVTLLSEKYHGESLWWLVTDEDGSLTLPYWVDHVGSKRTRWQCYELQESQVWQPPDLSWTTIQTS

SEQ ID NO: 261 - protein, 324 aa, synthetic construct
  REGION 1..324: note = Description of sequence: Unknown Bacteria sp.
MFHLFGNILTSYGTAANNRGENEGNITPLQKLNWKGEVHTTVSSEAIRWALRYYWQNVGYPVNRRWDENAQPVADYVLQDPNFDDIRFIDDDILGFMQAEAAKVEASDESGGDAEEASQDENKKSSKGKDKQTSPRKGKQKPKGKITARRGVLEVTRAVSMIPFMGNITFNAVSGVKGRNSLYATEIHATRYQYGFALTPDSLKDKSRIHAVLDGLISIGEVAGNHARFLYDFSPESIVLRWTHDFSPRFLYCFEQDELGNLSVSNLVRQVEVGDIDPKELWIGGLISQGLEDLGANVFPGVKSAVKALKQVIAEDLQLSSGGN

SEQ ID NO: 262 - protein, 360 aa, Geitlerinema sp.
MSIVASVVNPKIYLNLGDPSITWIHRAGIAGLWMTLKQLEKLHPIPAERPGNLTWLLTPRSISLEWQGQDFVVLDWLLKQSFQINKEGLISLTGLNTHSIKIETQINIHLGITGTFLQHNQVFKFDGGKSWSLIVDGIEIDVEYKKAVSYAHQDFAKQLCDKQGQLFGQSIGVAGWLYPGSVVRHVAFTKKTKFEEKPQLAFALLYAPVACRYFVLRSHIKLEHTQYALVIPEVVDLEIYAQEYWSLGNLDYKDFHVSSLGDAALRFLTYETIIELATPDQVKRCQVILFGTVVWSKQQKTRIEIGVVEATEIIDFIYKLSRIYFPEYQIVKYKNKNLIISNVFTGTIADNLVKGFPWWA

SEQ ID NO: 263 - protein, 233 aa, unidentified
  source 1..233: note = Cyanobacteria phylum
MIIDCSLPTQIPPESPLNLTKQRKLLTSELPYIELIFNLIGETVPLDHGYELFSAIAHFEPELHKLDTVGIHTIAGIPKPGVIQLTKNSRLRIRLPMDQVRLVYPLAGKPLRIGKHTIRLGIPEIYLLEPAEKLRSRIVIIQGYQEPESFLTAAQRQLEQLGIQGKAFITKNTDGSPKRRSIKIKRFTVVGFGLEIINLSNEDSLTLLRYGIGGKRKMGCGVFVPVKRKGNAS

SEQ ID NO: 264 - protein, 315 aa, Nostoc sp.
MLSFFPTLYPDELLYSALARYHIRSGNKIFKQTDLELFGYSSQQVCKITLTNHLNYLVKNLPLRSHQTVGNLLQNHTLYPFYATFLIPQEAWLLKDSMSKKLSGSILKLAKVANGSSGESKKFLKFCPQCLEQDTHQYGEPYWHRTHQVPGILVCPTHGVFLCDSSVAIESKGIHYYAASLDNCLTVTGLKSLTNQALKTLVTLANDINLLIHGNFAFKGLAWLRAQYQHYLVSKGLLQIFPAGKFKFDEYGFSKLIFDFYGQESLEALNPKLTQHQGKYFSHCLLGCDLNPSIDRITHILIIKYLANSIEIFFL

SEQ ID NO: 265 - protein, 360 aa, unidentified
  source 1..360: note = Cyanobacteria phylum
MTGYLARNVLKPEYASRSRQIYNAIKDGGGKNLENYVNLPTSASGLTLIGPSGMGKSTNFQNILNLYPQVILHPDHSVYQIVWLKVDCPHAGSLKGLCTDIFLAVDRLLGTNYFKKFGSSRNSEDYMLAQVAQIAHTHHLGVLVIDEMQNLVTARRSSSDMLNFLVKMDNTIGIPVIRVGTNEAFPILQGNFRNARRGTGEGSVIWDRMVNNDEWSFFMEGIWEYQWTKTPVPFSDEINEVFYNESQGIIDIAVKLYKMAQWRAISIGGKEIITTNLIRQAAKDGLYLVKPMLDAIRSGDKEWMIKYKDIAPLDTTEYQNKCLSELDSDDLKEIQRLARKQQNQQKVSPKLNYVILELLK

SEQ ID NO: 266 - protein, 480 aa, Bacillus akibai
MVKGLFVNDLIEWTLDLNVHIVERIIWIDEGYIIAFVIDINAKKGLPEPKKISEILEAISEGIAFKQQQDPWARIVRDENLTDREKEYRNKAWKIISSLVTEEPSIYYRNFRGSLVKKVVEKHNLERNKDKLVEKTVYGYLRRFWQRGKNINALIPDFINSSGKGKIRGQSSNKRGRPRKYAHEPDIGEGVNVTEEDRKIFRIAISKFYNNSKENYLTTAYELMVKNYYKEGIRYDENGVQKSILIPADKIPTITQFKYWYQLEQDDVTKTLTSRKGAKKFALENRAILGTSKMETIGPGSRYQIDATVADVYLVSKYNRDWIIGRPVIYVIIDVFSRIITGIYVGLEGPSWMGAMMALANVATDKTLFCQEYNINITEDEWPCNQMPDTILGDKGELAGMSVETLIPNLYVHIENAASYRADWKGLVERHFRIIHEYVKPFLPGYIDTDFRQRGARDYRLDGKLDIDQFTEIIVRLIVH

SEQ ID NO: 267 - protein, 272 aa, Desulfovibrio zosterae
MVRGRREWTQAKYERYIKEGRGRGSGKNYKPWLTIQDFPSKGRSSRIPGWKTNRVHHFFSDHEKRLFYLFEWSDIVTDIKEQFPLDNLDLAMSIATDIGIKYPVDTQTGTPYVLTSDFMLTVNRNGKQIQIARTVKQSTELEKKRVNEKLEIERQYYLAKGIDWGIITEKEISRLLAENVEWVHSAYRLEANSEMDTSQLHELSTILKSKLQEEDATINKITTTLDKEMNVNYGTSLYVFKHLIARKELIVDLVKIKLSACPSTEVIQKIVF

SEQ ID NO: 268 - protein, 360 aa, synthetic construct
  REGION 1..360: note = Description of sequence: Unknown origin
MCGIVGYIGTQAATDILLAGLEKLEYRGYDSAGIATVWEGEVNCVRAKGKLHNLRSKLEQIEAPAQIGIGHTRWATHGKPEEYNAHPHLDTAKRVAVVQNGIIENYRDLREELKAKGHQFVSETDTEVIPHLIAEFLKNIPPSSSSSPFLEAVRQAVNHLHGAFAIAVISADYPDELIVVRQQAPLVIGFGQGEFFCASDTPAIVAYTRAVLPLENGEIARLTPLGVEIYNFAGDRLKKQPRLLNFNPALVEKQGFKHFMLKEIHEQPGVVRASLDAYFNPAESTESPINLGLPADLYNDLEQIQILACGTSWHAALIGKYLLEQLAGISTQVHYASEYRYAPSPVTANTLIIGVTQSGE

SEQ ID NO: 269 - protein, 360 aa, synthetic construct
  REGION 1..360: note = Description of sequence: Unknown origin
MCGIVGYIGTQAATDILLAGLEKLEYRGYDSAGIATVWEGEVNCVRAKGKLHNLRSKLEQIETPAQIGIGHTRWATHGKPEEYNAHPHLDTAKRVAVVQNGIIENYRELREELKAKGHIFLSETDTEVIPHLIAEFLKNLPPSSSSSPFLEAIRQAVNHLKGAFAIAVISADYPDELIVVRQQAPLVIGFGQGELFCASDTPAIVAYTRAVLPLENGEIARLTPLGAEIYNFAGDRLKKQPRLLNFNPALVEKQGFKHFMLKEIHEQPGVVRASLDAYFNPAESTESPINLGLPADLYTDLEQIQIVACGTSWHAALIGKYLLEQLAGISTQVHYASEYRYAPSPVTANTLIVGVTQSGE

SEQ ID NO: 270 - protein, 272 aa, Desulfovibrio zosterae
MARSKQDWTQAKFERYSKEGRDQGNGKNYQPWIKIQDFPSKGRVSRPPGWKTNREHHLLSDNEKRLFYVFEWSDTIVDIREQFPLIDLDLAMSIAEEIGINYPKDPQSNTPRVLTTDFMLSVKQGKTIVQKARTFKLTKDLGSKSVAEKFELEKRYYAAKGIDWGIITEKEVPKLLAENVEWIHTAYKLEGNADIKLEELRNIANILKSRLQESDASINRITTSLDKNMNLESGTSLYLFRHLIANKEIIIDMLTTKISSCPSSREIKKIVF

SEQ ID NO: 271 - protein, 480 aa, Bacillus akibai
MINDLFVNDLIEWIDESGNTFIERVIWIDEGYIIAFVFNINTQTGFPEPKKISEIIEAISDGRALKLKSDPWARIVTEENLSEKEKELRDRAWEIISFIVIQEPSIYYRDYRGALVQQTIEKYNAGRSKGKLVEISVYKYLRRFWQRGKNKNALLPDYVNSGGKGKPKAFGEKKRGRPRKYVHAQEIGVGINVTEEDKKIFRFAIAKFYNNSKENFLTTAYDLMIKKYYSEDFYYDENGVKKSILISPDKRPTFTQFKYWHKVEQPDIRKTIISRKGSTKYALEHRAITGNSQMETIGPGSRYQIDATIADVYLISMYNRNWIIGRPVIYVVIDVFSRMITGVYVGLEGPSWAGAMMALANSAIDKVQFCREYGVEISEDEWPCRHIPDAILGDRGELAGMKVETLIPNLNVRIENAAPYRADWKGLVERQFRTIHGYVKPFVPGYIDTDFRQRGGHDYRLDGRLNIDEFTKVVIFLILQ

SEQ ID NO: 272 - protein, 360 aa, unidentified
  source 1..360: note = Cyanobacteria phylum
MIPNRKSAIVARYIDQKLLEYNTNPLIQALPPILSSEEFIDKVTRTPDFDEQERQLEAHYRFHCIERLSRYFDPQNKTVELQKVISVLIMTGYLARNILKPEYASRSRQIYNAIKDGGGKNLENYVNLPTSASGLTLIGPSGMGKSTNFQNILNLYPQIILHPEYSVYQIVWLMVDCPHAGSLKGLCTDIFLGVDKLLGTNYFKKFGSSRNSEDYMLAQVAQIAHTHHLGALVIDEMQNLVTARRSSSDMLNFLVKMDNTVGIPVIRVGTNEAFPILQGNFRNARRGTGEGSVIWDRMVNDDEWYFFMEGMWEYQCTKMHVPFCDEINEVFYDESQGIIDIAVKLYKMVQWRAISIGGKE

SEQ ID NO: 273 - protein, 315 aa, Nostoc sp.
MLSFFPTLYPDELLYSTLARYHIRSGNNSFKQTDLEVFGYSSQQVCKVTLTNNLNYLVKNLPLCSQQTVDNLLQNHTLYPFYGTFLMPQEAWLLKDSMSKKLSGSILEVAKVATGSSGESKKFLKFCPKCLEEDTQQYGEPYWHRTHQVPGILVCPTHGVVLCDSSVAVESKGIHYYAANLDNCLVITDVKSFTNQALKTLLTLTNDINLLIHSNFAFKGLVWLRTQYQRYLVNKGLLQIFPGGKFKFDEYAFSKLIFDFYGQESLEALNPKLTQHQGKYFSHCLLGCDLNPAVDRITHILMVKFLANSIEAFFQ

SEQ ID NO: 274 - protein, 233 aa, unidentified
  source 1..233: note = Cyanobacteria phylum
MVVDCSLLTQILSESPVNRTKPTEVLIPQLPYIDLIFNLIGETVPLDHGYELFSAIAHFEPELHKLNTVGIHTIAGIPKEGVIQLTKNSRLRIRLPVDQVRLVYPLAGKSLKIGKHTIRLGIPEIYLLEPAERLRSRIVIIQSYQEPESFLAAAQRQLEQLGIQGKAFITKNTDGSPKRRSIKIKGFTVVGFGLEIINLSNEDSLTLLRYGIGGKRKMGCGVFVPVKRKESAS

SEQ ID NO: 275 - protein, 360 aa, Geitlerinema sp.
MSVVASVVNPKIQLNLGEPNITWIHRAGIAGLWMTLKQLEKLYPIPAKRPGNLSWLLTPRSISLEWQGQDFVVLDWLLKQSFQINEEGLISLTGLNTHSINIETQINIHLGITGTFLQHNQVFKFDGGKSWSLIVDGRKIDIEYKKAVSYAHQHFAKQLCDKQGQLLGQSIGVAGWLYPGSVVRHVAFTKETRFEEKPQLAFALLYALVACHYFVLRSHTKPEHTQYALVIPEVVDLEIYAQEYWNLGNLDYKDFNVSSLGDAALRYLTYETIIELAIPDQVKRCQVILFGTVVWSKQQKTRIEIAVVEATGIINFIYKLSRISFPEYQIIQYKNKNFIISNLFRGTIADNLVKGFPWWA

SEQ ID NO: 276 - protein, 324 aa, synthetic construct
  REGION 1..324: note = Description of sequence: Unknown Bacteria sp.
MFHLFGNILTSYGTAANNRGENEGNITPLQKLNWKGEVHTLVSSEAIRWALRYYWQNVGYPVNRRWDENAQPVADHVWQDPNFDDIRFIDDDVLGFMQAEAAKAEASDESGGDAEQASQDENKKISKGKDKQTSPRKGKQKPKGKITARRGVLEVTRAVSMIPFTGDITFNAVSGVKGRNSLYATEIHATRYQYGFALTPDSLKDKSRIHPVLDGLISIGEVAGNHARFLYDFSPESIVLRWTHDFSPRFLYCFEQDELGNLSISNLVRQVEVGDIDPKELWIGGLISHGLEDLGANVFPGVKSAVKALKQVIAEDLQLSSGSD

SEQ ID NO: 277 - protein, 171 aa, unidentified
  source 1..171: note = Cyanobacteria phylum
MLLSLIGETDRYKHCGTQLAIALLSKPEKSIVIRTFRRFKTKDIHDPKNAKPDYQELLTNIEFVVWVKTDKAKPTLPELLQQAFANPASINRFGGLCLGESRDLINTVTLLPEKYYGESLWWLVTDEDGSLTLPYWVDHVGSKKTRWQCYELQESQVWQPPDLSWTTIQTS

SEQ ID NO: 278 - protein, 360 aa, Microcoleus sp.
MLGFFPAPYPDEILYSILARYHIRSGNTSPKIALLELFNSQDVIATVDLPSNLNSLIENLQFISNYQVEDLIYKYTLYPFYSVFLPPKRASQVMDSMKADFGGDIHTRTGIMASSLKMQRYLRFCPKCLEEDLQNYGESYWHRLHQTPGVLVCPIHGVTLQDSTIPMQGFNRHEYYAASQENCPVVPIQQAYSQETLKKLRLLTQDISWLITSNLFSRELEWFQRKYIALLTEKDFATATGRVNQKRLLDNFLFFYGAEVLEAVNSMVNYENEQNWLFSIVRKHRKSFHPIRHLLIIRFLTNSIEEFFNTDYQYKPFGEGSWLCLNAAVDHYLKPVITNLVVTHCLDNKKPLGTFSCSCG

SEQ ID NO: 279 - protein, 360 aa, synthetic construct
  REGION 1..360: note = Description of sequence: Unknown origin
MCGIVGYIGTQAATEILLAGLEKLEYRGYDSAGIATIWEGDVQCVRAKGKLQNLRSKLEHLETPAQIGIGHTRWATHGKPEEYNAHPHLDTAMRVAVVQNGIIENYRELREELKSKGHQFRSETDTEVIPHLIAEIIKKSPSSLLEAVSQAVTRLEGAFALAVISADYPDELIVVRQQAPLVIGFGQGEFFCASDTPAIVAYTRAVLPLDNGEIARLTPLGVEVYNFAGDRLKKQPRLLNLNPTMVEKQGFKHFMLKEIYEQPGVVRASLEAYFNNDGNSAGSSHSPINLGLPESFYADIEQIQIVACGTSWHAALVGKYLLEQLAGISTQVHYASEYRYAPSPLMANTLIIGVTQSGET

SEQ ID NO: 280 - protein, 272 aa, Desulfovibrio zosterae
MARTKQEWTQAKFERYMKEGRGQGSGKNYQPWIKIQDFPSKGRVSRPPGWKTNREHHLFSDHEKRLFYLFEWSDSIIDIREQFPLLDLDLAINIATEMGIDYPKDPNNDTPYVLTTDFMLSVKQGNQIVEKARTFKLVKDLGTKSVAEKFELEKRYYAAKGIDWGIITEKEIPMFLVKNVEWIHSSYKLEAAAEIKVDELHNIANILKSKLQESDSTINKITNSLDKEMNLEVGTSLYIFKHLLANKKIIMDMQTRTISSSISSREIKKIIF

SEQ ID NO: 281 - protein, 480 aa, Bacillus akibai
MNKNLFVNDLIEWVDELGNNFVERILWIDEGYVIAFTIDINVKTGFPVSKRVSDIQAAISEGHALKLKTDPWARIVRDEDLSAKEKEIRDKYWDIISPIVIQEPSIYYRDKRGSLIKQVVEKYNHNRSEGKIIERSVYQIIRRYWQRGKDKNSLLPDYANSGGKGKVRASGEKKRGRPRKYAHEPEIGIGINITDEDKKIFRLAVTKFYHNQKENFLTTAYDLMIKNYYSEETIYDDNGVKKHILIPPDKRPTLTQFKYWYDVEQGDIRKKIISRRGAKKYALEHRAITGNSTMETIGPGSRYQIDATIADVYLVSAYNRNWIVGRPVIYVVIDVFSRMITGVYVGFEGPSWIGAMMALANAASNKVAFCKEYGIEITEDEWACHHIPDAILGDRGELAGMKVETLIPNLGIRIENAAPYRADWKGLVERHFGIFHGHIKPFVPGYIDKDFRERGARDYRLDSKLDIEQFTEIIIKIILY

SEQ ID NO: 282 - protein, 360 aa, unidentified
  source 1..360: note = Cyanobacteria phylum
MCALLMQGYIARNISKPEYARRSRQIYEAIKAGDGKNLDVYVNVPTSTSASGLTLIGPSGMGKSTNFINILNLYPQVILHPEYSTYQIVWLKVDCPHAGSLKGLCTDIFVGIDRLLGTNYFKKFGASRNSEDYMLAQVAQIAHTHHLGVLVIDEMQNLVTARRSCSDMLNFLVKMDNIIGVPVIRIGTNEAFPILQGNFRNARRGTGEGSVIWDRMRNDDEWYFFMETMWEYQWTKISTDFSDELNDVFYDESQGIIDIAIKLYKMVQWRAISLSGKEEINVIKLYKMVQWRAISLSGKEEINVNLIKQSAKDGLFLVKPMLNAIRSGDKEWMIKYKDIAPIDTTEYYNNCLSNLETKDL

SEQ ID NO: 283 - protein, 315 aa, Nostoc sp.
MLSFFPNLYPDELLYSALARYHIRSGNKTFKQTDLELFSYSSQQLCKVTLTNNLNYLIKNLSLFSQLTVSDLLQNHTLYPFYSTFLIPQQAWQLQDSMKNKLSGSILEVAKVANYPTENLTKFLKFCPVCLEKDTQEYGEPYWHRIHQIPGILVCPTHRIILHDSSVAVDSKGIHYHAASPENCLCNQNQVKYTDSTVEKLLILAQDIWDTSNINISFKGLAWLRNQYQSYLTNQKFMNLLPGGKFRFKQKSFSNSIRDCYGQEFLEIINPNLIKNPDKYFSSYLLGCDLNPVIDRITHILIIKFLANSVERFFT

SEQ ID NO: 284 - protein, 222 aa, unidentified
  source 1..222: note = Cyanobacteria phylum
MSDNFHVSTQNHATEVLNPQLMYVDLSFRVIGQTLPIDHGYGLYAALTHLQDKLHSLDDLSIQTIPGIPDGNDSLHLNNHSRLRIRLPVDKIPLVYPFAGKSLTIGRHKIRLEIPQMYLLQAVEKLRSRIVVIKGYEEPDAFLAAAQRQLERLGIKGIASIPTKADGKPERRSIKIKKFTVVGFGLEVTNLSDEDSITLQKYGVGGKQKMGCGVFVPVNEKR

SEQ ID NO: 285 - protein, 360 aa, Geitlerinema sp.
MINTTANSKIQLNLGDSSTTLLHRAGIAGFWMTLKQLEKIYPNPAQRTGNLTWVLTPHSISLNWEGQDFAALDWLLKQSFQISDEGLISLTGLNLYAMDIERQIIIHQGITGTFLQHNKFFKSSNKKLKPLIVDGKKFVTEYKSAISYAHQHFAIQLCDESGQFLQEPIGIVGWLYPGAVVRHYAFKEQTQFEEKPEYALALLFAPVACHYFILRSHTKPQSTKYILVVPEVNNLELYAQHYWTLSNLSYRDFHVSSLRDAGLKLLTNQATIQIKQNCVKRCQVFSFGKLIWSGKQKVRTETATLELTEKVSFYYKLSRSCFPNYRIVYYGNQHFLLTSLFQGIIANNLGLGLPWWNDFG

SEQ ID NO: 286 - protein, 332 aa, synthetic construct
  REGION 1..332: note = Description of sequence: Unknown Bacteria sp.
MSHLFGNILTIYGTAANNRGENEGNITTLQKINWKGEVHTTVSAEAIRWALRYYWQNNGHEVNRRWDENAQPLADHILQDLNFNNVRFIDDDVLGFMQTEAAKVEAGDQSGGEVQEASAGEIQNVSQDEEVTSSKGKRKKTSQRKGRERPQGRVTAKRGVLEVTRAVSLIPYEGNITFNAVSGKKGRTSLYGTEVHATRYQYGFALTPNSLQDKSRINAVLNGLISIGEVGGNHARFLYDFSPDSIILRWTHDFSPRFLYCFEQDEWGNISVPDLLRRVEAEDIDPKELWIGGAIARDLGNLGANVFPGVKKVVEELLKVMTRDLQLPPRSS

SEQ ID NO: 287 - protein, 173 aa, unidentified
  source 1..173: note = Cyanobacteria phylum
MLLSLLGEVDRYKHCGVKLAIALISEPAKSTVIRTFHRLKTKNIHNPKNNKPDYQELLTNIEFVVWVDAGLDKAKPNLVQRLEEAFTNPGSINRFGGLCLGESRDLVNDITLLPKNYYAESMRWLIRDEYGLLTLPYWVDHVGSRGTRWLSYEIQECQVLQPPELSWTLVQSD

SEQ ID NO: 288 - protein, 360 aa, Microcoleus sp.
MVSFFPQPYPDELLYNVIARYHIRSGNTSPKITLQELFNSQSTIATVDLPSNLNNLIQNIQFISNYKVDDLIYKHTLYPLYSPFLPVNRSSQVLESMKGDYGGDIHTRAGIMASSITMPKYFQFCPTCWEEDLKNYGEPYWHRIHQIPGVLVCPFHGEVLQDSLAPLHGFNKHEYYAASTDNCSITKEPSIFSSDTLKKLLVLAKDFWWLLNSEIPCQEPQWFRKQYTNLMIEQGFATATGRIYQKKLLDNFLFFYGREMLSAVDSIVNHNDSNNWVTSIVRKHRKSFHPLRHLLMMRFLKTSVEEFFQQDTEYKPFGEPPWLCLNAAAEHYLQPVITNLVITLCCDTKKPVGTFSCSCG

SEQ ID NO: 289 - protein, 360 aa, unidentified
  source 1..360: note = Cyanobacteria phylum
MCALLMQGYIARNISKPDYARRSRQIYEAIKAGDGKNLDVYVNVPTSTSASGLTLIGPSGMGKSTNFINILNLYPQVILHPEYSTYQIVWLKVDCPHAGSLKGLCTDIFLGIDRLLGTNYFKKFGASRNSEDYMLAQVAQIAHTHHLGVLVIDEMQNLVTARRSCSDMLNFLVKMDNIIGVPVIRIGTNEAFPILQGNFRNARRGTGEGSVIWDRMRNDDEWYFFMETMWEYQWTKISTDFSDEFNDVFYDESQGIIDIAIKLYKMVQWRAISLSGKEEINVNLIKKSAKDGLFLVKPMLNAIRSGDKEWMIKYKDIAPIDTTEYYNNCLSNLETKDLQEISRLAKKQQTENGHLSATQR

SEQ ID NO: 290 - protein, 480 aa, Bacillus akibai
MNNNLFVNDLIEWVDESGNNFVERILWIDEAYIIAFTIDINIKTGFPVSKRVSDIQEAINEGHALKLRTDPWARIVRDEDLSAKEKEIRDKYWDIISPIVIQEPSIYYRDKRGSLIKQVIENYNHNRTEGKLIERSVYQIIRRYWQRGKDKNSLLPDYANSGGKGKVKASGEKKRGRPRKYAHEPEIGVGINITNEDKKIFRLAVTKFYHNRKENFLTTAYDLMVKNYYSEETIYDENGVKKHILISPDKRPTLTQFKYWYEVEQGDIRKKIISRRGVKKYALEHRAITGNSTMETIGPGSRYQIDATIADVYLVSAYNRNWIVGRPVIYVVIDVFSRMITGVYVGLEGPSWIGAMMALANAASDKVAFCKEYGIEITEDEWPCHHIPDAILGDRGELAGMKVETLIPNLGIRIENAAPYRADWKGLVERHFGIIHGHIKPFVPGYIDKDFRERGARDYRLDSKLDIEQFTEIIIKIILY

SEQ ID NO: 291 - protein, 272 aa, Desulfovibrio zosterae
MARTKQEWTQAKFERYIKEGRGQGSGKNYQPWIKIQDFPSKGRVSRPPGWKTNREHHLFSDNEKRLFYLFEWSDSIIDIREQFPLLDLDLAINIATEMGIDYPKDANNETPYVLTTDFMLSVKQGKQIVEKARTFKLVKDLGTKSIAEKFELEKRYYAAKGIDWGIITEKEIPMFLVKNVEWIHSSYKLEAAAEINVDELHNIANILKSKLQERDSTINKITNLLDKEMNLEVGTSLYIFKHLLANKKIIMDMQTSAISSSISAREIQRIIF

SEQ ID NO: 292 - protein, 360 aa, synthetic construct
  REGION 1..360: note = Description of sequence: Unknown origin
MCGIVGYIGTQAATEILLSGLEKLEYRGYDSAGIATIWEGDVQCVRAKGKLQNLRSKLEQVETPAQIGIGHTRWATHGKPEEYNAHPHLDTAMRVAVVQNGIIENYRELREELKAKGHLFRSETDTEVIPHLIAEILKTSPSSLLEAVRQAVNLLEGAFAIAVISADYPDELIVVRQQAPLVIGFGQGELFCASDTPAIVAYTRAVLPLENGEIARLTPLGVEIYNFAGERLKKQPRLLNLSPTMVEKQGFKHFMLKEIYEQPGVVRAILEAYFSNENNSSSHTQSPINLGLPESFYADIEQIQIVACGTSWHAALVGKYLLEQLAGISTQVNYASEYRYAPSPLIANTLIIGVTQSGET

SEQ ID NO: 293 - protein, 360 aa, Microcoleus sp.
MVSFFPKPYPDEILYSVITRYHIRCGNTSPKITLQELFNSQTTVATVDLPSNLNNLIQNLQFISNYQVEDFIYKHTLYPLYSPFLPANRSSQILESMKGDYGGDIHTRAGIMASSITMPKYFRFCSTCQEEDLKNYGELYWHRIHQIPGVLVCPFHGEFLQNSLAPLHGFNKHEYYAASTDNCSITREPSIFSTDTLKKLQVLAKDFWWLINSEILSQEPQWFRKQYTNLMIEQGLATATGRIHQTRLLDNFLFFYGREMLSAVDSMVTHNDSSNWVTSIARKHRKSFHPLRHLLMMRFLKTSVEEFFKQDTEYKPFGKSPWLCLNAAAEHYLQPVITNLVITLCSDTKKPVGTFSCSCG

SEQ ID NO: 294 - protein, 173 aa, unidentified
  source 1..173: note = Cyanobacteria phylum
MLLSLVGEVNRYKHCGVKLAIALISEPAKSTVIRTFHRLKTKNIHDPKNNKPDYQELLTNIEFVVWVDAGVDKAKPNLVQRLEEAFTNPASINRFGGLCLGESRDLVNDITLLPKNYYAELMRWLIQDEYGLLTLPYWVDHVGSRGTRWLSYEIQECQVLQPPELSWTSVQSD

SEQ ID NO: 295 - protein, 332 aa, synthetic construct
  REGION 1..332: note = Description of sequence: Unknown Bacteria sp.
MSHLFGNILTIYGTAANNRGENEGNITTLQKINWKGEVNTTVSAEAIRWALRYYWQNSGYEVNRRWDENAQPLAAHILQDINFNNVRFIDDDVLGFMQTEAARVEAGDQSGGEVQEASAGEIQNVSQDEEATSSKGKRKKTSQRKGRERPQGRVTAKRGVLEVTRAVSLIPYGGNITFNAVSGTKGRTSLYGTEVHATRYQYGFALTPNSLQDKSRINAVLNGLISIGEVGGNHARFLYDFSPDSIILRWTHDFSPRFLYCFEQDEWGNISVPDLVRRVEAEDIDPKELWIGGAIAKDLGNLGANVFPGVKKVVEELLKVITRDLQFPPRSS

SEQ ID NO: 296 - protein, 360 aa, Geitlerinema sp.
MINTTANSKIQLNLGDSSTTLLHRAGIAGFWMTLKQLENIYPNPAQRTGNLTWVLTPHSINLNWEGQDFTALDWLLKQSFQISDEGLIALTGLNPQTMDFERRIIIHQGITGTFLQHNKFFKSSNKKLKSIIVDGKKFVTEYKSAISYAHQHFAKQLCDESGQFLQEPIGIVGWLYPGAVVRHFAFAKQTKFEENPEYALALLFAPVACHYFIWRSHTKPQSTKYILVVPEVNNLEIYAQHCWNLSNLSYRDFHVSSLRDAGLKFLTDETAVQVNQNCVKRCQVFSFGTLIWSEQQKVRTETAILELTEKVSFYYKLGRSCFPHCRIVYYQNQHFILTSLVQGIIANNLGLGSPWWNDFG

SEQ ID NO: 297 - protein, 222 aa, unidentified
  source 1..222: note = Cyanobacteria phylum
MADHFHLSTQNHVTGVLNHQLIYVDLSFRVIGQTLPIDHGYGLYAALTHLQDKLHSLDDLSIQTIPGIPDGKDSLHLNNHSRLRIRLPVDKIPLVYTFAGKSLTIGRHKISLEIPQMYLLQAVEKLRSRIVVIKGYEEPDAFLAAAQRQLERLGIQGIASIPTKADGKPERRSIKIKKFTVVGFGLEVTNLRDEDSITLQKYGVGGKQKMGCGVFVPVNEKR

SEQ ID NO: 298 - protein, 315 aa, Nostoc sp.
MLSFFPNLYPDELLYSALARYHIRSGNKTFKQTDLELFGYSSQQLCKVTLTNNLNYLVKNLSLFSQLTVSDLLQNHTLYPFYSTFLIPQQAWQLQDSMRNKLSGSILEVAKVVNHPTENSTNFLKFCPVCLEKDTQEYGEPYWHRIHQIPGILVCPTHRIILHDSSVAVDSKGIHYHAASPENCLYNQNQVKYTDSTVEKLLILAQDIWDTSNINISFKGLAWLRNQYQYYLINQKFMNLLPGGKFRFNQKFFSNSICKFYGQEFLEIINPKLIRNSEKYFSSYLLGCDLNPVIDRITHILIIKFLANSVERFFT

SEQ ID NO: 299 - protein, 360 aa, Microcoleus sp.; identical to SEQ ID NO: 293

SEQ ID NO: 300 - protein, 173 aa, unidentified; identical to SEQ ID NO: 294
  source 1..173: note = Cyanobacteria phylum

SEQ ID NO: 301 - protein, 332 aa, synthetic construct; identical to SEQ ID NO: 295
  REGION 1..332: note = Description of sequence: Unknown Bacteria sp.

SEQ ID NO: 302 - protein, 360 aa, Geitlerinema sp.; identical to SEQ ID NO: 296

SEQ ID NO: 303 - protein, 222 aa, unidentified; identical to SEQ ID NO: 297
  source 1..222: note = Cyanobacteria phylum

SEQ ID NO: 304 - protein, 315 aa, Nostoc sp.; identical to SEQ ID NO: 298

SEQ ID NO: 305 - protein, 360 aa, unidentified; identical to SEQ ID NO: 289
  source 1..360: note = Cyanobacteria phylum

SEQ ID NO: 306 - protein, 480 aa, Bacillus akibai; identical to SEQ ID NO: 290

SEQ ID NO: 307 - protein, 272 aa, Desulfovibrio zosterae; identical to SEQ ID NO: 291

SEQ ID NO: 308 - protein, 360 aa, synthetic construct; identical to SEQ ID NO: 292
  REGION 1..360: note = Description of sequence: Unknown origin

SEQ ID NO: 309 - protein, 360 aa, Microcoleus sp.
MVSFFPQPYPDEILYSVIARYHIRSGNTSPKITLQELFNSQSTVATVDLPSNLNNLIQNLQFISNYQIEDLIYKHTLYPLYSPFLPVNRSSQVLESMKGDYGGDIHTRAGIMASSITMPKYFRFCPTCWEEDLKNYGEPYWHRIHQTPGVLVCPFHGEVLQDSLVTLHGFNKHEYYAASTENCSITKEPSIFSPDTLNKLLVLAKDFWWLLNSEIPCQEPQWFRKQYTNLMIEQSLATATGRIYQKKLLDHFLLFYGREMLSAVDSMVNHNDSSNWVTSIVRKHRKSFHPLRHLLMMRFLKTSVEEIFKQETEYKPFGEPPWLCLNAAAEHYLQPVITNLVITLCCDTKKPVGTFFCACG

SEQ ID NO: 310 - protein, 210 aa, unidentified
  source 1..210: note = Cyanobacteria phylum
MTTIALKVEVPIACFRQSRAREYGETYPVPPPSTVYGMLLSLVGEVDRYKHCGVKLAIALLSEPEKSTVIRTFHRFKTKNIHDSKNNKPDYQELLTNIEFIVWVDAGVDKSKPNLVERLEKALTNPASINRFGGLCLGESRDLVNDVTLLPENYNTESMRWLIRDEYGLLTLPYWVDHVGSRETRWLSYEIQECPVFQPPELSWTSVQSN

SEQ ID NO: 311 - protein, 324 aa, synthetic construct
  REGION 1..324: note = Description of sequence: Unknown Bacteria sp.
MFHLFGNILTNYGTAANNRGENEGNTSTLQKLIWKGEVHSAVSSEAIRWALRYYWQNAGYPVNRSWDENAQPVPDHIWQDPNFDDVRFIDDDVLGFMQAEAAKVEAAVESEVEIQNSTQDEDKKISQGKEKETAKRKRKEKPKGKITTRRGVLEVTRAVSTIPYAGDLTFNAMSGKKGRTSLYSTEVHATRYQYGFALTPNSLKEKSRINAVLDGLISIGEVAGNHARFLYDFSPDSIILRWTHDFSPRLLYCFEEDELRNISTPDLVRRVESGDIDPKELWIGGAIYSTLEDLGANVFPGVKTTVEALKQVITQDLQLSLRSS

SEQ ID NO: 312 - protein, 360 aa, Geitlerinema sp.
MKTAANPKIQLNLGDPSMTLLHRAGVAGLWMTLKKLEKIYPTPAQRIGNLTWLLTPRYISLDWEGQDFVVLDWLLKQSFQISDEGLISLTGLDSQNMDIQNQLIIHQGITATFTQHNKFFKSAGQQSKQLIIDGIQLRVDYKKAASYAHQHFAKHLCDEYGQFLEEPIGIRGWLYPGAVVRHYAFKEQTAFEEKAEYALALLFAPVACQYFVLRSHTEPSHPNYVLVVPEVIDLELYAQYLWCLSNLDYKHFHVSTLGDAGLKFLTYETTKMATSNFLNRCKVVCFSTKVWSEQQKTRAEIAIIELNSSVSYLYKLSCICFPNCQFVLYENQYFLVTSLIKGIIANNLVMGFSWWDNWIS

SEQ ID NO: 313 - protein, 227 aa, unidentified
  source 1..227: note = Cyanobacteria phylum
MVIDSNLVTQLPSESSSKPRELLMSQLPYMDLTFSLMGETLPFDHGYELFSAIAHFEPKLHTLDTLGIHTIAGIPKDGVINLTQNSRLRIRIPVNQVRLVYPLAGKSLRIGKHTIRLGIPDIYLLQPAEKLRSRIVVIRGYEEPETFLVVAQRQLEQLGIQAIVSIPTKANGQPIRRTIKIKRFTVVGFGLEVINLSDEDSLRLQIHGVGGKQKMGCGLFMPIKEKQ

SEQ ID NO: 314 - protein, 316 aa, Nostoc sp.
MLSFFPTLYPDELLYSTLARYHIRSGNKSFKQTDLELFGYSSQQICKITLTNNLNYLVKNLPLRSQQKVENLLQNHTLYPFYATLLMPQEAWLLKNSMSKKFSGSILDFAKVANNSSDDSKKFLKFCPECLEKDTQKYGEPYWHRIHQVPGILVCPIHRVVLHNSCIEVESKGIHYHAASIDNCLLANNVKKLTNKTVETLSTLAHDINWLIHSSFAFQGLAWLRAQYQRYLLDKGLLKTFAGGKFKFDDYGFAQLIYEFYGQESLEAINPRLAQHQGKYFSHCLFGCDVNPAIDRITHILMIKFLANSIEEFFQS

SEQ ID NO: 315 - protein, 360 aa, unidentified
  source 1..360: note = Cyanobacteria phylum
MTGYLSRNLLKPEYANRSRQIYNAIGNGGGKNLENYVNLPTSASGLTLIGPSGIGKTTNFKNILELYPQIIFHPDYSVYQIVWLRVDCPKTLKGLCIDTFAGIDKLLGTNLLKKFNPARTSEDYLLAQLAQTAHTHHLGVLVIDEIQNLATAKRNRDEILNFLVKLDNTIGIPVIRVGTNEAISFLQGNLRNARRNTGEGSAMWDRMGNDDEWCYFLEGIWRYQWTKTPVDFSHEIKEVFYDESQGIIDIAIKLYKMVQWRAISIGDQEIITVDLIRQVAKEGLYLVKPMLDAIRLGDREWMIKYKDIAPLDITEYHNRCVSEIDAKDLTEIKRLAKKQQNQQKVSPKLNYVISELLKLE

SEQ ID NO: 316 - protein, 480 aa, Bacillus akibai
MIHDLFVNDLIEWIDESGNNCIERVLWIDEGYVIAFVYDINAKTGFPEARKVSEIREAISDGRALKLKSDPWARIVRDEDLSEKEKELRDRAWQIILFIVTQEPSIYYRDYRGSLVQQVIEKYNAGRTEGELVPITVYKYLRRFWQRGKNKNALLPDYANSGGKGKPKASGDKKRGRPRKYAQVPEIGIGRNVTEEDKRIFRFAIAKFYKNSKENFLTTAYDLMIKEYYAEDFYYDDNGVKKSILIPPDKRPTFTQFKYWYEIEQTDIRKTIISRKGSRKYALENRAITGTSQMETIGPGSRYQIDATIADVYLVSLYNPNWIIGRPVIYVVIDVFSHMITGVYVGLEGPSWTGAMMALANTATDKVKFCQEYGIEIYEDEWPCRHIPDTILGDRGELAGMNVETLIPNLDVRIENAAPYRADWKGLVERQFRTIHGYVKPFVPGYIDTDFRQRGGHDYRLDGRLNINEFTKIVIFLILQ

SEQ ID NO: 317 - protein, 280 aa, Desulfovibrio zosterae
MQASTGVFMARSKQDWTQAKFERYCKEGRGEGSGKDYKPWIKIQDFPSKGRVSRPPGWKTKREHHLFSDNEKRLFYVFEWSDAIVDIREQFPLLDLDLAISIADEMGINYPKDPQSNTPHVLTTDFMLSVKQSKNIVQKARTFKLTKDLGSKSVAEKFELEKRYFAAKGIDWGIITEKEVPKQLAENIEWIHTAYKLEENADINLEELRNIANILKFRLQESDASINKITTALDRDMNIESGTSLYLFRHLLARKEIIMDMLITKISSCPSSKEIQKIIF

SEQ ID NO: 318 - protein, 360 aa, synthetic construct
  REGION 1..360: note = Description of sequence: Unknown origin
MCGIVGYIGTQAATEILLAGLEKLEYRGYDSAGIATVWEGDVNCVRAKGKLHNLRSKLEQLATSSQIGIGHTRWATHGKPEEYNAHPHLDTAMRIAVVQNGIIENYRELRDELKQKGHEFRSETDTEVIPHLIAEFLKNLPSPALPTSSSSLLEAIRQAVNHLQGAFAIAVISADYPDELIVVRQQAPLVIGFGQGEFFCASDTPAIVSHTRAVLPLENGEIARLTPLGVEIYNFAGDRLKKQPRLLNLNPTMVEKQGFKHFMLKEIYEQPGVVRASLEAYFNPDINTDESFTSPINLGLSEEIYADLEQIHIVACGTSWHAALVGKYLLEQLAGISTQVHYASEYRYAPSPLTANTLII

SEQ ID NO: 319 - protein, 360 aa, Microcoleus sp.
MVSFFPQPYPDEILYSVIARYHIRSGNTSPKITLQELFNSQTTVATVDLPSNLSNLIQNLQFISNYQVEDLICKHTLYPLYSPFLPANRSSQVLESMKGDYGGDIHTRAGIMASSITMPKYFQFCPTCWEEDLKNYGEPYWHRIHQIPGVLVCPFHGEVLQDSLTPLHGFNKHEYYAASTDNCSITKEPSIFSSDTLKKLLVLAKDFWWLLNSEIPCQEPQWFRKQYTNLMIEQGFATATGRIYQNKLLDNFLFFYGQEMLSAVDSIVNHNDSNNWVTSIVRKHRKSFHPLRHLLMMRFLKTSVEEFFQKDTEYKPFGEPPWLCLNAAAEHYLQPVITNLVITLCCDTKKPVGTFSCSCG

SEQ ID NO: 320 - protein, 210 aa, unidentified
  source 1..210: note = Cyanobacteria phylum
MTTIVLKVEVPIACFRQSHAREYSETYPVPPPSTVYGMLLSLVGEVDRYKHCGVKLAIALLSEAEKSTVIRTFHRFKTKNIHDSKNNRPDYQELLTNIEFVVWVDVGVDKAQPNLVERLAEALTNPASISRFGGLCLGESRDLVNDVTLLPENYRAESMRWLIRDEYGLLTLPYWVDHVGSRGTRWLCYEVQECQVFQPPELSWTLIQSD

SEQ ID NO: 321 - protein, 324 aa, synthetic construct
  REGION 1..324: note = Description of sequence: Unknown Bacteria sp.
MFHLFGNILTNYGTAANNRGENEGNTTTLQKLIWKGEVHSTVSSEAIRWALRYYWQNAGYPVNRRWDENAQPVPDHILQDPNFDDIRFIDDDVLGFMQAEAAKVEAAVESEVEIQNSTQDEDKKLSQGKEKETAKRKRKEKPKGKITTRRGVLEVTRAVSTIPYAGDLTFNAMSGKKGRTSLYSTEVHATRYQYGFALTPNSLKEKSRINAVLDGLISIGEVAGNHARFLYDFSPDSIILRWTHDFSPRLLYCFEEDELRNISTPDLVRRVEAGDIDPKELWIGGAISSTLEDLGANVFPGVKTTVEALKQVITQDLQLSLKSS

SEQ ID NO: 322 - protein, 360 aa, Geitlerinema sp.
MMKRAANPKIQLNLGDPSMTLLHRAGVAGLWMTLKKLEKIYPTPAQRIGNLTWLLTPRCISLDWEGQDFTVLDWLLKQSFQISHEGLISLTGLDSQNMDIQNQLIIHQGITATFTQHNKFFKSAGQQSKQLIIDGIQLRVDYKKAASYAHQHFAKHLCDEYGQFLEEPIGIRGWLYPGAVVRHYAFKEQTAFEEKAEYALALLFAPVACQYFVLRSHTEPSHTNYVLLVPEVIDLELYAEYLWCLSNLDYKHFHVSSLGDAGLKFLTYETTQMPTSNFLKRCQIISFSTKAWSERQKTRAEIATIELNSSIGYFYKLSCICFPECQVILYKNQHCLLPSLIKGIIANNLAMGWPWWANWI

SEQ ID NO: 323 - protein, 226 aa, unidentified
  source 1..226: note = Cyanobacteria phylum
MIVDSNLLTQLPSESSSKPKELLMSQFPYMDLTFSLIGETLPFDHGYELFSAIAHFEPKLHTLDTLGIHTIAGIPKDGVINLTQNSRLRIRIPVNQVRLVYPLAGKSLRIGKHTIRLGIPDICLLQPAEKLRSRIVVIRGHEEPETFLVVAQRQLEQLGIQAIVSIPTKANGKPIRRTIKIKRFTVVGFGLEVTKLSDEDSLTLQIHGVGGKQKMGCGLFMPIKER

SEQ ID NO: 324 - protein, 316 aa, Nostoc sp.
MLSFFPTLYPDELLYSTLARYHIRSGNKSFKQTDLELFGYSSQQVCKITLTNNLSYLVKNLPLRSQQKVENLLQNHTLYPFYATFLMPQEAWLLKNSMSKKLSGSILDVAKVATNASGDSKKFLKFCPNCLEKDTQKYGEPYWHRIHQVPGILICPTHSVLLQDSCVEVESKGIHYHAPSFDNCLVATKVKSFTDNALKILFTLANDINGLIHSNFSFKGLAWLRSQYQRYLFDKGLLQTFSGGKFKFDEYGFAQRIFEFYGQESLEAINPRLAQHQGKYFSHCLFGCDVNPAIDSITHILMIKFLANSIEEFFQS

SEQ ID NO: 325 - protein, 360 aa, unidentified
  source 1..360: note = Cyanobacteria phylum
MTGYLSRNLLKPEYASRSRQIYNAIGNGGGKNLETYVNLPTSASGLTLIGPSGIGKTTNFQNILNLYPQIIFHPDYSVYQIVWLRVDCPKTLKGLCIDTFAGIDKLLGTNLLKKFNPARTSEDYLLAQLAQTAHTHHLGVLVIDEIQNLATAKRNRDEILNFLVKLDNTIGIPVIRVGTNEAISFLQGNLRNARRNTGEGSRMWDRMVNNDEWCYFLEGIWEYQWTKTPVHFSDEIKEVFYEESQGIIDIAIKLYKMVQWRAISISGQEIITIELIRQVAKEGLYLVKPMLDAIRSGDREWMIKYKDIAPLDITEYHNKCLSEIDAKDLTEIRRLARKQQNQQKISPKLNHVILELLNLE

SEQ ID NO: 326 - protein, 480 aa, Bacillus akibai
MINDLFVNDLIKWIDESGNNFIERVLWIDEGYVIAFVYDINAKTGFPKPKKVSEIIEAISDGCALKLKSDPWARIVRDEDLSEKEKELRDRAWQIISFIVTQEPSIYSRNYRGALVQQVIEKYNAGRTEGELVPITVYKYLRRFWQRGKNKNALLPDYANSGGKGKPKASGDKKRGRPRKYAQIPEIGIGRNVTEEDKRIFRCAIAEFYNNSKENFLTTAYDLMVKEYYSEDFYYDENGVKKSILIPPDKRPTFTQFKYWYEVEQTDIRKTIISRKGSRKYALENRAITGTSQMETIGPGSRYQIDATIADVYLVSLYNPNWIIGRPVIYVVIDVFSRMITGVYVGLEGPSWTGAMMALANAATDKVKFCQEYDVEISEDEWPCRHIPDSILGDRGELAGMKVETLIPNLDVRIENAAPYRADWKGLVERQFRTIHGYVKPFVPGYIDTDFRQRGGHDYRLDGRLNIDEFTKIVIYLILQ

SEQ ID NO: 327 - protein, 280 aa, Desulfovibrio zosterae
MQASTGVFMARSKQDWTQAKFERYCKEGRGQGSGKDYKPWIKIQDFPSKGRVSRPPGWKANREHHLFSDNEKRLFYVFEWSDAIVDIREQFPLLDLDLAISIADEMGINYPKDPQSNTPYVLTTDFMLSVKQGKNIVQKARTFKLTKDLASKSVAEKFELEKRYFAAKGIDWGIITEKEVPKQLAENVEWIHTAYKLEENADINLEELHNIANILKFRLQESDASINKITTALDRDMNIESGTSLYLFRHLIARKEIIMDMLVNKISSCPSSKEIQKIVF

SEQ ID NO: 328 - protein, 360 aa, synthetic construct
  REGION 1..360: note = Description of sequence: Unknown origin
MCGIVGYIGTQAATEILLSGLEKLEYRGYDSAGIATVWEGEVNCIRAKGKLHNLRSKLEQLANPAQIGIGHTRWATHGKPEEHNAHPHMDTAMRIAVVQNGIIENYRELREELKSKGHQFRSETDTEVIPHLIAEYFQNLPSPPLPTSPSPFLEAVRQAANHLEGAFAIAVIAADYPDELIVVRQQAPLVIGFGQGEFFCASDTPAIVAYTRAVLPLENGEIARLTPLGVEIYNFAGDRLKKQPRMLNLNPTMVEKQGFKHFMLKEIYEQPGVVRASLEAYFDHDKVNLGLPEDFYSDLEQIHIVACGTSWHAALVGKYLLEQLAGISTQVHYASEYRYAPSPLTPNTLIIGVTQSGETA

SEQ ID NO: 329 - protein, 360 aa, synthetic construct; identical to SEQ ID NO: 217
  REGION 1..360: note = Description of sequence: Unknown origin

SEQ ID NO: 330 - protein, 360 aa, Microcoleus sp.; identical to SEQ ID NO: 227

SEQ ID NO: 331 - protein, 210 aa, unidentified; identical to SEQ ID NO: 226
  source 1..210: note = Cyanobacteria phylum

SEQ ID NO: 332 - protein, 324 aa, synthetic construct; identical to SEQ ID NO: 225
  REGION 1..324: note = Description of sequence: Unknown Bacteria sp.

SEQ ID NO: 333 - protein, 360 aa, Geitlerinema sp.; identical to SEQ ID NO: 224

SEQ ID NO: 334 - protein, 227 aa, unidentified; identical to SEQ ID NO: 223
  source 1..227: note = Cyanobacteria phylum

SEQ ID NO: 335 - protein, 316 aa, Nostoc sp.; identical to SEQ ID NO: 222

SEQ ID NO: 336 - protein, 360 aa, unidentified; identical to SEQ ID NO: 221
  source 1..360: note = Cyanobacteria phylum

SEQ ID NO: 337 - protein, 480 aa, Bacillus akibai; identical to SEQ ID NO: 219

SEQ ID NO: 338 - protein, 272 aa, Desulfovibrio zosterae; identical to SEQ ID NO: 218

SEQ ID NO: 339 - protein, 360 aa, unidentified
  source 1..360: note = Cyanobacteria phylum
MKGYLARNLLKPEYASRSRQIYHAMEDGGGKNLEKYVNLPTSASGLTLIGPSGMGKSTNFQNILNLYDQVIFHPDYSVYQIVWLKVDCPKTLKGLCTDIFTEIDKLLGSKLFNKFSSSRSSEDYLLAQLAQIAHTHHLGVLVIDEIQNLVTTKRSRDEILNFLVKLDNIIGIPVIRIGTNEAISILQSNFRNARRSTGEGSVIWDRMLNNDEWCFFMEGMWEYQWTKTPVPFSEQLKEIFYYESQGIIDIAVKLYKMVQWRAISIGGGEVITIDLIRQAAKDGLYFVKPMLDAIRSGDKEWMIKYKDIAPLDTTEYQNKCLSGLDYNDLKEIQRLARKQQNQQKVSPKLNWIIARLLELE

SEQ ID NO: 340 - protein, 315 aa, Nostoc sp.
MLSFFPALYPDELLYSTLARYHIRSGNKSFKQTDLELFGYSSQQVCKITLTNNLNYLVINLPLGSQQTVDNLLQNHTLYPFYATFLMPQEAWLLKNSMSKKLSGSILDIVKVATNFSGDSKKFLKFCPECLEKDTQKYGEPYWHRIHQVPGILVCPTHSVVLHDSSVGVESKGIYYHAASMDNCLLPSKVKIFTNKTLQTLFILANEINWLIHSNFTFRGLAWLRAQYQYYLVHKGLLQTFPGGKFKFDEFGFTQLIFEFYGQEFLEALNPKLTQHQGKYFSYCLFGCDLNPAIDRITHILMIKVLANSIEVFFQ

SEQ ID NO: 341 - protein, 233 aa, unidentified
  source 1..233: note = Cyanobacteria phylum
MIVDCSLPTQITSESPLKLTESRELLTSQLLYIDLIFNLIGQTVPLDHGYELFSAIAHFEPKLHKLDTVGIHTIAGIPKEGVIQLTKNSRLRIRLPVEQVGLVYPLAGKQLRIGKHTIRLGIPEIYLLEPAERLRSRIVIIQGYQEPESFLAAAQRQLEQLGIQAKAFITKNTDGSPKRRSIKIKRFTVVGFGLEIINLSNEDSLTLLRHGIGGKRKMGCGVFLPVKRKENAT

SEQ ID NO: 342 - protein, 360 aa, Geitlerinema sp.
MSVLASVVNPKIQLNLGDPSITWIHRAGIAGLWMTLKQLEKLYPIAAERPGNLTWLLAPRSISLEWQGQDFIVLDWLLKQSFQINEDGLISLTGLNRHSINIETQINIHLGITATFLQHNQVFKFDGEKSWPLIVDGMEIDVEYKKVVSYAHQHFAKQLCDKQGQLLEQPIGVAGWLYPGSVVRHVAFTKETKFEEKPELAFALLYAPVSCHYFVLRSPTQLQHPQYVLVIPEVTNLEVYAQQYWSLGNLDYKYSHVSSLGDAALRFLTYKTVAIPNQVKRCQVILFGTVAWSKQQKTRTEIAVVKVTEIIDIVYKLSCISFSEYKIINYKNKNFIISNMLKGIIADNLIKGFLWWNDLY

SEQ ID NO: 343 - protein, 324 aa, synthetic construct
  REGION 1..324: note = Description of sequence: Unknown Bacteria sp.
MFHLFGNILTNYGTAANNRGENQGNITTLQKLIWKGEVHTTISSEAIRWALRYYWQNVGYPVNRRWDENTQPIADHLWQDLNFDAIRFIDDDVLGFMQAEGAKVEASDEPEGGTEEPSQDENKKSSKGKNKQTSQSKGKQKPKGKITARRGVLEVTRAVSIIPFTGDITFNAVSGVKGRNSLYATEIHATRYQYGFALTPDSLKDKSRIYPVLDGLISIGEVAGNHARFLYDFSPESIVLRWTHDFSPRFLYCFEQDDLGNLSVSNLVRQIEVGDIDPKELWIGGLISQGLEDLGANVFPGVKSAVKALKQVIAEDLQLSSGSN

SEQ ID NO: 344 - protein, 171 aa, unidentified
  source 1..171: note = Cyanobacteria phylum
MLLSLIGETDRYKHCGTQLAIALLSKPEKSIVIRTFHRFKTKDIHDPKNAKPDYQELLTNIEFVVWVKTDKAKPTLAERLQQAFTNPTSINRFGGLCLGESRDLVNSVTLLPENYHAELLWWLVTDEDGWLTLPYWVDHVGSRRTRWQCYELQESQVWQPPDLSWTTIQTS

SEQ ID NO: 345 - protein, 360 aa, Microcoleus sp.
MLGFFPAPYPDEILYSILARYHIRSGNTSPKITLRELFNSQDVIATVDLPSNLNSLIENLKFISNYQVEDLIYKYTLYPFYSVFLPPKRATQVMESMKADFGGDIHSRAGIMASSLKMQRYLRFCPKCLEEDLQKYGESYWHRLHQTPGVLVCPVHCVTLQDSTIPIQGFNRHEYHAASQENCPVVSVQQIYSQQSLKKLRVLAQDILWLINSNLLSRELEWFQRKYIALLIEKGFATATRRVNQKRLLDNFLFFYGAEILEAVNSMVNYKNEQNWLFSIFRKHRKSFHPIRHLLVIRFLTNSIEEFFNTDYQYKPFGEGPWLCFNAAVDHYLKPVITNLVVTHCLDNKKPLGTFSCFCG

SEQ ID NO: 346 - protein, 360 aa, synthetic construct
  REGION 1..360: note = Description of sequence: Unknown origin
MCGIVGYIGTQAATDILLAGLEKLEYRGYDSAGIATVWEGEVNCVRAKGKLHNLRSKLEQIETPAQIGIGHTRWATHGKPEEYNAHPHMDNAMRVAVVQNGIIENYRELREELKAKGHVFRSETDTEVIPHLIAELLKNFSPSSPSSPLLEAIRQAVNHLQGAFALAVVCADYPDELIVVRQQAPLVIGFGQGEFFCASDTPAIVAYTRAVLPLENGEIARLTPLGVEIYNFAGDRLKKQPRLLNLNPTMVEKQGFKHFMLKEIHEQPGVVRASLEAYFNNVEENVRSPINLGLPEDLYTDLEQIHIVACGTSWHAALIGKYLIEQLAGISTQVHYASEYRYAPSPLTSNTLIIGVTQSG

SEQ ID NO: 347 - protein, 272 aa, Desulfovibrio zosterae
MVRGRREWTQAKYERYIKEGRGRGSGKDYKPWLTIQDFPSKGRSSRTPGWKTNRVHHFFSDHEKRLFYLFEWSDIVTDIREQFPLDNLDLAMSIATDIGIKYPIDTQSGTPHVLTSDFMLTVNHNGKRTQIARTVKQSTELEKKRVNEKLEVERRYYLAIGIDWGIITEKEISRLLAENVEWIHSAYRLEANSEMDTVQLRELSKILKSKLQEEDATINKITIALDEEMNINYGTSLYVFKHLIARKELIVDLVKIKLSACPSAKVIEKIIF

SEQ ID NO: 348 - protein, 360 aa, Arcobacter sp.
MAMSSIYDVRLITLSIAIAVLASYTALDLAGRVTAAKASAQVAWLIGGAMVMGIGIWSMHFVAMLAFCLPIPITYDMLTVLVSMLPAAIASGGALFLASRPVLSIWQLLVGGTLMGIGIASMHYIGMLAMRIQATATYNPVLFILSIAIAIGASILGLNIAFQLRTQTNTTRRWAKVSGALFIGGAIAGMHYTGMGAVRFTPTNPTAISGLEKTNDSLTLLGVSIGIATLVILGFALLTSFIDRRLSAQTKLLVQQEAQAKLSQLFTEITLQIRRSLKLDDVINTTVHEVRLALKTDRVVMYRFNSDWSGTIIAESIAQGWTKTLGKTVNDPFRENYIEMYKNGRVRATNDIYEAGFTEC

SEQ ID NO: 349 - protein, 480 aa, Bacillus akibai
MAQDLFVNDLIEWMLDSKTHVIERIVWIDEGYIIAFVIDINAKKGLPEAKKISDICDAISEGIAFKQQRDPWARIIRDENLTDREKEYRDKAWEIISSLVTEEPLIYYRNFRGSLVKQVVEKYNLEKNKDKLVEKTVYGYLRRFWQRGKNINALIPDFINSSGKGKIRGQSSKKRGRPKKYAHDPDIGEGVNVTEEDRMIFRIAISKFYNNPKQNYLTTAYDLMVKNYYKEGIRYDENGVQKSILTPTDKIPTITQFKYWYQLEQDDVTKTLTSRKGAKRVALENRAILGTSKMETIGPGSRYQIDATVADVYLVSKYNRNWIIGRPVIYVIIDVFSRIIAGVYVGLEGPSWTGAMMALANVATDKTLFCKEYNINITEDEWPCNQMPDAILGDRGELAGMTVETLIPNLYVRIENAASYRADWKGLVERHFRIIHEYVKPFLPGYVDTDFRQRGARDYRLDGKLDIDQFTEIIVHLIVH

SEQ ID NO: 350 - protein, 360 aa, synthetic construct; identical to SEQ ID NO: 217
  REGION 1..360: note = Description of sequence: Unknown origin

SEQ ID NO: 351 - protein, 272 aa, Desulfovibrio zosterae; identical to SEQ ID NO: 218

SEQ ID NO: 352 - protein, 480 aa, Bacillus akibai; identical to SEQ ID NO: 219

SEQ ID NO: 353 - protein, 458 aa, Bacillus akibai; identical to SEQ ID NO: 220

SEQ ID NO: 354 - protein, 360 aa, unidentified; identical to SEQ ID NO: 221
  source 1..360: note = Cyanobacteria phylum

SEQ ID NO: 355 - protein, 316 aa, Nostoc sp.; identical to SEQ ID NO: 222

SEQ ID NO: 356 - protein, 227 aa, unidentified; identical to SEQ ID NO: 223
  source 1..227: note = Cyanobacteria phylum

SEQ ID NO: 357 - protein, 360 aa, Geitlerinema sp.; identical to SEQ ID NO: 224

SEQ ID NO: 358 - protein, 324 aa, synthetic construct; identical to SEQ ID NO: 225
  REGION 1..324: note = Description of sequence: Unknown Bacteria sp.

SEQ ID NO: 359 - protein, 210 aa, unidentified; identical to SEQ ID NO: 226
  source 1..210: note = Cyanobacteria phylum

SEQ ID NO: 360 - protein, 360 aa, Microcoleus sp.; identical to SEQ ID NO: 227

SEQ ID NO: 361 - protein, 227 aa, unidentified; identical to SEQ ID NO: 223
  source 1..227: note = Cyanobacteria phylum

SEQ ID NO: 362 - protein, 360 aa, Geitlerinema sp.; identical to SEQ ID NO: 224

SEQ ID NO: 363 - protein, 324 aa, synthetic construct; identical to SEQ ID NO: 225
  REGION 1..324: note = Description of sequence: Unknown Bacteria sp.

SEQ ID NO: 364 - protein, 210 aa, unidentified; identical to SEQ ID NO: 226
  source 1..210: note = Cyanobacteria phylum

SEQ ID NO: 365 - protein, 360 aa, Microcoleus sp.; identical to SEQ ID NO: 227

SEQ ID NO: 366 - protein, 360 aa, synthetic construct; identical to SEQ ID NO: 217
  REGION 1..360: note = Description of sequence: Unknown origin

SEQ ID NO: 367 - protein, 272 aa, Desulfovibrio zosterae; identical to SEQ ID NO: 218

SEQ ID NO: 368 - protein, 480 aa, Bacillus akibai; identical to SEQ ID NO: 219

SEQ ID NO: 369 - protein, 360 aa, unidentified; identical to SEQ ID NO: 221
  source 1..360: note = Cyanobacteria phylum

SEQ ID NO: 370 - protein, 316 aa, Nostoc sp.; identical to SEQ ID NO: 222

SEQ ID NO: 371 - protein, 227 aa, unidentified; identical to SEQ ID NO: 223
  source 1..227: note = Cyanobacteria phylum

SEQ ID NO: 372 - protein, 360 aa, Geitlerinema sp.; identical to SEQ ID NO: 224

SEQ ID NO: 373 - protein, 324 aa, synthetic construct; identical to SEQ ID NO: 225
  REGION 1..324: note = Description of sequence: Unknown Bacteria sp.

SEQ ID NO: 374 - protein, 210 aa, unidentified; identical to SEQ ID NO: 226
  source 1..210: note = Cyanobacteria phylum

SEQ ID NO: 375 - protein, 360 aa, Microcoleus sp.; identical to SEQ ID NO: 227

SEQ ID NO: 376 - protein, 360 aa, synthetic construct; identical to SEQ ID NO: 217
  REGION 1..360: note = Description of sequence: Unknown origin

SEQ ID NO: 377 - protein, 272 aa, Desulfovibrio zosterae; identical to SEQ ID NO: 218

SEQ ID NO: 378 - protein, 480 aa, Bacillus akibai; identical to SEQ ID NO: 219

SEQ ID NO: 379 - protein, 360 aa, unidentified; identical to SEQ ID NO: 221
  source 1..360: note = Cyanobacteria phylum

SEQ ID NO: 380 - protein, 316 aa, Nostoc sp.; identical to SEQ ID NO: 222

SEQ ID NO: 381 - protein, 227 aa, unidentified; identical to SEQ ID NO: 223
  source 1..227: note = Cyanobacteria phylum

SEQ ID NO: 382 - protein, 360 aa, Geitlerinema sp.; identical to SEQ ID NO: 224

SEQ ID NO: 383 - protein, 324 aa, synthetic construct; identical to SEQ ID NO: 225
  REGION 1..324: note = Description of sequence: Unknown Bacteria sp.

SEQ ID NO: 384 - protein, 210 aa, unidentified; identical to SEQ ID NO: 226
  source 1..210: note = Cyanobacteria phylum

SEQ ID NO: 385 - protein, 360 aa, Microcoleus sp.; identical to SEQ ID NO: 227

SEQ ID NO: 386 - protein, 360 aa, synthetic construct; identical to SEQ ID NO: 217
  REGION 1..360: note = Description of sequence: Unknown origin

SEQ ID NO: 387 - protein, 272 aa, Desulfovibrio zosterae; identical to SEQ ID NO: 218

SEQ ID NO: 388 - protein, 480 aa, Bacillus akibai; identical to SEQ ID NO: 219

SEQ ID NO: 389 - protein, 360 aa, unidentified; identical to SEQ ID NO: 221
  source 1..360: note = Cyanobacteria phylum

SEQ ID NO: 390 - protein, 316 aa, Nostoc sp.; identical to SEQ ID NO: 222

SEQ ID NO: 391 - protein, 360 aa, Microcoleus sp.
MVSFFPKPYPDEILYSVIARYHIRSGNTSPKITLQELFNSQTTVATVDLPSNLNNLIQNLQFISNYQVEDLIYKHTLYPLYSPFLPANRSSQVLESMKGDYGGDIHTRAGIMASSITMPKYFRFCPTCWEEDLKNYGEPYWHRIHQIPGVLVCPFHDEVLQDSLAPLHGFNKHEYYAASTDKCSITKEPSIFSPDTLKKLLVLAKDFWWLLNSEIPCQEPQWFRKQYTNLMIEQGLATATGRIYQKKLLDNFLFFYGREMLSAVNSIVTHNDSSNWVTSIVRKHRKSFHPLRHLLMMRFLKTSVEEFFKQDNKYKPFGEPPWLCLNAAAEHYLQPVITNLVITLCCDTKKPVGTFSCTCG

SEQ ID NO: 392 - protein, 210 aa, unidentified
  source 1..210: note = Cyanobacteria phylum
MTTIVVKVEVPIACFRQSHAREYAETYPVPPPSTVYGMLLSLVGEVDRYKHCGVKLAIALLSKPEKSTVIRTCHRFKTKNIHESKNNKPDYQDLLTNIEFVVWVDAGADKAKPNLAQRLEEAFTNPASINRFGGLCLGESRDLVNDVSLLPENYYAESMRWLIRDDYGLLTLPYWVDHVGSRGTRWLCYEIQEFQVFQPPELSWTSIQSD

SEQ ID NO: 393 - protein, 324 aa, synthetic construct
  REGION 1..324: note = Description of sequence: Unknown Bacteria sp.
MFHLFGNILTTYGTAANNRGENEGNTTTLQKLIWKGEVHSTVSSEAIRWALRYHWQNAGYPVNRRWDENAQPVADHIWEDPNFDDIRFIDDDVLGFMQAEAAKVEAADESEVEIQNSTQDENQTTSQGEEKKTSQRKRKQRPKGRISARRGVLEVTRAVSTIPYAGDITFNSMSGKKGRTSLYATEVHATRYQYGFALTPNRLKDKSRIHAVLDGLISIGDVAGNHARFFYDFSPDSIILRWTHDFSPRLLYCFEEDELRNISTPDLVRRVEAGDIDPKELWIGGAIASTLENLGANLFPGVKATVEALKQVIIEDLQLSLRSS

SEQ ID NO: 394 - protein, 360 aa, Geitlerinema sp.
MKTAANPKIQLNLGDPSMTLLHRAGVAGLWMTLKKLEKIYPTAAQRIGNFTWLLTPSNISLYWEGQDFTVLDWLLKHSFQISDEGLISLTGLDSQNMDIQTQIIIHQGITATFIQHNKFFKSAGQQSKQLIIDGIQLRVDYKKAASYAHQHFAKHLCDQYGQLLQEPIGIRGWLYPGAVVRHYAFKEQTAFEEKAEYALALLFAPVACQYFVLRSHTEPSHTNYVLVVPEVIDLELYAQYLWYLSKLDYKHFHVSSLGDAGLKFITYEAKQLDNFNFIKRCQVISFSTKAWSERQKTRAEIAIIELNSSVSYLYKLSCICFPNCQVVIYKNQHFLLPSLMKGIIANNLAMGWPWWANWIS

SEQ ID NO: 395 - protein, 227 aa, unidentified
  source 1..227: note = Cyanobacteria phylum
MIVDGNLLRQIPSESPPKPRELLMSQLPYMDLTFNLIGETLPFDHGYELFSAIAHFEPKLHTLDTLGIHTIAGIPKDGVINLTQNSRLRVRIPVNQVHLVYPLAGKSLRIGKHTIRLGIPDICLLQPAEQLRSRIVVIRGHEEPETFLVVAQRQLEQLGIQATASIPTKANGKPIRKTIKIKRFTVVGFGLEVTNLSNEDSLTLQIHGVGGKQKMGCGLFMPIQERQ

SEQ ID NO: 396 - protein, 315 aa, Nostoc sp.
MLSFFPTLYPDELLYSTLARYHIRSGNKSFKQTDLELFGYSSQQVCKITLTNNLNYLVKNLPLRSQQKVENLLQNHTLYPFYATFLMPQEAWLLKNSISKKLSGSILDVAKVATNSIGDSKKFLKFCPECLEKDTQKYGEPYWHRIHQVPGILVCPTHGIVLQDSCVKVESKGVHYHAASVDNCLVACKVKNLTNNTVKTLFTLVNDINWLIHSNFAFQGLAWLRAQYQRYLVDKGLLQTFPGGKFKFDEYGFAQLIFEFYGQESLEAINPKLTQHQGKYFSHCLFGCDLSPAIDRITHILLIKFLANSIEEFFQ

SEQ ID NO: 397 - protein, 459 aa, Bacillus akibai
MTGYLARNILKPEYASRSRQIYNAIKDGGGKNLENYVNLPTSASGLTLIGPSGMGKSTNFQNILNLYTQVILHPEYSVYQIVWLKVDCPHAGSLKGLCTDIFLAVDRLLGTNYFKKFGSSRNSEDYMLAQVAQIAHTHHLGVLVIDEMQNLVTARRRSSDMLNFLVKMDNTVGIPVIRVGTNEAFPILQGNFRNARRGTGEGSVIWDRMVNDDEWYFFMEGMWEYQWTKTPVPFSDEINEVFYDESQGIIDIAVKLYKMVQWRAISIGGKEIITIELIRQAAKDGLYLVKPMLDAIRSGDKEWMIKYKDIAPLDTTEYQNKCLSELDSKDLKEIRRLARKQQNQQKVSPKLNYVILELLNLEVEPAKAKECAEQVIVSSDEDAEIPSLVKKAYALALQGGQVNESLTKTNTKKAKLKPNYQENDIRRIVENANKEKKSAYEDLKLAGIIKNPVEDFINI

SEQ ID NO: 398 - protein, 360 aa, unidentified
  source 1..360: note = Cyanobacteria phylum
MTGYLARNILKPEYASRSRQIYNAIKDGGGKNLENYVNLPTSASGLTLIGPSGMGKSTNFQNILNLYTQVILHPEYSVYQIVWLKVDCPHAGSLKGLCTDIFLAVDRLLGTNYFKKFGSSRNSEDYMLAQVAQIAHTHHLGVLVIDEMQNLVTARRRSSDMLNFLVKMDNTVGIPVIRVGTNEAFPILQGNFRNARRGTGEGSVIWDRMVNDDEWYFFMEGMWEYQWTKTPVPFSDEINEVFYDESQGIIDIAVKLYKMVQWRAISIGGKEIITIELIRQAAKDGLYLVKPMLDAIRSGDKEWMIKYKDIAPLDTTEYQNKCLSELDSKDLKEIRRLARKQQNQQKVSPKLNYVILELLN

SEQ ID NO: 399 - protein, 480 aa, Bacillus akibai
MINDLFVNDLIEWIDESGNNFIERVLWIDEGYIIAFVYDINAKTGFPEAKKVSEIREAISDGRALKLKSDPWARIVRDEDLSEKEKELRDRAWQIISFIVTQEPSIYYRDYRGTLVQQVIEKYNAGRTEGELVPITVYKYLRRFWQRGKNKNALLPDYANSGGKGKPKSSGDKKRGRPRKYAQVPEIGVGRNVTEEDKRIFRFATAKFYNNSKENFLTTAYDLMIKEYYAEDFYYDDNGVKKSILIPPDKRPTFTQFKYWYEVEQTDIRKTMISRKGSRKYALENRAITGTSQMETIGPGSRYQIDATIADVYLVSLYNRNWIIGRPVIYVVIDVFSRMITGVYIGLEGPSWTGAMMALANAATEKVKFCREYGIEISEDEWPCKHIPDTILGDRGELAGMTVETLIPNLNVRIENTAPYRADWKGLVERQFRIIHGYVKPFVPGYIDTDFRQRGGHDYRLDGRLNIDEFTKIVIFLILQ

SEQ ID NO: 400 - protein, 280 aa, Desulfovibrio zosterae
MQASTGVFMARSKQDWTQAKFERYCKEGRGQGSGKNYQPWIKIQDFPSKGRVSRPPGWKTNREHHLFSDNEKRLFYVFEWSDAIVDIREQFPLLDLDLAINIADEMGINYPKDPQSNTPYVLATDFMLSVQQGKNIVQKARTFKLTKDLGSKSVAEKFELEKRYFAAKGIDWGIITEKEVPKQLAENVEWIHTAYKLEENADINLEELRNIANILKFRLQESDASINKITTVLDKEMNIESGTSLYLFRHLIARKEIIMDMLVNKISSCPSSKEIQKIIF

SEQ ID NO: 401 - protein, 360 aa, synthetic construct
  REGION 1..360: note = Description of sequence: Unknown origin
MCGIVGYIGTQTATEILLAGLEKLEYRGYDSAGIATVWEGEVNCVRAKGKLYNLRSKLEQSATPAQIGIGHTRWATHGKPEEHNAHPHLDTAMRVAVVQNGIIENYRELREELKQKGHQFRSQTDTEVIPHLIAEYLQHLPSPSHPLTPSDFLEAVRQAANHLEGAFAIAVISADYPDELVVVRQQAPLVIGFGQGEFFCASDTPAIVAYTRAVLPLENGEIARLTPLGVEIYNFAGDRLKRQPRMLNLNPTMVEKQGFKHFMLKEIYEQPGVVRASLEAYFDHDTVNLDLPQDFYADLEQIHIVACGTSWHAALVGKHLLEQLAGISTQVHYASEYRYAPSPLTANTLIIGVTQSGETA

SEQ ID NO: 402 - protein, 360 aa, unidentified; identical to SEQ ID NO: 339
  source 1..360: note = Cyanobacteria phylum

SEQ ID NO: 403 - protein, 315 aa, Nostoc sp.; identical to SEQ ID NO: 340

SEQ ID NO: 404 - protein, 233 aa, unidentified; identical to SEQ ID NO: 341
  source 1..233: note = Cyanobacteria phylum

SEQ ID NO: 405 - protein, 360 aa, Geitlerinema sp.; identical to SEQ ID NO: 342

SEQ ID NO: 406 - protein, 324 aa, synthetic construct; identical to SEQ ID NO: 343
  REGION 1..324: note = Description of sequence: Unknown Bacteria sp.

SEQ ID NO: 407 - protein, 171 aa, unidentified; identical to SEQ ID NO: 344
  source 1..171: note = Cyanobacteria phylum

SEQ ID NO: 408 - protein, 360 aa, Microcoleus sp.; identical to SEQ ID NO: 345

SEQ ID NO: 409 - protein, 360 aa, synthetic construct; identical to SEQ ID NO: 346
  REGION 1..360: note = Description of sequence: Unknown origin

SEQ ID NO: 410 - protein, 272 aa, Desulfovibrio zosterae; identical to SEQ ID NO: 347

SEQ ID NO: 411 - protein, 480 aa, Bacillus akibai; identical to SEQ ID NO: 349

SEQ ID NO: 412 - protein, 315 aa, Nostoc sp.
MLSFFPILYPDELLYSTLARYHIRSGNKNFKQTDLELFGYSSQQVCRITLTNNLNYLVKNLSLLSPPTVDNLLQNHTLYPFYTTFLKTQEAWLLKDSMSKKLSGSILDVAKVATNSSGDSKRFLKFCPECLENDIQKYGEPYWHRIHQVPGILVCPTHSVVLHDSCVEVESKGVHYHAASVDNCLVTTNIKKFTNQTLETLLTLANDINWLIHSNFAFQGLAWLRTQYQHYLVNKGLLQSFPGGKFKFDEYGFAQLIFEFYGQESLEAVNTKLTHHQGKYFSHCLFGCDLEPAIDRVAHILMMKFLANSIEEFLQ

SEQ ID NO: 413 - protein, 459 aa, Bacillus akibai
MTGYLARNILKPEYASRSRQIYSAIKDGGGKNLENYVNLPTSASGLTLIGPSGMGKSTNFQNILNLYPQVILHPEYSVYQIVWLKVDCPHAGSLKGLCTDIFLAVDRLLGTNYFKKFGSSRNSEDYMLAQVAQIAHTHHLGVLVIDEMQNLATARRKSSDMLNFLVKMDNTIGIPVIRVGTNEAFPILQGNFRNARRGTGEGSVIWDRMINDDEWYFFMDGMWEYQWTKTLVPFSDEINEVFYDESQGIIDIAVKLYKIVQWRAISIGGKENITIDLIRQAAKDGLYLVKPMLDAIRSGDKEWMTKYKDIAPLDTTEYQNKCLSEIDAKDLTDIRRLARKQQNQPKVSPKLNYVILELLNLEVEPVKAKECVEQVIASSDEDADISSLLKKAYALALQGGQINESLAKTNIKKAKLKPKYQENDIRRIVENANKDKKSAYEGLKLASIIKNPVENFINT

SEQ ID NO: 414 - protein, 360 aa, unidentified
  source 1..360: note = Cyanobacteria phylum
MTGYLARNILKPEYASRSRQIYSAIKDGGGKNLENYVNLPTSASGLTLIGPSGMGKSTNFQNILNLYPQVILHPEYSVYQIVWLKVDCPHAGSLKGLCTDIFLAVDRLLGTNYFKKFGSSRNSEDYMLAQVAQIAHTHHLGVLVIDEMQNLATARRKSSDMLNFLVKMDNTIGIPVIRVGTNEAFPILQGNFRNARRGTGEGSVIWDRMINDDEWYFFMDGMWEYQWTKTLVPFSDEINEVFYDESQGIIDIAVKLYKIVQWRAISIGGKENITIDLIRQAAKDGLYLVKPMLDAIRSGDKEWMTKYKDIAPLDTTEYQNKCLSEIDAKDLTDIRRLARKQQNQPKVSPKLNYVILELLN

SEQ ID NO: 415 - protein, 272 aa, Desulfovibrio zosterae
MVRGTREWTQAKFERYKKEGRGRGSGKDYKPWLTIQDFSSKGRSSRTPGWKTNRVHHFFSDHEKRLFYLFEWSDTVTDIREQFPLSDLDLAMSIATDMGVKYPIDTQSGTPYVLTSDFMLTVNHNGKQVQIARTVKQSTELEKKRVNEKLEIERQYYLAKGIDWGIITEKEILRRLAENVEWVHSAYRLEANSEMDVAQLRELSTILKSKLQEEDTTINRITTALDKEMNINLGTSLYVFKHLIAKKEIIVDLLEIKLSACPSTQVIKKIIF

SEQ ID NO: 416 - protein, 360 aa, synthetic construct
  REGION 1..360: note = Description of sequence: Unknown origin
MCGIVGYIGTQAATEILLAGLEKLEYRGYDSAGIATVLEGDIHCVRAKGKLYNLREKLQREVNPATIGIGHTRWATHGKPEEYNAHPHTDTAKRIAVVQNGIIENYRELREELKAKGHEFRSETDTEVIPHLIAELMAQQAQQAAATAQPFPFLEAVRQAVTKTGTDTELIPQLIAELMAQQVHNSASMPQQFPFLEAVRQAVTKAGTDTEAIPQLMAEVMTQQVHNSAVTSQHSSFLEAVRQAVNKLEGAFAIAVICADYPDELIVARQQAPLTIGFGQGEFFCASDTPALVPHTRAVLTLENGELARMTPIGVEVYNFAGDRLKKSPRTLSWNPVQVEKQGFKHFMLKEIYEQPGVVR

SEQ ID NO: 417 - protein, 182 aa, unidentified
  source 1..182: note = Cyanobacteria phylum
MPPPSTVYGMLLSLVGEMNRYKHCGVKLAIALLSEPEKSTVIRTFHRFKTKNIHDPKNTKPDYQELLTNIEFIAWVDAGVDKAKPNLVQRLEEAFTNPASINRFGGLCLGESRDLVNDVTLLPENYYAESMRWLIRDEYSSLTLPYWVDHVGSRGTRWLRYGMQESQVFQPPELAWTSVQSD

SEQ ID NO: 418 - protein, 316 aa, synthetic construct
  REGION 1..316: note = Description of sequence: Unknown Bacteria sp.
MFHLFGNILTSYGTAANNRGENEGNTTTLQKLVWKGEVHSTVSSEAIRWALRYFWQNAGYPVNRSWDENAQPVATHIWQNPSFDDIRFIDDDVLGFMQAEAAKVEAASESEAQVQQSSEDDNKKSSQRKRKEKPKGRITARRGVLEVTRAVSTIPYAGDLTFNAMSGKKGRTSLYATEVHATRYQYGFALTPNSLKDKSRINAVLDGLISIGEVAGNHARFLYDFSPDSIILRWTHDFSPRLLYCFEEDELRNVSVPDLVRRVEVGDIDPKELWIGGAISRTLEDLGANVFPGVKATAEALKQVITQDLQLSPRSS

SEQ ID NO: 419 - protein, 360 aa, Geitlerinema sp.
MVLAESKKWVVEYSCQLKKGHSIANARKQMKRAANPKIQLNLGDPSITLLHRAGMAGLWMTLKKLKQIYPTPAQRIGRLTWQLTPCSISLDWEGQDFTVLDWLLKQSFQISDEGLISLTGLNPEKMDIQTKIIIHQGITETFTQHNKFFKSAGQEFKQLTIDGVELRVDYKKAAWYAHQHFAKYLCDQYGQLLQELVGITGWLYPGAVVRHYAFQKETKFEEKTEHALALLFAPVACQYFAIRAHTNPQDTKYVLVVPEVIDLELCAQYFWSLNNLNYKDFHVSSLGDAGLKFLTYETTQQATSNFIKRCQAISFSTVPWSKQQKTRTEIVFIELNSSVSSLYKLSCICFSKYQLVFYKN

SEQ ID NO: 420 - protein, 227 aa, unidentified
  source 1..227: note = Cyanobacteria phylum
MILDSNLLTQIHSESPPKPRELLMSQLPYMDLTFNLIGETLPFDHGYELFSAIAHFEPKLHQLDTLGIHTIAGIPKDGVINLTQNSRLRIRIPVNQVRLVYPLAGKSLRIGKHTIRLGIPDIYLLQPAKQLRSRIVVIRGYEEPETFLAVAQRQLEQLGIQAIASILTKANGKPARRTIKIKRFTVVGFGLKVSNLSDEDSLALQIHGVGGKQKMGCGIFVPIKERP

SEQ ID NO: 421 - protein, 360 aa, synthetic construct
  REGION 1..360: note = Description of sequence: Unknown origin
MCGIVGYIGTQAATEILLAGLEKLEYRGYDSAGIATVWEGEVNCVRAKGKLHNLRSKLEQVETPAQIGIGHTRWATHGKPEEYNAHPHMDTAMRIAVVQNGIVENYRELREQLKQKGHQFRSETDTEVIPHLIAEFLKNPPSVPQSFSPSDLLEAIRQAVNHLEGAFAIAVISADYPDELIVVRQQAPLVIGFGQGEFFCASDTPAIVSYTRAVLPLENGEIARLTPLGVEIYNFAGDRLKKQPRMLNLNPTMVEKQGFKHFMLKEIHEQPGVVRASLEAYFNNLGDSNQSPINLGLPESLYADLEQIHIVACGTSWHAALVGKHLIEQLAGISTQVHYASEYRYAPSPLTANTLIVGVT

SEQ ID NO: 422 - protein, 272 aa, Desulfovibrio zosterae
MARTKQEWTQAKFERYMKEGRGQGHRKNYQPWIKIQDFPSKGRVSRPPGWKTNREHHLFSDHEKRLFYLFEWSDNIVDIREQFPLLDLDLAINIATEMGIEYPKDVNNDTPYVLTTDFMLSVKQGNQIVDKARTFKLVKDLGIKSVAEKFELEKRYYAAKGIDWGIITEKEIPMFLVKNVEWIHSSYKLEAAAEINVDDLHNIANILRSKLQESDSSINKITNCLDKEMNLEVGTSLYIFKHLLANKKIIMDMQTSAIYSSISALEIQKIIF

SEQ ID NO: 423 - protein, 501 aa, Effusibacillus lacus
MNNNLFVNDLIEWIDESGNNFVERILWIDEAYIIAFTIDINIKTGFPVSKKVSDIQEAINEDRALKLRTDPWARIVRDEDLSAKEKEIRDKYWDIISSIVIQEPSIYYRDKRGLLIKQVIEKYNNNRNEGKLVERSVYQIIRRYWQRGKDKNSLLPDYANSGGKGKIRASGEKKRGRPRKYAYEPDIGVGINITDEDKKIFRLAVTKFYHNQKENFLTTAYDLMIKNYYSEETIYDENGVKKHILISPDKRPTLTQFKYWYDVEQGDIRKKIISRKGAKKYALEHRAITGNSTMESIGPGSRYQIDATIADVYLVSAYNRNWIVGRPVIYVVIDVFSRMITGVYVGFEGPSWIGAMMALANAASDKVAFCKEYGIEITEDEWACHHIPDAILGDRGELAGMAVETLIPNLGIRIENAAPYRADWKGLVERHFGIIHGHIKPFVPGYIDKDFRERGARDYRLDSKLDIEQFTEIIIKIILYHNNENYLDNYPSSVSLREGDC

SEQ ID NO: 424 - protein, 247 aa, Effusibacillus lacus
MIADDVLPIPRELWQWGIANRSGRLRTIPEDIVKLNLMPTENATITERGIKFKGMYYTCEKAKNEFWFEKARSNSLSRLEKKLEISYDIRKPDYIYLRSPDGRNFEKCFLLESESKYFNKNLHEIEYLLAFEELQRQKNKGLKQQQKVDLMAEIESVVNKAKKMTEETKDDKLSNRKRTAGIRDNRSAEKSKRRKIEGFELEKNNNENTSNTVDTESQKIEQPQSLQPNYLDILRQKREERKREQDK

SEQ ID NO: 425 - protein, 360 aa, unidentified
  source 1..360: note = Cyanobacteria phylum
MCALLMQGYIARNISKPEYARRSRQIYEAIKARDGKNLDVYVNVPTSTSASGLTLIGPSGMGKSTNFINILNLYPQVILHPEYSTYQIVWLKVDCPHAGSLKGLCTDIFLGIDRLLGTNYFKKFGASRNSEDYMLAQVAQIAHTHHLGVLVIDEMQNLVTARRSCSDMLNFLVKMDNIIGVPVIRIGTNEAFPILQGNFRNARRGTGEGSVIWDRMRNDDEWYFFMETMWEYQWTKISTDFSDEFNDVFYDESQGIIDIAIKLYKMVQWRAISLGGKEAINANLIKQSAKDGLFLVKPMLSAIRSGDKEWMIKYKDIAPIDTTEYYNNCLSNLETKDLKEISRLAKKQQTENGHLSATQR

SEQ ID NO: 426 - protein, 315 aa, Nostoc sp.
MLSFFPTLYPDELLYSALARYHIRSGNKTFKQTDLELFGYSSQQLCKVTLTNNLNYLVKNLSLFSQLTVSDLLQNHTLYPFYASFLIPQQAWQLQDSMRKKLGGSILEVAKVANHPTDNSTKFLKFCPVCLEQDTQKYGEPYWHRIHQIPGILVCPTHRVVLHDSSVAVDSKGIHYYAASPENCLTNQNQVKYADNTVEKLLILAHDIWNTSTINIPFKGLTWLRNQYQFYLINQKFMTLLPGGNFRFNEKSFSNSICDFYGQEFLEIINPNLIKNPDKYFSGYLLACDLNPVIDRITHILIIKFLVNSVERFFT

SEQ ID NO: 427 - protein, 222 aa, unidentified
  source 1..222: note = Cyanobacteria phylum
MGDNFHVSTQSHATEVLNPQLIYVDLSFRLIGQTLPIDHGYALYAALTHLQDKLHSLDDLSIQTIPGIPDGNDSLHLNNHSRLRIRLPVDKIPLVYPFAGKSLTIGRHKIHLEIPQMYLLQAVEKLRSRIVVIKGYEEPKGFLAAAQRQLEKLGVQGIASIPTKADGKPERRSIKIKKFTVVGFGLEVTNLNDEDSITLQKYGIGGKQKMGCGVFVPVNEKR

SEQ ID NO: 428 - protein, 360 aa, Geitlerinema sp.
MINTTSNSKIQLNLSNSSTTLLHRAGIAGFWMTLKQLEQIYPNPAQRTGNLTWVLTPHSISLNWEGQDFAALDWLLKQSFQISDEGLISLTGLNPQASHIERKIIIHQGITGTFLQHNKFFKFSDKKLKSIIVDGRKFVTEYKSAMSYAHQHFAKKLCDESGQLLQESIGIVGWLYPGAVVRHSAFPKQTKFEEKPEYALALLFVPVACHYFILRSHTKPQSTNYILVVPEVNNLELYAQHCWNLSNLSYRDFHVSSLRDAGLKFLTNEAAIQIKQNCVKRCQVFSFGTLIWSEQQKVRTETATLELTEKMSFYYKLSRSCFSNYRIIDYENQHFILTSLVQGIIANNLGLGLPWWNDFG

SEQ ID NO: 429 - protein, 332 aa, synthetic construct
  REGION 1..332: note = Description of sequence: Unknown Bacteria sp.
MSHLFGNILTIYGTAANNRGENEGNITTLQKINWKGEVHTTVSAEAIRWALRYYWQNNGYEVNRRWDENAQPLADHIWQDTNFNSVRFIDDDVLGFMQAEAARVEAGDQSGNQVQEASAGEIQNVSQDEEPTSSKDKGKKTSQRKGRERPQGRVTAKRGVLEVTRAVSLIPYTGNITFNAVSGTKGRTSLYGTEVHATRYQYGFALTPNSLQDKSRINAVLNGLISIGEVGGNHARFLYDFSPDSIILRWTHDFSPRFLYCFEQDEWGNISVPDLVRRVEAEDIDPRELWIGGAIVRDLGNLGANVFPGVKKVVEELLKVITRDLQLPPRSS

SEQ ID NO: 430 - protein, 173 aa, unidentified
  source 1..173: note = Cyanobacteria phylum
MLLSLVGEENRYTHCGVKLAIALITEPAKSTVIRTFHRLKTKNIHDPKNNKPDYQELLTNIEFVVWVNAGVDKAKPNLVQRLEEAFTNPASINRFGGLCLGESRDLVNDITLLPKNYYAESMRWLIKDEHGLLTLPYWIDHVGSRGTRWLRYELQECQVLQPPELSWTSVQSN

SEQ ID NO: 431 - protein, 360 aa, Microcoleus sp.; identical to SEQ ID NO: 237

SEQ ID NO: 432 - protein, 208 aa, unidentified
  source 1..208: note = Cyanobacteria phylum
MTTVALKVEVPIACFRQSRAREYAETYPVPPPSTVYGMLLSLIGETDRYKHCGTQLAIALLSKPEKSIVIRTFRRFKTKDIHDPKNAKPDYQELLTNIEFVVWVKTDKAKPTLAELLQQAFANPASINRFGGLCLGESRDLVNTVTLLPEKYHSESLWWLVTDEDGSLTLPYWVDHVGSKRTRWQCYELQESQVWQPPDLSWTTIQTS

SEQ ID NO: 433 - protein, 324 aa, synthetic construct
  REGION 1..324: note = Description of sequence: Unknown Bacteria sp.
MFHLFGNILTSYGTAANNRGENEGNITPLQKLIWKGEVHTTISSEAIRWALRYYWQNVGYPVNRRWNENAQPVADHVWQDPNFDDIRFIDDDVLGFMQTEAAKVEASDESEGESEGASQNENKKSSKGKDKQTSPRKGKQKPKGKITARRGVLEVTRAVSMIPFMGDITFNAVSGVKRQNSLYATEIHATRYQYGFALTPDSLKDKSRIHAVLDSLISIGEVAGNHARFLYDFSPESIVLRWTHDFSPRFLYCFEQDELGNLSVSNLVRQVEVGDIDPKELWIGGLISQGLEDLGANVFPGVKSAVKALKQVIAEDLQLSPGSD

SEQ ID NO: 434 - protein, 224 aa, Geitlerinema sp.
MLFGTVIWSKQQRTRIEIAVVEATEIIDFIYKLSRLCFPERKIIKYKNKNLIISDVFTGAIADNLVKGFPWWANLYKIFKNKSLLKLITNDGVYKMIQNSEWNLESQKLFIKACHEALKKIYAKIYGRTKEGQYAQIERENIRILSQLGRCTNAENFRKFIAEFWGRAGQLYILEKHWEELLPLTSGIMDWKVARDLTFIALASYPKSNMVEKQILEISDSNSE

SEQ ID NO: 435 - protein, 143 aa, synthetic construct
  REGION 1..143: note = Description of sequence: Unknown Bacteria sp.
MGVSVDKNSFSLIYPLAGKSLKIGKHTIRLGIPEIYLLEPAERLRSRIVIIQGYQKPESFLAAAQRQLEQLGIQGKAFITKNTDGSPKRRSIKIKRFTVVGFGLEIINLSNEDSLTLLRYGIGGKRKMGCGVFVPVKRKGNAS

SEQ ID NO: 436 - protein, 231 aa, unidentified
  source 1..231: note = Cyanobacteria phylum
MQGYIARNILKPEYTRRSRQIYDAIKDGGGKTLENYVYVPTSASGLTLIGPSGMGKSTNLLNILSLYPQVILHPEYTTFQIVWLKVDCPRSLKGLCTDIFLAVDRLLGTSYFKKFGSRGNSEDYMLAQVAQIAHTHHLGVLVIDEMQNLVNARRNRDDLLNFLVKMDNTIGVPVIRVGTNEAFPILQGNFRNARRGTGQGSVIWERMLQDDDWDFFLTGIWEYQWTKTPSV

SEQ ID NO: 437 - protein, 480 aa, Bacillus akibai
MHENLFINDLIEWESDGNTQLIERIIWIDEGYIIAFVLDVNSKKGLPEPKKVSEILEAMSEGLAFKHKQDPWARIIREENLTDRDKEYRDKAWKIISSLVVQEPSIYYRNIRGSLVRQVIKSYNEGREKDKLVERTVYGYMRRFWQRGKTKNALIPDFINSSGKGKIRGDSNKKRGRPKKYSYDPEIGEGVNVTEEDRRIFRIAISRFYDNPKENYFTTAYELMVKEFYKEGIRYDENGVRKSILTPTEKIPTITQFKYWYQIEQYDVTKTLTSRIGAKKFALENRAILGTSKMETIGPGSRYQIDATVGDVYLVSKYNRNWIIGRPVIYVIIDVFSRMITGFYVGLEGPSWIGAMMALTNVAADKVMLCQEYNIDIAEEQWSCNQMPDAILGDRGELAGMAVEPLIPNLYVRIENAASYRADWKGLVERHFRTIHEYVKPFLPGYVDTDFRQRGARDYRLDSKLDIDQFAEIIIRLIIF

SEQ ID NO: 438 - protein, 281 aa, Desulfovibrio zosterae
MRSLSGVIFMVRGRGEWTQAKFERYRKEGRGRGSGKDYKPWITIQDFPSKGRSSRTPGWKSNRVHHFLSDQEKRLFYLFEWSDVVTDTREQFPLDDLDLAMSIATEMGIKYPVDLQSGTPYVLTTDFMLTVNQNGKQVQIARTVKQSTELEKKRVIEKLELERRYYLAKSIDWGIVTENEIPRLLAENVEWIHSAYKLEANSEMDIAQLHELATILKSRLQEHDTAINRVTTALDKEMNIKSGTSLYIFKHLIAAKKIIIDMLETKISACPSSKVIEKIIF

SEQ ID NO: 439 - protein, 360 aa, synthetic construct
  REGION 1..360: note = Description of sequence: Unknown origin
MCGIVGYIGTQAATDILLAGLEKLEYRGYDSAGIATVWEGEVNCVRAKGKLHNLRSKLEQIETPAQIGIGHTRWATHGKPEEYNAHPHLDTAKRVAVVQNGIIENYRDLREELKGKGHQFVSETDTEVIPHLIAEFLKHIPPSSTSSPFLEAIRQAVNHLHGAFAIAVISADYPDELIVVRQQAPLVIGFGQGEFFCASDTPAIVAYTRAVLPLENGEIARLTPLGVEIYNFAGDRLKKQPRLLNFNPAMVEKQGFKHFMLKEIHEQPGVVRASLDAYFNPHESTESPINLGLPADFYTDLEQIQIVACGTSWHAALIGKYLIEQLAEISTQVHYASEYRYAPSPVTANTLIIGVTQSGE

SEQ ID NO: 440 - protein, 224 aa, synthetic construct
  REGION 1..224: note = Description of sequence: tnsB gene
MVSQLQQRNDYSFADYSNSCWFIDFALLDINIADTNGSLIGRPYLTTAIDIYSQCCMGLFLSLKSPNHQAITQALRHAILPKSYDSEYNLRHEWITYGIPAGLQLNNSSLTNSYLLQDKLSQLGISIYSPKSGSRLNDSFTEKLFSEINRYLSSDALSNQISTDNNSFLTLGVLEKILVLYFVDNFNQQIHNDSSHQTRQERWESGLGSASKMIEENFIHTLLT

SEQ ID NO: 441 - protein, 581 aa, synthetic construct
  REGION 1..581: note = Description of sequence: tnsB gene
MYQQLQDSYPANDDGAVELQKHQNSTKTSSKLPSEKLITDDVKLRMEVIQSLTEPCDRKTYSEKKKEAAEKLGVTIRQVERLLKKWREEGLVGLATTRADKGKYRLEQEWVDFIINTYTNGNKKGKQMTRHQVFLKVKGEAKEKGLKKGEYPSHQSIYRILDKHIEGKERKDNARSPGYSGEKLTHMTRDGRELEVEGSNDVWQCDHTRLDVMLVDEYGVLDRPWLTIVIDSYSRCVMGFYLGFDHPSSQIDALALHHAILPKSYSSEYTLRHEWVAYGKPNYFYTDGGKDFTSIHTTEQVAVQIGFSCALRRRPSDGGIVERFFKTLNEQVLNTLPGYTGSNVQQRPENVDKNACLTLKNLEMVLVRYIVDEYNQHTDARMKDQSRIGRWEAGSMVEPYLYNELDLAICLMKQERRKVQKYGCIQFENLTYRADHLRGRDGETVALRYDPADVTTLLVYEINADGTEEFLDYAHAQSLETEHLSLRELKAINKRLKEASEEINNDSILEAMLDRQAFVEQTVKQNRKQRRQAASEQVNPVEPVAKKFAVPEPKEVETDSEPDMELPNYEVRYMDEFFEED

SEQ ID NO: 442 - protein, 275 aa, synthetic construct
  REGION 1..275: note = Description of sequence: tnsC gene
MTDAKPLDFIQEPTREIQAHIERLSRAPYLELNQVKSCHTWMYELVISRMTGLLVGESRSGKTVTCKAFRNNYNNLRQGQEQRIKPVVYIQISKNCGSRELFVKILKALNKPSNGTIADLRERTLDSLEIHQVEMLIIDEANHLKIETFSDVRHIYDEDSLKISVLLVGTTSRLLAVVKRDEQVVNRFLEKFEIDKLEENQFKQMIQVWERDVLRLPEESKLASGESFKLLKQSTNKLIGRLDMILRKAAIRSLLRGYKKVDQGVLKEIITATKF

SEQ ID NO: 443 - protein, 173 aa, synthetic construct
  REGION 1..173: note = Description of sequence: tniQ gene
MRESINENKQFWLIRVEPLEGESISHFLGRFRREKGNKFSAPSGLGDVAGLGAVLARWEKFYFNPFPTHQELEALASVVQVDVDRLRQMLPPLGVSMKHSPIRLCGACYAESPCHKIEWQFKKTVGCDRHQLRLLSKCPVCEKPFPVPALWVDGICNRCFTPFAEMAQYQKHY

SEQ ID NO: 444 - protein, 643 aa, synthetic construct
  REGION 1..643: note = Description of sequence: cas12k gene
MSVITIQCRLIASEATRSYLWQLMAQKNTPLINELIEQLGIHPEIEQWLKKGKLPDGVVKPLCDSLITQESFANQPKRFNKSAIEVVEYIYKSWLALQKERQQTIDRKEHWLKMLKSDVELEQESKCTLDAIRSQATKILPKYLAQSEQNNNQTQSQNKKKSKKSKTKNENSTLFDILFKAYDKAKNPLNRCTLAYLLKNNCQVSQKDEDPNQYALRRSKKEKEIERLKKQLQSRKPNGRDLTGREWQQTLIMATSSVPESNDEANIWQKRLLKKDISLPFPIRFRTNEDLIWSKNEEGRICVSFSGEGLNDHIFEIYCGNRQIHWFQRFLEDQNIKNDNNDQHSSALFTLRSAILAWQENKQHKENSLPWNTRRLTLYCTLDTRLWTTDGTEKVKQEKVDEFTQQLANMEQKENLNQNQQNYVKRLQSTLNKLNNAYPRHNHDLYQGKPSILVGVSLGLEKPATLAIVDSSTNIVLAYRSIKQLLGDNYKLLNRQRQQQQRNSHERHKAQKSNMPNKLSESDLGKYIDNLLAQAIIALAKNYQAGSIVLPTMKNVRESIQSEIEARAVKRCPNYKEGQQQYAKQYRQSIHRWSYNRLMQFIQSQAVKANISIEQGPQPIRGSSQEKARDLAIAAYYLRQNKS

SEQ ID NO: 445 - protein, 197 aa, synthetic construct
  REGION 1..197: note = Description of sequence: tnsB gene
MGEQTRFQRWESGLIVAPDAISERDLDICLMKQTRRMIQRGGYLQFENLMYRGEYLAGYAGESVVLRYDPKDITTLLVYYQKGHKEEFLARAYAQDLETEQLSLDEAKAMSRKIRQAGKMISNRSMLAEVRDRETFVTQKKTKKERQKAEQAVVQKAKKPVPVEPEEEIEVTSFDSEPEYQMPEVFDYEQMREDYGW

SEQ ID NO: 446 - protein, 278 aa, synthetic construct
  REGION 1..278: note = Description of sequence: tnsC gene
MTSKQAQAVAQQLGDIPVNDEKIQAEIQRLNRKSFIPLEQVQMLHDWLDAKRQARQSGRVLGESRTGKTMGCDAYRLRHKPKQESGKPPNVPVAYIQIPQECGAKELFGVIIEHLKYQMTKGTVAEIRDRTMRVLKGCGVEMLIIDEADRFKPKTFAEVRDIFDKLEIAVILVGTDRLDAVIKRDEQVYNRFRACHRFGKFSGEDFKRTVEIWEKKVLQLPVASNLSGKTMLKTLGEATGGYIGLLDMILRESAIRALKKGLQKVDLETLKEVAAEYK

SEQ ID NO: 447 - protein, 167 aa, synthetic construct
  REGION 1..167: note = Description of sequence: tniQ gene
MEVAEIQPWLFQIAPLEGESISHFLGRFRRANDLTPTGLGKAAGIGGAIARWEKFRFNPPPTPQQLEALAIVVGVDTDRLKQMLLPLGVGMKMEPIRLCAACYAKSPCHKIEWQLKVTQGCGRHKLTLLSECPNCGSRFKMPALWVDGWCQRCFLNFFEMTKYQKAI

SEQ ID NO: 448 - protein, 648 aa, synthetic construct
  REGION 1..648: note = Description of sequence: cas12k gene
MVLLDICVSKRMSQITIQCRLVASEFTRQQLWKLMAEKNTPLINELLLRVAQNPEFESWRQKGKHPSGIVKELCQPLKSHPCFIGQPGRFYTSAIAIVNYIYKSWFALMKRSQSQLEGKIRWWEMLKSDTELVEASGVTLESLRNKAAEILAELTPQSDTVKAQPAKGNKRKKTKKAKVAEGDHSISKTLFDTYRDTEDILTRCAISYLLKNGCKINNKEEDAEKFAKRRRKLEIQIERLRAKLKARIPKGRDLTDAKWLETLLLATDNVPESEEEAKSWQDSLLKKSSKVPFPVAYETNEDMTWFKNEHERICVKFNGLGEHTFQVYCDSRHLHWFQRFLEDQQIKQKSKNQHSSSLFTLRSGRIAWQEGDGKGDPWKVNRLILYCSVDTRLWTAEGTNLVRVEKAEEIAKTITQTKAKGELNVQQLAHIKRKNSSLARINNPFPRPSKPLYKGQSHILVAVSLGLEKPATVAVVDGSTSTVITYRSIKQLLGDNYKLLNQQRQQKHSLSHQRQIAQMLAAPNQMGESQLGQYVDRLLAREIVAIAQTYKAGSIVLPKLGDMREQVQSEIQAKAEQKSDLIEVQQKYAKQYRVSVYQWSYGQLLANIHSSAAKAGIVIEESKQPIRGSPQEKAKELAIAAYHSRQKS

SEQ ID NO: 449 - protein, 424 aa, synthetic construct
  REGION 1..424: note = Description of sequence: tnsB gene
MTVYRILQPLIDKVEKAKSIRSPGWRGSRLSIKTRDGNDLQVEHSNQVWQCDHTLVDVLLVDRHGKLLSRPWLTTVIDSYSRCIMGINLGYDAPSSKVVALALRHAILPKQYGSDYGLNEEWGTSGLPQHFYTDGGKDFRSNHLQQIGVQLGFVRHLRDRPSEGGSVERPFKTLNTELFSTLPGYTGSNIQQRPEEAENEACLTLHQLEKILVRYIVDNYNQRIDARMGDQTRFQRWESGLIAAPDLLSERELDICLMKQTRRHIQRGGYLQFENLMYRGENLAGYAGEKVVLRYDPRDITTIMIYCTEGDKEVFLTRAYAQDLETEELSLDEAKASSRKVREAGQAVNNRSILAEVREREVFPTQKKTKKERQKLEQTELKKSKQPIPIEPEELDEAVSTEVETEPEMPEVFDYEQMREDYGW

SEQ ID NO: 450 - protein, 282 aa, synthetic construct
  REGION 1..282: note = Description of sequence: tnsC gene
MSSKASTTEAQAIAQQLGDIPANNEKLQAEIQRLNRKGFVPLEQVKTLHDWLEGKRQSRQSGRVVGESRTGKTMGCDAYRLRNKPKQESGKPPTVPVAYIQIPQECGAKELFGVIMEHLKYQVTKGTVAEIRDRTLRVLKGCGVEMLIIDEADRFKPKTFAEVRDIFDKLEIAVILVGTDRLDAVIKRDEQVYNRFRSCHRFGKMSGEDFKRTVEIWEKQILKLPVASNLGSKTMLKTLGEATGGYIGLLDMILRESAIRALKKGLQKIDLDTLKEVTAEYR

SEQ ID NO: 451 - protein, 168 aa, synthetic construct
  REGION 1..168: note = Description of sequence: tniQ gene
MMEAEEIKPWLFQVEPLEGESISHFLGRFRLANDLTPSGLGQAAGLGGAIARWEKFRFNPPPSRQQLESLAVVVGVDTDRLEKMLPPAGVGMKMEPIRLCGACYGESPCHKIEWQFKETGGCGRHNLTLLSECPNCGARFKVPALWVDGWCHRCFTPFAEMVEHQKRI

SEQ ID NO: 452 - protein, 639 aa, synthetic construct
  REGION 1..639: note = Description of sequence: cas12k gene
MSQITIMCRLVANESTRQQLWQLMAELNTPLINELLVLISQHQDFETWQQKGKIPAGTVKQLCESLKTDPGFAGQPARFYASAIATVSYVYKAWMKVQKRLKSQLESKARWLSMLQSDEELIAIAGVDLDGLRNQARLILQQFAPEPSPQEDLQANKKQPKTEKSLSQTLLDTYESSEDILTRCAISYLLKNGCKVYEKPENSQKFSKHKDKLKVQIQRLIQKLEGRVPQGRNLTDTEWLETLILATEKIPQDEAEAKSWQDSLLKKSSSVIFPVSYESNEDMTWFRNQKGRICIKFNGISEHTFEIYCDSRQLHWFERFLSDQETKKNSKNQHSSALFTLRSARIGWHEQEIKNKQICRRKPTVNPWDIYHLTLYCTVDTRLWTAEGTAVVAAEKAEEIAKIITKTKEKDDLNEKQLAHIKRKNSTLERINNPYPRPSKPLYQANPHILVGVSLGLKKPATIAVVDVISQKVLTYCSIKQLLGKNYKLLNRHRQLKHNFAHKRKIAQTQAKQQQYLDSELGQYIDRLLAKQIIAIAKQYSASSIVLPQLNGMREQINSEIQAKAKEKCPESIEAQKKYAKQYRRSINQWSYGRLIESIISQGLQAGIAIEESKQAVQGSPQEKAKELAFVAYNSRKKS

SEQ ID NO: 453 - protein, 216 aa, synthetic construct
  REGION 1..216: note = Description of sequence: tnsB gene
MVSQLHRQNDSSLANYSNSCWFVDFTLLDINIADTTGSVIGKPYLTIVIDIYSQCCMGFCLSLKSPNNKVVTQALRHAILPKRYNSEYNLNHEWRTYGIPVGLCIDSSNFTNSYSLQEKVSQLGIRLHTHQPRFKSNYCTEKICLTIISNLQASRGLSGEDDFVTLEVIEKILVHYIVNNFNQQSSTYLSGSTRQEMWEAGLISMPQIIQEHELDI

SEQ ID NO: 454 - protein, 580 aa, synthetic construct
  REGION 1..580: note = Description of sequence: tnsB gene
MYQKLKNSCLSSEDDAVELQKHQNSTKPHKLPSEELITEQVKLRMEVIQSLIEPCDRKTYSAKKIEAAQKLGVSIRQVERLLQKWREQGLVGLTTTRSDKGKYRLEQEWIDFIINTYKEGNKGSKRMTRNQVFLKVKGRAKQFSLNKSEHPSHQSVYRILDEHIEQKERKEKARSPGYLGERLTHMTRDGRELEVEGSNDVWQCDHTRLDVRLVDEYGVLDRPWLTIVIDSYSRCLMGFYVGFDHPSSQIDTLALRHAILPKSYGSGYQLRHEWQAYGKPNYFYTDGGKDFTSIHTTEQVAVEIGFNCDLRRRPSDGGIVERFFKTLNLQVLNILPGYTGSNVQERPESTDKDACLTLKDLEIFLVRYIVDEYNPHTDARMKDQSRIGRWEAGLMADPYLYDQLDLAICLMKQERRKVQKYGCIQFENLVYRAEHLRGRDGETVAFRYDPADITTLLVYKMNADGTEDFLDYAHAQYLETERLSLRELKAINKRLKEASQEINNDSILEAMIDRQAFVEETVKRNRKQRRQAANEQVNPVEQVAKKFATPEQKEAEPESEADTELPKYEVRYMDEFFEEY

SEQ ID NO: 455 - protein, 279 aa, synthetic construct
  REGION 1..279: note = Description of sequence: tnsC gene
MTDAMSLAKQFGVIQELTPEIQAEIERLSRQPYLELDQVKRCHAWMYELVISRMTGLLLGESRCGKTVTCKAFANLYNKLRLTKGQRMKPVVYIQIPKNCGSRDFFMKILKALNKPSNGTISDLRERTLDSLVIHQVEMLIIDEANHLKFETFSDVRHIYDDDELKISVLLVGTTSRLLAIVKRDEQVVNRFLEQFELDRLEDTQFKQMIQIWERDVLRLPEESKLASGDNLKILKQATKKLIGRLDMILRKAAIRSLLRGQKKVDKDVLREVIAASKL

SEQ ID NO: 456 - protein, 173 aa, synthetic construct
  REGION 1..173: note = Description of sequence: tniQ gene
MEENIDEKRQIWLTRVEPFDGESISHFLGRFRREKGNKFSAPSGLGDVAGLGVVLARWEKFYFNPFPTHQELDALATVVELDAERLRQMLPPEGVGMKHSPIRLCGACYAQSVCHKIEWQFKKTVGCDRHQLRLLSKCPVCEKPFPIPALWMDGQCQRCFTSFAEMAEYQKPY

SEQ ID NO: 457 - protein, 641 aa, synthetic construct
  REGION 1..641: note = Description of sequence: cas12k gene
MSVITIQCRLVASEDTRRHLWQLMADKNTPLINELLEQVRIHPDMEQWLKKGKLPDGVIKPLCDSLSSQERFVNQPKRFYKSAIEVVEYIYKSWLALQKERQQKIDKKDHWLNMLKSDVELEQESNFSLNIIRAKAIEILPHYIAKFEETNKPDIKKENVNKTKKSKSKNNNYILFDILFKAYDQAKNPLNRCAIAYLLKNNCQVSEEEEDPNRYSLRRSKKVREIERLKEQLQSRIPSGRDLTGQEWLQSLLVATTTVPESENQVKIWQKYLLQNSSSIPFPVQFTSNEDLIWSKNEKGRICLSFSGKEFNNHIFEIYCDNKQIYWFQRFLEDQSIKRNNKDQHSSSLFTLRSGRLAWQDNKGNDLPWKIHRLTLYCSVDTRLWTIEGTQEFRQEKVDEITQKLADIEKKESLNKNQQIYVKRLNSTLTKINTAYPRPNQNLYQGKTSILIGVSLGLEKPATIAIVDCSTNKVLAYRSVKQLLGDNYKLLNRQRQQQQRNSHERHKAQKSNTQIKLSELELGKHIDNLLAQAIIMLAKNYQAGSIVLPTMKNVRESIQSEIEARALKRCPNYKEGQQQYAKQYRQSIHRWSYNRLIECIQSQAAKANISIEQGPQPIRDSPQEKARDLAIAAYHFRQNKS

SEQ ID NO: 458 - protein, 201 aa, synthetic construct; identical to SEQ ID NO: 89
  REGION 1..201: note = Description of sequence: Cas protein